SEQUENCE LISTING (120 sequences)

SEQ ID NO: 1 - protein, 232 aa, synthetic construct
  REGION 1..232: note = Synthetic construct
EPKSCDKTHTCPPCPAPELLGGPSVFLFPPKPKDTLMISRTPEVTCVVVDVSHEDPEVKFNWYVDGVEVHTAWTQPREEQYNSTYRVVSVLTVLHQDWLNGKEYKCKVSNKALPAPIEKTISKPKGRAQTPQVYTIPPPREQMSKKKVSLTCLVTNFFSEAISVEWESNGQPENNYKTTPPVLDSDGSFFLYSKLTVDKSRWQQGNVFSCSVMHEALHNHYTQKSLSLSPGK

SEQ ID NO: 2 - protein, 483 aa, synthetic construct
  REGION 1..483: note = Synthetic construct
MGWSCIILFLVATATGVHSQVQLVETGGGLIQPGGSLRMSCGTGRAMLGTHTMEVTVYHWVRQAPGKGLEWIAYIGSGGSVYDFFVWLRFTISRDNSKNTLYLQLNSLRAEDTAVYYCARVPLDCVLYRYGSFSVTLDIVQGWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHTAWTQPREEQYNSTYRVVSVLTVLHQDWLNGKEYKCKVSNKALPAPIEKTISKPKGRAQTPQVYTIPPPREQMSKKKVSLTCLVTNFFSEAISVEWESNGQPENNYKTTPPVLDSDGSFFLYSKLTVDKSRWQQGNVFSCSVMHEALHNHYTQKSLSLSPGK

SEQ ID NO: 3 - protein, 481 aa, synthetic construct
  REGION 1..481: note = Synthetic construct
MGWSCIILFLVATATGVHSQVQLVETGGGLIQPGGSLRMSCLLMWITQCFWVRQAPGKGLEWIAYIGSGGSLLMWITQCRFTISRDNSKNTLYLQLNSLRAEDTAVYYCARPESRLLEFYLAMPFATPMEAELARRSLAQWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHTAWTQPREEQYNSTYRVVSVLTVLHQDWLNGKEYKCKVSNKALPAPIEKTISKPKGRAQTPQVYTIPPPREQMSKKKVSLTCLVTNFFSEAISVEWESNGQPENNYKTTPPVLDSDGSFFLYSKLTVDKSRWQQGNVFSCSVMHEALHNHYTQKSLSLSPGK

SEQ ID NO: 4 - protein, 418 aa, synthetic construct
  REGION 1..418: note = Synthetic construct
SDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 5 - protein, 418 aa, synthetic construct
  REGION 1..418: note = Synthetic construct
SDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGISPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 6 - protein, 669 aa, synthetic construct
  REGION 1..669: note = Synthetic construct
MDWIWRILFLVGAATGAHSSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQAEPKSCDKTHTCPPCPAPELLGGPSVFLFPPKPKDTLMISRTPEVTCVVVDVSHEDPEVKFNWYVDGVEVHTAWTQPREEQYNSTYRVVSVLTVLHQDWLNGKEYKCKVSNKALPAPIEKTISKPKGRAQTPQVYTIPPPREQMSKKKVSLTCLVTNFFSEAISVEWESNGQPENNYKTTPPVLDSDGSFFLYSKLTVDKSRWQQGNVFSCSVMHEALHNHYTQKSLSLSPGK

SEQ ID NO: 7 - protein, 669 aa, synthetic construct
  REGION 1..669: note = Synthetic construct
MDWIWRILFLVGAATGAHSSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGISPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQAEPKSCDKTHTCPPCPAPELLGGPSVFLFPPKPKDTLMISRTPEVTCVVVDVSHEDPEVKFNWYVDGVEVHTAWTQPREEQYNSTYRVVSVLTVLHQDWLNGKEYKCKVSNKALPAPIEKTISKPKGRAQTPQVYTIPPPREQMSKKKVSLTCLVTNFFSEAISVEWESNGQPENNYKTTPPVLDSDGSFFLYSKLTVDKSRWQQGNVFSCSVMHEALHNHYTQKSLSLSPGK

SEQ ID NO: 8 - protein, 223 aa, synthetic construct
  REGION 1..223: note = Synthetic construct
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNF

SEQ ID NO: 9 - protein, 223 aa, synthetic construct
  REGION 1..223: note = Synthetic construct
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNF

SEQ ID NO: 10 - protein, 283 aa, synthetic construct
  REGION 1..283: note = Synthetic construct
MGWSCIILFLVATATGVHSDVLMTQSPLSLPVTPGEPASISCWNRQLYPEWTEAQRLDWYLQKPGQSPQLLIYANCSVYDFFVWLHYYSVRDTLLGPGRPYRGVPDRFSGSGSGTDFTLKISRVEAEDTGVYYCQCTEVRADTRPWSGPYILRNQDDRELWPRKFFFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 247 aa, synthetic construct
  REGION 1..247: note = Synthetic construct
MGWSCIILFLVATATGVHSDVLMTQSPLSLPVTPGEPASISCPGVLLKEFTVSGNILTIRLTAADHRWYLQKPGQSPQLLIYKVSNRFSGVPDRFSGSGSGTDFTLKISRVEAEDTGVYYCFQGSHVPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 12 - DNA, 2454 bp, synthetic construct
  misc_feature 1..2454: note = Synthetic construct
  source 1..2454: mol_type = other DNA
gttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttaccatgggatggagctgtatcatcctcttcttggtagcaacagctaccggagtccactcccaggtgcagctggtggagactgggggaggcttaatccagcctggagggtccctgagaatgtcctgcgggacaggcagggcaatgctgggcacacacaccatggaagtgactgtctaccattgggttcggcaggctccggggaaggggctggagtggatcgcatacattggtagtggtggtagtgtttatgatttttttgtgtggctccgattcaccatttccagagacaatagcaagaacaccctgtatttgcaattgaacagtctgagggctgaggacacagccgtgtattactgtgcccgagtccccctggattgtgttctgtatcgatatggttccttttccgtcaccctggacattgtccagggttggggccaaggaaccacggtcaccgtctccagcgcttccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcatacagcctggacacagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaacccaaagggagagcccagacaccacaggtgtacaccatacccccacctcgggaacaaatgtccaagaagaaggtcagcctgacctgcctggtcaccaacttcttctctgaagccatcagtgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgatctaaagggcgatatcgcccttaagggcgaattctgcagattcgagcatgcatctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 13 - DNA, 1836 bp, synthetic construct
  misc_feature 1..1836: note = Synthetic construct
  source 1..1836: mol_type = other DNA
gttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctagcttggtaccgagctcggatccaccatgggatggagctgtatcatcctcttcttggtagcaacagctaccggagtccactccgatgtgttgatgacccaatctccactctccctgcctgtcactcctggggagccagcctcgatctcttgctggaacaggcagctgtatccagagtggacagaagcccagagacttgactggtatctgcagaaaccaggccagtctccacagctcctgatctacgccaactgcagtgtttatgatttttttgtgtggctccattattattctgttagagatacattattaggaccaggacgcccctacaggggggtcccagacagattcagtggcagtggatcagggacagatttcacactcaagatcagcagagtggaggctgaggataccggagtgtattactgccagtgcacagaggtgcgagccgacacaaggccctggagtggtccctacatcctacgaaaccaggatgaccgtgagctgtggccaagaaaattcttcttcggtggaggcaccaaggtggaaatcaagcgtacggtagcggccccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccaccagggcctgagctcgcccgtcacaaagagcttcaacaggggagagtgttgactcgagcatgcatctagagggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 14 - DNA, 2448 bp, synthetic construct
  misc_feature 1..2448: note = Synthetic construct
  source 1..2448: mol_type = other DNA
gttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttaccatgggatggagctgtatcatcctcttcttggtagcaacagctaccggagtccactcccaggtgcagctggtggagactgggggaggcttaatccagcctggagggtccctgagaatgtcctgcctgttgatgtggatcacgcagtgcttttgggttcggcaggctccggggaaggggctggagtggatcgcatacattggtagtggtggttccctgttgatgtggatcacgcagtgccgattcaccatttccagagacaatagcaagaacaccctgtatttgcaattgaacagtctgagggctgaggacacagccgtgtattactgtgcccgaccggagagccgcctgcttgagttctacctcgccatgcctttcgcgacacccatggaagcagagctggcccgcaggagcctggcccagtggggccaaggaaccacggtcaccgtctccagcgcttccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcatacagcctggacacagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaacccaaagggagagcccagacaccacaggtgtacaccatacccccacctcgggaacaaatgtccaagaagaaggtcagcctgacctgcctggtcaccaacttcttctctgaagccatcagtgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgatctaaagggcgatatcgcccttaagggcgaattctgcagattcgagcatgcatctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 15 - DNA, 1728 bp, synthetic construct
  misc_feature 1..1728: note = Synthetic construct
  source 1..1728: mol_type = other DNA
gttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctagcttggtaccgagctcggatccaccatgggatggagctgtatcatcctcttcttggtagcaacagctaccggagtccactccgatgtgttgatgacccaatctccactctccctgcctgtcactcctggggagccagcctcgatctcttgcccaggggtgcttctgaaggagttcactgtgtccggcaacatactgactatccgactgactgctgcagaccaccgctggtatctgcagaaaccaggccagtctccacagctcctgatctacaaagtttccaaccgattttctggggtcccagacagattcagtggcagtggatcagggacagatttcacactcaagatcagcagagtggaggctgaggataccggagtgtattactgctttcaaggttcacatgttccgtggacgttcggtggaggcaccaaggtggaaatcaagcgtacggtagcggccccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccaccagggcctgagctcgcccgtcacaaagagcttcaacaggggagagtgttgactcgagcatgcatctagagggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 16 - DNA, 3018 bp, synthetic construct
  misc_feature 1..3018: note = Synthetic construct
  source 1..3018: mol_type = other DNA
gttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttgccaccatggactggatttggagaatcctcttcctggtgggagctgccacaggcgcacacagcagcgacaacggcccccagaaccagagaaacgcccccagaatcaccttcggcggccccagcgacagcaccggcagcaaccagaacggcgagagaagcggcgccagaagcaagcagagaagaccccagggcctgcccaacaacaccgccagctggttcaccgccctgacccagcacggcaaggaggacctgaagttccccagaggccagggcgtgcccatcaacaccaacagcagccccgacgaccagatcggctactacagaagagccaccagaagaatcagaggcggcgacggcaagatgaaggacctgagccccagatggtacttctactacctgggcaccggccccgaggccggcctgccctacggcgccaacaaggacggcatcatctgggtggccaccgagggcgccctgaacacccccaaggaccacatcggcaccagaaaccccgccaacaacgccgccatcgtgctccagctgccccagggcaccaccctgcccaagggcttctacgccgagggcagcagaggcggcagccaggcctcaagccgttccagcagcagatcccgcaacagctcccgcaacagcacccctggaagctcaaggggcacctctcctgcgcggatggctggtaacggcggggacgctgccctggccctgctgctgctggacagactgaaccagctggagagcaagatgagcggcaagggccagcagcagcagggccagactgtgaccaaaaaaagcgccgccgaggccagcaagaagcccagacagaagagaaccgccaccaaggcctacaacgtgacccaggccttcggcagaagaggccccgagcagacccagggcaacttcggcgaccaggagctgatcagacagggcaccgactacaagcactggccccagatcgcccagttcgcccccagcgccagcgccttcttcggcatgagcagaatcggcatggaggtgacccccagcggcacctggctgacctacaccggcgccatcaagctggacgacaaggaccccaacttcaaggatcaggtgatcctgctgaacaagcacatcgacgcctacaagaccttcccccccaccgagcccaagaaggacaagaagaagaaggccgacgagacccaggccctgccccagagacagaagaagcagcagacagtgacattgctgcccgccgccgacctggacgacttcagcaagcagctccagcagagcatgagcagcgccgacagcacccaggccgagcccaaatcgtgcgataaaacgcatacgtgccccccctgtccagccccagagttgctgggtggaccgagcgtgttcctgttcccccccaagcccaaggacaccctgatgatcagcagaacccccgaggtgacctgcgtggtggtggacgtgagccacgaggaccccgaggtgaagttcaactggtacgtggacggcgtggaggtgcacacagcctggacccagcccagagaggagcagtacaacagcacctacagagttgtgtccgtgctgaccgtgctgcaccaggactggctgaacggcaaggagtacaagtgcaaggtgagcaacaaggccctgcccgcccccatcgagaagaccatcagcaagcccaagggcagagcccagacaccccaggtgtacaccatacccccccctagagaacaaatgtccaagaagaaggtgagcctgacctgcctggtgaccaacttcttctctgaagccatcagtgtggagtgggagagcaacggccagcccgagaacaactacaagaccaccccccccgtgctggacagcgacggcagcttcttcctctacagcaagctgaccgtggacaagagcagatggcagcagggcaacgtgttctcttgcagcgtgatgcacgaggccctgcacaaccactacacccagaagagcctgagcctgagccccggcaagtgatgatctaaagggcgaattcgcccttaagggctaattctgcagattcgagcatgcatctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 17 - DNA, 1719 bp, synthetic construct
  misc_feature 1..1719: note = Synthetic construct
  source 1..1719: mol_type = other DNA
gttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctagcttggtaccgagctcggatccgccaccatggactggatttggagaatcctcttcctggtgggagctgccacaggcgcacacagcagagtgcagcccaccgagagcatcgtgagattccccaacatcaccaacctgtgccccttcggcgaggtgttcaacgccaccagattcgccagcgtgtacgcctggaacaggaagagaatcagcaactgcgtggccgactacagcgtgctctacaacagcgccagcttcagcaccttcaagtgctacggcgtgagccccaccaagctgaacgacctgtgcttcaccaacgtgtacgccgacagcttcgtgatcagaggcgacgaggtgagacagatcgcccccggccagaccggcaagatcgccgactacaactacaagctgcccgacgacttcaccggctgcgtgatcgcgtggaacagcaataacctggacagcaaggtgggcggcaactacaactacctctacagactgttcagaaagagcaacctgaagcccttcgagagagacatcagcaccgagatctaccaggccggcagcaccccctgcaacggcgtggagggcttcaactgctacttccccctccagagctacggcttccagcccaccaacggcgtgggctaccagccctacagagtggtggtcctgagcttcgagctgctgcacgcccccgccaccgtgtgcggccccaagaagagcaccaatctggtgaagaacaagtgcgtgaacttctgatgactcgagcatgcatctagagggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 18 - DNA, 3018 bp, synthetic construct
  misc_feature 1..3018: note = Synthetic construct
  source 1..3018: mol_type = other DNA
gttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttgccaccatggactggatttggagaatcctcttcctggtgggagctgccacaggcgcacacagcagcgacaacggcccccagaaccagagaaacgcccccagaatcaccttcggcggccccagcgacagcaccggcagcaaccagaacggcgagagaagcggcgccagaagcaagcagagaagaccccagggcctgcccaacaacaccgccagctggttcaccgccctgacccagcacggcaaggaggacctgaagttccccagaggccagggcgtgcccatcaacaccaacagcagccccgacgaccagatcggctactacagaagagccaccagaagaatcagaggcggcgacggcaagatgaaggacctgagccccagatggtacttctactacctgggcaccggccccgaggccggcctgccctacggcgccaacaaggacggcatcatctgggtggccaccgagggcgccctgaacacccccaaggaccacatcggcaccagaaaccccgccaacaacgccgccatcgtgctccagctgccccagggcaccaccctgcccaagggcttctacgccgagggcagcagaggcggcagccaggcctcaagccgttccagcagcagatcccgcaacagctcccgcaacagcacccctggaagctcaaggggcatctctcctgcgcggatggctggtaacggcggggacgctgccctggccctgctgctgctggacagactgaaccagctggagagcaagatgagcggcaagggccagcagcagcagggccagactgtgaccaaaaaaagcgccgccgaggccagcaagaagcccagacagaagagaaccgccaccaaggcctacaacgtgacccaggccttcggcagaagaggccccgagcagacccagggcaacttcggcgaccaggagctgatcagacagggcaccgactacaagcactggccccagatcgcccagttcgcccccagcgccagcgccttcttcggcatgagcagaatcggcatggaggtgacccccagcggcacctggctgacctacaccggcgccatcaagctggacgacaaggaccccaacttcaaggatcaggtgatcctgctgaacaagcacatcgacgcctacaagaccttcccccccaccgagcccaagaaggacaagaagaagaaggccgacgagacccaggccctgccccagagacagaagaagcagcagacagtgacattgctgcccgccgccgacctggacgacttcagcaagcagctccagcagagcatgagcagcgccgacagcacccaggccgagcccaaatcgtgcgataaaacgcatacgtgccccccctgtccagccccagagttgctgggtggaccgagcgtgttcctgttcccccccaagcccaaggacaccctgatgatcagcagaacccccgaggtgacctgcgtggtggtggacgtgagccacgaggaccccgaggtgaagttcaactggtacgtggacggcgtggaggtgcacacagcctggacccagcccagagaggagcagtacaacagcacctacagagttgtgtccgtgctgaccgtgctgcaccaggactggctgaacggcaaggagtacaagtgcaaggtgagcaacaaggccctgcccgcccccatcgagaagaccatcagcaagcccaagggcagagcccagacaccccaggtgtacaccatacccccccctagagaacaaatgtccaagaagaaggtgagcctgacctgcctggtgaccaacttcttctctgaagccatcagtgtggagtgggagagcaacggccagcccgagaacaactacaagaccaccccccccgtgctggacagcgacggcagcttcttcctctacagcaagctgaccgtggacaagagcagatggcagcagggcaacgtgttctcttgcagcgtgatgcacgaggccctgcacaaccactacacccagaagagcctgagcctgagccccggcaagtgatgatctaaagggcgaattcgcccttaagggctaattctgcagattcgagcatgcatctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 19 - DNA, 1719 bp, synthetic construct
  misc_feature 1..1719: note = Synthetic construct
  source 1..1719: mol_type = other DNA
gttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctagcttggtaccgagctcggatccgccaccatggactggatttggagaatcctcttcctggtgggagctgccacaggcgcacacagcagagtgcagcccaccgagagcatcgtgagattccccaacatcaccaacctgtgccccttcggcgaggtgttcaacgccaccagattcgccagcgtgtacgcctggaacaggaagagaatcagcaactgcgtggccgactacagcgtgctctacaacagcgccagcttcagcaccttcaagtgctacggcgtgagccccaccaagctgaacgacctgtgcttcaccaacgtgtacgccgacagcttcgtgatcagaggcgacgaggtgagacagatcgcccccggccagaccggcaatatcgccgactacaactacaagctgcccgacgacttcaccggctgcgtgatcgcgtggaacagcaataacctggacagcaaggtgggcggcaactacaactacctctacagactgttcagaaagagcaacctgaagcccttcgagagagacatcagcaccgagatctaccaggccggcagcaccccctgcaacggcgtgaagggcttcaactgctacttccccctccagagctacggcttccagcccacctacggcgtgggctaccagccctacagagtggtggtcctgagcttcgagctgctgcacgcccccgccaccgtgtgcggccccaagaagagcaccaatctggtgaagaacaagtgcgtgaacttctgatgactcgagcatgcatctagagggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 20 - DNA, 6321 bp, synthetic construct
  misc_feature 1..6321: note = Synthetic construct
  source 1..6321: mol_type = other DNA
gctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctagcttggtaccgagctcggatccaccatgggatggagctgtatcatcctcttcttggtagcaacagctaccggagtccactccgatgtgttgatgacccaatctccactctccctgcctgtcactcctggggagccagcctcgatctcttgctggaacaggcagctgtatccagagtggacagaagcccagagacttgactggtatctgcagaaaccaggccagtctccacagctcctgatctacgccaactgcagtgtttatgatttttttgtgtggctccattattattctgttagagatacattattaggaccaggacgcccctacaggggggtcccagacagattcagtggcagtggatcagggacagatttcacactcaagatcagcagagtggaggctgaggataccggagtgtattactgccagtgcacagaggtgcgagccgacacaaggccctggagtggtccctacatcctacgaaaccaggatgaccgtgagctgtggccaagaaaattcttcttcggtggaggcaccaaggtggaaatcaagcgtacggtagcggccccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccaccagggcctgagctcgcccgtcacaaagagcttcaacaggggagagtgttgactcgagcatgcatctagagggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccaggctagcgttcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttaccatgggatggagctgtatcatcctcttcttggtagcaacagctaccggagtccactcccaggtgcagctggtggagactgggggaggcttaatccagcctggagggtccctgagaatgtcctgcgggacaggcagggcaatgctgggcacacacaccatggaagtgactgtctaccattgggttcggcaggctccggggaaggggctggagtggatcgcatacattggtagtggtggtagtgtttatgatttttttgtgtggctccgattcaccatttccagagacaatagcaagaacaccctgtatttgcaattgaacagtctgagggctgaggacacagccgtgtattactgtgcccgagtccccctggattgtgttctgtatcgatatggttccttttccgtcaccctggacattgtccagggttggggccaaggaaccacggtcaccgtctccagcgcttccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcatacagcctggacacagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaacccaaagggagagcccagacaccacaggtgtacaccatacccccacctcgggaacaaatgtccaagaagaaggtcagcctgacctgcctggtcaccaacttcttctctgaagccatcagtgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgatctaaagggcgatatcgcccttaagggcgaattctgcagattcgagcatgcatctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctactgggcggttttatggacagcaagcgaaccggaattgccagctggggcgccctctggtaaggttgggaagccctgcaaagtaaactggatggctttcttgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatcaggtggcacttttcggggaaatgtgcgcggaacccctatttgtttatttttctaaatacattcaaatatgtatccgctcatgagacaataaccctgataaatgcttcaataatagcacgtgctaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctt

SEQ ID NO: 21 - DNA, 5071 bp, synthetic construct
  misc_feature 1..5071: note = Synthetic construct
  source 1..5071: mol_type = other DNA
gcgtataatggactattgtgtgctgatatgtacacctgaggacacaggtacaggactcagcttaaggctacatcttagcttacgtcactagagggtccacgtttagtttttaagatccattgatctcctaaacgctgcaagattcgcaacctggaatacttagcgctaggtcctagtgaagcgggactttttttctaaagtcgttgagaggaggagtcgtcagaccagatagctttgatgtcctgatcggaaggatcgttggccccgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctagcttggtaccgagctcggatccaccatgggatggagctgtatcatcctcttcttggtagcaacagctaccggagtccactccgatgtgttgatgacccaatctccactctccctgcctgtcactcctggggagccagcctcgatctcttgctggaacaggcagctgtatccagagtggacagaagcccagagacttgactggtatctgcagaaaccaggccagtctccacagctcctgatctacgccaactgcagtgtttatgatttttttgtgtggctccattattattctgttagagatacattattaggaccaggacgcccctacaggggggtcccagacagattcagtggcagtggatcagggacagatttcacactcaagatcagcagagtggaggctgaggataccggagtgtattactgccagtgcacagaggtgcgagccgacacaaggccctggagtggtccctacatcctacgaaaccaggatgaccgtgagctgtggccaagaaaattcttcttcggtggaggcaccaaggtggaaatcaagcgtacggtagcggccccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccaccagggcctgagctcgcccgtcacaaagagcttcaacaggggagagtgttgactcgagcatgcatcaagagggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccaggctagcgttcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttaccatgggatggagctgtatcatcctcttcttggtagcaacagctaccggagtccactcccaggtgcagctggtggagactgggggaggcttaatccagcctggagggtccctgagaatgtcctgcgggacaggcagggcaatgctgggcacacacaccatggaagtgactgtctaccattgggttcggcaggctccggggaaggggctggagtggatcgcatacattggtagtggtggtagtgtttatgatttttttgtgtggctccgattcaccatttccagagacaatagcaagaacaccctgtatttgcaattgaacagtctgagggctgaggacacagccgtgtattactgtgcccgagtccccctggattgtgttctgtatcgatatggttccttttccgtcaccctggacattgtccagggttggggccaaggaaccacggtcaccgtctccagcgcttccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcatacagcctggacacagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaacccaaagggagagcccagacaccacaggtgtacaccatacccccacctcgggaacaaatgtccaagaagaaggtcagcctgacctgcctggtcaccaacttcttctctgaagccatcagtgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgatctaaagggcgatatcgcccttaagggcgaattctgcagattcgagcatgcatatagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctactgggcggttttatggacagcaagcgaaccggaattgccagctggggcgccctctggtaaggttgggaagccctgcaaagtaaactggatggctttcttgccgccaaggatctgatggcgcaggggatcaagctctgatcaagagacaggatgagccgacccttagactctgtactcagttctataaacgagccattggatacgagatccgtagattgataagggacacggaatatccccggacgcaatagacggacagcttggtatcctgagcacagtcgcgcgtccgaatctagctctactttagaggccccggattctgatggtcgtagaccgcagaaccgattggagggatgctagcactagtgaggtgagattgtgttcggcatgcctcacaggcagatctatcagcacacaattgcccattatacgc

SEQ ID NO: 22 - DNA, 6207 bp, synthetic construct
  misc_feature 1..6207: note = Synthetic construct
  source 1..6207: mol_type = other DNA
gctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctagcttggtaccgagctcggatccaccatgggatggagctgtatcatcctcttcttggtagcaacagctaccggagtccactccgatgtgttgatgacccaatctccactctccctgcctgtcactcctggggagccagcctcgatctcttgcccaggggtgcttctgaaggagttcactgtgtccggcaacatactgactatccgactgactgctgcagaccaccgctggtatctgcagaaaccaggccagtctccacagctcctgatctacaaagtttccaaccgattttctggggtcccagacagattcagtggcagtggatcagggacagatttcacactcaagatcagcagagtggaggctgaggataccggagtgtattactgctttcaaggttcacatgttccgtggacgttcggtggaggcaccaaggtggaaatcaagcgtacggtagcggccccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccaccagggcctgagctcgcccgtcacaaagagcttcaacaggggagagtgttgactcgagcatgcatctagagggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccaggctagcgttcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttaccatgggatggagctgtatcatcctcttcttggtagcaacagctaccggagtccactcccaggtgcagctggtggagactgggggaggcttaatccagcctggagggtccctgagaatgtcctgcctgttgatgtggatcacgcagtgcttttgggttcggcaggctccggggaaggggctggagtggatcgcatacattggtagtggtggttccctgttgatgtggatcacgcagtgccgattcaccatttccagagacaatagcaagaacaccctgtatttgcaattgaacagtctgagggctgaggacacagccgtgtattactgtgcccgaccggagagccgcctgcttgagttctacctcgccatgcctttcgcgacacccatggaagcagagctggcccgcaggagcctggcccagtggggccaaggaaccacggtcaccgtctccagcgcttccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcatacagcctggacacagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaacccaaagggagagcccagacaccacaggtgtacaccatacccccacctcgggaacaaatgtccaagaagaaggtcagcctgacctgcctggtcaccaacttcttctctgaagccatcagtgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgatctaaagggcgatatcgcccttaagggcgaattctgcagattcgagcatgcatctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctactgggcggttttatggacagcaagcgaaccggaattgccagctggggcgccctctggtaaggttgggaagccctgcaaagtaaactggatggctttcttgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatcaggtggcacttttcggggaaatgtgcgcggaacccctatttgtttatttttctaaatacattcaaatatgtatccgctcatgagacaataaccctgataaatgcttcaataatagcacgtgctaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctt

SEQ ID NO: 23 - DNA, 6768 bp, synthetic construct
  misc_feature 1..6768: note = Synthetic sequence
  source 1..6768: mol_type = other DNA
gctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctagcttggtaccgagctcggatccgccaccatggactggatttggagaatcctcttcctggtgggagctgccacaggcgcacacagcagagtgcagcccaccgagagcatcgtgagattccccaacatcaccaacctgtgccccttcggcgaggtgttcaacgccaccagattcgccagcgtgtacgcctggaacaggaagagaatcagcaactgcgtggccgactacagcgtgctctacaacagcgccagcttcagcaccttcaagtgctacggcgtgagccccaccaagctgaacgacctgtgcttcaccaacgtgtacgccgacagcttcgtgatcagaggcgacgaggtgagacagatcgcccccggccagaccggcaagatcgccgactacaactacaagctgcccgacgacttcaccggctgcgtgatcgcgtggaacagcaataacctggacagcaaggtgggcggcaactacaactacctctacagactgttcagaaagagcaacctgaagcccttcgagagagacatcagcaccgagatctaccaggccggcagcaccccctgcaacggcgtggagggcttcaactgctacttccccctccagagctacggcttccagcccaccaacggcgtgggctaccagccctacagagtggtggtcctgagcttcgagctgctgcacgcccccgccaccgtgtgcggccccaagaagagcaccaatctggtgaagaacaagtgcgtgaacttctgatgactcgagcatgcatctagagggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccaggctagcgttcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttgccaccatggactggatttggagaatcctcttcctggtgggagctgccacaggcgcacacagcagcgacaacggcccccagaaccagagaaacgcccccagaatcaccttcggcggccccagcgacagcaccggcagcaaccagaacggcgagagaagcggcgccagaagcaagcagagaagaccccagggcctgcccaacaacaccgccagctggttcaccgccctgacccagcacggcaaggaggacctgaagttccccagaggccagggcgtgcccatcaacaccaacagcagccccgacgaccagatcggctactacagaagagccaccagaagaatcagaggcggcgacggcaagatgaaggacctgagccccagatggtacttctactacctgggcaccggccccgaggccggcctgccctacggcgccaacaaggacggcatcatctgggtggccaccgagggcgccctgaacacccccaaggaccacatcggcaccagaaaccccgccaacaacgccgccatcgtgctccagctgccccagggcaccaccctgcccaagggcttctacgccgagggcagcagaggcggcagccaggcctcaagccgttccagcagcagatcccgcaacagctcccgcaacagcacccctggaagctcaaggggcacctctcctgcgcggatggctggtaacggcggggacgctgccctggccctgctgctgctggacagactgaaccagctggagagcaagatgagcggcaagggccagcagcagcagggccagactgtgaccaaaaaaagcgccgccgaggccagcaagaagcccagacagaagagaaccgccaccaaggcctacaacgtgacccaggccttcggcagaagaggccccgagcagacccagggcaacttcggcgaccaggagctgatcagacagggcaccgactacaagcactggccccagatcgcccagttcgcccccagcgccagcgccttcttcggcatgagcagaatcggcatggaggtgacccccagcggcacctggctgacctacaccggcgccatcaagctggacgacaaggaccccaacttcaaggatcaggtgatcctgctgaacaagcacatcgacgcctacaagaccttcccccccaccgagcccaagaaggacaagaagaagaaggccgacgagacccaggccctgccccagagacagaagaagcagcagacagtgacattgctgcccgccgccgacctggacgacttcagcaagcagctccagcagagcatgagcagcgccgacagcacccaggccgagcccaaatcgtgcgataaaacgcatacgtgccccccctgtccagccccagagttgctgggtggaccgagcgtgttcctgttcccccccaagcccaaggacaccctgatgatcagcagaacccccgaggtgacctgcgtggtggtggacgtgagccacgaggaccccgaggtgaagttcaactggtacgtggacggcgtggaggtgcacacagcctggacccagcccagagaggagcagtacaacagcacctacagagttgtgtccgtgctgaccgtgctgcaccaggactggctgaacggcaaggagtacaagtgcaaggtgagcaacaaggccctgcccgcccccatcgagaagaccatcagcaagcccaagggcagagcccagacaccccaggtgtacaccatacccccccctagagaacaaatgtccaagaagaaggtgagcctgacctgcctggtgaccaacttcttctctgaagccatcagtgtggagtgggagagcaacggccagcccgagaacaactacaagaccaccccccccgtgctggacagcgacggcagcttcttcctctacagcaagctgaccgtggacaagagcagatggcagcagggcaacgtgttctcttgcagcgtgatgcacgaggccctgcacaaccactacacccagaagagcctgagcctgagccccggcaagtgatgatctaaagggcgaattcgcccttaagggctaattctgcagattcgagcatgcatctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctactgggcggttttatggacagcaagcgaaccggaattgccagctggggcgccctctggtaaggttgggaagccctgcaaagtaaactggatggctttcttgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatcaggtggcacttttcggggaaatgtgcgcggaacccctatttgtttatttttctaaatacattcaaatatgtatccgctcatgagacaataaccctgataaatgcttcaataatagcacgtgctaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctt

SEQ ID NO: 24 - DNA, 6768 bp, synthetic construct
  misc_feature 1..6768: note = Synthetic construct
  source 1..6768: mol_type = other DNA
gctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctagcttggtaccgagctcggatccgccaccatggactggatttggagaatcctcttcctggtgggagctgccacaggcgcacacagcagagtgcagcccaccgagagcatcgtgagattccccaacatcaccaacctgtgccccttcggcgaggtgttcaacgccaccagattcgccagcgtgtacgcctggaacaggaagagaatcagcaactgcgtggccgactacagcgtgctctacaacagcgccagcttcagcaccttcaagtgctacggcgtgagccccaccaagctgaacgacctgtgcttcaccaacgtgtacgccgacagcttcgtgatcagaggcgacgaggtgagacagatcgcccccggccagaccggcaatatcgccgactacaactacaagctgcccgacgacttcaccggctgcgtgatcgcgtggaacagcaataacctggacagcaaggtgggcggcaactacaactacctctacagactgttcagaaagagcaacctgaagcccttcgagagagacatcagcaccgagatctaccaggccggcagcaccccctgcaacggcgtgaagggcttcaactgctacttccccctccagagctacggcttccagcccacctacggcgtgggctaccagccctacagagtggtggtcctgagcttcgagctgctgcacgcccccgccaccgtgtgcggccccaagaagagcaccaatctggtgaagaacaagtgcgtgaacttctgatgactcgagcatgcatctagagggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccaggctagcgttcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttgccaccatggactggatttggagaatcctcttcctggtgggagctgccacaggcgcacacagcagcgacaacggcccccagaaccagagaaacgcccccagaatcaccttcggcggccccagcgacagcaccggcagcaaccagaacggcgagagaagcggcgccagaagcaagcagagaagaccccagggcctgcccaacaacaccgccagctggttcaccgccctgacccagcacggcaaggaggacctgaagttccccagaggccagggcgtgcccatcaacaccaacagcagccccgacgaccagatcggctactacagaagagccaccagaagaatcagaggcggcgacggcaagatgaaggacctgagccccagatggtacttctactacctgggcaccggccccgaggccggcctgccctacggcgccaacaaggacggcatcatctgggtggccaccgagggcgccctgaacacccccaaggaccacatcggcaccagaaaccccgccaacaacgccgccatcgtgctccagctgccccagggcaccaccctgcccaagggcttctacgccgagggcagcagaggcggcagccaggcctcaagccgttccagcagcagatcccgcaacagctcccgcaacagcacccctggaagctcaaggggcatctctcctgcgcggatggctggtaacggcggggacgctgccctggccctgctgctgctggacagactgaaccagctggagagcaagatgagcggcaagggccagcagcagcagggccagactgtgaccaaaaaaagcgccgccgaggccagcaagaagcccagacagaagagaaccgccaccaaggcctacaacgtgacccaggccttcggcagaagaggccccgagcagacccagggcaacttcggcgaccaggagctgatcagacagggcaccgactacaagcactggccccagatcgcccagttcgcccccagcgccagcgccttcttcggcatgagcagaatcggcatggaggtgacccccagcggcacctggctgacctacaccggcgccatcaagctggacgacaaggaccccaacttcaaggatcaggtgatcctgctgaacaagcacatcgacgcctacaagaccttcccccccaccgagcccaagaaggacaagaagaagaaggccgacgagacccaggccctgccccagagacagaagaagcagcagacagtgacattgctgcccgccgccgacctggacgacttcagcaagcagctccagcagagcatgagcagcgccgacagcacccaggccgagcccaaatcgtgcgataaaacgcatacgtgccccccctgtccagccccagagttgctgggtggaccgagcgtgttcctgttcccccccaagcccaaggacaccctgatgatcagcagaacccccgaggtgacctgcgtggtggtggacgtgagccacgaggaccccgaggtgaagttcaactggtacgtggacggcgtggaggtgcacacagcctggacccagcccagagaggagcagtacaacagcacctacagagttgtgtccgtgctgaccgtgctgcaccaggactggctgaacggcaaggagtacaagtgcaaggtgagcaacaaggccctgcccgcccccatcgagaagaccatcagcaagcccaagggcagagcccagacaccccaggtgtacaccatacccccccctagagaacaaatgtccaagaagaaggtgagcctgacctgcctggtgaccaacttcttctctgaagccatcagtgtggagtgggagagcaacggccagcccgagaacaactacaagaccaccccccccgtgctggacagcgacggcagcttcttcctctacagcaagctgaccgtggacaagagcagatggcagcagggcaacgtgttctcttgcagcgtgatgcacgaggccctgcacaaccactacacccagaagagcctgagcctgagccccggcaagtgatgatctaaagggcgaattcgcccttaagggctaattctgcagattcgagcatgcatctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctactgggcggttttatggacagcaagcgaaccggaattgccagctggggcgccctctggtaaggttgggaagccctgcaaagtaaactggatggctttcttgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatcaggtggcacttttcggggaaatgtgcgcggaacccctatttgtttatttttctaaatacattcaaatatgtatccgctcatgagacaataaccctgataaatgcttcaataatagcacgtgctaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctt

SEQ ID NO: 25 - DNA, 5518 bp, synthetic construct
  misc_feature 1..5518: note = Synthetic construct
  source 1..5518: mol_type = other DNA
gcgtataatggactattgtgtgctgatatgtacacctgaggacacaggtacaggactcagcttaaggctacatcttagcttacgtcactagagggtccacgtttagtttttaagatccattgatctcctaaacgctgcaagattcgcaacctggaatacttagcgctaggtcctagtgaagcgggactttttttctaaagtcgttgagaggaggagtcgtcagaccagatagctttgatgtcctgatcggaaggatcgttggccccgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctagcttggtaccgagctcggatccgccaccatggactggatttggagaatcctcttcctggtgggagctgccacaggcgcacacagcagagtgcagcccaccgagagcatcgtgagattccccaacatcaccaacctgtgccccttcggcgaggtgttcaacgccaccagattcgccagcgtgtacgcctggaacaggaagagaatcagcaactgcgtggccgactacagcgtgctctacaacagcgccagcttcagcaccttcaagtgctacggcgtgagccccaccaagctgaacgacctgtgcttcaccaacgtgtacgccgacagcttcgtgatcagaggcgacgaggtgagacagatcgcccccggccagaccggcaatatcgccgactacaactacaagctgcccgacgacttcaccggctgcgtgatcgcgtggaacagcaataacctggacagcaaggtgggcggcaactacaactacctctacagactgttcagaaagagcaacctgaagcccttcgagagagacatcagcaccgagatctaccaggccggcagcaccccctgcaacggcgtgaagggcttcaactgctacttccccctccagagctacggcttccagcccacctacggcgtgggctaccagccctacagagtggtggtcctgagcttcgagctgctgcacgcccccgccaccgtgtgcggccccaagaagagcaccaatctggtgaagaacaagtgcgtgaacttctgatgactcgagcatgcatcaagagggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccaggctagcgttcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttgccaccatggactggatttggagaatcctcttcctggtgggagctgccacaggcgcacacagcagcgacaacggcccccagaaccagagaaacgcccccagaatcaccttcggcggccccagcgacagcaccggcagcaaccagaacggcgagagaagcggcgccagaagcaagcagagaagaccccagggcctgcccaacaacaccgccagctggttcaccgccctgacccagcacggcaaggaggacctgaagttccccagaggccagggcgtgcccatcaacaccaacagcagccccgacgaccagatcggctactacagaagagccaccagaagaatcagaggcggcgacggcaagatgaaggacctgagccccagatggtacttctactacctgggcaccggccccgaggccggcctgccctacggcgccaacaaggacggcatcatctgggtggccaccgagggcgccctgaacacccccaaggaccacatcggcaccagaaaccccgccaacaacgccgccatcgtgctccagctgccccagggcaccaccctgcccaagggcttctacgccgagggcagcagaggcggcagccaggcctcaagccgttccagcagcagatcccgcaacagctcccgcaacagcacccctggaagctcaaggggcatctctcctgcgcggatggctggtaacggcggggacgctgccctggccctgctgctgctggacagactgaaccagctggagagcaagatgagcggcaagggccagcagcagcagggccagactgtgaccaaaaaaagcgccgccgaggccagcaagaagcccagacagaagagaaccgccaccaaggcctacaacgtgacccaggccttcggcagaagaggccccgagcagacccagggcaacttcggcgaccaggagctgatcagacagggcaccgactacaagcactggccccagatcgcccagttcgcccccagcgccagcgccttcttcggcatgagcagaatcggcatggaggtgacccccagcggcacctggctgacctacaccggcgccatcaagctggacgacaaggaccccaacttcaaggatcaggtgatcctgctgaacaagcacatcgacgcctacaagaccttcccccccaccgagcccaagaaggacaagaagaagaaggccgacgagacccaggccctgccccagagacagaagaagcagcagacagtgacattgctgcccgccgccgacctggacgacttcagcaagcagctccagcagagcatgagcagcgccgacagcacccaggccgagcccaaatcgtgcgataaaacgcatacgtgccccccctgtccagccccagagttgctgggtggaccgagcgtgttcctgttcccccccaagcccaaggacaccctgatgatcagcagaacccccgaggtgacctgcgtggtggtggacgtgagccacgaggaccccgaggtgaagttcaactggtacgtggacggcgtggaggtgcacacagcctggacccagcccagagaggagcagtacaacagcacctacagagttgtgtccgtgctgaccgtgctgcaccaggactggctgaacggcaaggagtacaagtgcaaggtgagcaacaaggccctgcccgcccccatcgagaagaccatcagcaagcccaagggcagagcccagacaccccaggtgtacaccatacccccccctagagaacaaatgtccaagaagaaggtgagcctgacctgcctggtgaccaacttcttctctgaagccatcagtgtggagtgggagagcaacggccagcccgagaacaactacaagaccaccccccccgtgctggacagcgacggcagcttcttcctctacagcaagctgaccgtggacaagagcagatggcagcagggcaacgtgttctcttgcagcgtgatgcacgaggccctgcacaaccactacacccagaagagcctgagcctgagccccggcaagtgatgatctaaagggcgaattcgcccttaagggctaattctgcagattcgagcatgcatatagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctactgggcggttttatggacagcaagcgaaccggaattgccagctggggcgccctctggtaaggttgggaagccctgcaaagtaaactggatggctttcttgccgccaaggatctgatggcgcaggggatcaagctctgatcaagagacaggatgagccgacccttagactctgtactcagttctataaacgagccattggatacgagatccgtagattgataagggacacggaatatccccggacgcaatagacggacagcttggtatcctgagcacagtcgcgcgtccgaatctagctctactttagaggccccggattctgatggtcgtagaccgcagaaccgattggagggatgctagcactagtgaggtgagattgtgttcggcatgcctcacaggcagatctatcagcacacaattgcccattatacgc

SEQ ID NO: 26 - protein, 418 aa, synthetic construct
  REGION 1..418: note = Synthetic construct
SLNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMFGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 27 - protein, 669 aa, synthetic construct
  REGION 1..669: note = Synthetic construct
MDWIWRILFLVGAATGAHSSLNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMFGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQAEPKSCDKTHTCPPCPAPELLGGPSVFLFPPKPKDTLMISRTPEVTCVVVDVSHEDPEVKFNWYVDGVEVHTAWTQPREEQYNSTYRVVSVLTVLHQDWLNGKEYKCKVSNKALPAPIEKTISKPKGRAQTPQVYTIPPPREQMSKKKVSLTCLVTNFFSEAISVEWESNGQPENNYKTTPPVLDSDGSFFLYSKLTVDKSRWQQGNVFSCSVMHEALHNHYTQKSLSLSPGK

SEQ ID NO: 28 - protein, 223 aa, synthetic construct
  REGION 1..223: note = Synthetic construct
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTYGVGYQPYRVVVLSFELLHAPATVCGPKKSTNLVKNKCVNF

SEQ ID NO: 29 - protein, 18 aa, synthetic construct
  REGION 1..18: note = Synthetic construct
GTGRAMLGTHTMEVTVYH

SEQ ID NO: 30 - protein, 9 aa, synthetic construct
  REGION 1..9: note = Synthetic construct
SVYDFFVWL

SEQ ID NO: 31 - protein, 16 aa, synthetic construct
  REGION 1..16: note = Synthetic construct
WNRQLYPEWTEAQRLD

SEQ ID NO: 32 - protein, 22 aa, synthetic construct
  REGION 1..22: note = Synthetic construct
VPLDCVLYRYGSFSVTLDIVQG

SEQ ID NO: 33 - protein, 29 aa, synthetic construct
  REGION 1..29: note = Synthetic construct
ANCSVYDFFVWLHYYSVRDTLLGPGRPYR

SEQ ID NO: 34 - protein, 32 aa, synthetic construct
  REGION 1..32: note = Synthetic construct
QCTEVRADTRPWSGPYILRNQDDRELWPRKFF

SEQ ID NO: 35 - protein, 9 aa, synthetic construct
  REGION 1..9: note = Synthetic construct
LLMWITQCF

SEQ ID NO: 36 - protein, 9 aa, synthetic construct
  REGION 1..9: note = Synthetic construct
SLLMWITQC

SEQ ID NO: 37 - protein, 29 aa, synthetic construct
  REGION 1..29: note = Synthetic construct
PESRLLEFYLAMPFATPMEAELARRSLAQ

SEQ ID NO: 38 - protein, 25 aa, synthetic construct
  REGION 1..25: note = Synthetic construct
PGVLLKEFTVSGNILTIRLTAADHR

SEQ ID NO: 39 - protein, 9 aa, synthetic construct
  REGION 1..9: note = Synthetic construct
PESRLLEFY

SEQ ID NO: 40 - protein, 14 aa, synthetic construct
  REGION 1..14: note = Synthetic construct
RLLEFYLAMPFATP

SEQ ID NO: 41 - protein, 9 aa, synthetic construct
  REGION 1..9: note = Synthetic construct
LEFYLAMPF

SEQ ID NO: 42 - protein, 12 aa, synthetic construct
  REGION 1..12: note = Synthetic construct
EFYLAMPFATPM

SEQ ID NO: 43 - protein, 9 aa, synthetic construct
  REGION 1..9: note = Synthetic construct
MPFATPMEA

SEQ ID NO: 44 - protein, 9 aa, synthetic construct
  REGION 1..9: note = Synthetic construct
LAMPFATPM

SEQ ID NO: 45 - protein, 14 aa, synthetic construct
  REGION 1..14: note = Synthetic construct
LLEFYLAMPFATPM

SEQ ID NO: 46 - protein, 25 aa, synthetic construct
  REGION 1..25: note = Synthetic construct
LLEFYLAMPFATPMEAELARRSLAQ

SEQ ID NO: 47 - protein, 15 aa, synthetic construct
  REGION 1..15: note = Synthetic construct
LKEFTVSGNILTIRL

SEQ ID NO: 48 - protein, 11 aa, synthetic construct
  REGION 1..11: note = Synthetic construct
KEFTVSGNILT

SEQ ID NO: 49 - protein, 12 aa, synthetic construct
  REGION 1..12: note = Synthetic construct
KEFTVSGNILTI

SEQ ID NO: 50 - protein, 10 aa, synthetic construct
  REGION 1..10: note = Synthetic construct
TVSGNILTIR

SEQ ID NO: 51 - protein, 9 aa, synthetic construct
  REGION 1..9: note = Synthetic construct
TVSGNILTI

SEQ ID NO: 52 - protein, 9 aa, synthetic construct
  REGION 1..9: note = Synthetic construct
KIADYNYKL

SEQ ID NO: 53 - protein, 10 aa, synthetic construct
  REGION 1..10: note = Synthetic construct
KLPDDFTGCV

SEQ ID NO: 54 - protein, 9 aa, synthetic construct
  REGION 1..9: note = Synthetic construct
ELLHAPATV

SEQ ID NO: 55 - protein, 20 aa, synthetic construct
  REGION 1..20: note = Synthetic construct
CPFGEVFNATRFASVTAWNR

SEQ ID NO: 56 - protein, 20 aa, synthetic construct
  REGION 1..20: note = Synthetic construct
RISNCVADYSVLYNSASFST

SEQ ID NO: 57 - protein, 18 aa, synthetic construct
  REGION 1..18: note = Synthetic construct
YLYRLFRKSNLKPFERDI

SEQ ID NO: 58 - protein, 20 aa, synthetic construct
  REGION 1..20: note = Synthetic construct
YQPYRVVVLSFELLHAPATV

SEQ ID NO: 59 - protein, 9 aa, synthetic construct
  REGION 1..9: note = Synthetic construct
ALNTPKDHI

SEQ ID NO: 60 - protein, 9 aa, synthetic construct
  REGION 1..9: note = Synthetic construct
LQLPQGTTL

SEQ ID NO: 61 - protein, 9 aa, synthetic construct
  REGION 1..9: note = Synthetic construct
LLLDRLNQL

SEQ ID NO: 62 - protein, 9 aa, synthetic construct
  REGION 1..9: note = Synthetic construct
GMSRIGMEV

SEQ ID NO: 63 - protein, 9 aa, synthetic construct
  REGION 1..9: note = Synthetic construct
ILLNKHIDA

SEQ ID NO: 64 - protein, 20 aa, synthetic construct
  REGION 1..20: note = Synthetic construct
GNGGDAALALLLLDRLNQLE

SEQ ID NO: 65 - protein, 20 aa, synthetic construct
  REGION 1..20: note = Synthetic construct
KHWPQIAQFAPSASAFFGMS

SEQ ID NO: 66 - protein, 9 aa, synthetic construct
  REGION 1..9: note = Synthetic construct
MLGTHTMEV

SEQ ID NO: 67 - protein, 15 aa, synthetic construct
  REGION 1..15: note = Synthetic construct
VLYRYGSFSVTLDIV

SEQ ID NO: 68 - protein, 20 aa, synthetic construct
  REGION 1..20: note = Synthetic construct
METDTLLLWVLLLWVPGSTG

SEQ ID NO: 69 - protein, 19 aa, synthetic construct
  REGION 1..19: note = Synthetic construct
MDWIWRILFLVGAATGAHS

SEQ ID NO: 70 - protein, 196 aa, synthetic construct
  REGION 1..196: note = Synthetic construct
PNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVC

SEQ ID NO: 71 - protein, 1273 aa, synthetic construct
  REGION 1..1273: note = Synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 72 - protein, 27 aa, synthetic construct
  REGION 1..27: note = Synthetic construct
GYIPEAPRDGQAYVRKDGEWVLLSTFL

SEQ ID NO: 73 - protein, 7 aa, synthetic construct
  REGION 1..7: note = Synthetic construct
CCGGGSG

SEQ ID NO: 74 - protein, 14 aa, synthetic construct
  REGION 1..14: note = Synthetic construct
GGGSGGGSGGGSGS

SEQ ID NO: 75 - protein, 12 aa, synthetic construct
  REGION 1..12: note = Synthetic construct
GGGSGGGSGGGS

SEQ ID NO: 76 - protein, 232 aa, synthetic construct
  REGION 1..232: note = Synthetic construct
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 77 - protein, 12 aa, synthetic construct
  REGION 1..12: note = Synthetic construct
EPKSCDKTHTCP

SEQ ID NO: 78 - protein, 113 aa, synthetic construct
  REGION 1..113: note = Synthetic construct
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 79 - protein, 107 aa, synthetic construct
  REGION 1..107: note = Synthetic construct
GQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 80 - protein, 330 aa, synthetic construct
  REGION 1..330: note = Synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHTAWTQPREEQYNSTYRVVSVLTVLHQDWLNGKEYKCKVSNKALPAPIEKTISKPKGRAQTPQVYTIPPPREQMSKKKVSLTCLVTNFFSEAISVEWESNGQPENNYKTTPPVLDSDGSFFLYSKLTVDKSRWQQGNVFSCSVMHEALHNHYTQKSLSLSPGK

SEQ ID NO: 81 - protein, 243 aa, synthetic construct
  REGION 1..243: note = Synthetic construct
METDTLLLWVLLLWVPGSTGRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNF

SEQ ID NO: 82 - protein, 670 aa, synthetic construct
  REGION 1..670: note = Synthetic construct
METDTLLLWVLLLWVPGSTGSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQAEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 83 - protein, 272 aa, synthetic construct
  REGION 1..272: note = Synthetic construct
METDTLLLWVLLLWVPGSTGRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFGSGYIPEAPRDGQAYVRKDGEWVLLSTFL

SEQ ID NO: 84 - protein, 670 aa, synthetic construct; identical to SEQ ID NO: 82
  REGION 1..670: note = Synthetic construct

SEQ ID NO: 85 - protein, 475 aa, synthetic construct
  REGION 1..475: note = Synthetic construct
METDTLLLWVLLLWVPGSTGRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 86 - protein, 438 aa, synthetic construct
  REGION 1..438: note = Synthetic sequence
METDTLLLWVLLLWVPGSTGSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 87 - protein, 243 aa, synthetic construct; identical to SEQ ID NO: 81
  REGION 1..243: note = Synthetic sequence

SEQ ID NO: 88 - protein, 438 aa, synthetic construct; identical to SEQ ID NO: 86
  REGION 1..438: note = Synthetic construct

SEQ ID NO: 89 - protein, 242 aa, synthetic construct
  REGION 1..242: note = Synthetic construct
MDWIWRILFLVGAATGAHSRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNF

SEQ ID NO: 90 - protein, 669 aa, synthetic construct
  REGION 1..669: note = Synthetic construct
MDWIWRILFLVGAATGAHSSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQAEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 91 - protein, 271 aa, synthetic construct
  REGION 1..271: note = Synthetic construct
MDWIWRILFLVGAATGAHSRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFGSGYIPEAPRDGQAYVRKDGEWVLLSTFL

SEQ ID NO: 92 - protein, 474 aa, synthetic construct
  REGION 1..474: note = Synthetic construct
MDWIWRILFLVGAATGAHSRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 93 - protein, 437 aa, synthetic construct
  REGION 1..437: note = Synthetic construct
MDWIWRILFLVGAATGAHSSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 94 - protein, 263 aa, synthetic construct
  REGION 1..263: note = Synthetic construct
MDWIWRILFLVGAATGAHSPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGGGSGGGSGGGSGSGYIPEAPRDGQAYVRKDGEWVLLSTFLGTGGGSG

SEQ ID NO: 95 - protein, 234 aa, synthetic construct
  REGION 1..234: note = Synthetic construct
MDWIWRILFLVGAATGAHSPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGGGSGGGSGGGSCCGGGSG

SEQ ID NO: 96 - protein, 263 aa, synthetic construct; identical to SEQ ID NO: 94
  REGION 1..263: note = Synthetic construct

SEQ ID NO: 97 - protein, 474 aa, synthetic construct
  REGION 1..474: note = Synthetic construct
MDWIWRILFLVGAATGAHSRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFEPKSCDKTHTCPPCPAPELLGGPSVFLFPPKPKDTLMISRTPEVTCVVVDVSHEDPEVKFNWYVDGVEVHTAWTQPREEQYNSTYRVVSVLTVLHQDWLNGKEYKCKVSNKALPAPIEKTISKPKGRAQTPQVYTIPPPREQMSKKKVSLTCLVTNFFSEAISVEWESNGQPENNYKTTPPVLDSDGSFFLYSKLTVDKSRWQQGNVFSCSVMHEALHNHYTQKSLSLSPGK

SEQ ID NO: 98 - protein, 477 aa, synthetic construct
  REGION 1..477: note = Synthetic construct
MGWSCIILFLVATATGVHSQVQLVETGGGLIQPGGSLRMSCGTGRAMLGTHTMEVTVYHWVRQAPGKGLEWIAYIGSGGSVYDFFVWLRFTISRDNSKNTLYLQLNSLRAEDTAVYYCARWNRQLYPEWTEAQRLDWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 99 - protein, 247 aa, synthetic construct
  REGION 1..247: note = Synthetic construct
MGWSCIILFLVATATGVHSDVLMTQSPLSLPVTPGEPASISCWNRQLYPEWTEAQRLDWYLQKPGQSPQLLIYKVSNRFSGVPDRFSGSGSGTDFTLKISRVEAEDTGVYYCGTGRAMLGTHTMEVTVYHFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 100 - protein, 477 aa, synthetic construct
  REGION 1..477: note = Synthetic construct
MGWSCIILFLVATATGVHSQVQLVETGGGLIQPGGSLRMSCGTGRAMLGTHTMEVTVYHWVRQAPGKGLEWIAYIGSGGSVYDFFVWLRFTISRDNSKNTLYLQLNSLRAEDTAVYYCARWNRQLYPEWTEAQRLDWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHTAWTQPREEQYNSTYRVVSVLTVLHQDWLNGKEYKCKVSNKALPAPIEKTISKPKGRAQTPQVYTIPPPREQMSKKKVSLTCLVTNFFSEAISVEWESNGQPENNYKTTPPVLDSDGSFFLYSKLTVDKSRWQQGNVFSCSVMHEALHNHYTQKSLSLSPGK

SEQ ID NO: 101 - protein, 483 aa, synthetic construct
  REGION 1..483: note = Synthetic construct
MGWSCIILFLVATATGVHSQVQLVETGGGLIQPGGSLRMSCGTGRAMLGTHTMEVTVYHWVRQAPGKGLEWIAYIGSGGSVYDFFVWLRFTISRDNSKNTLYLQLNSLRAEDTAVYYCARVPLDCVLYRYGSFSVTLDIVQGWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 102 - protein, 283 aa, synthetic construct; identical to SEQ ID NO: 10
  REGION 1..283: note = Synthetic construct

SEQ ID NO: 103 - protein, 481 aa, synthetic construct
  REGION 1..481: note = Synthetic construct
MGWSCIILFLVATATGVHSQVQLVETGGGLIQPGGSLRMSCLLMWITQCFWVRQAPGKGLEWIAYIGSGGSLLMWITQCRFTISRDNSKNTLYLQLNSLRAEDTAVYYCARPESRLLEFYLAMPFATPMEAELARRSLAQWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 104 - protein, 242 aa, synthetic construct
  REGION 1..242: note = Synthetic construct
MDWIWRILFLVGAATGAHSRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTYGVGYQPYRVVVLSFELLHAPATVCGPKKSTNLVKNKCVNF

SEQ ID NO: 105 - protein, 242 aa, synthetic construct
  REGION 1..242: note = Synthetic construct
MDWIWRILFLVGAATGAHSRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNF

SEQ ID NO: 106 - protein, 5 aa, synthetic construct
  REGION 1..5: note = Synthetic construct
GGGGS

SEQ ID NO: 107 - protein, 5 aa, synthetic construct
  REGION 1..5: note = Synthetic construct
GGGSG

SEQ ID NO: 108 - protein, 5 aa, synthetic construct
  REGION 1..5: note = Synthetic construct
GGSGG

SEQ ID NO: 109 - protein, 5 aa, synthetic construct
  REGION 1..5: note = Synthetic construct
GSGGG

SEQ ID NO: 110 - protein, 6 aa, synthetic construct
  REGION 1..6: note = Synthetic construct
GSGGGP

SEQ ID NO: 111 - protein, 5 aa, synthetic construct
  REGION 1..5: note = Synthetic construct
GGEPS

SEQ ID NO: 112 - protein, 7 aa, synthetic construct
  REGION 1..7: note = Synthetic construct
GGEGGGP

SEQ ID NO: 113 - protein, 12 aa, synthetic construct
  REGION 1..12: note = Synthetic construct
GGEGGGSEGGGS

SEQ ID NO: 114 - protein, 8 aa, synthetic construct
  REGION 1..8: note = Synthetic construct
GGGSGGGG

SEQ ID NO: 115 - protein, 4 aa, synthetic construct
  REGION 1..4: note = Synthetic construct
GGGS

SEQ ID NO: 116 - protein, 5 aa, synthetic construct
  REGION 1..5: note = Synthetic construct
TVLRT

SEQ ID NO: 117 - protein, 6 aa, synthetic construct
  REGION 1..6: note = Synthetic construct
TVSSAS

SEQ ID NO: 118 - protein, 7 aa, synthetic construct
  REGION 1..7: note = Synthetic construct
TVLSSAS

SEQ ID NO: 119 - protein, 15 aa, synthetic construct
  REGION 1..15: note = Synthetic construct
NCSVYDFFVWLHYYS

SEQ ID NO: 120 - protein, 17 aa, synthetic construct
  REGION 1..17: note = Synthetic construct
TGRAMLGTHTMEVTVYH

(19) United States
(12) Patent Application Publication
(10) Pub. No.: US 2026/0325626 A1
(43) Pub. Date: Oct. 22, 2026

(54) NUCLEIC ACIDS ENCODING A POLYPEPTIDE COMPRISING A MODIFIED FC REGION OF A HUMAN IGG1 AND AT LEAST ONE HETEROLOGOUS ANTIGEN

(71) Applicant: Scancell Limited, Oxford (GB)

(72) Inventors: Linda Gillian Durrant, Oxford (GB); Mireille Vankemmelbeke, Oxford (GB); Victoria Anne Brentville, Oxford (GB); Rachael Louise Metheringham, Oxford (GB)

(73) Assignee: Scancell Limited, Oxford (GB)

(21) Appl. No.: 19/718,070

(22) Filed: Jun. 24, 2026

Related U.S. Application Data
(63) Continuation of application No. 18/022,778, filed on Aug. 25, 2021.

(30) Foreign Application Priority Data
Aug. 26, 2020 (GB) 2013385.6
Feb. 2, 2021 (GB) 2101435.2

(51) Int. Cl. A61K 39/215; A61K 39/00; A61P 31/14; A61P 35/00; C07K 14/005; C07K 14/47; C07K 16/28; C07K 16/40

(52) U.S. Cl. CPC A61K 39/215; A61K 39/001156; A61K 39/001188; A61K 39/00119; A61K 39/001192; A61P 31/14; A61P 35/00; C07K 14/005; C07K 14/4748; C07K 16/283; C07K 16/40; A61K 2039/505; A61K 2039/53; C07K 2317/20; C07K 2317/34; C07K 2317/524; C07K 2317/526; C07K 2317/53; C07K 2317/565; C07K 2317/76; C07K 2319/30; C12N 2770/20022; C12N 2770/20034

(57) ABSTRACT

The present invention relates to nucleic acids and peptides encoded by those nucleic acids. In particular, the peptides comprise a modified IgG1 Fc region and one or more heterologous epitopes, which may be B- or T-cell epitopes. A nucleic acid of the invention may encode a polypeptide comprising: (i) a modified Fc region of a human IgG1, and (i) at least one heterologous antigen, wherein (a) the modified Fc region comprises at least the part of Fc that is capable of binding to CD64 and/or TRIM21, (b) at least one residue of the Fc region is modified to the corresponding residue from a mouse IgG3 antibody and (c) the modified Fc region has enhanced avidity for Fc-gamma receptor (FcγR) when compared to the corresponding wildtype Fc region.

### RELATED APPLICATIONS

This application is a national stage filing under 35 U.S.C. § 371 of International Patent Application No. PCT/EP2021/073542, filed Aug. 25, 2021, which claims priority under 35 U.S.C. § 119 or 365 to Great Britian application number 2101435.2, filed Feb. 2, 2021, and Great Britian application number 2013385.6, filed Aug. 26, 2020. The contents of these applications are incorporated herein by reference in their entireties.

### REFERENCE TO AN ELECTRONIC SEQUENCE LISTING

The contents of the electronic sequence listing (S2239.70002US00-SUBSEQ-ZJG.txt; Size: 242,575 bytes; and Date of Creation: Jun. 22, 2026) are herein incorporated by reference in their entirety.

The present invention relates to nucleic acids and peptides encoded by those nucleic acids. In particular, the peptides comprise a modified IgG1 Fc region and one or more heterologous epitopes, which may be B- or T-cell epitopes. The nucleic acids and peptides of the invention can be used as a vaccine that stimulates high avidity CD8 T cells, Th1 CD4 T-cells and strong antibody responses. For cancer vaccines, stimulation of potent cellular immunity including both CD4 and CD8 T-cell responses is vital. For infectious disease vaccines, stimulation of both cellular immunity and neutralising antibodies are important.

The immune system is a powerful defence mechanism that can be employed to combat harmful pathogens and malignancies. The adaptive immune system in particular can be educated through vaccination to target altered, mutated or over-expressed self-antigens that are characteristic of malignant disease and invading foreign pathogens such as viruses.

The concept that the immune system can recognise and eliminate cancerous cells is referred to as immune surveillance [1, 2]. Immunological differences between cancer and self can be detected naturally and lead to elimination of cancerous cells in many individuals. This theory suggests that the immune system is naturally capable of detecting and eliminating cancer cells. Studies in immunocompetent mice show rejection of tumours and the protection gained can be adoptively transferred to mice via transfer of the T cells [3]. In mice, knockout models showed that interferon gamma (IFNγ) and lymphocytes are important in reducing the incidence of carcinogen-induced sarcoma and spontaneous epithelial carcinomas [4]. Patients with spontaneously regressing melanoma showed signs of tumour-specific clonal T cell expansion providing evidence of immune surveillance [5]. Recent work has demonstrated that the mutations accrued by tumours during the transformation process can generate neo-antigens that can be efficiently targeted by T cells [6]. This work all highlights that the fact that the immune system is capable of distinguishing between tumour and self but cancer still develops in many immunologically healthy individuals. This is due to the high mutation frequency of tumours which can lead to immune equilibrium where the tumour avoids the original immune response but the immune response can adapt and still attack the tumour. Eventually the tumour escapes in a process termed “immune editing” and grows despite the immune response [7].

Immune therapies seek to recruit immune cells into the tumour microenvironment and enhance anti-tumour immunity. There are two major reasons that could prevent this from occurring. Firstly, tumours do not present the correct environmental conditions to prime an effective immune response. In the absence of co-stimulation induced by signals such as Toll-like receptors (TLRs) or damage-associated molecular pattern molecules (DAMPS) to stimulate danger or sufficient presentation of tumour specific epitopes to prime responses, high avidity T cells capable of tumour lysis are not generated [8, 9]. The stimulation of low avidity T cells that cannot kill tumour cells is believed to be the reason for the failure of many early tumour vaccines. Strategies to address the lack of T cell priming and induce tumour-specific immune responses include vaccines. Efficient anti-cancer immunotherapy relies on effective targeting of antigens that can be recognised by high avidity T cells. Many antigens expressed on tumours are also expressed on normal tissues and the T cells recognising them are subject to thymic tolerance. This leaves a repertoire of low avidity T cells that can be stimulated by high doses of immunogen but which can never see sufficient target on the tumour cells to kill them. The goal is to find antigens that are not thymically expressed and are found in high abundance on tumour cells but not healthy tissues.

Viruses can be broadly classified as being either ‘non-enveloped’ or ‘enveloped’. Functionally, the viral envelope enables entry of a virus into its host. Viral glycoproteins on the envelope surface recognise and bind to receptor sites on the host's cell membrane. This leads to fusion of the viral envelope with the host's membrane, entry and release of the viral genome and infection of the host. Early in a viral infection both CD4 and CD8 T-cell responses are generated to locate and kill virally infected cells and prevent further replication of the virus. Later in the infection virus neutralising antibodies (VNabs) are produced to help prevent reinfection. The combination of memory T-cell responses and VNabs are both important in preventing new infections to the same or related viruses. The same phenomenon also applies to other pathogen infections Vaccines need to stimulate an immune response prior to exposure to the virus/pathogen and are designed to enable the host to respond quickly and efficiently to remove low viral load and to prevent any morbidity associated with the virus. As such, a vaccine needs to stimulate high avidity T-cells that recognise low antigen load and neutralising antibodies which prevent viral entry into a cell. T-cells can recognise viral proteins as they will all be presented in the context MHC on the cell surface of infected cells. However, the neutralising antibodies need to bind to the viral proteins which contact receptors on host cells and allow their entry into the cell. The most efficient viral vaccines are attenuated viruses which stimulate potent T cells and antibody responses but are associated with a low morbidity. There are already several licensed attenuated virus vaccines including smallpox, measles and polio. However, many viruses have evolved to evade immune recognition and are therefore not suitable as attenuated viral vaccines. This may be overcome by using an inactivated virus which is achieved using chemicals such as formaldehyde or heat inactivation. Such vaccines can stimulate antibody responses but require large quantities of the virus/pathogen and are very poor at stimulating high avidity T-cells response. A similar approach is to use virus like particles which assemble and look like viruses but cannot replicate. Again, these vaccines induce strong antibody responses and have been licensed to prevent HPV infection. An alternative to an attenuated virus is to use a hybrid virus. Viruses such as measles or adenovirus are genetically modified to produce proteins from a heterologous virus. These viruses are weakened or disabled so that they cannot cause disease; they can either still replicate within cells or they have had genes deleted that render them incapable of replicating. The viral vectors tend to have a good safety profile and an Ebola vaccine has recently been approved. However, the existence of any pre-existing immunity to the viral vector (measles, adenovirus) may impact their effectiveness and limits the ability to boost waning immune responses. They can also be only used for a single virus as once an immune response to the carrier virus has been established they cannot be used as a carrier for a new virus. The advantage of viral vaccines is that they can generate a high level of protein expression, inducing a strong antibody response. This can also be achieved using protein vaccines. However, protein vaccines also require adjuvants to simulate a potent immune response; in addition multiple doses are often required. Both protein and heterologous viral vaccines produce high levels of antigen but this stimulates low avidity T cell responses that will only kill cells with a high viral load which is dangerous as these cells may be lysed by the virus and spill large quantities of virus into the host before the low avidity T-cell has had time to react.

U.S. Pat. No. 7,067,110B1 discloses the use of an Fc-antigen fusion protein whereby whole antigen or antigen domains are fused to the hinge-CH2-CH3 domains of an antibody can enhance both antibody and cellular immunity. WO 2002/058728 discloses that targeting FcγRI with a polypeptide human IgG1 Fc fused to an antigen can stimulate high avidity T-cell responses. WO2008/116937 discloses a nucleic acid which comprises a non-specific promoter and at least one sequence that encodes a recombinant heavy chain of an immunoglobulin molecule, wherein the heavy chain has at least one heterologous T cell epitope therein such that the heavy chain cannot take its native conformation when the nucleic acid is expressed. Disruption of the primary antibody structure, inhibition of folding and/or limiting secretion to either just heavy chain or very low amounts of intact antibody, stimulates strong helper and antigen-specific T cell responses.

In a first aspect, the present invention provides a nucleic acid which encodes a polypeptide comprising:
(i) a modified Fc region of a human IgG1, and
(i) at least one heterologous antigen,
   wherein (a) the modified Fc region comprises at least the part of Fc that is capable of binding to CD64, (b) at least one residue of the modified Fc region is modified to the corresponding residue from a mouse IgG3 antibody and (c) the modified Fc region has enhanced avidity for Fc-gamma receptor (FcγR) when compared to the corresponding wildtype Fc region.

In a second aspect, the present invention provides a nucleic acid which encodes a polypeptide comprising:
(i) a modified Fc region of a human IgG1, and
(i) at least one heterologous antigen,
   wherein (a) the modified Fc region comprises at least the part of Fc that is capable of binding to TRIM21, (b) at least one residue of the Fc region is modified to the corresponding residue from a mouse IgG3 antibody and (c) the modified Fc region has enhanced avidity for Fc-gamma receptor (FcγR) when compared to the corresponding wildtype Fc region.

In a third aspect, the present invention provides a nucleic acid which encodes a polypeptide comprising:
(i) a modified Fc region of a human IgG1, and
(i) at least one heterologous antigen,
   wherein (a) the modified Fc region comprises at least the part of Fc that is capable of binding to CD64 and/or TRIM21, (b) at least one residue of the Fc region is modified to the corresponding residue from a mouse IgG3 antibody and (c) the modified Fc region has enhanced avidity for Fc-gamma receptor (FcγR) when compared to the corresponding wildtype Fc region.

In a fourth aspect, the present invention provides a vector comprising the nucleic acid of the first aspect.

In a fifth aspect, the present invention provides a polypeptide encoded by the nucleic acid of the first aspect or a vector of the second aspect.

The inventors have unexpectedly found that transferring certain mouse IgG3 (mIgG3) Fc residues into the hIgG1 Fc region of an antigen-Fc fusion protein improves the immunogenicity of the antigen. MIgG3 is the only isotype among the mIgGs that forms non-covalent oligomers, strongly influencing their biological activity [10], and increasing functional affinity to polyvalent antigens. Non-covalent interactions between adjacent mIgG3 Fc regions are thought to underpin this increased functional affinity, via prolonging target occupancy and reducing dissociation rates [11, 12]. The tendency of mIgG3 oligomerisation was initially identified by Grey et al. [13] who showed that binding to multivalent antigens promoted mIgG3 intermolecular interactions, which resulted in increased functional affinity to antigen [14, 15], a characteristic termed ‘intermolecular cooperativity’ [12, 15, 16]. It was determined that the phenomenon depended on Fc as IgG3 F(ab′)2 fragments did not bind to the antigen cooperatively [14]. It is surprising that improved immunogenicity is seen in the present invention as oligomerised hIgG1 Fc would not be expected to bind to FcγRI on dendritic cells. Unexpectedly the inventors have shown that Fc modification in accordance with the invention improves both the cellular and humoral response to the heterologous antigen(s). This suggests that enhanced avidity for FcγRI results in more efficient antigen presentation. In addition, as the Fc modified polypeptide of the invention binds initially in a monomeric form, it cannot bind to the low affinity FcRIIb and FcRIIIb inhibitory receptors which would result in inhibition of immune responses. The creation of an improved vaccine, with enhanced immunogenicity, through establishing intermolecular cooperativity binding, may lead to superior clinical utility.

The modified Fc region may have avidity for Fc-gamma receptor (FcγR), preferably FcγRI, that is enhanced by at least about 10% when compared to a corresponding wildtype human IgG1 Fc region. The modified Fc region may have avidity for Fc-gamma receptor (FcγR), preferably FcγRI, that is enhanced by at least about 20%, at least about 30%, at least about 40%, at least about 50%, at least about 60%, at least about 70%, at least about 80%, at least about 90% or at least about 100% when compared to a corresponding wildtype human IgG1 Fc region.

The polypeptide of the present invention preferably has enhanced immunogenicity and/or non-covalent oligomerisation when compared to a corresponding peptide comprising an unmodified wildtype human IgG1 region and at least one heterologous antigen.

The immunogenicity and/or non-covalent oligomerisation of the polypeptide of the present invention may be enhanced by at least about 20%, at least about 30%, at least about 40%, at least about 50%, at least about 60%, at least about 70%, at least about 80%, at least about 90% or at least about 100% when compared to a corresponding polypeptide comprising an unmodified wildtype human IgG1 region and at least one heterologous antigen.

In the present invention, the modified Fc region comprises the part of Fc that binds to CD64 and/or TRIM21. It may comprise CH2 and CH3, and optionally may further comprise the hinge region. One or more residues of the human IgG1 CH2 and/or the CH3 domain may be replaced with the corresponding residues from the mouse IgG3 CH2 and/or CH3 domain. In some aspects of the invention, the at least one residue of the Fc region is selected from the CH2 and/or the CH3 domains. In some aspects, the at least one residue is selected from the CH2 domain. In some aspects, the at least one residue is selected from the CH3 domain.

Dissection of the combined CH2-CH3 region through subdomain analysis has revealed that it is possible to enhance avidity of the Fc region of a human IgG1 for FcγR by transferring a discontinuous section comprising mIgG3 residues 286-306 and 339-378-23 residues in total—containing elements of CH2 and CH3. The 23 residues are: N286T, K288W, K290Q, A339P, Q342R, P343A, R344Q, E345T, L351I, S354P, D356E, E357Q, L358M, T359S, N361K, Q362K, K370T, G371N, Y373F, P374S, S375E, D376A, A378S. These residues are required for increased non-covalent oligomerisation through intermolecular cooperativity due to the combined effect of directly interacting as well as conformational residues, the latter potentially creating a permissive framework. Further details of the modification to the Fc region are set out in PCT/EP2020/071724, the contents of which are fully incorporated by reference.

The Fc region of a human IgG1 may have modifications to one or more of the following residues of the Fc region: N286, K288, K290, A339, Q342, P343, R344, E345, L351, S354, D356, E357, L358, T359, N361, Q362, K370, G371, Y373, P374, S375, D376, A378. The modifications may be one or more of N286T, K288W, K290Q, A339P, Q342R, P343A, R344Q E345T, L351I, S354P, D356E, E357Q L358M, T359S, N361K, Q362K, K370T, G371N, Y373F, P374S, S375E, D376A, A378S.

The modified Fc region of a human IgG1 may comprise modifications at 1, 2, 3, 4, 5, 6, 7, 8, 9, 10, 11, 12, 13, 14, 15, 16, 17, 18, 19, 20, 21, 22 or 23 residues selected from positions N286, K288, K290, A339, Q342, P343, R344, E345, L351, S354, D356, E357, L358, T359, N361, Q362, K370, G371, Y373, P374, S375, D376, A378. Preferably, the modified Fc region of a IgG1 antibody comprises modifications at all 23 residues.

The modified Fc region of a human IgG1 may comprise 1, 2, 3, 4, 5, 6, 7, 8, 9, 10, 11, 12, 13, 14, 15, 16, 17, 18, 19, 20, 21, 22 or 23 modifications selected from N286T, K288W, K290Q, A339P, Q342R, P343A, R344Q E345T, L351I, S354P, D356E, E357Q L358M, T359S, N361K, Q362K, K370T, G371N, Y373F, P374S, S375E, D376A, A378S. Preferably, the modified Fc region of a IgG1 antibody comprises all 23 modifications.

The modified Fc region may comprise modifications to one or more of the following residues of the Fc region: N286, K288, K290, Q342, P343, E345, L351, T359, N361, Q362, G371, P374, S375, D376, A378. The modifications may be one or more of N286T, K288W, K290Q, Q342R, P343A, E345T, L351I, T359S, N361K, Q362K, G371N, P374S, S375E, D376A, A378S.

The modified Fc region may comprise modifications at 1, 2, 3, 4, 5, 6, 7, 8, 9, 10, 11, 12, 13, 14 or 15 residues selected from positions N286, K288, K290, Q342, P343, E345, L351, T359, N361, Q362, G371, P374, S375, D376, A378. Preferably the modified Fc region of a human IgG1 antibody comprises modifications at all 15 residues.

The modified Fc region may comprise 1, 2, 3, 4, 5, 6, 7, 8, 9, 10, 11, 12, 13, 14 or 15 modifications selected from N286T, K288W, K290Q, Q342R, P343A, E345T, L351I, T359S, N361K, Q362K, G371N, P374S, S375E, D376A, A378S. Preferably the modified Fc region comprises all 15 modifications.

The modified Fc region may comprise modifications to one or more of the following residues of the Fc region: Q342, P343, E345, N361, Q362, P374, D376. The modifications may be one or more of Q342R, P343A, E345T, N361K, Q362K, P374S, D376A.

The modified Fc region may comprise modifications at 1, 2, 3, 4, 5, 6 or 7 residues selected from positions Q342, P343, E345, N361, Q362, P374, D376. Preferably, the modified Fc region comprises modifications at all 7 residues.

The modified Fc region may comprise 1, 2, 3, 4, 5, 6 or 7 modifications selected from Q342R, P343A, E345T, N361K, Q362K, P374S, D376A. Preferably, the modified Fc region comprises all 7 modifications.

The modified Fc region may comprise the amino acid sequence provided in SEQ ID NO: 1, or an amino acid sequence having at least 90% identity to SEQ ID NO: 1. SEQ ID NO: 1 is the amino acid sequence of an exemplary modified Fc region, “iv1” (see Table 4).

The structure of the polypeptide of the present invention may be of an antibody heavy chain sequence or substantial portion thereof. The structures and locations of immunoglobulin domains may be determined by reference to www.imgt.org/.

Throughout the specification the residue numbering refers to the standardised IMGT® system for the numbering of antibody sequences, as disclosed in Lefranc et al., 2009 [17]. Other suitable numbering systems are known to the skilled person. Other suitable numbering systems may be used to identify corresponding residues between the modified human IgG1 antibody-antigen fusion protein thereof. Any numbering system which allows identification of corresponding residues is suitable for use with the present invention. The numbering system used herein is not limiting on the scope of the invention, but is used simply to identify the relevant residues which may be modified. The term “corresponding residue” is intended to mean the residue in the equivalent position, structurally or functionally, in the two or more antibodies or antigen-binding fragments thereof that are being compared. In some cases, corresponding residues may be identified by sequence alignment. In some cases, corresponding residues may be identified by structural comparison.

In some aspects of the invention the polypeptide may comprise at least 10 amino acid residues of an Fc-region, at least 20 amino acid residues of an Fc-region, at least 30 amino acid residues of an Fc-region, at least 40 amino acid residues of an Fc-region, at least 50 amino acid residues of an Fc-region, at least 75 amino acid residues of an Fc-region, at least 100 amino acid residues of an Fc-region, at least 200 amino acid residues of an Fc-region, at least 300 amino acid residues of an Fc-region, at least 400 amino acid residues of an Fc-region or at least 500 amino acid residues of an Fc-region. Preferably the polypeptide comprises the entire Fc-region of human IgG1.

The at least one heterologous antigen may be linked (directly or via a linker) to the N-terminus of the modified human IgG1 Fc region. It is less preferred if the at least one heterologous antigen is linked (directly or via a linker) to the C-terminus of the modified human IgG1 Fc region. Where the heterologous antigen is a polypeptide, it may be linked to the modified Fc region at the N- or, more preferably, the C-terminus of the heterologous antigen. This format of polypeptide of the invention may be used where the heterologous antigen is a relatively large molecule, such as a viral or bacterial protein or immunogenic fragment thereof. In a preferred such format, the C-terminus of the heterologous antigen is linked, optionally via a linked, to the N terminus of the modified Fc region.

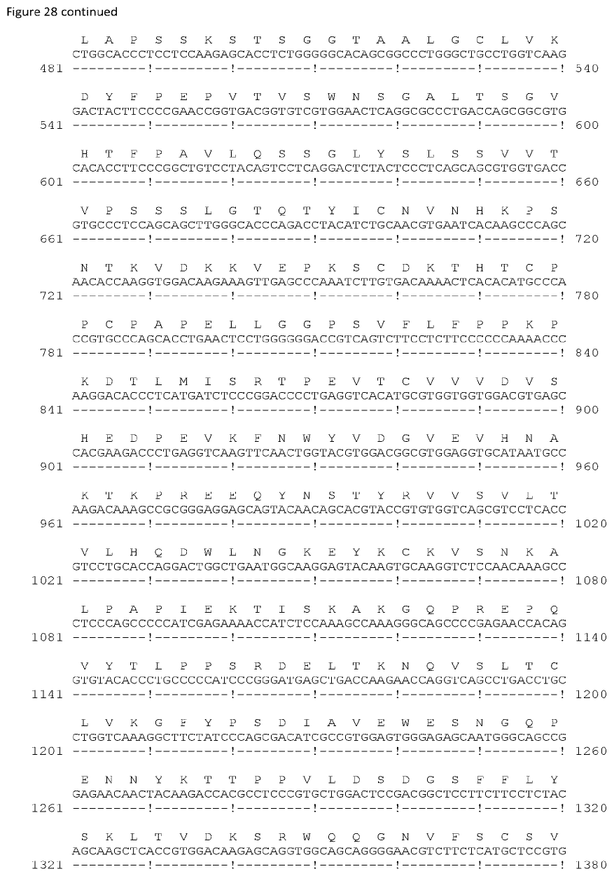

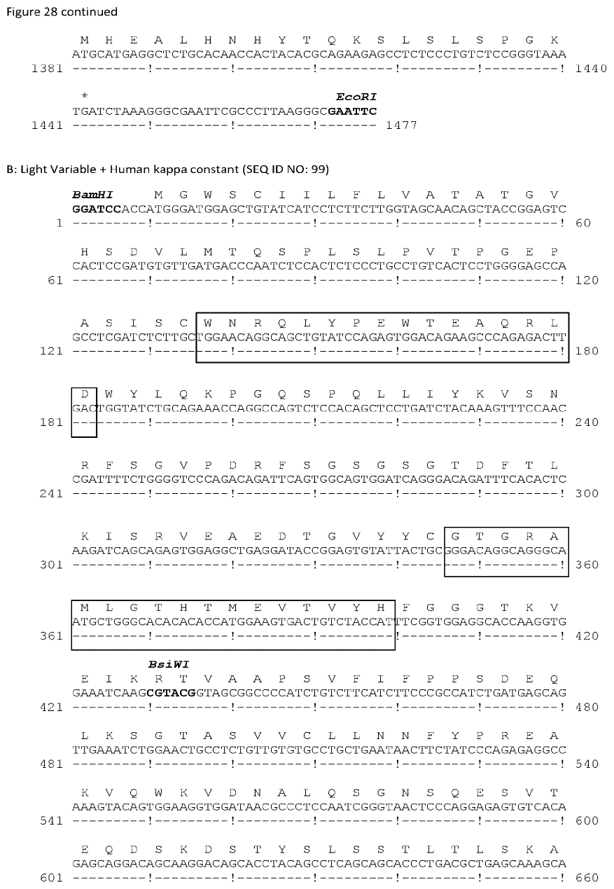

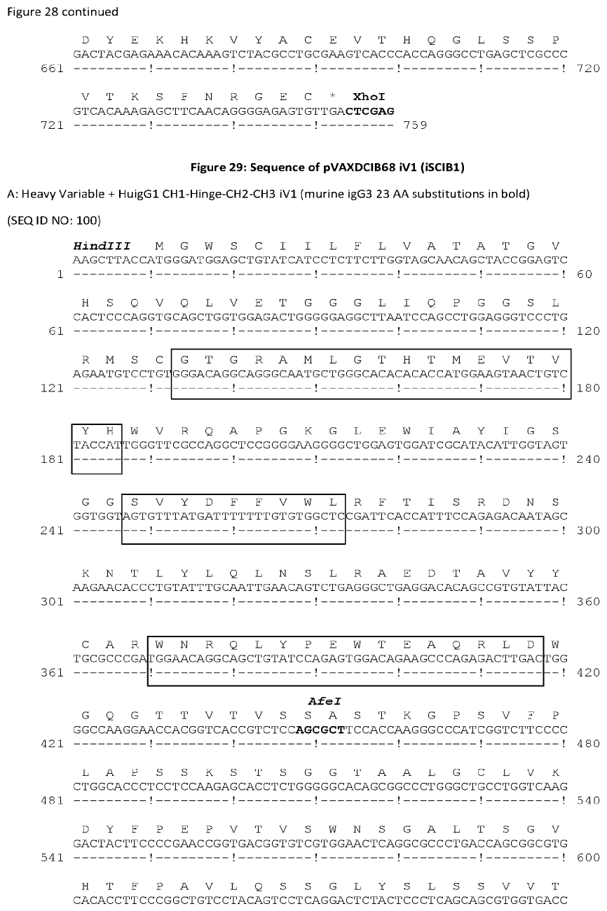

In some aspects of the invention the at least one heterologous antigen comprises one or more epitopes selected from the epitopes set out in any one of FIGS. 28-33.

In some aspects of the invention the at least one heterologous antigen comprises one or more epitopes selected from the epitopes set out in Table 2 or Table 3.

In some aspects of the invention the at least one heterologous antigen comprises one or more epitopes selected from:

(a)
(SEQ ID NO: 29)
GTGRAMLGTHTMEVTVYH;
(b)
(SEQ ID NO: 30)
SVYDFFVWL;
and
(c)
(SEQ ID NO: 31)
WNRQLYPEWTEAQRLD.

In some aspects of the invention the at least one heterologous antigen comprises one or more epitopes selected from:

(a)
(SEQ ID NO: 29)
GTGRAMLGTHTMEVTVYH;
(b)
(SEQ ID NO: 30)
SVYDFFVWL;
(c)
(SEQ ID NO: 31)
WNRQLYPEWTEAQRLD;
and
(d)
(SEQ ID NO: 32)
VPLDCVLYRYGSFSVTLDIVQG.

In some aspects of the invention the at least one heterologous antigen comprises one or more epitopes selected from:

(a)
(SEQ ID NO: 29)
GTGRAMLGTHTMEVTVYH;
(b)
(SEQ ID NO: 30)
SVYDFFVWL;
(c)
(SEQ ID NO: 31)
WNRQLYPEWTEAQRLD;
(d)
(SEQ ID NO: 32)
VPLDCVLYRYGSFSVTLDIVQG;
(e)
(SEQ ID NO: 33)
ANCSVYDFFVWLHYYSVRDTLLGPGRPYR;
and
(f)
(SEQ ID NO: 34)
QCTEVRADTRPWSGPYILRNQDDRELWPRKFF.

In some aspects of the invention the at least one heterologous antigen comprises one or more epitopes selected from:

(a)
(SEQ ID NO: 35)
LLMWITQCF;
(b)
(SEQ ID NO: 36)
SLLMWITQC;
(c)
(SEQ ID NO: 37)
PESRLLEFYLAMPFATPMEAELARRSLAQ;
and
(d)
(SEQ ID NO: 38)
PGVLLKEFTVSGNILTIRLTAADHR.

In some aspects of the invention the at least one heterologous antigen comprises one or more epitopes selected from:

(a)
(SEQ ID NO: 35)
LLMWITQCF;
(b)
(SEQ ID NO: 36)
SLLMWITQC;
(c)
(SEQ ID NO: 37)
PESRLLEFYLAMPFATPMEAELARRSLAQ;
(d)
(SEQ ID NO: 39)
PESRLLEFY;
(e)
(SEQ ID NO: 40)
RLLEFYLAMPFATP;
(f)
(SEQ ID NO: 41)
LEFYLAMPF;
(g)
(SEQ ID NO: 42)
EFYLAMPFATPM;
(h)
(SEQ ID NO: 43)
MPFATPMEA;
(i)
(SEQ ID NO: 44)
LAMPFATPM;
(j)
(SEQ ID NO: 45)
LLEFYLAMPFATPM;
(k)
(SEQ ID NO: 46)
LLEFYLAMPFATPMEAELARRSLAQ;
(l)
(SEQ ID NO: 38)
PGVLLKEFTVSGNILTIRLTAADHR;
(m)
(SEQ ID NO: 47)
LKEFTVSGNILTIRL;
(n)
(SEQ ID NO: 48)
KEFTVSGNILT;
(o)
(SEQ ID NO: 49)
KEFTVSGNILTI;
(p)
(SEQ ID NO: 50)
TVSGNILTIR;
and
(q)
(SEQ ID NO: 51)
TVSGNILTI.

In some aspects of the invention the at least one heterologous antigen comprises one or more epitopes selected from:

(a)
(SEQ ID NO: 52)
KIADYNYKL;
(b)
(SEQ ID NO: 53)
KLPDDFTGCV;
(c)
(SEQ ID NO: 54)
ELLHAPATV;
(d)
(SEQ ID NO: 55)
CPFGEVFNATRFASVTAWNR;
(e)
(SEQ ID NO: 56)
RISNCVADYSVLYNSASFST;
(f)
(SEQ ID NO: 57)
YLYRLFRKSNLKPFERDI;
(g)
(SEQ ID NO: 58)
YQPYRVVVLSFELLHAPATV;
(h)
(SEQ ID NO: 59)
ALNTPKDHI;
(i)
(SEQ ID NO: 60)
LQLPQGTTL;
(j)
(SEQ ID NO: 61)
LLLDRLNQL;
(k)
(SEQ ID NO: 62)
GMSRIGMEV;
(l)
(SEQ ID NO: 63)
ILLNKHIDA;
(m)
(SEQ ID NO: 64)
GNGGDAALALLLLDRLNQLE;
and
(n)
(SEQ ID NO: 65)
KHWPQIAQFAPSASAFFGMS.

As used herein, the term “immunogenic fragment” is a portion of an antigen or protein that is smaller than an entire antigen or protein and is capable of eliciting a humoral and/or cellular immune response specific to that fragment in a host animal (e.g., a human). Fragments of the protein can be produced using techniques known in the art, such as recombination, by proteolytic digestion, or by chemical synthesis. An internal or terminal fragment of a polypeptide can be generated by removing one or more nucleotides from one end (for a terminal fragment) or both ends (for an internal fragment) of a nucleic acid encoding a polypeptide.

Linker sequences are usually flexible, in that they are made up primarily of amino acids such as glycine, alanine and serine, which do not have bulky side chains likely to restrict flexibility. Alternatively, linkers with greater rigidity may be desirable. Usable or optimum lengths of linker sequences may be easily determined. Often the linker sequence will be less than about 12, such as less than 10, or from 2-10 amino acids in length. The linker may be 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 amino acids in length. Examples of suitable linkers that may be used include, but are not limited to: GGGGS (SEQ ID NO: 106), GGGSG (SEQ ID NO: 107), GGSGG (SEQ ID NO: 108), GSGGG (SEQ ID NO: 109), GSGGGP (SEQ ID NO: 110), GGEPS (SEQ ID NO: 111), GGEGGGP (SEQ ID NO: 112), GGEGGGSEGGGS (SEQ ID NO: 113) and GGGSGGGG (SEQ ID NO: 114). Additional linkers may include sequences having one or more of the following sequence motifs: GGGS (SEQ ID NO: 115), TVLRT (SEQ ID NO: 116), TVSSAS (SEQ ID NO: 117) and TVLSSAS (SEQ ID NO: 118). A preferred linker used in the present invention is the Ig hinge.

Alternatively, the polypeptide of the present invention may comprise an antibody variable region into which the or each heterologous antigen is inserted or substituted. The polypeptide of the present invention may comprise a human IgG1 heavy chain comprising the modifications in the Fc region thereof. It is preferred if the or each heterologous antigen is substituted into one or more of the CDRs of the variable region. Although all CDRs can be used for substitution for a heterologous antigen, a preferred CDR is CDR3. This format of polypeptide of the invention may be used where the heterologous antigen is, for example, a cancer antigen.

The antibody variable may be a heavy chain variable region comprising the following heterologous antigens substituted into the CDR1, CDR2 and CDR3 respectively:

(a)
(SEQ ID NO: 29)
GTGRAMLGTHTMEVTVYH,
(SEQ ID NO: 30)
SVYDFFVWL
and
(SEQ ID NO: 32)
VPLDCVLYRYGSFSVTLDIVQG;
or
(b)
(SEQ ID NO: 35)
LLMWITQCF,
(SEQ ID NO: 36)
SLLMWITQC
and
(SEQ ID NO: 37)
PESRLLEFYLAMPFATPMEAELARRSLAQ.

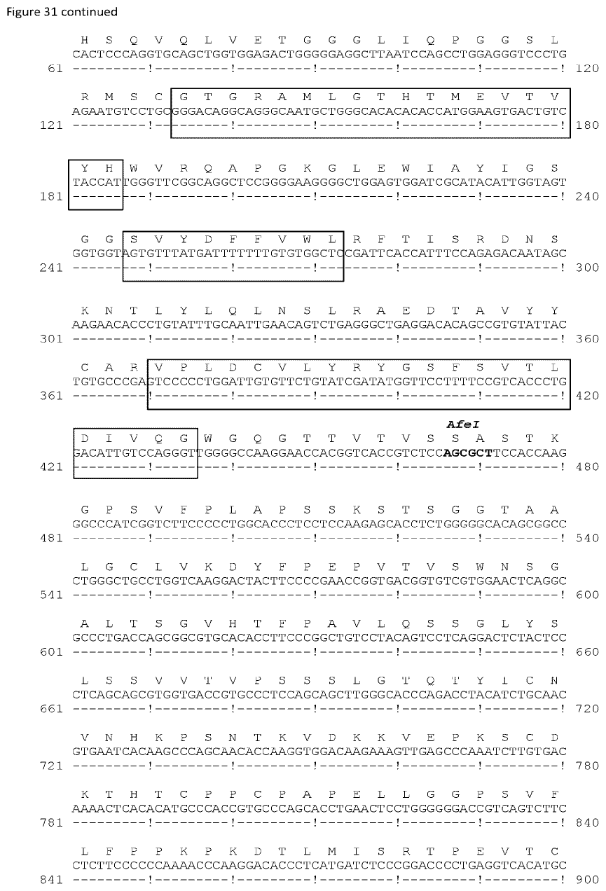

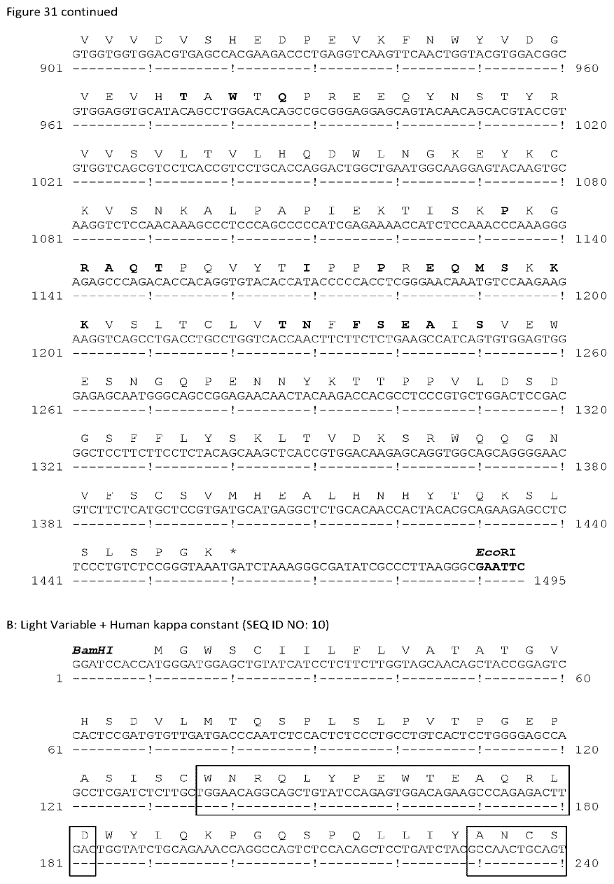

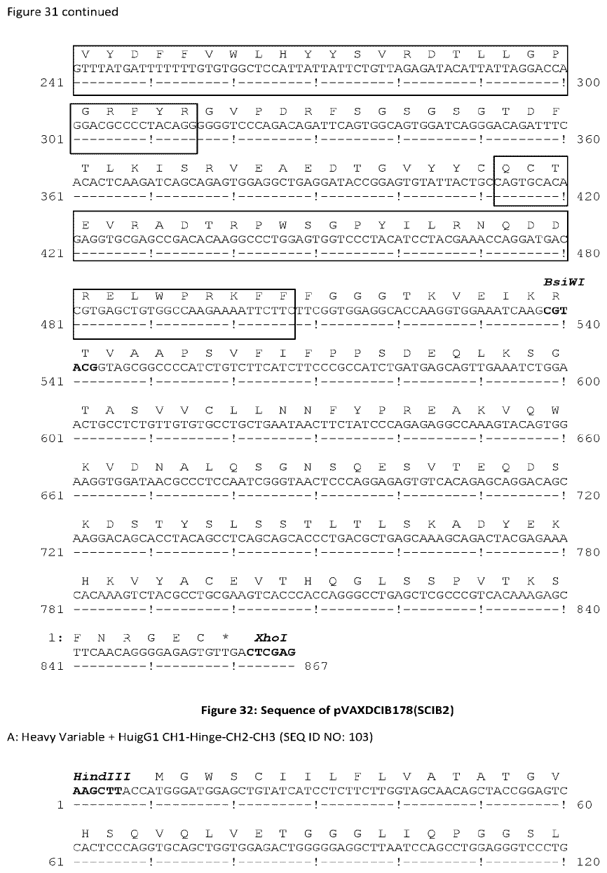

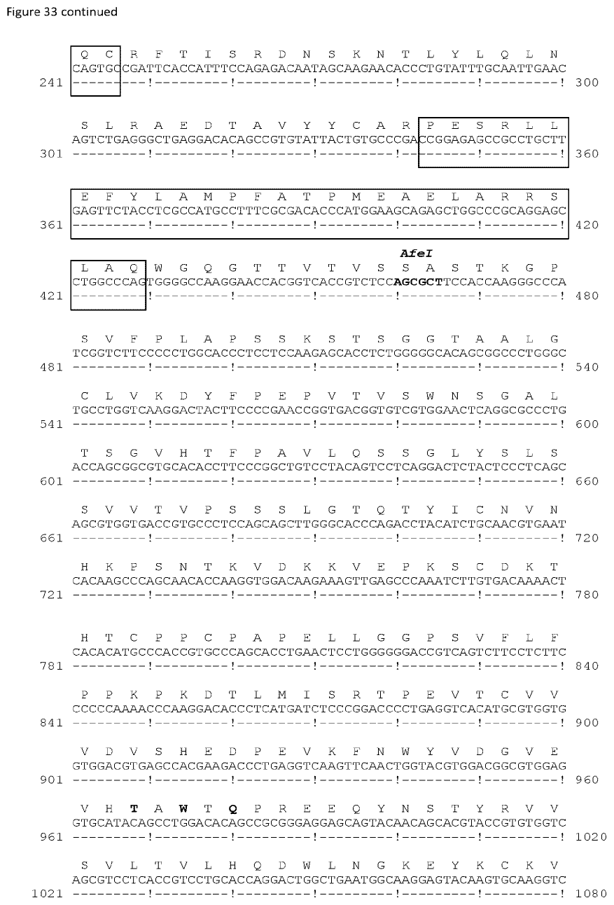

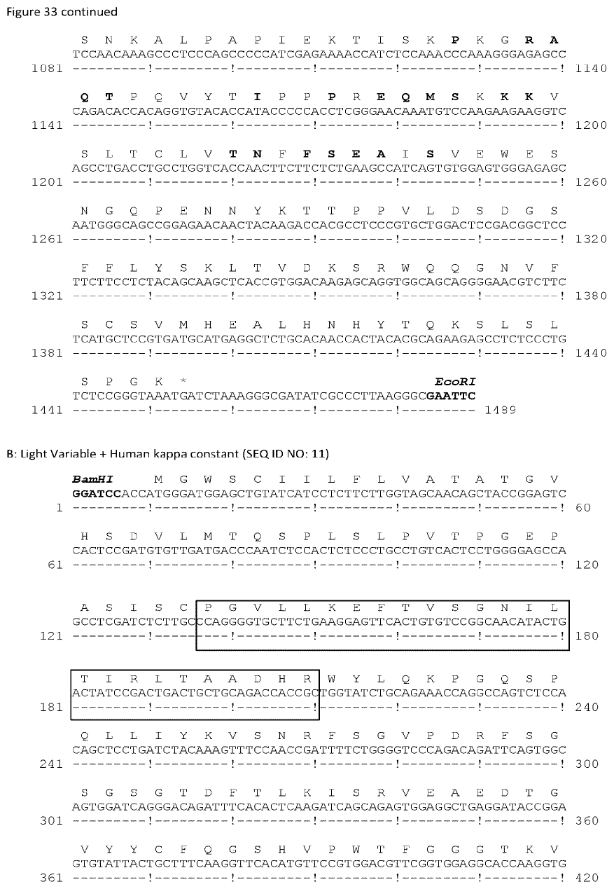

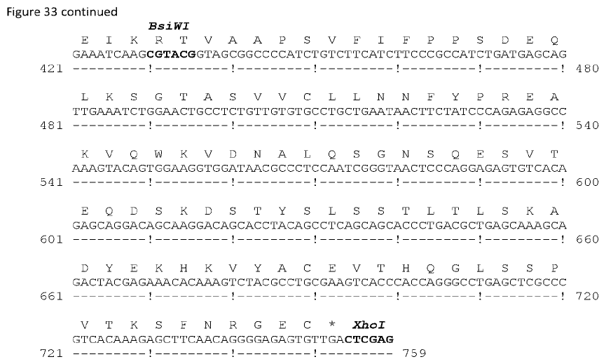

The polypeptide encoded by the nucleic acid of the invention may comprise the amino acid sequence provided in SEQ ID NO: 2 or in SEQ ID NO: 3. SEQ ID NO: 2 and SEQ ID NO: 3 are the amino sequences of the whole antibody heavy chain encoded by the iSCIB1plus (see FIG. 31) and iSCIB2 (see FIG. 33) vectors respectively.

The heterologous antigen may be the N protein of a coronavirus or an immunogenic fragment thereof. Preferably, the N protein is from SARS-CoV-2. For example, the N protein may be from lineage A Wuhan strain SARS-CoV-2, B.1.351 variant SARS-CoV-2 or B.1.617.2 variant SARS-CoV-2. The N protein may comprise the amino acid sequence provided in SEQ ID NO:4 (Wuhan strain). The N protein may comprise the amino acid sequence provided in SEQ ID NO:5 (B.1.351 variant). The N protein may comprise the amino acid sequence provided in SEQ ID NO:26 (B.1.1.7 variant).

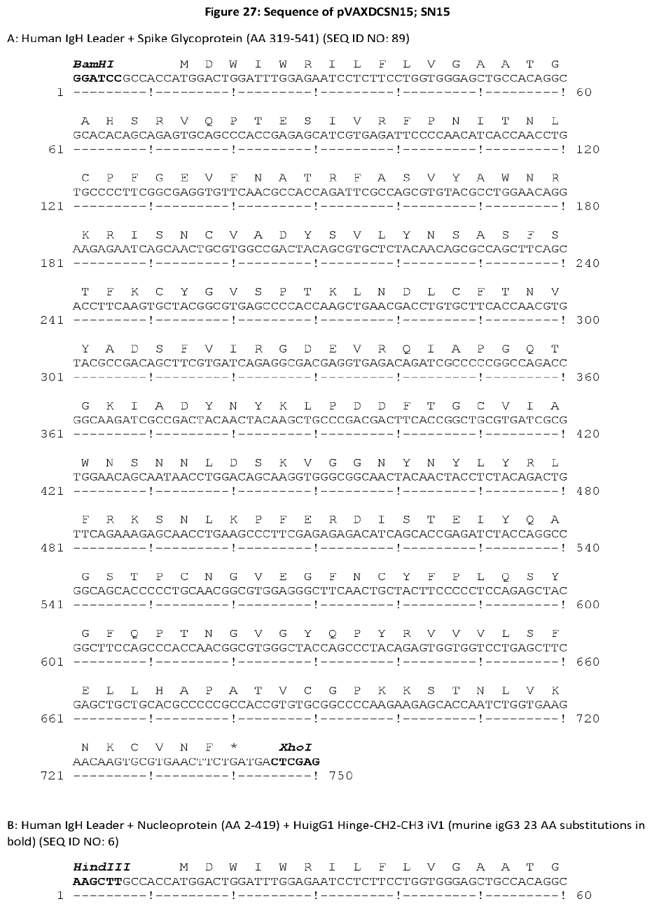

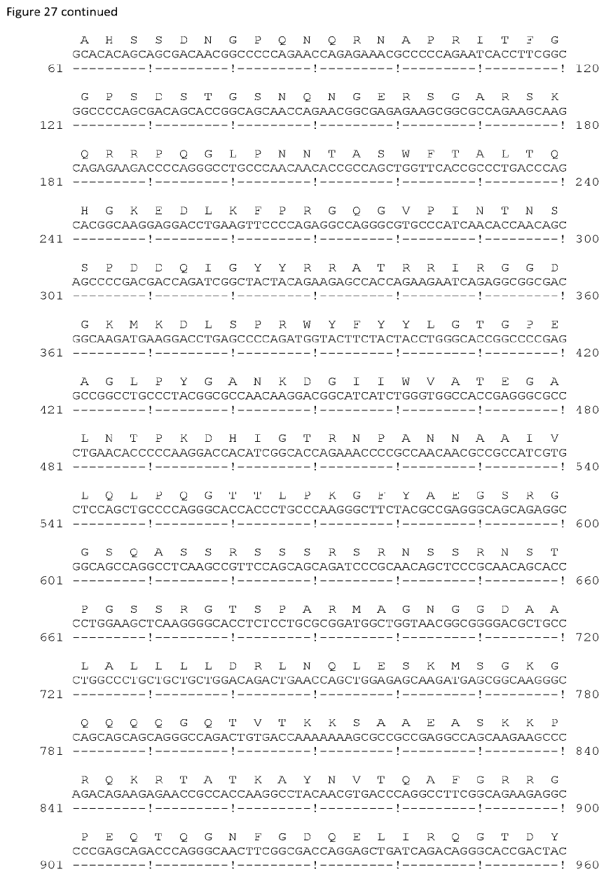

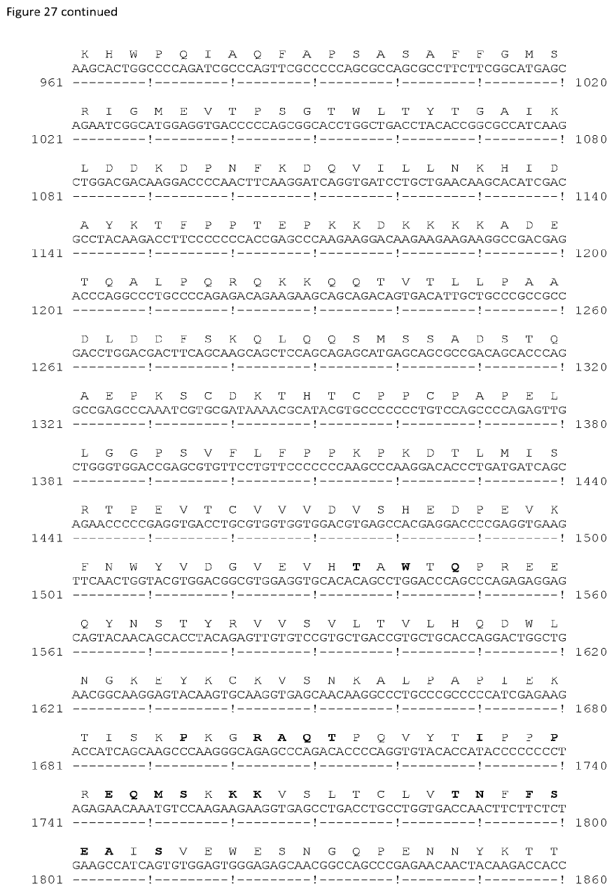

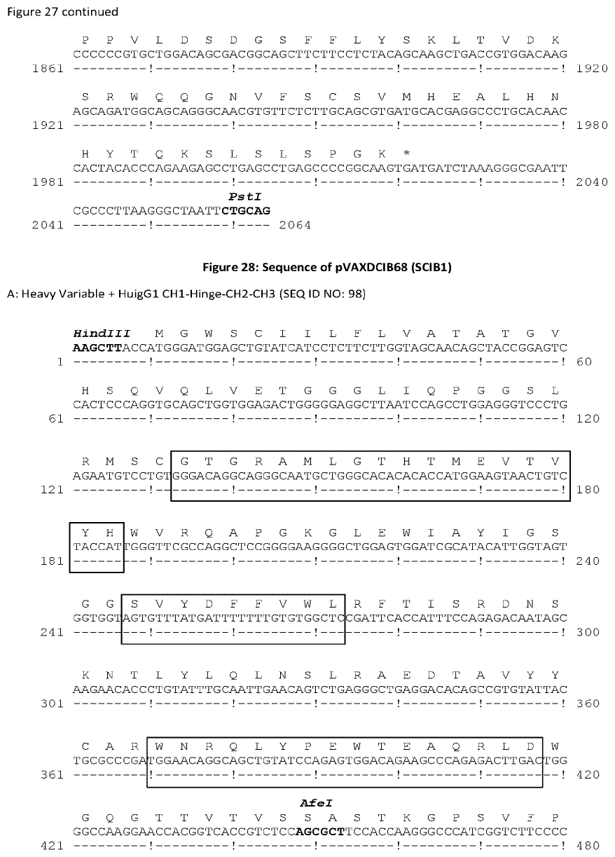

Preferably, the polypeptide encoded by the nucleic acid of the invention comprises the amino acid sequence provided in SEQ ID NO: 6. SEQ ID NO: 6 is the amino acid sequence of the N protein (Wuhan strain) fused to the iv1 modified Fc region encoded by the “SN15” vector (see FIG. 27).

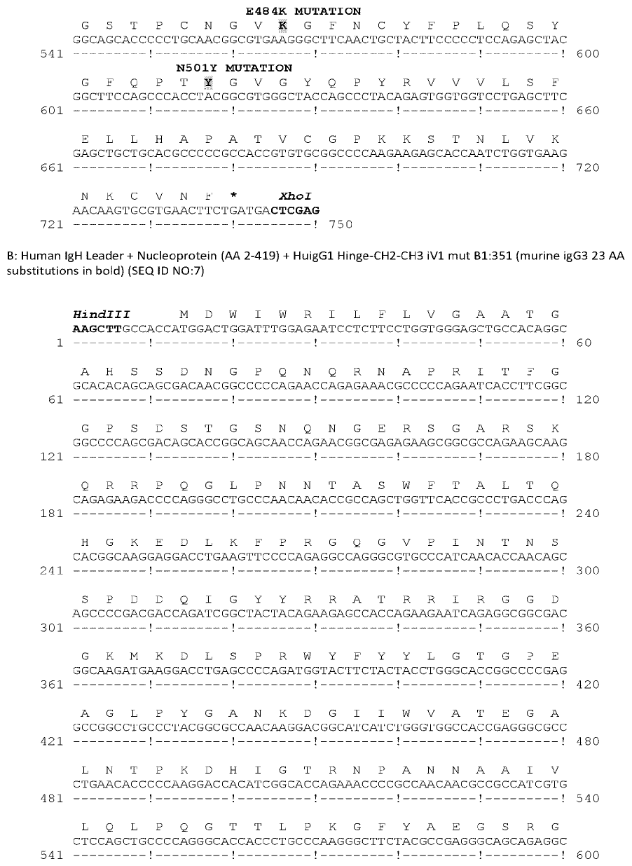

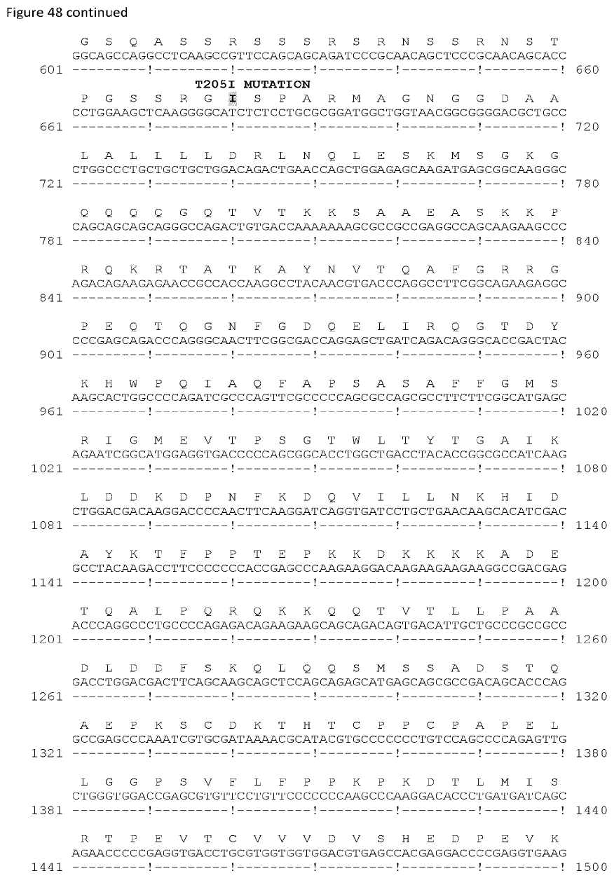

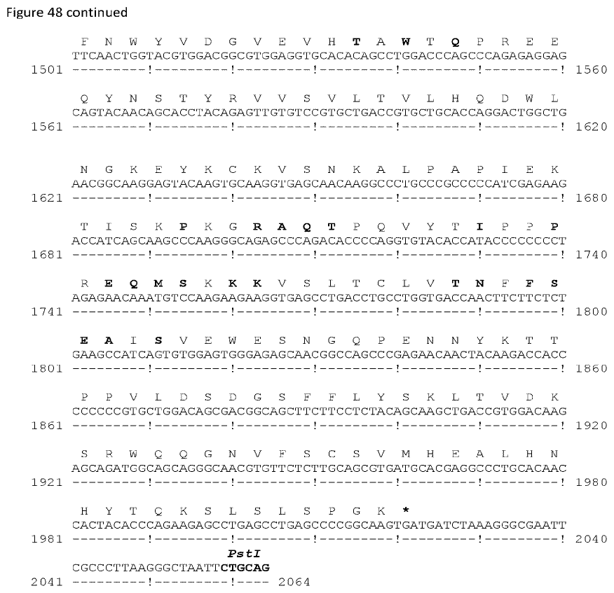

Preferably, the polypeptide encoded by the nucleic acid of the invention comprises the amino acid sequence provided in SEQ ID NO: 7. SEQ ID NO: 7 is the amino acid sequence of the N protein (B.1.351 variant) fused to the iv1 modified Fc region encoded by the “SN17” vector (see FIG. 48).

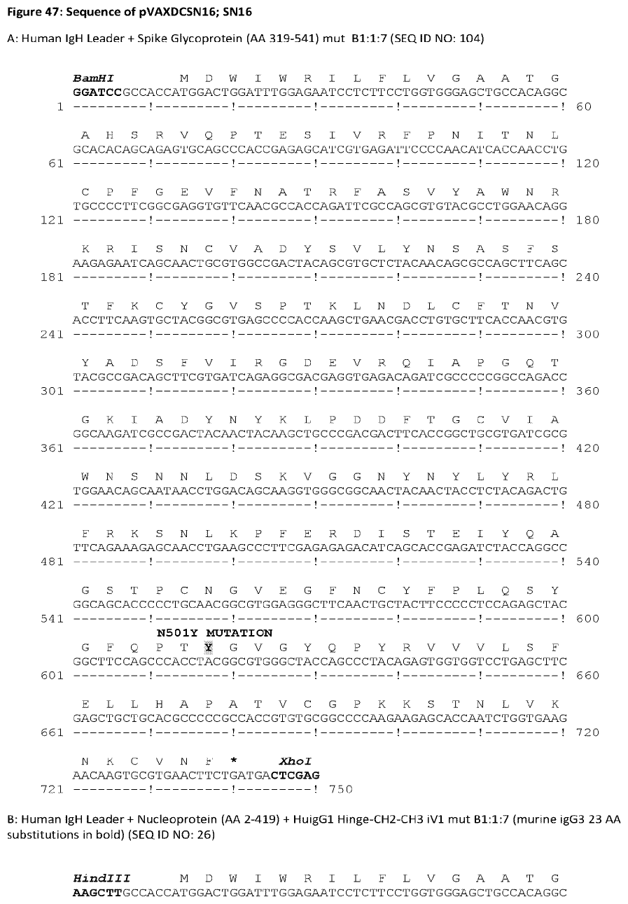

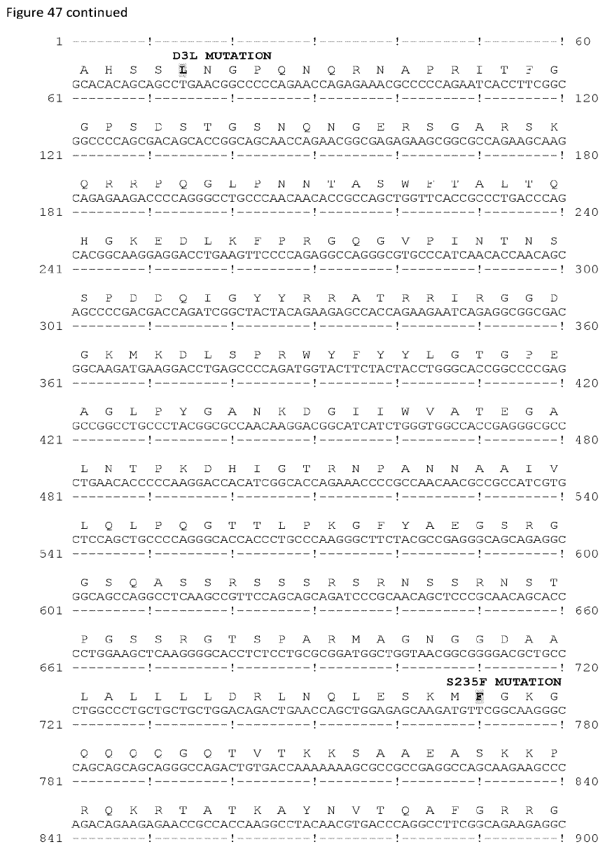

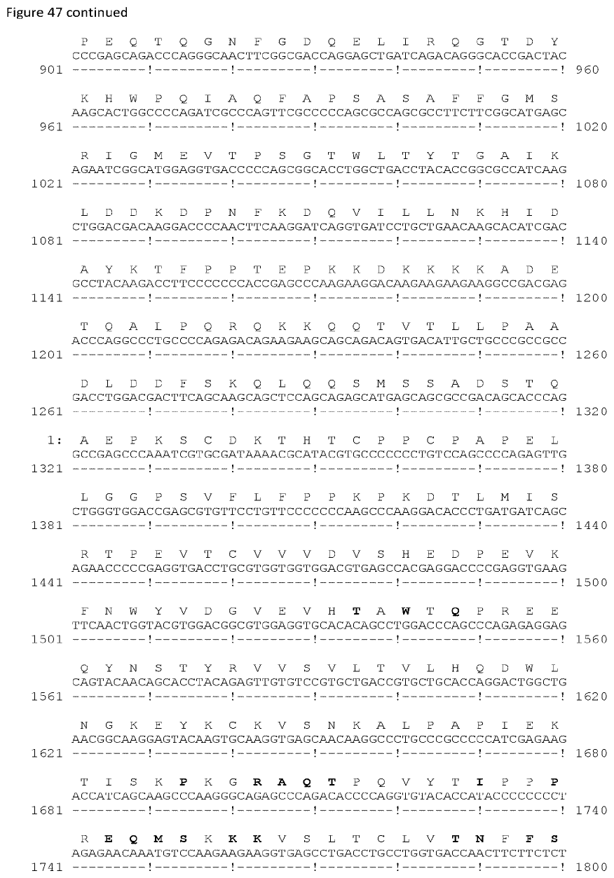

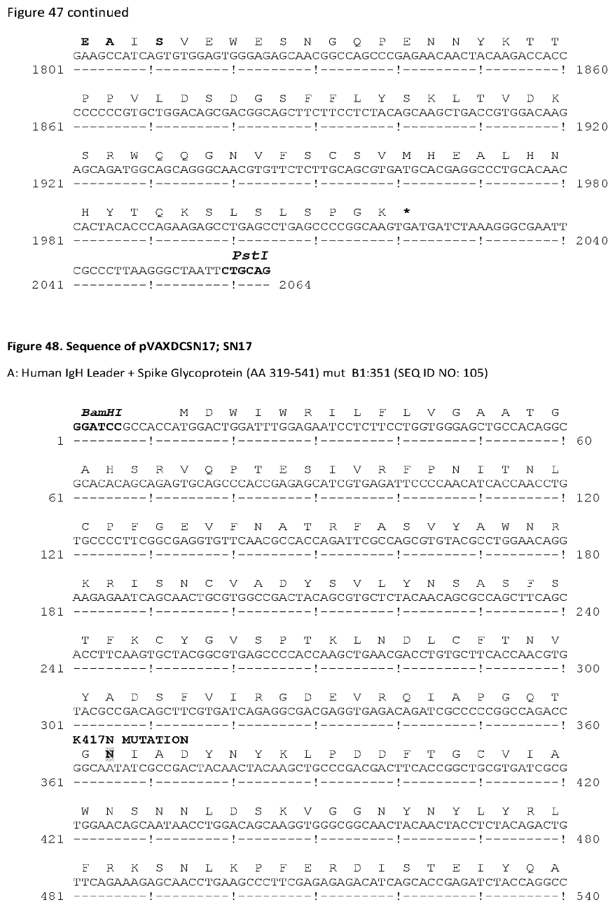

The polypeptide encoded by the nucleic acid of the invention may comprise the amino acid sequence provided in SEQ ID NO: 27. SEQ ID NO: 27 is the amino acid sequence of the N protein (B.1.1.7 variant) fused to the iv1 modified Fc region encoded by the “SN16” vector (see FIG. 47).

The nucleic acid of the invention may be provided in combination (separately or linked) with a second nucleic acid encoding a second polypeptide comprising at least one heterologous antigen. The second polypeptide may be an antibody light chain. The light chain may have one or more heterologous antigens inserted or substituted therein. The or each heterologous antigen may be substituted into one or more of the CDRs of the antibody light chain. Although all CDRs can be used for substitution for a heterologous antigen, a preferred CDR is CDR3.

The antibody light chain encoded by the second nucleic acid may comprise the following heterologous antigens substituted into the CDR1, CDR2 and CDR3 respectively:

(SEQ ID NO: 31)
WNRQLYPEWTEAQRLD,
(SEQ ID NO: 33)
ANCSVYDFFVWLHYYSVRDTLLGPGRPYR
and
(SEQ ID NO: 34)
QCTEVRADTRPWSGPYILRNQDDRELWPRKFF.

The antibody light chain encoded by the second nucleic acid may comprise the sequence PGVLLKEFTVSGNILTIRLTAADHR (SEQ ID NO: 38) substituted into the CDR2.

The antibody light chain encoded by the second nucleic acid may comprise the amino acid sequence provided in SEQ ID NO: 10 or SEQ ID NO: 11. SEQ ID NOs: 10 and 11 are the amino acid sequences of the antibody light chains encoded in the iSCIB1plus (FIG. 31) and iSCIB2 (FIG. 33) vectors respectively.

Preferably, the polypeptide encoded by the nucleic acid of the invention comprises the amino acid sequence provided in SEQ ID NO: 2 and the antibody light chain encoded by the second nucleic acid comprises the amino acid sequence provided in SEQ ID NO: 10.

Preferably, the polypeptide encoded by the nucleic acid comprises the amino acid sequence provided in SEQ ID NO: 3 and the antibody light chain encoded by the second nucleic acid comprises the amino acid sequence provided in SEQ ID NO: 11.

In some aspects of the invention the second nucleic acid encodes the receptor binding domain of SARS-Cov-2. The receptor binding domain may comprise the amino acid sequence provided in SEQ ID NO: 8 (Wuhan strain RBD). The receptor binding domain may comprise the amino acid sequence provided in SEQ ID NO: 9 (B.1.351 variant RBD). The receptor binding domain may comprise the amino acid sequence provided in SEQ ID NO: 28 (B.1.1.7 variant RBD).

Preferably, the polypeptide encoded by the nucleic acid of the invention comprises the amino acid sequence provided in SEQ ID NO: 6 and the second nucleic acid encodes a receptor binding domain comprising the amino acid sequence provided in SEQ ID NO: 8.

Preferably, the polypeptide encoded by the nucleic acid comprises the amino acid sequence provided in SEQ ID NO: 7 and the second nucleic acid encodes a receptor binding domain comprising the amino acid sequence provided in SEQ ID NO: 9.

The polypeptide encoded by the nucleic acid may comprise the amino acid sequence provided in SEQ ID NO: 27, and the second nucleic acid encodes a receptor binding domain comprising the amino acid sequence provided in SEQ ID NO: 28.

The polypeptide encoded by the nucleic acid sequence of the invention may comprise an amino acid sequence that is at least 90% identical to any one of the above recited sequences. For example, the polypeptide encoded by the nucleic acid sequence may comprise an amino acid sequence that is at least 90%, at least 91%, at least 92%, at least 93%, at least 94%, at least 95%, at least 96%, at least 97%, at least 98%, or at least 99% identical to any one of the above recited sequences.

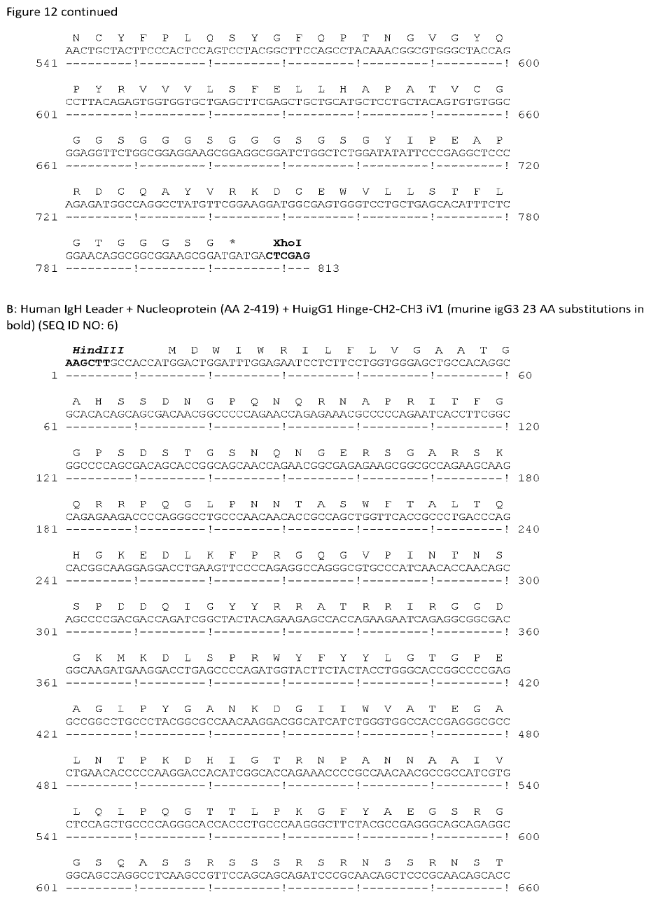

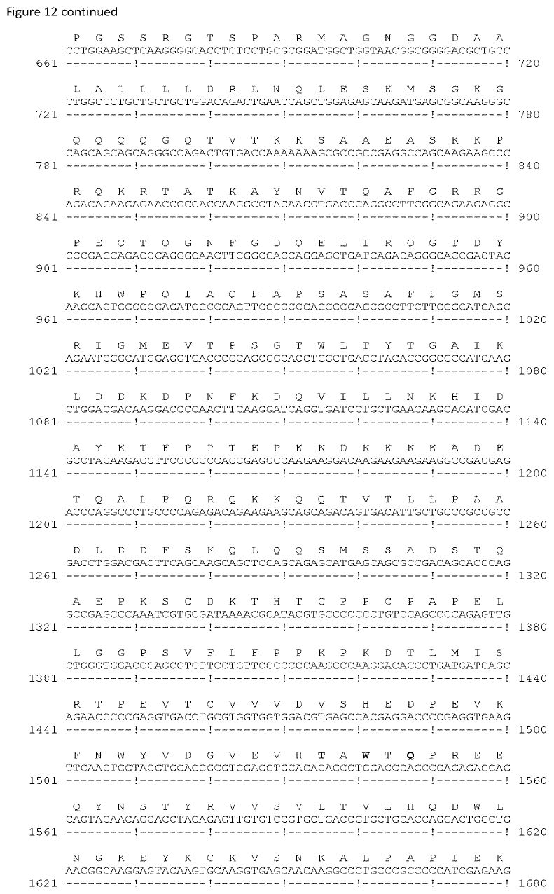

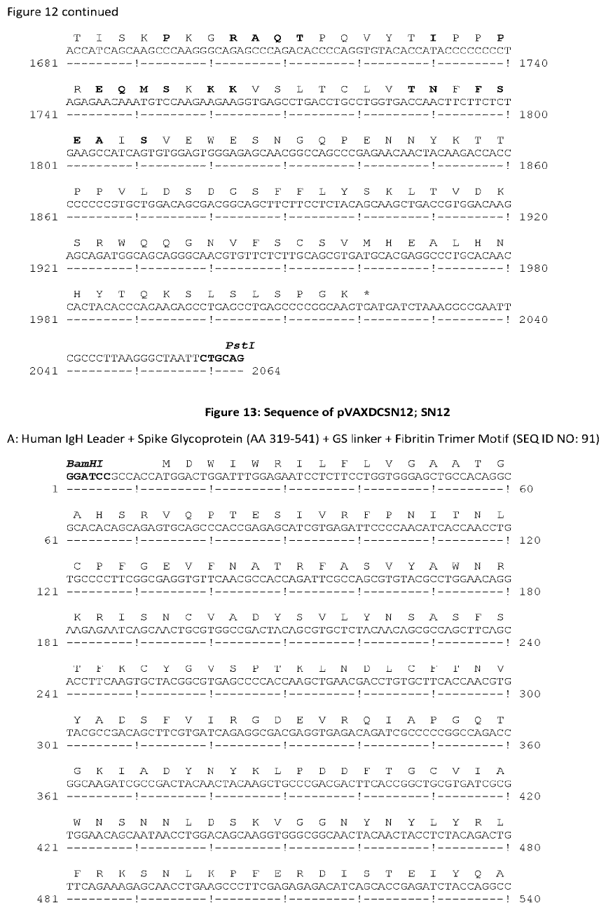

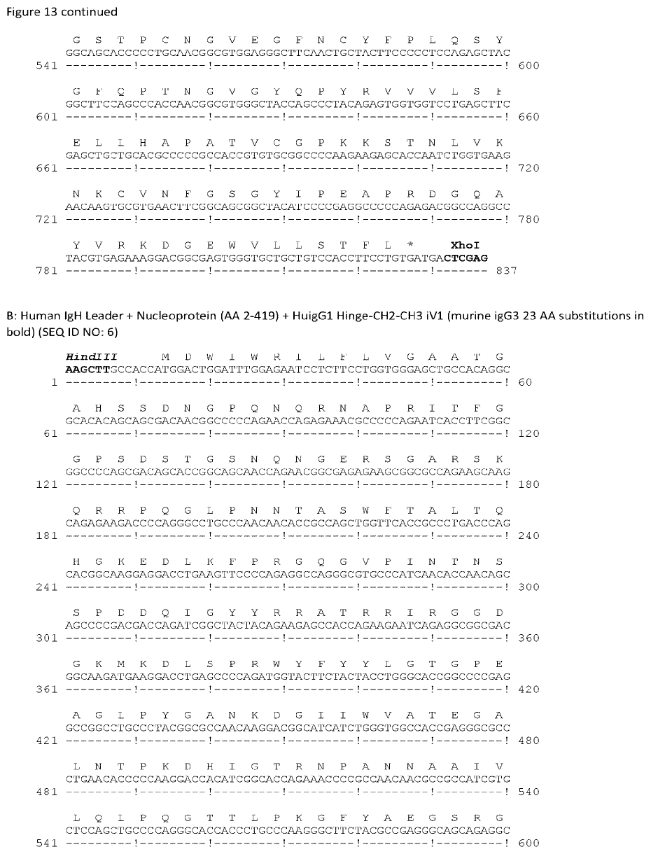

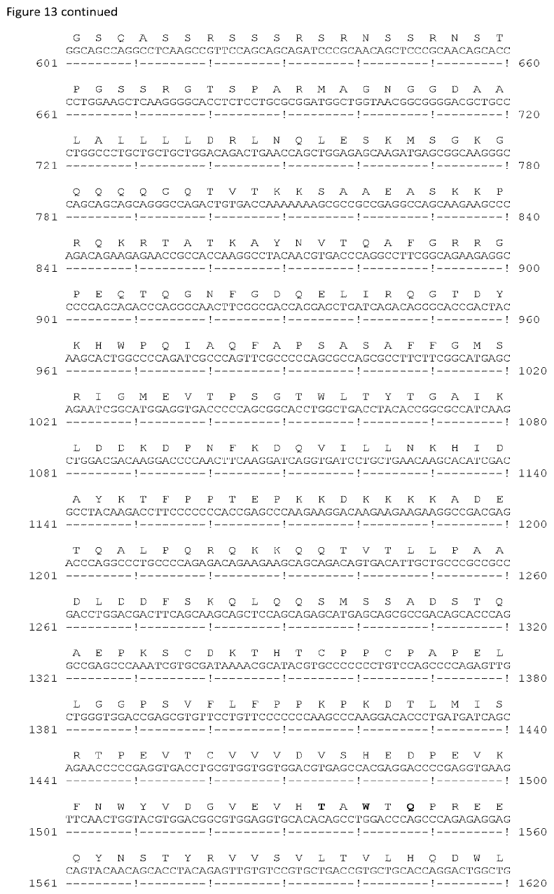

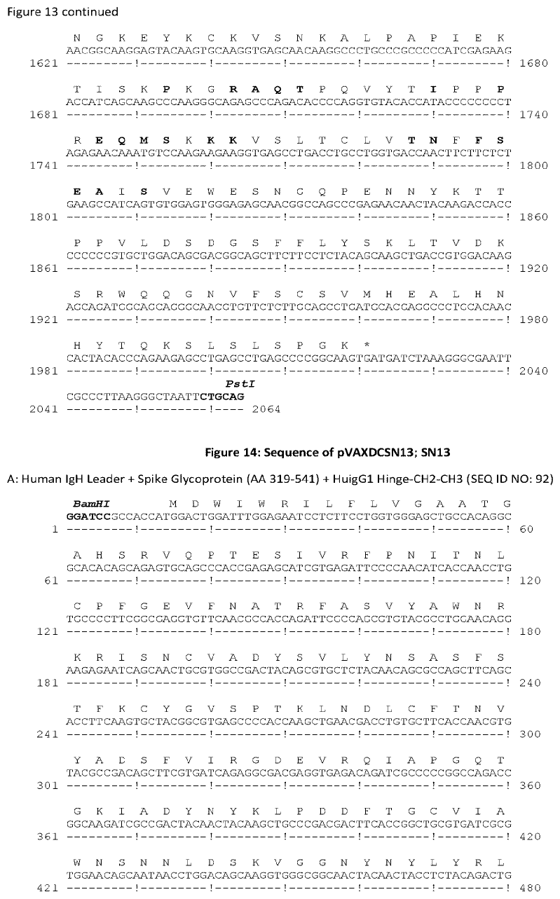

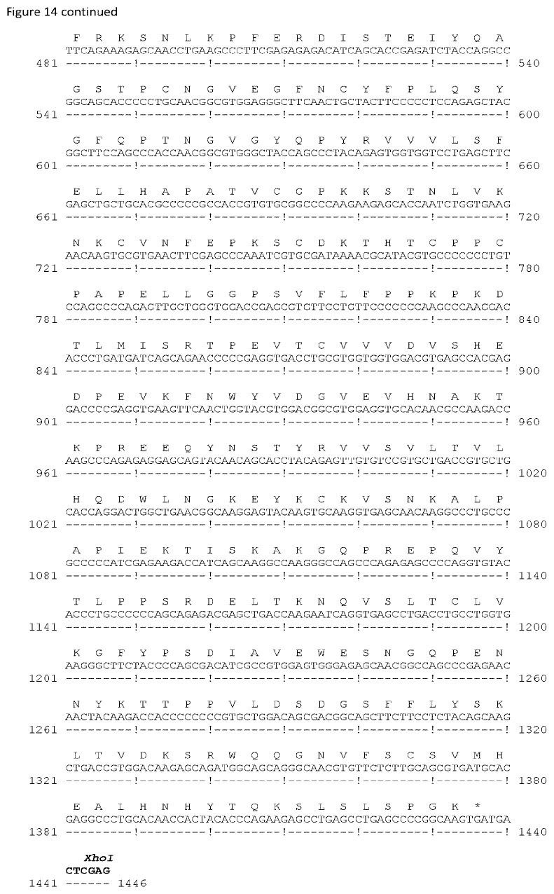

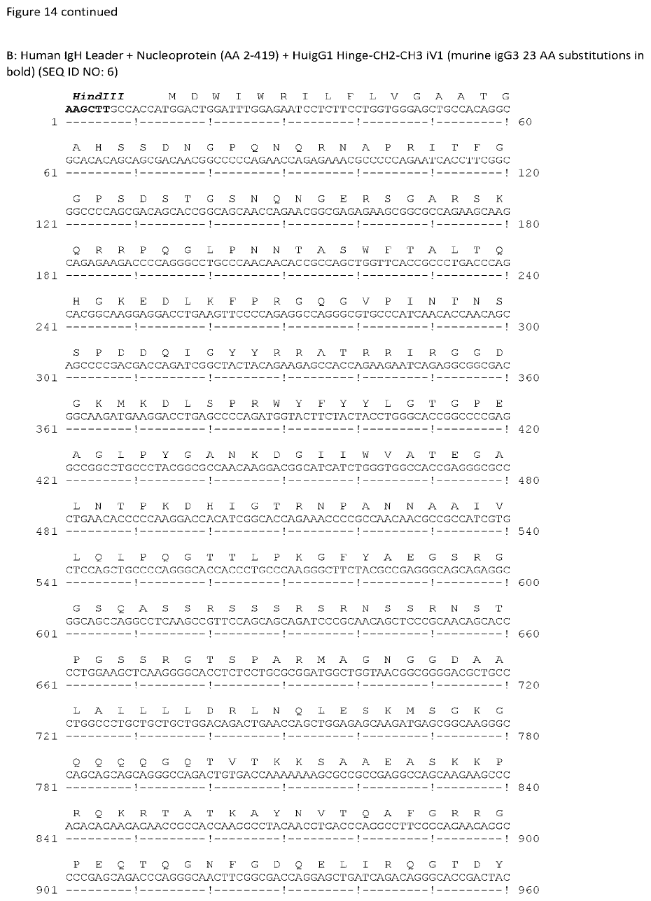

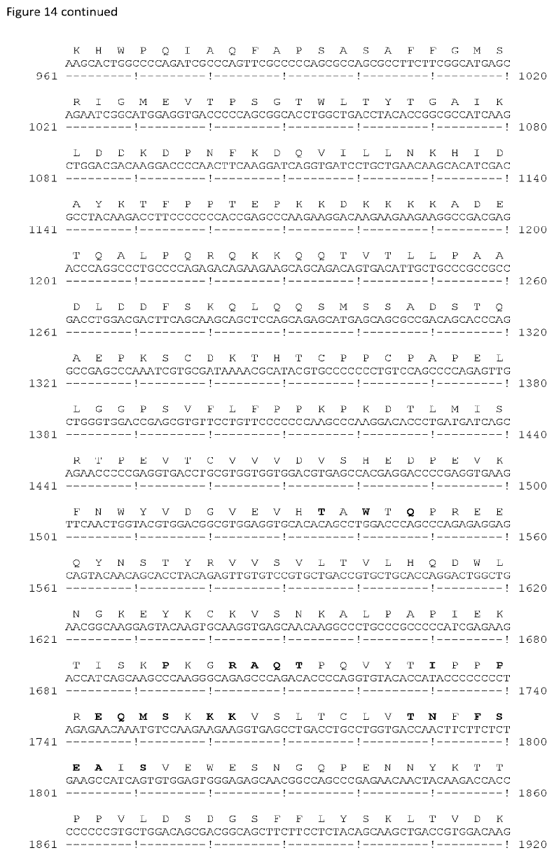

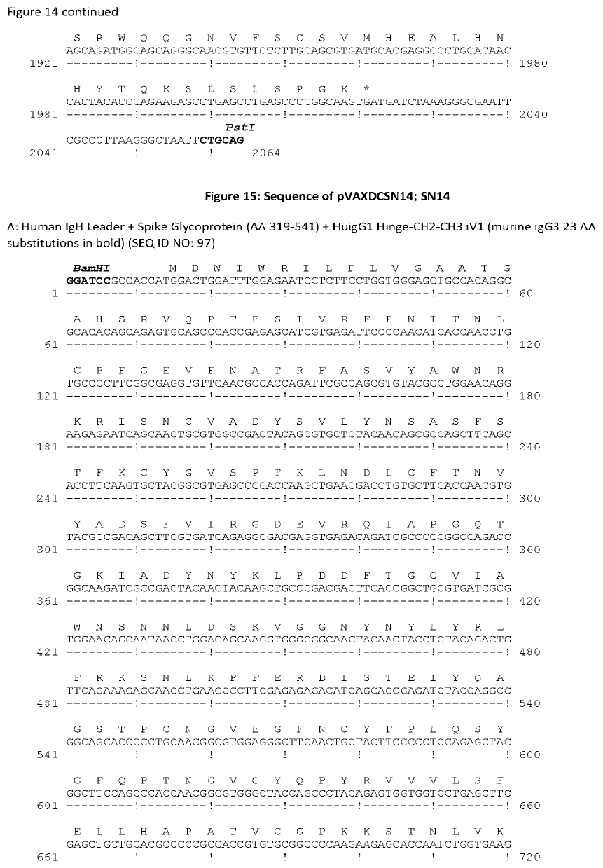

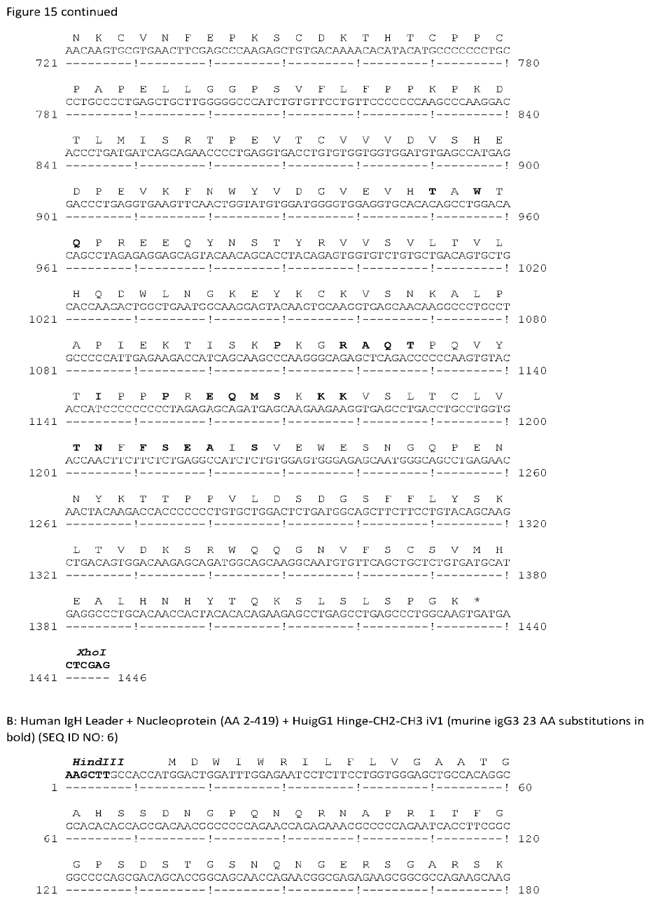

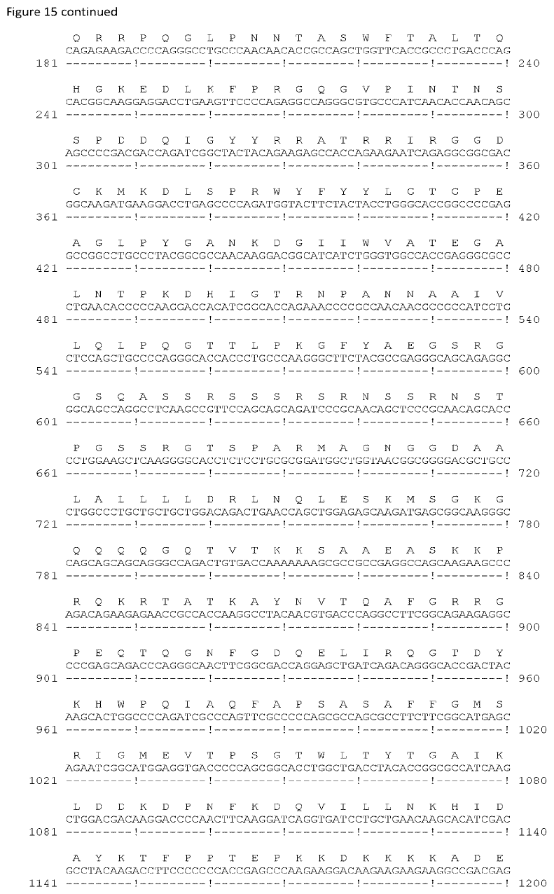

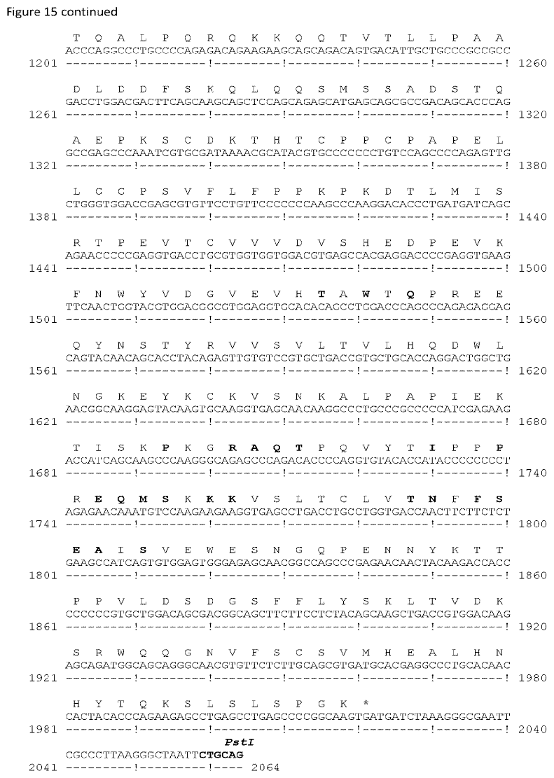

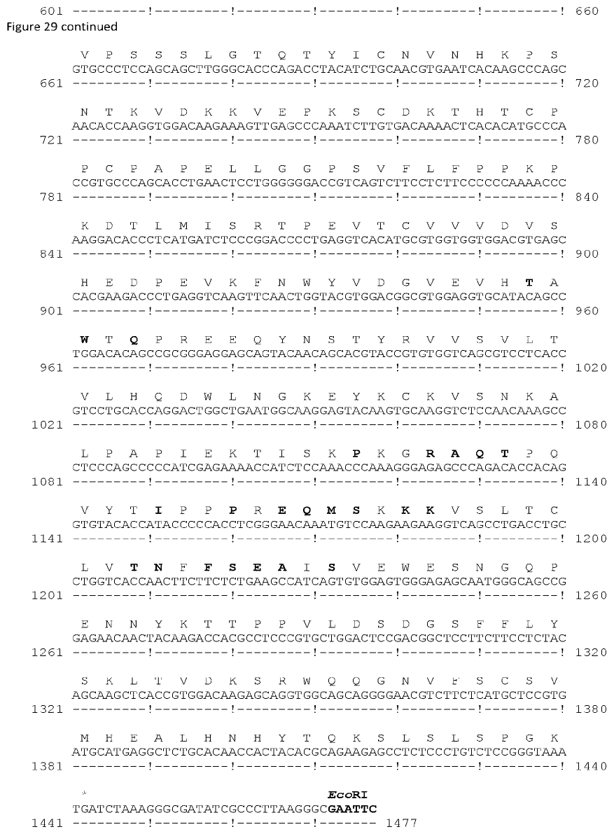

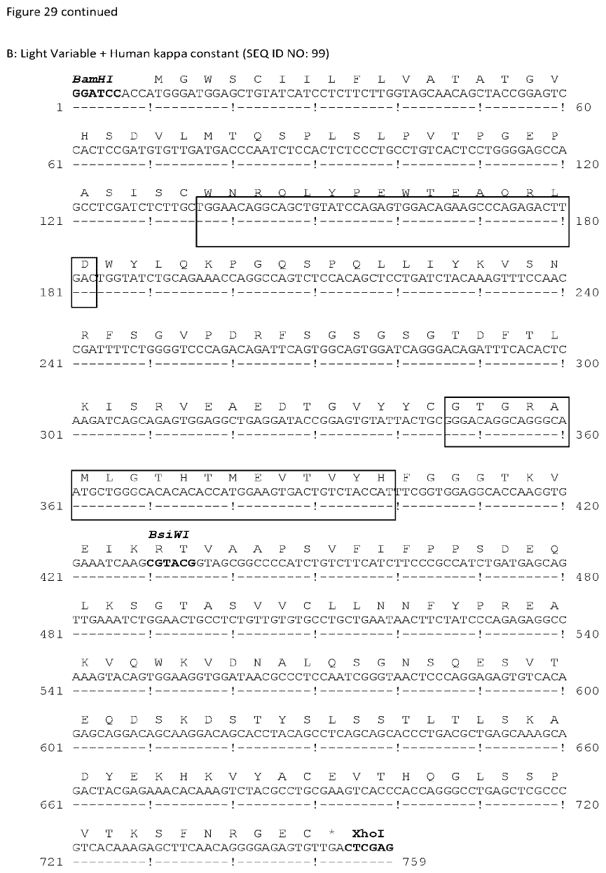

The present invention provides one or both of the amino acid sequences disclosed in FIG. 27, 28, 29, 30, 31, 32, 33, 2, 3, 4, 5, 6, 7, 8, 9, 10, 11, 12, 13, 14, 15, 47, 48, 54, 55, 56, 57, 58 or 59, respectively. Preferred polypeptides in accordance with the invention are set out in FIG. 12b (optionally in combination with FIG. 12a), FIG. 13b (optionally in combination with FIG. 13a), FIG. 14b (optionally in combination with FIG. 14a), FIG. 15b (optionally in combination with FIG. 15a), FIG. 27b (optionally in combination with FIG. 27a), FIG. 29a (optionally in combination with FIG. 29b), FIG. 31a (optionally in combination with FIG. 31b) and FIG. 33b (optionally in combination with FIG. 33a). Particularly preferred sequences are disclosed in FIGS. 27, 31, 33, 48 and 54 to 59.

The present inventors have shown that immunisation with SN15, encoding lineage A Wuhan strain N protein and RBD, gives strong VNAbs against this strain but also cross reacts with B.1.351 and B.1.617.2 variant RBDs. Similarly, the inventors have shown that immunisation with SN17, encoding B.1.351 variant N protein and RBD, gives strong VNAbs against this strain but also cross reacts with lineage A Wuhan strain and B.1.617.2 variant RBDs.

The avidity of a polypeptide of the invention comprising a modified Fc region and the corresponding polypeptide comprising a wildtype Fc region may be determined by Surface Plasmon Resonance (e.g. BIACORE® 3000/T200, GE HEALTHCARE®), for example by injecting increasing concentrations (0.3 nmol/L-200 nmol/L) of a polypeptide of the invention across a CM5 chip comprising an appropriate ligand (such as FcγRI) and fitting the data to an appropriate binding model using appropriate software (e.g. BIAevaluation 4.1). Corresponding experiments using the same conditions can be conducted for the corresponding polypeptide having the wildtype Fc region. In some aspects of the invention, the polypeptide of the invention comprising a modified Fc region shows greater functional affinity to ligand than the corresponding polypeptide having the wildtype Fc when the Surface Plasmon Resonance data indicates that the polypeptide of the invention binds more tightly to the ligand-coated CM5 chip. As mentioned the ligands may comprise Fc receptor, particularly Fcγ receptor. All of the Fcγ receptors (FcγR) belong to the immunoglobulin superfamily and are the most important Fc receptors for inducing phagocytosis of opsonized (marked) microbes. This family includes several members, FcγRI (CD64), FcγRIIA (CD32), FcγRIIB (CD32), FcγRIIIA (CD16a), FcγRIIIB (CD16b), which differ in their antibody affinities due to their different molecular structure. FcγRI binds to IgG more strongly than FcγRII or FcγRIII does. FcγRI also has an extracellular portion composed of three immunoglobulin (Ig)-like domains, one more domain than FcγRII or FcγRIII has. This property allows FcγRI to bind a sole IgG molecule (or monomer), but all Fcγ receptors must bind multiple IgG molecules within an immune complex to be activated. In some aspects of the invention, a preferred receptor is FcγRI (CD64). In some aspects of the invention, a preferred receptor is TRIM21. In some aspects of the invention, the polypeptides of the invention may be capable of binding to CD64 and/or TRIM21. TRIM21 is the cytosolic antibody receptor and E3 ubiquitin ligase. It detects antibody inside cells and mediates their rapid proteosomal degradation. If antibodies are modified to express T cell epitopes within their variable regions or heterologous antigens are linked to Fc and are administered via a DNA plasmid which can directly transduce antigen presenting cells, the protein would be translated within the cells and be targeted by TRIM21.

The BIACORE® CM5 chip, coated with an anti-His antibody, comprises carboxymethylated dextran covalently attached to a gold surface. Molecules are covalently coupled to the sensor surface via amine, thiol, aldehyde or carboxyl groups. Interactions involving small organic molecules, such as drug candidates, through to large molecular assemblies or whole viruses can be studied. A high binding capacity gives a high response, advantageous for capture assays and for interactions involving small molecules. High surface stability provides accuracy and precision and allows repeated analysis on the same surface. Other suitable chips are known to the skilled person and the Surface Plasmon Resonance protocols can be adapted by standard techniques known in the art.

The immunogenicity of a polypeptide in accordance with the invention may be determined. The improved properties of the polypeptide may be measured relative to the corresponding properties of a corresponding polypeptide that does not comprise the modified residues in the Fc-region. Since the improved functional properties are a relative measure, the precise method used to determine the immunogenicity, or any other functional property of the polypeptide of the invention, does not affect the relative change in that functional property.

Whilst not wishing to be bound by theory, the ability of the polypeptide of the invention to provide enhanced immunogenicity may be a direct consequence of the modified human IgG1 Fc binding to cell surface receptor. The polypeptide and/or nucleic acid of the present invention will usually be administered in the form of a pharmaceutical composition, which may comprise at least one component in addition to the nucleic acid/polypeptide.

Polypeptides of the invention comprise at least one heterologous antigen. As used herein, “heterologous antigen” is intended to mean an antigen which is heterologous to the modified Fc region. The antigen may be a T cell antigen or a B cell antigen. Some polypeptides in accordance with the invention comprise both T and B cell antigens. The antigen may be comprised in a relatively large molecule, such as a viral or bacterial protein or immunogenic fragment thereof. Alternatively, it may be an amino acid sequence making up the antigen or the epitope within the antigen. Examples of such antigens/epitopes are set out in Tables 2 and 3 herein.

The antigen may be from a cancer or may be from an infectious disease. In some aspects the antigen is from a cancer. In some aspects the antigen is from an infectious disease. The antigen may stimulate high avidity CD8 T cells, Th1 CD4 T-cells and/or strong antibody responses. Where the invention is to be used as a cancer vaccine, stimulation of potent cellular immunity including both CD4 and CD8 T-cell responses is vital. Where the invention is to be used as a vaccine for infectious disease, stimulation of both cellular immunity and neutralising antibodies is important.

Polypeptides, nucleic acids and vectors of the present invention can be used as a vaccine against viral infection and in particular against coronavirus infection. The coronaviruses (CoV) are members of the subfamily Coronavirinae (family Coronaviridae; order Nidovirales), which are classified into four genera, Alphacoronavirus (alpha-CoV), Betacoronavirus (beta-CoV), Gammacoronavirus (gamma-CoV), and Deltacoronavirus (delta-CoV) [18, 19]. Gamma-CoV and delta-CoV generally infect birds, although some can cause infection in mammals. The alpha-CoV and beta-CoV viruses are known to infect and cause disease in both humans and animals. The SARS-CoV (beta-CoV), 229E (alpha-CoV), HKU1 (beta-CoV), NL63 (alpha-CoV) and OC43 (beta-CoV) viruses can all cause infections in humans [18], typically causing upper respiratory infections and some relatively minor symptoms [20]. The beta-CoV are the most pathogenic viruses in humans, this group also includes SARS-CoV-2, MERS-CoV, and SARS-CoV [18, 21, 22]; all have caused outbreaks in the 21^{st} century. Among the coronaviruses, SARS-CoV-2 shows the greatest homology with SARS-CoV, demonstrating 79% genetic similarity [23]. SARS-CoV-2 is most similar to the bat coronavirus RaTG13, with 98% similarity [24].

The genome of CoVs is a single-stranded positive-sense RNA (+ssRNA) with 5′-cap structure and 3′-poly-A tail. The genomes of RNA viruses are typically less than 10 kb in length, but the CoV genome is the largest known for RNA viruses, being roughly 30 kb. The genomic viral RNA is used as template to directly translate polyprotein 1a/1ab, that encodes the non-structural proteins (nsps) to form the replication-transcription complex (RTC) in a double-membrane vesicles (DMVs) [25]. A nested set of sub genomic RNAs (sgRNAs) are then synthesised by RTC in a manner of discontinuous transcription [26]. The sub genomic messenger RNAs (mRNAs) possess common 5′-leader and 3′-terminal sequences. Transcription termination and subsequent acquisition of a leader RNA occurs at transcription regulatory sequences, located between open reading frames (ORFs). These minus strand sgRNAs serve as the template for the production of sub genomic mRNAs [27, 28]. The genome and sub genomes of a typical CoV contain at least six ORFs. The first ORFs (ORF1a/b), about two-thirds of the whole genome length, encodes the 16 nsps (nsp1-16), the other ORFs of the genome near the 3′-terminus encodes at least four main structural proteins: spike (S), membrane (M), envelope (E), and nucleocapsid (N) proteins. In addition to these four main structural proteins, different CoVs encode additional special structural and accessory proteins, such as HE protein, 3a/b protein, and 4a/b protein.

In common with other coronaviruses that cause respiratory infections, SARS-Cov-2 is transmitted primarily via respiratory droplets; the transmission rate for SARS-Cov-2 seems to be higher when compared with both SARS-CoV and MERS. In some people, the infection is asymptomatic, and these individuals are thought to be potential sources of SARS-CoV-2 infection [29], causing the rapid spread of SARS-CoV-2. In those who develop COVID-19 disease following infection, pneumonia appears to be the most common manifestation, other symptoms also include fever, cough, shortness of breath, and bilateral infiltrates are visible on chest imaging [30].

Once infected with SARS-CoV-2, the median incubation period is approximately 4-5 days but it can be 14 days before symptoms appear [31-34], with 97.5% of symptomatic patients developing symptoms within 11.5 days [32]. The viral load peaks within 5-6 days of symptoms appearing; this is significantly earlier compared to the SARS virus which peaks at 10 days after symptoms develop [35-38]. In those patients that develop acute respiratory distress syndrome, this occurs around 8-9 days after symptoms start [30, 39]. SARS-CoV-2 causes an aggressive inflammatory response causing damage to the airways [40], the severity of disease is not only due to the viral infection but the host's immune response. The most common cause of death is respiratory failure (70% cases); in addition the release of cytokines induces a cytokine storm effect causing secondary infections [41] and sepsis, leading to multi organ failure and death.

The first step in infection is the virus binding to a host cell through its target receptor. SARS-CoV-2 uses a densely glycosylated spike (S) protein to gain entry into the host cells. The S protein is a trimeric class I fusion protein that exists in a metastable prefusion conformation that undergoes a structural rearrangement to fuse the viral membrane with the host cell membrane [42, 43]; this process is triggered when the S1 subunit binds to a host cell receptor. The SARS-CoV-2 virus binds to the angiotensin converting enzyme 2 (ACE2) receptor [24]; the serine protease TMPRSS2 is also reported to play an important role in host cell entry [44]. The virus targets the airway epithelial cells, alveolar epithelial cells, vascular endothelial cells and macrophages in the lung, all of which express ACE2 [24, 45]. The S1 subunit consists of an amino-terminal domain and a receptor-binding domain (RBD). The RBD binds to ACE2 triggering endocytosis of the SARS-CoV-2 virion and exposes it to endosomal proteases [46]. The S2 subunit consists of a fusion peptide (FP) region and two heptad repeat regions: HR1 and HR2 [47, 48]. Within the endosome, the S1 subunit is cleaved away, exposing the fusion peptide, which inserts itself into the host membrane. The S2 region then folds in on itself to bring the HR1 and HR2 regions together. This leads to membrane fusion and releases the viral package into the host cytoplasm.

Once the SARS-CoV-2 virus has gained entry into the host cell, four structural proteins are then required for virus assembly, the S, M, E, and N proteins. Homotrimers of S proteins make up the spikes on the viral surface and they are responsible for attachment to host receptors [49, 50], M protein shapes the virions and binds to the N protein [51, 52]. The E protein plays a role in virus assembly and subsequent release and is important in disease pathology [53, 54]. The N protein contains two domains that can each bind virus RNA genome via different mechanisms. It is reported that N protein can bind to nsp3 protein to help tether the genome to the replication-transcription complex (RTC), and package the encapsulated genome into virions [20, 55, 56].

The RBDs of SARS-CoV and SARS-CoV-2 have 72% homology in their amino acid sequences and a highly similar tertiary protein structure. Computational modelling and biophysical measurements indicate that the SARS-CoV-2 RBD binds to ACE2 with higher affinity when compared to RBD from SARS-CoV [57, 58]. The SARS-CoV-2 S protein also contains a furin-like cleavage site, similar to MERS-CoV and human coronavirus OC43, but which is not found in SARS-CoV [59]. These characteristics are likely to contribute to the increased infectivity observed with the SARS-CoV-2 and have ultimately helped the virus spread.

Approximately 80% of SARS-CoV-2 infected individuals present with no symptoms or mild infection [30] suggesting an effective immunity has been established. As with SARS-CoV and MERS, most patients exhibiting severe symptoms showed evidence of peripheral lymphopenia, thought to be due to the migration of lymphocytes out of the blood to the site of infection in the lungs [60, 61]. Th1 cytokines such as IL-6, IFNγ, IP-10 and MCP-1 made by CD4 T cells in response to the viral infection recruit other T cells and monocytes [60] and, in most cases, infection is successfully cleared. However, in some cases infection progresses and manifests as a more severe lung pathology. This is thought to be due to a dysfunctional immune response and a subsequent cytokine storm effect with high levels of pro-inflammatory cytokines including IL-2, IL-7, IL-10, G-CSF, IP-10, MCP-1, MIP-1α and TNFα [30, 40, 62], likely mediated by macrophages and elevated levels of inflammatory monocytes. This cytokine storm is responsible for the majority of associated severe pathologies although it is unclear to date if persistence of viral infection is required to drive the ongoing immune mediated damage.

Detailed information on immune responses to SARS-CoV-2 has yet to be gathered but analogies can be drawn from SARS-CoV and MERS. SARS-CoV and MERS are both known to interfere with the Type I and type III IFN response to viral infection which in turn leads to an increase in inflammatory neutrophils and macrophages to the site of infection [63, 64]. It is highly likely that SARS-CoV-2 also possesses similar mechanisms of immune subversion. An increased disease severity correlates with increased neutrophils and decreased lymphocytes [65, 66]. In the case of MERS, the infection of macrophages and DCs can also lead to the down regulation of MHC class I and II which could lead to the dramatic reduction in T-cell priming at the site of infection [67]. It remains to be determined if similar effects are caused by the SARS-CoV-2 virus and if this contributes to the failure to control viral persistence.

Decreases in peripheral T lymphocytes in severe SARS-CoV and MERS disease cases suggest an important role for these cells in anti-viral immunity. It has been known for some time that a T-cell mediated immune response is vital for the control of viral infections [68]. The T lymphocyte decrease associated with severe disease is also correlated with a reduction in the polyfunctionality of CD4 T-cells, specifically those making IFNγ [69]. A suboptimal T-cell response combined with persistent antigenic activation can lead to a functionally exhausted state [70]. Interestingly, SARS-CoV-2 patients show a correlation of disease severity with increased T cell exhaustion, reduced functional diversity [71] and decreased activation [72] suggesting that an impaired T cell response may in part be responsible for the disease progression and severity. It is possible that this may be an effect of immune subversion by the virus or viral driven T cell exhaustion. The severe disease cohorts are often also characterised by the predomination of Th2 cytokines (IL-4, IL-5 and IL-10) rather than Th1 cytokines implying that the polarisation to a Th1 type response is beneficial [73]. The presence of a Th2 cytokine response also promotes lung eosinophil infiltration and worsens the pathology [74]. In SARS-CoV infections, strong T-cell responses have also been associated with higher titre virus neutralising antibodies [73]. However, the current literature indicates that although T cell responses are crucial for viral clearance care needs to be taken in order not to exacerbate lung pathology as high T-cell levels at the site of infection can also be associated with severe disease [75]. A number of reports using murine models indicate that a strong Th1 response alongside virus specific CD8 response and virus-specific neutralising antibodies are necessary for successful control of SARS-CoV and MERS [76, 77]. The same is likely to be the case for SARS-CoV-2.

In murine models of SARS-CoV infection T-cells, specifically CD4 T-cells, have been hypothesised to be important for the control of infection. Indeed, in mouse models depletion of CD4 T-cells resulted in prolonged viral persistence and increased lung pathology [78]. Adoptive T-cell transfer studies in mice also showed beneficial effects of prior SARS-CoV specific immunity in the prevention of infection [79].

It is too early for longitudinal data to be available on immune responses in survivors of SARS-CoV-2 but evidence from SARS-CoV patients that recover show the presence of long lived memory T-cell responses to both S and N antigens up to 11 years post infection [80-82] implying immunological memory is possible to betacoronaviruses. Mouse models have also demonstrated that memory CD8 T-cell responses efficiently provide protection from SARS-CoV infection [76]. Several bioinformatics approaches have identified potential immunogenic regions of SARS-CoV-2 proteins and likely HLA restrictions [83-86]. Although no T-cell epitopes have been defined to date within SARS-CoV-2, it has been demonstrated that S, N and M viral antigens are also targets for CD4 and CD8 T-cell mediated immunity ([87] Preprint). Grifoni et al. identified responses in SARS-CoV-2 patients with 100% of patients showing co-dominant CD4 responses to spike, M and N proteins. Predominantly Th1 responses were observed and these strong Th1 responses, as well as CD8 responses, correlated well with antibody responses to the S protein. Approximately 70% of patients showed CD8 responses with dominance to S and M proteins but responses to all other antigens were also observed. Interestingly, evidence of cross reactivity of responses between other human coronaviruses was observed since 40-60% of unexposed individuals showed responses to the SARS-CoV-2 antigens tested in the study by Grifoni et al. ([87] Preprint).

Studies on SARS and MERS showed that virus-specific antibodies were detectable in 80-100% of patients at 2 weeks after symptom onset, but circulating titres were generally short-lived, gradually declining and becoming undetectable in most recovered individuals after a number of years [88, 89]. In the case of SARS-CoV-2, acute anti-viral responses correlate with increased antibody-secreting cells (ASCs), follicular helper T-cells (T_{FH} Cells), activated CD4+ T-cells and CD8+ T-cells and SARS-CoV-2 IgM and IgG antibodies detectable in the blood before symptomatic recovery [90]. Memory B-cells as well as T-cell responses contribute to long-term protection, although the former are not as readily maintained and detected in circulation and residue primarily in secondary lymphoid tissues [76, 88, 91]. Similarly, a recent study analysing the dynamics of the antibody response suggests that most SARS-CoV-2 patients (>95%) seroconvert within 19 days of contracting the disease, with antibodies detected as early as towards the end of the first week post-onset of symptoms [89, 92]. In a small cohort of patients with serum samples available 14 days or longer after symptom onset, rates of seropositivity were 94% for anti-N IgG (n=15), 88% for anti-N IgM (n=14), 100% for anti-RBD IgG (n=16), and 94% for anti-RBD IgM (n=15). IgG titres for both N and RBD correlated with virus neutralisation titre [93].

Globally, however, SARS-CoV-2 antibody responses and their effect on disease progression remain poorly understood, as demonstrated by reports suggesting that early high serum IgG antibody titres correlate with severity of the disease [94]. Specifically, an early rise in anti-S protein titres has been linked to worse outcomes through the induction of ARDS (acute respiratory distress symptom) which is characterised by the polarisation of alveolar macrophages towards a highly inflammatory, instead of wound healing, phenotype via an antibody effector function mediated pathway [95]. Similarly during SARS-CoV-1, rapid but transient anti-S and anti-N titres have been shown to correlate with worse outcomes [96]. Recovered patients showed a peak in titres on average 20 days post-onset of symptoms as opposed to only 14.7 days for the deceased patients.

Recent studies have shown that antibodies to the nucleocapsid protein can bind N protein either released during viral lysis or expressed on the surface of infected cells and the immune complex can be taken up by antigen presenting cells where TRIM21 targets N protein for cytosolic degradation and generates cytotoxic T cells against N peptides [97]. This is a rapid response and mediated viral clearance prior to the establishment of neutralising antibodies. This could be a primary method of action of a vaccine containing an N protein linked to modified Fc, whereby the modified Fc shows enhanced binding to TRIM21. Furthermore SN11, which expresses the N protein fused to modified Fc gave significantly better T-cell responses to N protein and also gave superior responses to RBD, S1 and peptide RBD 417-425 than a similar construct expressing the same RBD construct but N-Fc. This suggests that the modified N-Fc is acting like an adjuvant and activating the APCs to also enhance the T-cell response to other antigens.

Although non-neutralising antibodies can mediate in vivo protection, neutralising antibodies have received most attention [98]. For instance, polyclonal IgG concentrated from the serum of vaccinated and challenged non-human primates (NHPs) was able to protect naive NHPs against Ebola virus (EV) challenge and early studies indicated that development of an antibody response was associated with survival from EV disease [99, 100], culminating in a cocktail of three monoclonal antibodies being developed against Ebola by Regeneron. During the SARS/MERS virus life cycle, neutralising antibodies inhibit viral docking (RBD-targeting), membrane fusion (S-protein-targeting) or egress (M-protein targeting). Importantly, antibodies or plasma from convalescent patients are currently being evaluated as one of the treatment options available to COVID-19 patients—over 70% of patients who recovered from mild COVID-19 have measurable neutralising antibodies that persisted upon revisit to the hospital—further corroborating the notion that SARS-CoV-2 antibody responses play a role in disease resolution [101].

The surface S glycoprotein, which is critical for virus entry through engaging the host receptor and mediating virus-host membrane fusion, is the major antigen of coronaviruses. Other structural proteins include the E, M and N proteins, the latter being produced first and more abundantly than the others. Immunisation of rabbits with VLPs encoding a combination of all four proteins has shown that the S and N proteins are more immunogenic compared to the other two, with N protein titres rising more rapidly but more transiently compared to S protein titres [102]. The SARS-CoV-2 receptor-binding domain (RBD) is most prone to eliciting a potent neutralising response, in a similar way as has been shown for the MERS-CoV and SARS-CoV-1 RBD [103]. These facts show that the RBD in the S1 subunit is a major vulnerable site for antibody recognition and neutralisation—including a range of conformational epitopes, but this is also the region where most naturally occurring mutations of the S protein occur [103-105]. Hence cross-reactivity analysis of SARS-CoV-2 serum antibodies revealed that the majority of antibodies directed to the S protein did not cross react with SARS-CoV-1, in contrast to N directed antibodies that do display some cross reactivity

In rare cases, pathogen-specific antibodies can promote pathology, resulting in a phenomenon known as extrinsic antibody-dependent enhancement (ADE). ADE has been well characterized in in vitro studies of several flaviviruses including Zika and Dengue virus as well as respiratory syncytial virus (RSV) [106]. The process appears to be governed by low quality and/or low quantity non-neutralising antibodies binding to virus particles through their Fab domains, with their Fc domain engaging Fc receptors (FcRs) expressed on monocytes or macrophages, thereby facilitating viral entry and infection. ADE is mediated by the engagement of Fc receptors (FcRs) expressed on different immune cells, including monocytes, macrophages and B-cells, enabling viral cell entry in the absence of their canonical receptor or endosomal pH or proteases. For coronaviruses, previous studies have shown that the immunisation of mice with inactivated whole SARS-CoV2, the immunisation of rhesus macaques with MVA-encoded S protein and the immunisation of mice with DNA vaccine encoding full-length S protein could induce ADE or eosinophil-mediated immunopathology to some extent [95, 107]. The latter phenomenon appears to be driven by the combination of the quality of neutralising antibodies being produced as well as the temporal antibody response (faster titres lead to more severe disease), through a mechanism involving skewing of alveolar macrophages from wound healing to a pro-inflammatory phenotype (IL-8 production) and proinflammatory monocyte/macrophage recruitment and accumulation; a process at least partly driven by Fc gamma effector functions [95].

The establishment of pre-existing immunity to the virus has some value to eliminate the virus before immune responses become dysfunctional. In order to build up population immunity to SARS-CoV-2 we either need to allow ‘herd immunity’ to develop or we need to employ vaccination techniques. Both of these rely on the establishment of long-lived memory responses. Vaccines have been produced against several diseases caused by coronaviruses for animal use, including for infectious bronchitis virus in birds, canine coronavirus and feline coronavirus (FCoV) [108]. However, the possibility of vaccine-enhanced disease (VED) hinders development of vaccines against respiratory viruses, including FCoV. The pathology associated with VED in FCoV is akin to the acute lung damage caused by COVID-19 in a subset of patients. COVID-19 vaccines currently being developed focus on subunit vaccines that predominantly encode the S protein and stimulate VNAbs and T cell responses; it remains to be determined if they stimulate durable memory responses or avoid immune pathology.

Previous projects to develop vaccines for viruses in the family Coronaviridae that affect humans have been aimed at SARS and MERS. Vaccines against both SARS [109] and MERS [110] have been tested in non-human animal models. Currently there is no cure or protective vaccine for SARS that has been shown to be both safe and effective in humans [111].

In light of information so far, immunity stimulated by vaccination will likely need to comprise of both potent virus specific CD8 and CD4 T-cell responses as well as neutralising antibody responses. The hallmark of potent anti-viral T cell responses includes high functional avidity combined with polyfunctionality. Several factors have been implicated in the regulation of functional avidity, e.g. the cytokines IL-12 and IL-15 [112, 113], CD8αβ expression [114-116], TCR affinity [117], the level of co-stimulatory molecules expressed by antigen presenting cells [112, 118] and the maturation state of DCs. The challenge is therefore to find a vaccine approach which mimics these conditions. Large immune complexes can be cross-presented by the low affinity FcγRIIa (FcγRIV in mice), but only if the inhibitory FcγRIIb receptor is blocked or down-regulated [119]. The challenge is therefore to make a small genetic complex that targets the high affinity FcγRI and displaces potentially occupying serum IgG.

Effective vaccine strategies must demonstrate: (i) the ability to protect against heterologous viral variants that arise during independent emergence events—of note, many S-targeted antibodies have significantly reduced neutralisation titres against heterologous spike glycoproteins; (ii) the ability to elicit robust immune responses in elderly populations that are difficult to immunise and at increased risk for SARS-CoV2-induced morbidity and mortality; and (iii) avoidance of adverse vaccine outcomes, such as the vaccine-induced immune pathology that has been demonstrated following vaccination with the SARS N protein [120]. Strategies are generally directed at eliciting neutralising antibodies, a proven correlate with in vivo protection; however this can come at a cost of only covering a narrow range of virus reactivity (i.e. not broadly neutralising). Antibodies that bind more conserved parts of the virus tend to be less neutralising (in in vitro assays), relying instead on FcR engagement for affording in vivo protection, but could cover a broader range of protection. The risk of ADE argues against the use of the full S protein, in spite of it carrying more antigenic determinants, compared to the RBD [107]. Structurally, the SARS-CoV-2 RBD, like that of SARS-CoV-1, is exposed in both known states of the S protein trimer, namely a closed state where each RBD contacts symmetrically its analogues on the other protomer and an open state in which at least one RBD domain is extended to contact ACE2. The RBD is also easier to produce and generates higher levels of neutralising antibodies, a large proportion of which were directed against conformational epitopes and not necessarily associated with the ACE2 binding site [104, 105].

Certain polypeptides, nucleic acids and vectors in accordance with the invention aim to induce high affinity antibodies to the RBD as it has been shown that higher affinity antibodies with stronger neutralising ability carry reduced risk of ADE [107]. At the same time, the N protein is the earliest protein expressed and is more abundant compared to the S or M protein [89]. Combined with its more conserved nature and thus greater likelihood for heterologous protection, this also makes a valid target for the present invention.

The present invention can target both a virus N protein and the key RBD of the S protein to generate CD8 T cells, CD4 T cells and VNAbs. The S protein is presented as a trimer in its interaction with ACE2 so to optimise eliciting antibodies that have the neutralising phenotype. The inventors have multimerised the RBD, and this may be used in the vaccine for the prevention or treatment of SARS-CoV-2.

One polypeptide of the present invention comprises the N protein or an immunogenic fragment thereof fused to the modified human IgG3 Fc region. The N protein or immunogenic fragment thereof may comprise amino acids 2-419 or 138-146. The modified human IgG3 Fc region may comprise the Hinge-CH2-CH3 regions having the murine IgG3 modifications described herein. One example is shown in FIG. 12b. This polypeptide may be in combination with a second polypeptide comprising the RBD or an immunogenic fragment thereof. The RBD may comprise amino acids 319-541 or 330-525 (Accession Number YP_009724390). The RBD may (a) alone, (b) attached to a trimerization domain, such as fibritin trimer fold on or disulphide bridge motif, for example via a glycine serine linker or (c) fused in frame with (i) the Hinge-CH2-CH3 domain of the HuIgG1 constant domain (Accession Number P01857) or (ii) the variant Hinge-CH2-CH3iV1 in accordance with the invention. Examples of these are shown in Table 4 herein.

In addition to infectious diseases, such as viral infections (as described above) and bacterial infections, the invention can be used to target tumour antigens. Examples of such antigens are set out in Table 3 herein.

Tumours accumulate mutations that drive growth and metastases. These mutations represent unique epitopes that avoid thymic selection. They are termed “neo-epitopes” and are specific to individual tumours and are not found on normal tissues [121]. Lennerz et al. identified in a mixed lymphocyte-tumour cell culture from one patient with long-term survival from melanoma responses to eight antigens, five of which were neo-antigens [122]. This is an early study that showed that neo-antigens are associated with responses in long-term survivors. This has led researchers to develop personalised vaccines against identified neo-epitopes. Not all mutations stimulate a T cell response but there is a correlation between the frequency of mutations and the likelihood of presenting a T cell epitope. Indeed, patients with tumours that have a higher mutation rate often show better responses to checkpoint blockade therapies suggesting endogenous neo-epitope responses are uncovered by the checkpoint blockade [123]. As most mutations do not stimulate an immune response, selection of the most appropriate epitope to target can be difficult. However, significant progress is being made in the appropriate selection of candidate epitopes [124].

In human melanoma, mass spectrometry has been used to identify neo-epitopes directly from primary tumours leading to the identification of a number of potential targets [125]. Therapies targeting these neo-epitopes are being translated into the clinic and showing efficient induction of specific immune responses [126-128]. Several groups have treated patients with vaccines targeting multiple neo-epitopes. Sahin et al. have shown that multiple neo-epitope specific responses can be generated in patients after treatment with intranodal delivery of a RNA polyepitope vaccine [127].

They demonstrated a decrease in metastatic events and sustained progression free survival. Ott et al. also treated six melanoma patients with a peptide polyepitope vaccine combined with the adjuvant HILTONOL® (a stabilisation of poly IC with poly-L-lysine double-stranded RNA). They saw efficient neo-epitope specific T cell responses with reduced recurrence rate [128]. In addition, patients with recurrent disease post vaccination exhibited complete regression after subsequent anti-programmed cell death (PD-1) therapy which was associated with expansion of neo-epitope specific T cell responses. Interestingly the neo-epitopes identified in these studies were recognised by both CD8 and CD4 T cells suggesting an important role for CD4 T cell responses in addition to CD8 responses in humans. This confirmed previous data obtained in mouse models by Kreiter et al. [129]. One disadvantage of targeting neo-antigens is they are expensive as are patient specific and there can be huge variability both within a tumour sample and between tumours in the same patient; this can lead to outgrowth of tumours which no longer express the mutation [130]. To overcome “driver” mutations that are key in the tumourigenesis process (e.g. BRAFV600E), or other common mutations, they can be specifically targeted; however, these are much rarer and do not always stimulate T cell responses.

High avidity T cells specific for self-antigens are routinely deleted in the thymus during development leaving a low avidity repertoire. Therefore, antigens that show limited normal expression are likely to act as better targets since they may not have been subject to the same degree of tolerance. The detection of T cells specific to self-antigens in regressing cancer patients suggests that thymic tolerance is not always complete. Regressing cancer patients made responses predominantly to antigens with restricted expression in normal tissue such as the differentiation antigen TRP-2 and the cancer testis antigen NY-ESO-1 [131, 132]. Tumour associated antigens (TAA) including NY-ESO-1 [131, 133] and the melanoma antigen MAGE-1 [134] make good targets for the immune response suggesting that they have subverted immune tolerance.

Once an appropriate antigen has been selected, it is important to consider how best to present it to the immune system. Stimulation of T cells requires the processing and presentation of antigen by professional antigen presenting cells (APCs) such as dendritic cells (DCs) along with appropriate activating costimulatory signals. Activating costimulatory signals include those provided by TLR ligands [reviewed in 135]. Preclinical studies examining linkage of the peptide vaccine directly to TLR ligands are beginning to show promise. These are thought to more efficiently target both epitope and TLR to DCs leading to increased DC maturation and expression of costimulatory molecules, secretion of cytokines and chemokines and formation of an antigen depot within DCs to allow prolonged presentation of the peptide [136, 137]. In addition to direct linkage, studies have investigated the use of amphiphilic peptides combined with TLR ligands that assemble into nanostructures and are showing promise in preclinical studies [138, 139]. It is also important to consider the dose of antigen provided by the vaccine. A low dose can be enough to select for the highest affinity T cell receptor (TCR) and thus high avidity CD8 T cells [140] but may not be sufficient to stimulate CD4 T cells whose epitope target demonstrates lower affinity MHC-II binding.

It is widely accepted that the generation of high frequency T cell responses is not necessarily an indication of the induction of an effective immune response. It is apparent from previous published work that T cell functional avidity is a better indicator of clinical response [141-145]. The term functional avidity is often confused with affinity. Affinity is most often classified as a measure of the strength of binding of the peptide MHC molecule to the T cell receptor (TCR) whereas functional avidity is a measure of the combination of stimulation via TCR, co-stimulatory molecules, adhesion molecules and cytokines and is indicative of the overall strength of interaction between T cell and target and its functional outcome [146]. In both viral infection and tumour models, only high avidity cytotoxic T lymphocytes (CTL) mediate viral clearance and tumour eradication [117, 141, 143, 147, 148]. During the generation of an immune response in vivo CTL can show a range of functional avidities both at the clonal and polyclonal level. Although avidity has been shown to be important in both viral and tumour settings, the mechanisms by which high and low avidity CTL are generated in vivo remains unclear as the TCR cannot undergo somatic hypermutation. It has been demonstrated in vitro that culturing of TCR transgenic CTL in the presence of high or low dose of antigen leads to polarisation of low and high avidity responses respectively [141, 143].

Peptide vaccines encoding tumour epitopes have shown promise in animal models in early studies, stimulating specific T cell responses and tumour therapy in mice. Translation of these peptide vaccines into the clinic has been less successful with responses being short lived and minimal clinical efficacy. Early vaccines concentrated on the stimulation of CD8 T cell responses with short (<15 amino acids) peptides. However, more recent studies focus on the use of longer peptide sequences that can stimulate both CD4 and CD8 T cell responses to avoid problems with tolerisation previously seen with shorter peptide sequences [149]. Longer peptide sequences are beginning to show promising results in clinical studies [150, 151]. Peptides encoding neo-epitopes are also beginning to show some potential with detection of robust immune responses and evidence of improved overall survival [152, 153]. The study by Ott et al. (2017) demonstrated enhanced neo-epitope specific responses after vaccination with 20 long peptides peptide mixed with the TLR3 ligand HILTONOL® stabilized dsRNA therapeutic viral [128].

Synthetic peptides have also been used as part of DC based vaccines. Many studies have been performed where DCs cultured in vitro have been pulsed with peptides, proteins or tumour lysates. These have shown stimulation of efficient immune responses in preclinical studies [reviewed in 154]. Despite stimulating immune responses, DC vaccines have shown limited efficacy in the clinic. Sipuleucel-T (Provenge®), the only approved therapeutic autologous cell based vaccine to date, has shown a modest survival benefit of 3 months but the cost and time of production have severely limited its use [155]. This is the major limiting factor of most DC and autologous cell-based vaccines. In addition, a DC vaccine incorporating neo-epitope peptides showed promising expansion of both existing and de novo neo-epitope specific responses [126]. The use of immature DCs can impact their immunogenicity and lead to tolerance induction therefore the activation state of DCs used needs to be closely monitored. The extensive culture methods to manufacture DC based vaccines may also impact on their immunogenicity in vivo. Recent work focusing on the isolation of DC subsets ex vivo with minimal in vitro manipulation has shown promising results [156].

The nucleic acid of the invention may be DNA, cDNA, or RNA such as mRNA, obtained by cloning or produced wholly or partly by chemical synthesis. For therapeutic use, the nucleic acid is preferably in a form capable of being expressed in the subject to be treated. The nucleic acid of the present invention may be recombinant or provided as an isolate, in isolated and/or purified form. It may be free or substantially free of nucleic acid flanking the gene in the human genome, except possibly one or more regulatory sequence(s) for expression. Where nucleic acid according to the invention includes RNA, reference to the sequences shown herein should be construed as reference to the RNA equivalent, with U substituted for T.

Nucleic acids of the present invention can be readily prepared by the skilled person, for example using the information and references contained herein and techniques known in the art (for example, see Sambrook et al., (1989) [157], and Ausubel et al., (1992) [158], given the nucleic acid sequences and clones available. These techniques include (i) the use of the polymerase chain reaction (PCR) to amplify samples of such nucleic acid, e.g. from genomic sources, (ii) chemical synthesis, or (iii) preparing cDNA sequences. DNA encoding the polypeptide may be generated and used in any suitable way known to those of skill in the art, including by taking encoding DNA, identifying suitable restriction enzyme recognition sites either side of the portion to be expressed, and cutting out said portion from the DNA. The portion may then be operably linked to a suitable promoter in a standard commercially available expression system. Another recombinant approach is to amplify the relevant portion of the DNA with suitable PCR primers. Modifications to the sequences can be made, e.g. using site directed mutagenesis, to lead to the expression of modified peptide or to take account of codon preferences in the host cells used to express the nucleic acid.

In order to obtain expression of the nucleic acid sequences, the sequences can be incorporated into a vector having one or more control sequences operably linked to the nucleic acid to control its expression. The vectors may include other sequences such as promoters or enhancers to drive the expression of the inserted nucleic acid, nucleic acid sequences so that the polypeptide is produced as a fusion and/or nucleic acid encoding secretion signals so that the polypeptide produced in the host cell is secreted from the cell. If desired, polypeptide can then be obtained by transforming the vectors into host cells in which the vector is functional, culturing the host cells so that the polypeptide is produced and recovering the polypeptide from the host cells or the surrounding medium. Prokaryotic and eukaryotic cells are used for this purpose in the art, including strains of E. coli, yeast, and eukaryotic cells such as insect cells, and animal cells, for example, COS, CHO cells, Bowes Melanoma and other suitable human cells. Where the present invention relates to nucleic acid(s) encoding the heavy and light chains of an antibody, the respective nucleic acids may be present in the same expression vector, driven by the same or different promoters, or in separate expression vectors.

The nucleic acids of the present invention may be used to stimulate an immune response against the at least one heterologous antigen in a patient such as a mammal, including human. Helper and/or cytotoxic T cell responses may be stimulated. The T cell response against a particular epitope obtained by the present invention may have a higher avidity than that obtained by immunisation with the same epitope as a simple peptide, or by immunisation with the same epitope encoded within an antigen either as a peptide or a nucleic acid. The nucleic acids of the invention may be administered as a combination therapy, i.e. a nucleic acid encoding the light chain and nucleic acid encoding the heavy chain. The nucleic acid may be administered intravenously, intradermally, intramuscularly, orally or by other routes. Intradermal or intramuscular administration is preferred because these tissues contain dendritic cells.

A further aspect of the invention provides a vector comprising the nucleic acid of the invention. Vectors may be used for expression of the nucleic acid in order to obtain the polypeptide of the invention, or they may be used as a treatment (e.g. vaccine) in and of themselves.

Exemplary vectors include iSCIB1plus (see FIG. 31), iSCIB2 (see FIG. 33), SN15 (see FIG. 27) and SN17 (see FIG. 48).

The vector of the invention may comprise the nucleotide sequences provided in:
(a) SEQ ID NO: 12 and SEQ ID NO: 13; or
(b) SEQ ID NO: 14 and SEQ ID NO: 15.

SEQ ID NO: 12 and SEQ ID NO: 13 are the nucleotide sequences of the whole iSCIB1plus heavy and light chain expression cassettes respectively, both sequences including a CMV promoter and BGH polyA signal. SEQ ID NO: 14 and SEQ ID NO: 15 are the nucleotide sequences of the whole iSCIB2 heavy and light chain expression cassettes respectively, both sequences including a CMV promoter and BGH polyA signal.

The vector of the invention may comprise the nucleotide sequences provided in SEQ ID NO: 16 and SEQ ID NO: 17.

SEQ ID NO: 16 and SEQ ID NO: 17 are the nucleotide sequences of the whole SN15 N protein-Fc and S protein expression cassettes respectively, both sequences including a CMV promoter and BGH polyA signal.

The vector of the invention may comprise the nucleotide sequences provided in SEQ ID NO: 18 and SEQ ID NO: 19.

SEQ ID NO: 18 and SEQ ID NO: 19 are the nucleotide sequences of the whole SN17 N protein-Fc and S protein expression cassettes respectively, both sequences including a CMV promoter and BGH polyA signal.

The vector of the invention may comprise the nucleotide sequence provided in SEQ ID NO: 20, SEQ ID NO: 21 or SEQ ID NO: 22. Preferably, the vector of the invention consists of the nucleotide sequence provided in SEQ ID NO: 20, SEQ ID NO: 21 or SEQ ID NO: 22.

SEQ ID NO: 20 is the whole iSCIB1plus nucleotide sequence in plasmid format. SEQ ID NO: 21 is the whole iSCIB1plus nucleotide sequence in doggybone (dbDNA) format. SEQ ID NO: 22 is the whole iSCIB2 nucleotide sequence in plasmid format.

The vector of the invention may comprise the nucleotide sequence provided in SEQ ID NO: 23. Preferably, the vector of the invention consists of the nucleotide sequence provided in SEQ ID NO: 23. SEQ ID NO: 23 is the whole SN15 vector nucleotide sequence, in plasmid format.

The vector of the invention may comprise the nucleotide sequence provided in SEQ ID NO: 24 or SEQ ID NO: 25. Preferably, the vector of the invention consists of the nucleotide sequence provided in SEQ ID NO: 24 or SEQ ID NO: 25. SEQ ID NO: 24 is the whole SN17 vector nucleotide sequence, in plasmid format. SEQ ID NO: 25 is the whole SN17 vector nucleotide sequence, in doggybone (dbDNA) format.

The vector of the invention may be DNA. Plasmid DNA vaccines offer a number of advantages over other vaccine modalities as they have intrinsic adjuvant activity resulting in recruitment of large numbers of inflammatory cells to the site of immunisation. The mechanisms underlying DNA vaccine-induced immunity are complex and have yet to be fully elucidated but are thought to involve promiscuous and discriminative DNA sensors expressed by APCs (Table 1). CpG motifs signal through TLR9 to promote the activation and maturation of DCs [159]. Interestingly, DNA vaccine activity was still observed in TLR9 knockout mice implicating additional endosomal and cytosolic DNA sensors that mediate adjuvant activity [160]. DNA sensors such as TBK-1 and STING activate TLR-Independent pathways and induce type I interferons [161]. More recently the helicase DDX41 was identified as a new DNA sensor in myeloid dendritic cells [162]. In addition RIG-1 can also stimulate type I IFNs by sensing cytosolic DNA in association with RNA polymerase III [163]. DNA-dependent activator of IFN regulatory factors (DAI/DLM-1/ZBP1) is a cytosolic DNA sensor and an activator of innate immunity [164].

TABLE 1
Sensors of DNA that provide powerful adjuvanticity.

| Nucleic acid | DNA sensor | References |
|---|---|---|
| CpG | TLR9 | [159] |
| dsDNA | STING/TBK1 | [161] |
| dsDNA | DDX41 | [162] |
| DNA/RNA polymerase III | RIG-1 | [163] |
| Cytoplasmic DNA | DAI | [164] |
| Cytoplasmic DNA | AIM2 | [160] |
| Cytoplasmic DNA | IFI16 | [165] |
| CpG, dsDNA | HMGB-1 | [166] |

HMGB-1 has emerged as promiscuous sensor for nucleic acid mediated induction of innate immune responses [166]. HMGB-1 (a chromatin binding protein), has a range of functions depending on its subcellular and extracellular localisation, redox state, and interaction with other cell surface receptors. As an intracellular complex, HMGB-1 associated nucleic acids stimulate through TLR and cytosolic mediated sensors, type I IFNs, activate pro-inflammatory cytokines and induce the inflammasome (though AIM2) (reviewed in [160]). Extracellular release of HMGB-1 has a range of consequences, including sustaining tumour cell autophagy (by competing with bcl-2 for beclin-1 binding, as well as recruitment and activation of immune cells [167]). Recently, the different redox states of HMGB-1 have been shown to play an important role in HMGB-1 activity [167]. HMGB-1 contains three cysteines at positions C23, C45 and C106 that can be modified. The reduced all-thiol form of HMGB-1 is a chemoattractant that mediates leukocyte recruitment. The disulphide form has cytokine (but not chemokine) activity. The fully oxidised form of HMGB-1 induced by reactive oxygen species is inactive. HMGB-1 also forms complexes with cytokine and other immune receptors including RAGE, TLR4, TLR2, CD24, TIM-3, thrombospondin and TREM1 [168, 169]. HMGB-1/DNA complexes bind to RAGE and induce a switch from apoptosis to autophagy [169]. HMGB-1/CXCL12 binds to CXCR4 mediating recruitment of inflammatory cells [170].

Preferably, the vector of the invention is a DNA plasmid or doggybone (dbDNA) vector. Such vectors and their methods of production will be well known in the art. In particular, dbDNA vectors, and methods of production, are described in WO 2010/086626.

The vector of the present invention may be RNA. There are two categories of mRNA vaccines, those using non-replicating mRNA and those using self-replicating RNA. The invention contemplates both. The non-replicating mRNA vaccines contain only the transcript for the antigen of interest, whereas the self-replicating RNA vaccines in addition to the antigen of interest also include transcripts for the RNA replication machinery required for mRNA amplification. The self-replicating RNA vaccines induce the production of a large amount of antigen from only a small dose; this has the advantage that development and manufacturing of the vaccine is less complicated and much cheaper compared to other platforms. However, RNA vaccines have a short half-life and therefore do not sustain the production of the antigen giving short lived protection. For both non-replicating and self-replicating mRNA vaccines, the mRNA must be formulated to prevent degradation or be enclosed within a carrier which protects the mRNA from degradation by nucleases prior to uptake by host cells. A number of different carriers have been successfully used [171-174], these are mainly based on lipid-nanoparticles that encapsulate the mRNA and facilitate cellular uptake. The present invention contemplates the use of these mRNA vaccines.

The present invention provides
a vaccine comprising polypeptide, nucleic acid and/or vector of the present invention, optionally in combination with an adjuvant,
a polypeptide, nucleic acid, vector and/or vaccine of the present invention for use in medicine,
a polypeptide, nucleic acid, vector and/or vaccine of the present invention for use in the treatment of cancer or an infectious disease
a method for treating cancer or an infectious disease, comprising administering to a subject in need of such treatment a polypeptide, nucleic acid, vector and/or vaccine of the present invention.

As used herein, the term “treatment” includes any regime that can benefit a human or non-human animal. The treatment may be of an inherited or acquired disease. Preferably, the treatment is of a condition/disorder associated with cell proliferation such as cancer or infectious disease. Examples of types of cancer that can be treated with the nucleic acid include any solid tumour, colorectal cancer, lung, breast, gastric, ovarian, uterine, liver, kidney, pancreatic, melanoma, bladder, head and neck, brain, oesophageal, pancreatic, and bone tumours, as well as soft tissue cancers, and leukaemia's. Examples of infectious diseases that can be treated with the invention include infection with bacteria or viruses, such as coronaviruses, HIV, Hepatitis C, or any infection that requires T cell immunity for clearance and neutralising mAbs to prevent re-infection.

In some aspects, the present invention provides a nucleic acid, peptide, vector and/or vaccine described herein for use in the prevention or treatment of cancer in a subject, optionally wherein the cancer is melanoma.

In other aspects, the present invention provides a nucleic acid, peptide, vector and/or vaccine described herein for use in the prevention or treatment of an infectious disease in a subject, optionally wherein the infectious disease is COVID-19.

Two or more different nucleic acids vectors peptides and/or vaccines may be administered to the subject. Preferably, both of the following nucleic acids are administered to the subject:
(a) a nucleic acid encoding a polypeptide comprising the amino acid sequence provided in SEQ ID NO: 6, in combination with a second nucleic acid encoding a receptor binding domain comprising the amino acid sequence provided in SEQ ID NO: 8; and
(b) a nucleic acid encoding a polypeptide comprising the amino acid sequence provided in SEQ ID NO: 7, in combination with a second nucleic acid encoding a receptor binding domain comprising the amino acid sequence provided in SEQ ID NO: 9.

The nucleic acid, polypeptide and/or vector may be employed in combination with a pharmaceutically acceptable carrier or carriers. Such carriers may include, but are not limited to, saline, buffered saline, dextrose, liposomes, water, glycerol, ethanol and combinations thereof.

The nucleic acids, polypeptides and/or vectors useful in the invention can be formulated in pharmaceutical compositions. These compositions may comprise, in addition to one or more of the above substances, a pharmaceutically acceptable excipient, carrier, buffer, stabiliser or other materials well known to those skilled in the art. Such materials should be non-toxic and should not interfere with the efficacy of the active ingredient. The precise nature of the carrier or other material may depend on the route of administration, e.g. intradermal, oral, intravenous, cutaneous or subcutaneous, nasal, intramuscular, intraperitoneal routes. The formulation is preferably nucleic acid as a stable dry powder precipitated onto the surface of microscopic gold particles and suitable for injection via a gene gun or a solution of DNA mixed with GET peptides. The formulation may be suitable for intradermal or intramuscular administration using electroporation. The formulation may be suitable for administration using needle-free injection.

The compositions comprising, or for the delivery of, nucleic acids are preferably administered to an individual in a “therapeutically effective amount”, this being sufficient to show benefit to the individual. The actual amount administered, and rate and time-course of administration, will depend on the nature and severity of what is being treated. Prescription of treatment, e.g. decisions on dosage etc, is within the responsibility of general practitioners and other medical doctors, and typically takes account of the disorder to be treated, the condition of the individual patient, the site of delivery, the method of administration and other factors known to practitioners. The nucleic acids of the invention are particularly relevant to the treatment of existing cancer and in the prevention of the recurrence of cancer after initial treatment or surgery. Examples of the techniques and protocols mentioned above can be found in Remington's Pharmaceutical Sciences, 16^{th} edition, Oslo, A. (ed), 1980.

Preferably, the nucleic acid of the invention stimulates helper and/or cytotoxic T cells that can significantly kill virally infected cells or generate VNAbs to prevent viral entry when administered to a human in an effective amount. The optimal dose can be determined by physicians based on a number of parameters including, for example, age, sex, weight, severity of the condition being treated, the active ingredient being administered and the route of administration. For example, a dose of 1-1000 μg of DNA is sufficient to stimulate both helper and cytotoxic T cell responses.

A composition may be administered alone or in combination with other treatments, either simultaneously or sequentially dependent upon the condition to be treated. Other cancer treatments include other monoclonal antibodies, other chemotherapeutic agents, other radiotherapy techniques or other immunotherapy known in the art.

The dose of nucleic acid will be dependent upon the properties of the agent employed, e.g. its binding activity and in vivo plasma half-life, the concentration of the polypeptide in the formulation, the administration route, the site and rate of dosage, the clinical tolerance of the patient involved, the pathological condition afflicting the patient and the like, as is well within the skill of the physician. For example, doses of 200 μg of nucleic acid per patient per administration are preferred, although dosages may range from about 10 μg to 8 mg per dose. Different dosages are utilised during a series of sequential inoculations; the practitioner may administer an initial inoculation and then boost with relatively smaller doses of nucleic acid.

A further aspect of the present invention provides a host cell containing a nucleic acid as disclosed herein. The nucleic acid of the invention may be integrated into the genome (e.g. chromosome) of the host cell. Integration may be promoted by inclusion of sequences that promote recombination with the genome in accordance with standard techniques. The nucleic acid may be on an extra-chromosomal vector within the cell, or otherwise identifiably heterologous or foreign to the cell.

A still further aspect provides a method, which comprises introducing the nucleic acid of the invention into a host cell. The introduction, which may (particularly for in vitro introduction) be generally referred to without limitation as “transformation”, may employ any available technique. For eukaryotic cells, suitable techniques may include calcium phosphate transfection, DEAE-Dextran, electroporation, liposome-mediated transfection and transduction using retrovirus or other virus, e.g. vaccinia or, for insect cells, baculovirus. For bacterial cells, suitable techniques may include calcium chloride transformation, electroporation and transfection using bacteriophage. As an alternative, direct injection of the nucleic acid could be employed.

Marker genes such as antibiotic resistance or sensitivity genes may be used in identifying clones containing nucleic acid of interest, as is well known in the art.

The introduction may be followed by causing or allowing expression from the nucleic acid, e.g. by culturing host cells (which may include cells actually transformed although more likely the cells will be descendants of the transformed cells) under conditions for expression of the gene, so that the encoded polypeptide (or peptide) is produced. If the polypeptide is expressed coupled to an appropriate signal leader peptide it may be secreted from the cell into the culture medium. Following production by expression, a polypeptide or peptide may be isolated and/or purified from the host cell and/or culture medium, as the case may be, and subsequently used as desired, e.g. in the formulation of a composition which may include one or more additional components, such as a pharmaceutical composition which includes one or more pharmaceutically acceptable excipients, vehicles or carriers (e.g. see below).

The pharmaceutical composition may comprise, in addition to active ingredient, pharmaceutically acceptable excipient, diluent, carrier, buffer, stabiliser or other materials well known to those skilled in the art. Such materials should be non-toxic and should not interfere with the efficacy of the active ingredient. The precise nature of the carrier or other material will depend on the route of administration, which may be oral, or by injection, e.g., intradermal or intramuscular.

It is envisaged that injections will be the primary route for therapeutic administration of the compositions although delivery through a catheter or other surgical tubing is also used. Some suitable routes of administration include intravenous, subcutaneous, intraperitoneal and intramuscular administration. Liquid formulations may be utilised after reconstitution from powder formulations. Preferred routes of administration are intradermal or intramuscular administration.

The nucleic acid, vector, peptide and/or vaccine of the invention may be administered to the subject using needle-free injection. As will be known by those skilled in the art, needle-free injectors (also known as “jet injectors”) use a narrow, high-pressure stream of liquid that penetrates the outermost layer of the skin (stratum corneum) to deliver a composition to underlying tissues of the epidermis or dermis (i.e., intradermal injection), fat (i.e., subcutaneous injection), or muscle (i.e., intramuscular injection).

For intravenous injection, or injection at the site of affliction, the active ingredient will be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection. Preservatives, stabilisers, buffers, antioxidants and/or other additives may be included, as required.

Pharmaceutical compositions for oral administration may be in tablet, capsule, powder or liquid form. A tablet may comprise a solid carrier such as gelatin or an adjuvant. Liquid pharmaceutical compositions generally comprise a liquid carrier such as water, petroleum, animal or vegetable oils, mineral oil or synthetic oil. Physiological saline solution, dextrose or other saccharide solution or glycols such as ethylene glycol, propylene glycol or polyethylene glycol may be included. Where the formulation is a liquid it may be for example, a physiologic salt solution containing non-phosphate buffer at pH 6.8-7.6, or a lyophilised powder.

The composition may also be administered via microspheres, liposomes, other microparticulate delivery systems or sustained release formulations placed in certain tissues including blood. Suitable examples of sustained release carriers include semi-permeable polymer matrices in the form of shared articles, e.g., suppositories or microcapsules. Implantable or microcapsular sustained release matrices include polylactides (U.S. Pat. No. 3,773,919; EP-A-0058481) copolymers of L-glutamic acid and gamma ethyl-L-glutamate [43], poly (2-hydroxyethyl-methacrylate). Liposomes containing the polypeptides are prepared by well-known methods: [175, 176]; EP-A-0052522; EP-A-0036676; EP-A-0088046; EP-A-0143949; EP-A-0142541; JP-A-83-11808; U.S. Pat. Nos. 4,485,045 and 4,544,545. Ordinarily, the liposomes are of the small (about 200-800 Angstroms) unilamellar type in which the lipid content is greater than about 30 mol. % cholesterol, the selected proportion being adjusted for the optimal rate of the polypeptide leakage. The composition may be administered in a localised manner to a tumour site or other desired site or may be delivered in a manner in which it targets tumour or other cells.

The polypeptides of the present invention may be generated wholly or partly by chemical synthesis. The polypeptide can be readily prepared according to well-established, standard liquid or, preferably, solid-phase peptide synthesis methods, general descriptions of which are broadly available (see, for example, J. M. Stewart and J. D. Young, (1984) [177], in M. Bodanzsky and A. Bodanzsky, (1984) [178]; or they may be prepared in solution, by the liquid phase method or by any combination of solid-phase, liquid-phase and solution chemistry, e.g., by first completing the respective peptide portion and then, if desired and appropriate, after removal of any protecting groups being present, by introduction of the residue X by reaction of the respective carbonic or sulfonic acid or a reactive derivative thereof.

Another convenient way of producing the polypeptide according to the present invention is to express the nucleic acid encoding it, by use of nucleic acid in an expression system. The present invention further provides an isolated nucleic acid encoding the polypeptides of the present invention. Nucleic acid includes DNA and RNA. The skilled person will be able to determine substitutions, deletions and/or additions to such nucleic acids which will still provide the polypeptide of the present invention.

The present invention also provides constructs in the form of plasmids, vectors, transcription or expression cassettes which comprise at least one nucleic acid as described above. The present invention also provides a recombinant host cell which comprises one or more constructs as above. As mentioned, a nucleic acid encoding the polypeptide of the invention forms an aspect of the present invention, as does a method of production of the polypeptide which method comprises expression from encoding nucleic acid therefor. Expression may conveniently be achieved by culturing under appropriate conditions recombinant host cells containing the nucleic acid. Following production by expression the polypeptide may be isolated and/or purified using any suitable technique, then used as appropriate. Systems for cloning and expression of a polypeptide in a variety of different host cells are well known. Suitable host cells include bacteria, mammalian cells, yeast and baculovirus systems. Mammalian cell lines available in the art for expression of a heterologous polypeptide include Chinese hamster ovary cells, HeLa cells, baby hamster kidney cells, NSO mouse melanoma cells and many others. A common, preferred bacterial host is E. coli.

The expression of polypeptides in prokaryotic cells such as E. coli is well established in the art. For a review see for example [179]. Expression in eukaryotic cells in culture is also available to those skilled in the art as an option for production of the polypeptides of the invention, see for recent review, for example [180, 181].

Suitable vectors can be chosen or constructed, containing appropriate regulatory sequences, including promoter sequences, terminator sequences, polyadenylation sequences, enhancer sequences, marker genes and other sequences as appropriate. Vectors may be plasmids, viral e.g., ‘phage, or phagemid, as appropriate. For further details see, for example, [157]. Many known techniques and protocols for manipulation of nucleic acid, for example in preparation of nucleic acid constructs, mutagenesis, sequencing, introduction of DNA into cells and gene expression, and analysis of proteins, are described in detail in Ausubel et al., 1992 [182].

Thus, a further aspect of the present invention provides a host cell containing nucleic acid in accordance with the invention. A still further aspect provides a method comprising introducing such nucleic acid into a host cell. The introduction may employ any available technique. For eukaryotic cells, suitable techniques may include calcium phosphate transfection, DEAE-Dextran, electroporation, liposome-mediated transfection and transduction using retrovirus or other virus, e.g., vaccinia or, for insect cells, baculovirus. For bacterial cells, suitable techniques may include calcium chloride transformation, electroporation and transfection using bacteriophage. The introduction may be followed by causing or allowing expression from the nucleic acid, e.g., by culturing host cells under conditions for expression of the gene. In one embodiment, the nucleic acid of the invention is integrated into the genome (e.g., chromosome) of the host cell. Integration may be promoted by inclusion of sequences which promote recombination with the genome, in accordance with standard techniques. The present invention also provides a method which comprises using a construct as stated above in an expression system in order to express the polypeptide as above.

The Fragment crystallizable region (Fc region) is the tail region of an antibody that interacts with cell surface receptors called Fc receptors and some proteins of the complement system. This property allows antibodies to activate the immune system. In IgG antibody isotypes, the Fc region is composed of two identical protein fragments, derived from the second and third constant domains of the antibody's two heavy chains. The Fc regions of IgGs comprise a highly conserved N-glycosylation site.

The Fc region of an IgG consists of a paired set of antibody HC domains, each of which has a CH2 fused to a CH3, which form a structure of about 50 kDa. The name “Fragment, crystallizable” (Fc) comes from the fact that after cleavage of serum-derived myeloma IgG fractions with papain, the only fragment that could be crystallized was the paired CH2-CH3 fragment.

Within the Fc, the two CH3 domains bind each other tightly, whereas the two CH2 domains have no direct protein-protein contact with one another. An oligosaccharide is bound to asparagine-297 (N297) within each of the two CH2 domains, filling part of the space between the two CH2s. In some crystal structures, hydrogen bonding has been observed between the two carbohydrate chains, directly and through bridging water molecules. While the antibody appears to be a highly segmented molecule, it has been demonstrated that the structure of the Fc can impact the binding of the antigen-binding fragments (Fabs) to the targeted antigens and, similarly, that the content of the variable chain in the FAbs can impact binding of the Fc to various receptors. Recently circular dichroism studies have confirmed significant structural coupling between the FAb arms and the Fc of the IgG. Thus the IgG molecule is a highly complex molecule in which the different domains significantly interact, even at long distances.

Avidity refers to the accumulated strength of multiple affinities of individual non-covalent binding interactions, such as between a protein receptor and its ligand, and can also be referred to as ‘functional affinity’. As such, avidity is distinct from intrinsic affinity, which describes the strength of a single interaction. However, because individual binding events increase the likelihood of other interactions to occur (i.e. increase the local concentration of each binding partner in proximity to the binding site), avidity should not be thought of as the mere sum of constituent affinities but as the combined effect of all affinities participating in the biomolecular interaction. The utility of the distinction between “intrinsic affinity” and “functional affinity” arises from the different emphasis involved in each term. The former is most useful when the structural relationship between the antibody combining site and the complementary region of the ligand is under scrutiny or when kinetic mechanisms of the specific interaction are under investigation. On the other hand, the latter is particularly significant when the quantitative measurement of the enhancement of affinity is being examined, as in the present invention.

Both ‘functional affinity’ and ‘intrinsic affinity’ refer to formally identical reversible processes as follows:

F₁+L₁ [symbol] 1:1 complex (monovalent)

Abₙ+Lₘ [symbol] 1:1 complex (multivalent)

where F₁ is a monovalent antibody fragment, L₁ a monovalent ligand, Abₙ is a multivalent antibody, and Lₘ is a multivalent ligand with m groups. In each instance, two kinetic units combine reversibly to form one unit and each process can, in principle, be characterized by an association constant. Since the association constant is a measure of the thermodynamic affinity both processes can therefore be assigned quantitative values of the affinity.

The present invention also extends to variants of any peptide sequences disclosed herein. As used herein the term “variant” relates to proteins that have a similar amino acid sequence and/or that retain the same function. For instance, the term “variant” encompasses proteins or polypeptides which include one or more amino acid additions, deletions, substitutions or the like. An example of a variant of the present invention is a protein comprising a peptide as defined below, apart from the substitution of one or more amino acids with one or more other amino acids. Amino acid substitutions may be made to, for example, reduce or eliminate liabilities in the amino acid sequences.

The skilled person is aware that various amino acids have similar properties. One or more such amino acids of a substance can often be substituted by one or more other such amino acids without eliminating a desired activity of that substance.

Thus, the amino acids glycine, alanine, valine, leucine and isoleucine can often be substituted for one another (amino acids having aliphatic side chains). Of these possible substitutions it is preferred that glycine and alanine are used to substitute for one another (since they have relatively short side chains) and that valine, leucine and isoleucine are used to substitute for one another (since they have larger aliphatic side chains which are hydrophobic). Other amino acids which can often be substituted for one another include: phenylalanine, tyrosine and tryptophan (amino acids having aromatic side chains); lysine, arginine and histidine (amino acids having basic side chains); aspartate and glutamate (amino acids having acidic side chains); asparagine and glutamine (amino acids having amide side chains); and cysteine and methionine (amino acids having sulphur containing side chains). Substitutions of this nature are often referred to as “conservative” or “semi-conservative” amino acid substitutions.

Using the three letter and one letter codes the naturally occurring amino acids may be referred to as follows: glycine (G or Gly), alanine (A or Ala), valine (V or Val), leucine (L or Leu), isoleucine (I or lie), proline (P or Pro), phenylalanine (F or Phe), tyrosine (Y or Tyr), tryptophan (W or Trp), lysine (K or Lys), arginine (R or Arg), histidine (H or His), aspartic acid (D or Asp), glutamic acid (E or Glu), asparagine (N or Asn), glutamine (Q or Gln), cysteine (C or Cys), methionine (M or Met), serine (S or Ser) and Threonine (T or Thr). Where a residue may be aspartic acid or asparagine, the symbols Asx or B may be used. Where a residue may be glutamic acid or glutamine, the symbols Glx or Z may be used. References to aspartic acid include aspartate, and glutamic acid include glutamate, unless the context specifies otherwise.

Amino acid deletions or insertions can also be made relative to the amino acid sequence for the fusion protein referred to below. Thus, for example, amino acids which do not have a substantial effect on the activity of the polypeptide, or at least which do not eliminate such activity, can be deleted. Such deletions can be advantageous since the overall length and the molecular weight of a polypeptide can be reduced whilst still retaining activity. This can enable the amount of polypeptide required for a particular purpose to be reduced—for example, dosage levels can be reduced.

In some embodiments, the following amino acids can be exchange for each other for conservative amino acid substitutions:

| Class | Exchangeable amino acids |
|---|---|
| Aliphatic | Glycine, Alanine, Valine, Leucine, |
| | Isoleucine |
| Hydroxyl or | Serine, Cysteine, Threonine, |
| Sulphur/Selenium-containing | Methionine |
| Aromatic | Phenylalanine, Tyrosine, |
| | Tryptophan |
| Basic | Histidine, Lysine, Arginine |
| Acidic and their Amide | Aspartate, Glutamate, |
| | Asparagine, Glutamine |

Therefore, references to “conservative” amino acid substitutions refer to amino acid substitutions in which one or more of the amino acids in the sequence of the antibody (e.g. in the CDRs or in the VH or VL sequences) is substituted with another amino acid in the same class as indicated above. Conservative amino acid substitutions may be preferred in the CDR regions to minimise adverse effects on the function of the antibody. However, conservative amino acid substitutions may also occur in the framework regions.

Amino acid changes relative to the sequence given below can be made using any suitable technique e.g. by using site-directed mutagenesis or solid-state synthesis.

It should be appreciated that amino acid substitutions or insertions within the scope of the present invention can be made using naturally occurring or non-naturally occurring amino acids, although naturally occurring amino acids may be preferred. Whether or not natural or synthetic amino acids are used, it may be preferred that only L-amino acids are present.

The invention also provides
A nucleic acid vaccine whereby the antigen is a tumour specific or tumour associated antigen.
A nucleic acid vaccine whereby the antigen is a viral or bacterial antigen.
A nucleic acid vaccine whereby the antigens are two viral proteins that generate both T cell responses and VNAbs.
A nucleic acid vaccine whereby the antigens are two viral proteins to generate CD8 T cells, CD4 T cells and VNAbs.
A nucleic acid vaccine whereby the antigens are a virus nucleocapsid protein and the key receptor-binding domain of the spike protein to generate both T cell responses and VNAbs.
A nucleic acid vaccine whereby the antigens are a virus nucleocapsid protein and the key receptor-binding domain of the spike protein to generate CD8 T cells, CD4 T cells and VNAbs.
A nucleic acid vaccine whereby the antigens are a coronavirus nucleocapsid protein and the key receptor-binding domain of the spike protein to generate both T cell responses and VNAbs.
A nucleic acid vaccine whereby the antigens are both coronavirus nucleocapsid protein and the key receptor-binding domain of the spike protein to generate CD8 T cells, CD4 T cells and VNAbs.
A nucleic acid vaccine whereby the antigens are both the SARS-CoV-2 coronavirus nucleocapsid protein and the key receptor-binding domain of the spike protein to generate both T cell responses and VNAbs.
A nucleic acid vaccine whereby the antigens are both SARS-CoV-2 coronavirus nucleocapsid protein and the key receptor-binding domain of the spike protein to generate CD8 T cells, CD4 T cells and VNAbs.
A nucleic acid vaccine as set out above wherein low dose is delivered to elicit high avidity CD8 T cells.
A nucleic acid vaccine as set out above wherein the receptor binding domain is trimerised with fibritin.
A nucleic acid vaccine as set out above wherein the receptor binding domain is trimerised with fibritin linked with a flexible GS linker.
A nucleic acid vaccine as set out above wherein the receptor binding domain is stabilised with an extra disulphide bond.
A nucleic acid vaccine encoding the COVID-19 spike receptor binding domain aa 319-541 with a human heavy chain leader sequence and the nucleocapsid protein linked to the hinge and CH2 and CH3 domains of human IgG1 Fc with a human heavy chain leader sequence.
A nucleic acid vaccine encoding the COVID-19 spike receptor binding domain aa 319-541 linked to fibritin with a human heavy chain leader sequence and the nucleocapsid protein linked to the hinge and CH2 and CH3 domains of human IgG1 Fc with a human heavy leader sequence.
A nucleic acid vaccine encoding the COVID-19 spike receptor binding domain aa 319-541 linked to the hinge and CH2 and CH3 domains of human IgG1 Fc with a human heavy chain leader sequence and the nucleocapsid protein with a human heavy chain leader sequence.
A nucleic acid vaccine encoding the COVID-19 spike receptor binding domain aa 319-541 with a human heavy chain leader sequence and the nucleocapsid protein with a human heavy chain leader sequence.
A nucleic acid vaccine encoding the COVID-19 spike receptor binding domain aa 330-525 with a GS linker and fibritin with a human heavy chain leader sequence and the nucleocapsid protein linked to the hinge and CH2 and CH3 domains of human IgG1 Fc with a human heavy chain leader sequence.
A nucleic acid vaccine encoding the COVID-19 spike receptor binding domain aa 330-525 with a GS linker and additional cysteine residues with a human heavy chain leader sequence and the nucleocapsid protein linked to the hinge and CH2 and CH3 domains of human IgG1 Fc with a human heavy chain leader sequence.
A modified IgG1 antibody or antigen binding fragment thereof comprising one or more residues of an Fc region of an immunoglobulin and a binding region, wherein one or more residues of the Fc region are modified to the corresponding residue from a mouse IgG3 antibody and wherein the modified IgG1 antibody or antigen binding fragment thereof has enhanced functional affinity when compared to a corresponding IgG1 antibody or antigen binding fragment thereof comprising wildtype Fc region residues.
A nucleic acid vaccine comprising a modified IgG1 antibody or antigen binding fragment thereof comprising one or more residues of an Fc region of an immunoglobulin and a binding region, wherein one or more residues of the Fc region are modified to the corresponding residue from a mouse IgG3 antibody and wherein the modified IgG1 antibody or antigen binding fragment thereof has enhanced functional affinity when compared to a corresponding IgG1 antibody or antigen binding fragment thereof comprising wildtype Fc region residues.
A modified IgG1 antibody or antigen binding fragment thereof comprising one or more residues of an Fc region of an immunoglobulin and a binding region, wherein one or more residues of the Fc region are modified to the corresponding residue from a mouse IgG3 antibody and wherein the one or more residues of the Fc region are selected from: Q342, P343, E345, N361, Q362, P374, D376, optionally wherein the one or more modified residues of the Fc region are selected from: Q342R, P343A, E345T, N361K, Q362K, P374S, D376A.
A nucleic acid vaccine comprising a modified IgG1 antibody or antigen binding fragment thereof comprising one or more residues of an Fc region of an immunoglobulin and a binding region, wherein one or more residues of the Fc region are modified to the corresponding residue from a mouse IgG3 antibody and wherein the one or more residues of the Fc region are selected from: Q342, P343, E345, N361, Q362, P374, D376, optionally wherein the one or more modified residues of the Fc region are selected from: Q342R, P343A, E345T, N361K, Q362K, P374S, D376A.
A modified IgG1 antibody or antigen binding fragment thereof comprising one or more residues of an Fc region of an immunoglobulin and a binding region, wherein one or more residues of the Fc region are modified to the corresponding residue from a mouse IgG3 antibody and wherein the one or more residues of the Fc region are selected from: N286, K288, K290, Q342, P343, E345, L351, T359, N361, Q362, G371, P374, S375, D376, A378, optionally wherein the one or more modified residues of the Fc region are selected from: N286T, K288W, K290Q, Q342R, P343A, E345T, L351I, T359S, N361K, Q362K, G371N, P374S, S375E, D376A, A378S.
A nucleic acid vaccine comprising a modified IgG1 antibody or antigen binding fragment thereof comprising one or more residues of an Fc region of an immunoglobulin and a binding region, wherein one or more residues of the Fc region are modified to the corresponding residue from a mouse IgG3 antibody and wherein the one or more residues of the Fc region are selected from: N286, K288, K290, Q342, P343, E345, L351, T359, N361, Q362, G371, P374, S375, D376, A378, optionally wherein the one or more modified residues of the Fc region are selected from: N286T, K288W, K290Q, Q342R, P343A, E345T, L351I, T359S, N361K, Q362K, G371N, P374S, S375E, D376A, A378S.
A modified IgG1 antibody or antigen binding fragment thereof comprising one or more residues of an Fc region of an immunoglobulin and a binding region, wherein one or more residues of the Fc region are modified to the corresponding residue from a mouse IgG3 antibody and wherein the one or more residues of the Fc region are selected from: N286, K288, K290, A339, Q342, P343, R344, E345, L351, S354, D356, E357, L358, T359, N361, Q362, K370, G371, Y373, P374, S375, D376, A378, optionally wherein the one or more modified residues of the Fc region are selected from: N286T, K288W, K290Q, A339P, Q342R, P343A, R344Q E345T, L351I, S354P, D356E, E357Q L358M, T359S, N361K, Q362K, K370T, G371N, Y373F, P374S, S375E, D376A, A378S.
A nucleic acid vaccine comprising a modified IgG1 antibody or antigen binding fragment thereof comprising one or more residues of an Fc region of an immunoglobulin and a binding region, wherein one or more residues of the Fc region are modified to the corresponding residue from a mouse IgG3 antibody and wherein the one or more residues of the Fc region are selected from: N286, K288, K290, A339, Q342, P343, R344, E345, L351, S354, D356, E357, L358, T359, N361, Q362, K370, G371, Y373, P374, S375, D376, A378, optionally wherein the one or more modified residues of the Fc region are selected from: N286T, K288W, K290Q, A339P, Q342R, P343A, R344Q, E345T, L351I, S354P, D356E, E357Q L358M, T359S, N361K, Q362K, K370T, G371N, Y373F, P374S, S375E, D376A, A378S.
A modified IgG1 antibody or antigen binding fragment thereof comprising one or more residues of an Fc region of an immunoglobulin and a binding region, wherein one or more residues of the Fc region are modified to the corresponding residue from a mouse IgG3 antibody and wherein the one or more residues of the Fc region are selected from: N286, K288, K290, E294, Y300, V305, A339, Q342, P343, R344, E345, L351, S354, D356, E357, L358, T359, N361, Q362, K370, G371, Y373, P374, S375, D376, A378, optionally wherein the one or more modified residues of the Fc region are selected from: N286T, K288W, K290Q, E294A, Y300F, V305A, A339P, Q342R, P343A, R344Q E345T, L351I, S354P, D356E, E357Q L358M, T359S, N361K, Q362K, K370T, G371N, Y373F, P374S, S375E, D376A, A378S.
A nucleic acid vaccine comprising a modified IgG1 antibody or antigen binding fragment thereof comprising one or more residues of an Fc region of an immunoglobulin and a binding region, wherein one or more residues of the Fc region are modified to the corresponding residue from a mouse IgG3 antibody and wherein the one or more residues of the Fc region are selected from: N286, K288, K290, E294, Y300, V305, A339, Q342, P343, R344, E345, L351, S354, D356, E357, L358, T359, N361, Q362, K370, G371, Y373, P374, S375, D376, A378, optionally wherein the one or more modified residues of the Fc region are selected from: N286T, K288W, K290Q, E294A, Y300F, V305A, A339P, Q342R, P343A, R344Q E345T, L351I, S354P, D356E, E357Q L358M, T359S, N361K, Q362K, K370T, G371N, Y373F, P374S, S375E, D376A, A378S.

Preferred features of each aspect of the invention are as for each of the other aspects mutatis mutandis. The prior art documents mentioned herein are incorporated to the fullest extent permitted by law.

In describing the embodiments of the invention, the terminology is not intended to be limited to the specific terms so selected, and it is understood that each specific term includes all technical equivalents which operate in a similar manner to accomplish a similar purpose.

### EXAMPLES

The present invention will now be described further with reference to the following examples and the accompanying drawings. Future data collection from healthy volunteers immunised, using electroporation, with the novel pDNA vaccine, engineered to induce both VNAbs and potent T cells have been informed as a direct result of the example outcomes.

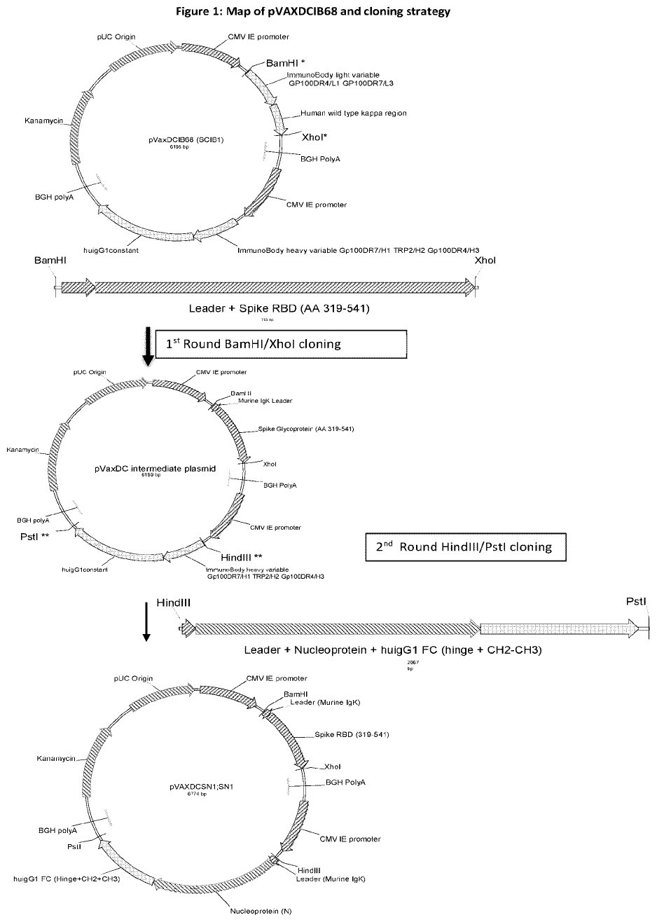

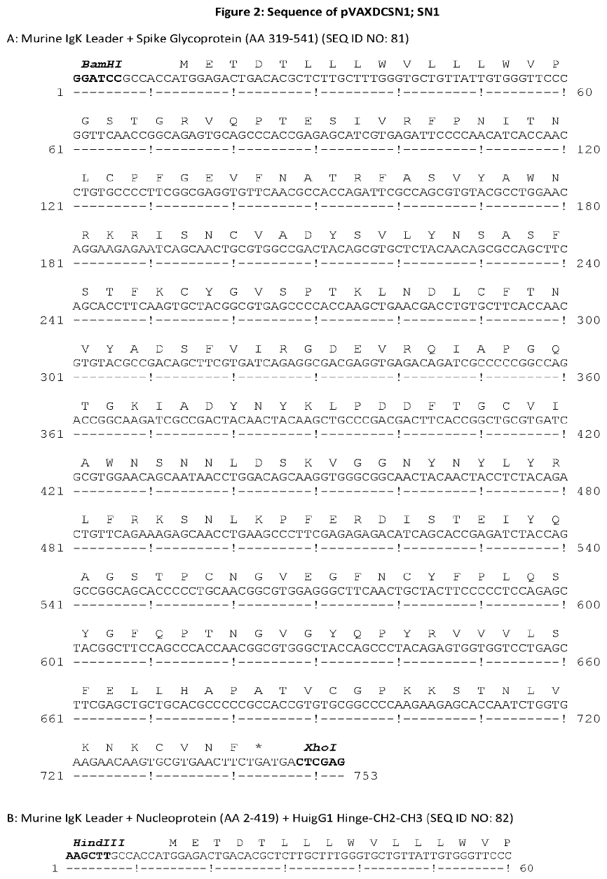

FIG. 1: Map of pVAXDCIB68 and cloning strategy

Depicting cloning sites BamHI*/XhoI* and HindIII**/PstI** utilised for excision of light and heavy chain and replacement with S and N sections in first* and second** round of cloning respectively.

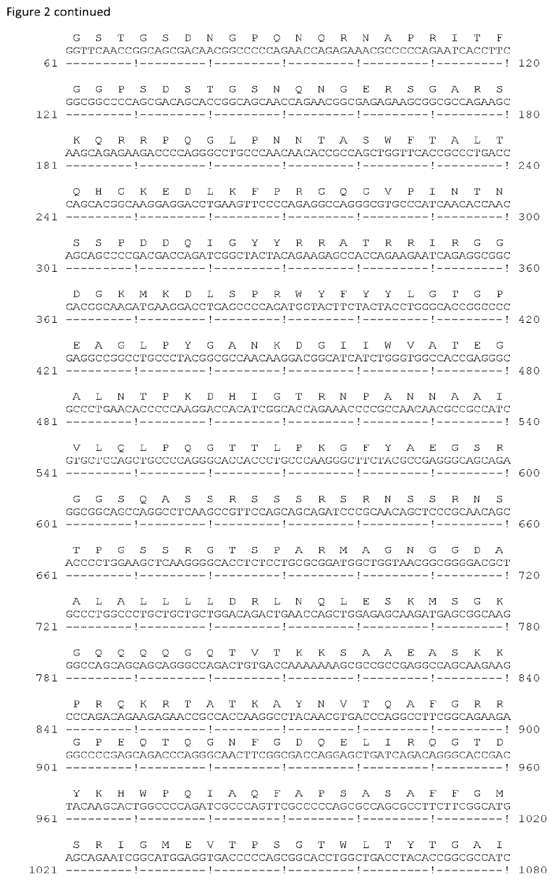

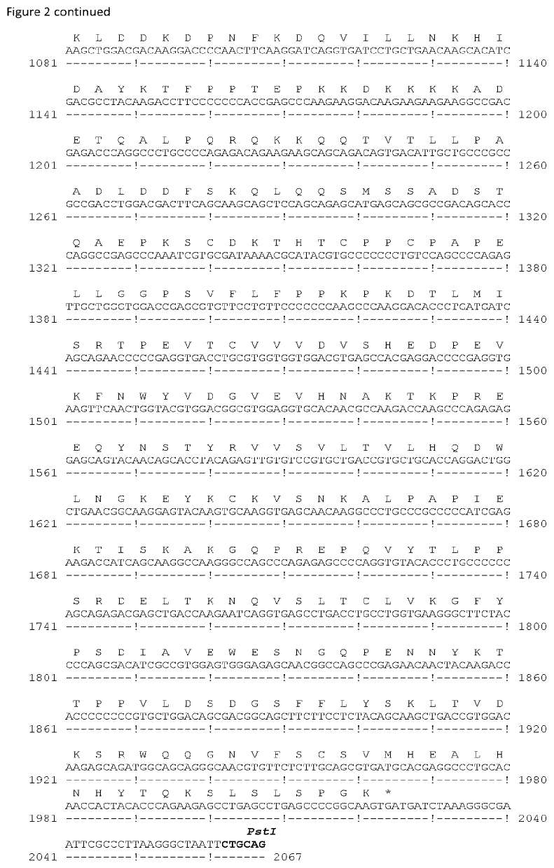

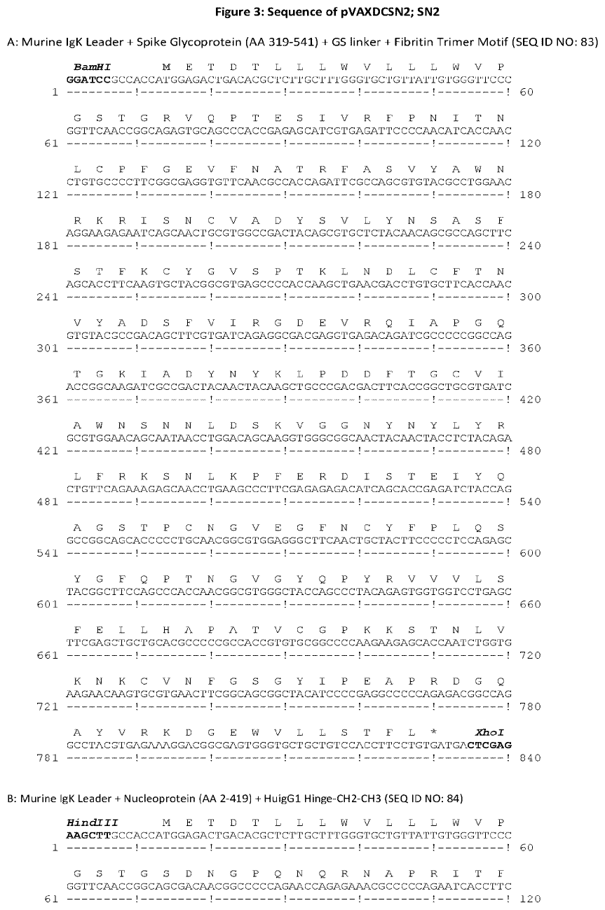

FIG. 2: Sequence of pVAXDCSN1; SN1

Nucleotide and amino acid sequence of the S glycoprotein and N full length chains within the expression vector pVAXDC. The S chain encodes RBD amino acids 319-541 and a murine IgK leader. The nucleoprotein chain encodes amino acids 2-419 fused in frame with the human IgG1 hinge-CH2-CH3 along with the murine IgK leader. The stop codon is depicted by an asterisk. The BamHI/XhoI and HindIII/PstI restriction sites utilised in transfer of both chains are highlighted.

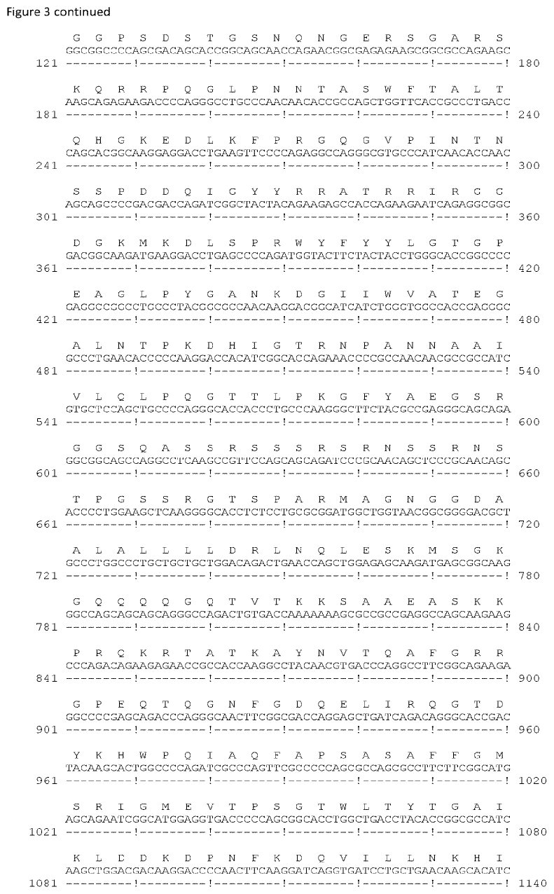

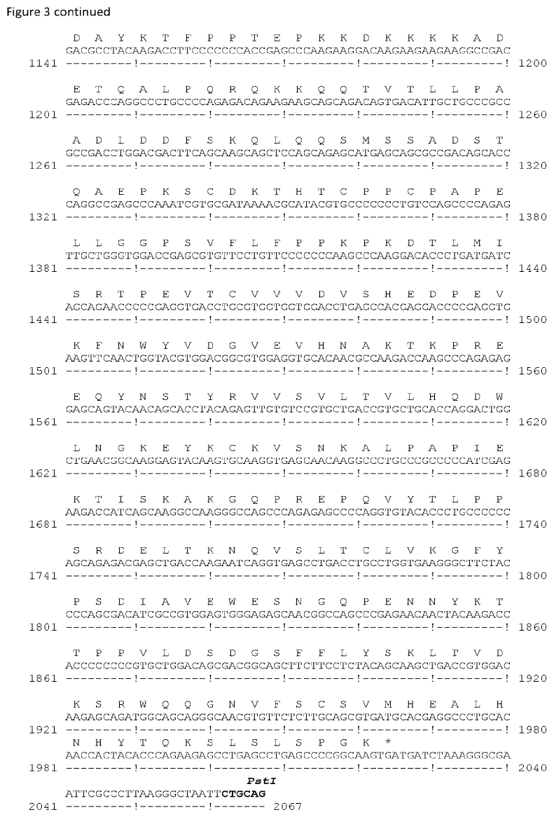

FIG. 3: Sequence of pVAXDCSN2; SN2

Nucleotide and amino acid sequence of the S and N full length chains within the expression vector pVAXDC. Both chains contain a murine IgK leader. The S chain encodes RBD amino acids 319-541 linked via a glycine serine to a fibritin trimer motif. The N chain encodes amino acids 2-419 fused in frame with the human IgG1 hinge-CH2-CH3. The stop codon is depicted by an asterisk. The BamHI/XhoI and HindIII/PstI restriction sites utilised in transfer of both chains are highlighted.

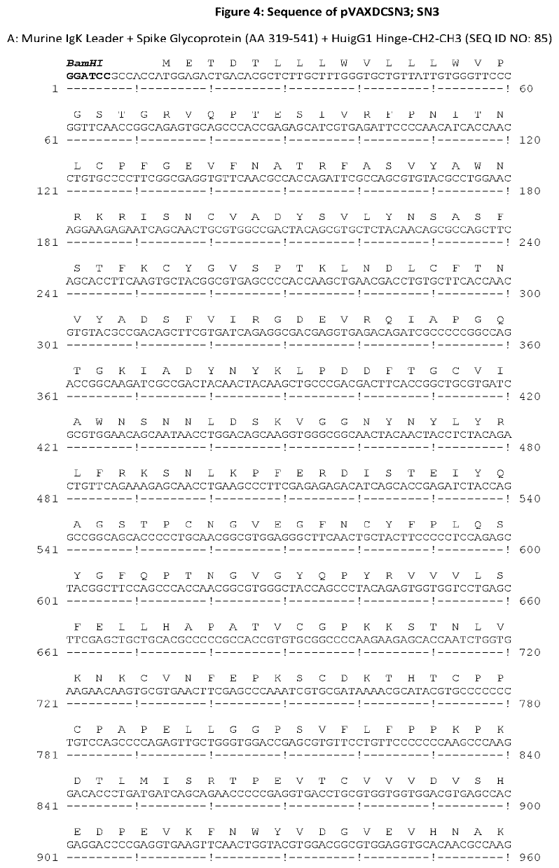

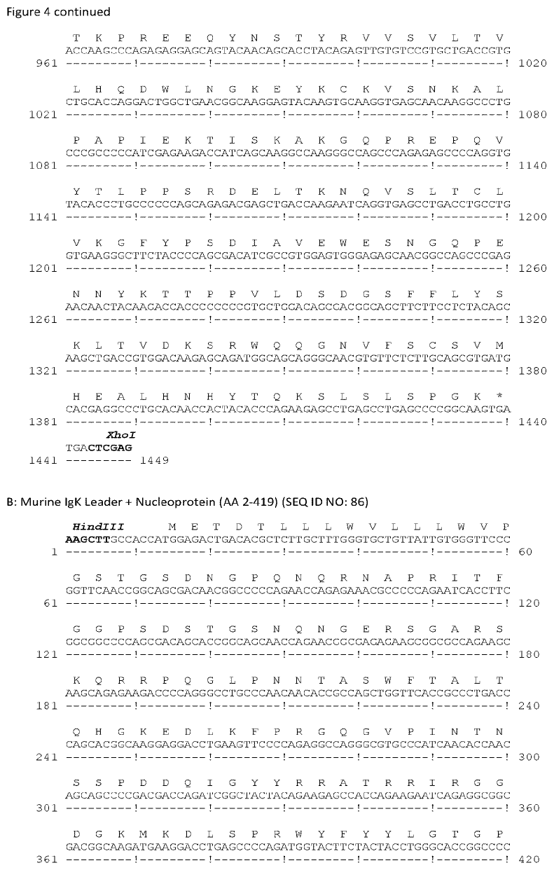

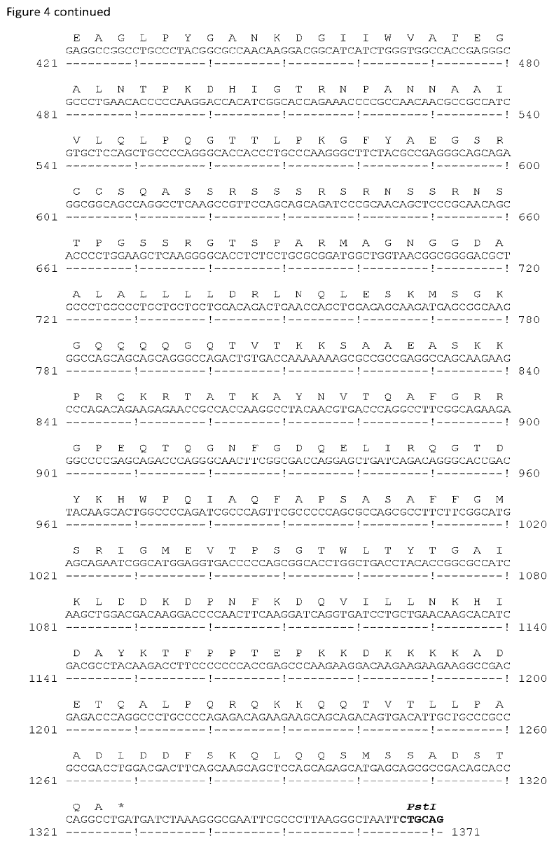

FIG. 4: Sequence of pVAXDCSN3; SN3

Nucleotide and amino acid sequence of the S and N full length chains within the expression vector pVAXDC. Both chains contain a murine IgK leader. The S chain encodes RBD amino acids 319-541 fused in frame with the human IgG1 hinge-CH2-CH3. The N chain encodes amino acids 2-419. The stop codon is depicted by an asterisk. The BamHI/XhoI and HindIII/PstI restriction sites utilised in transfer of both chains are highlighted.

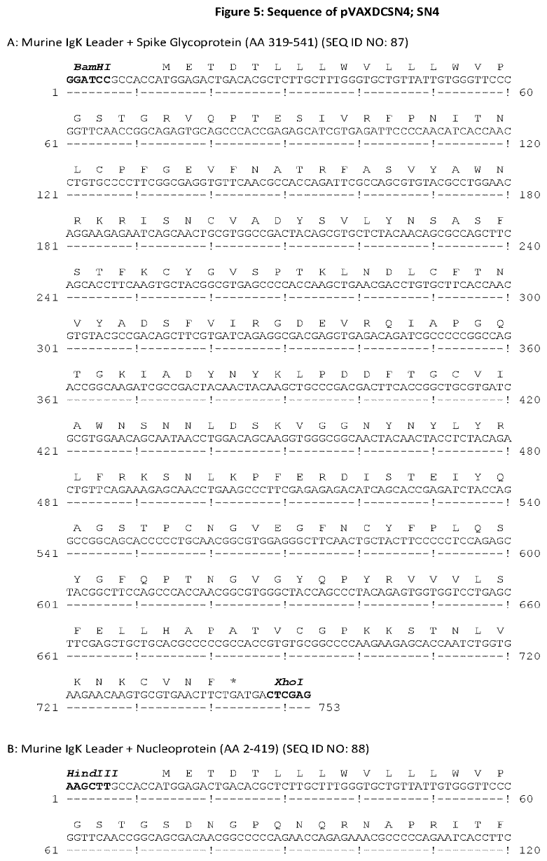

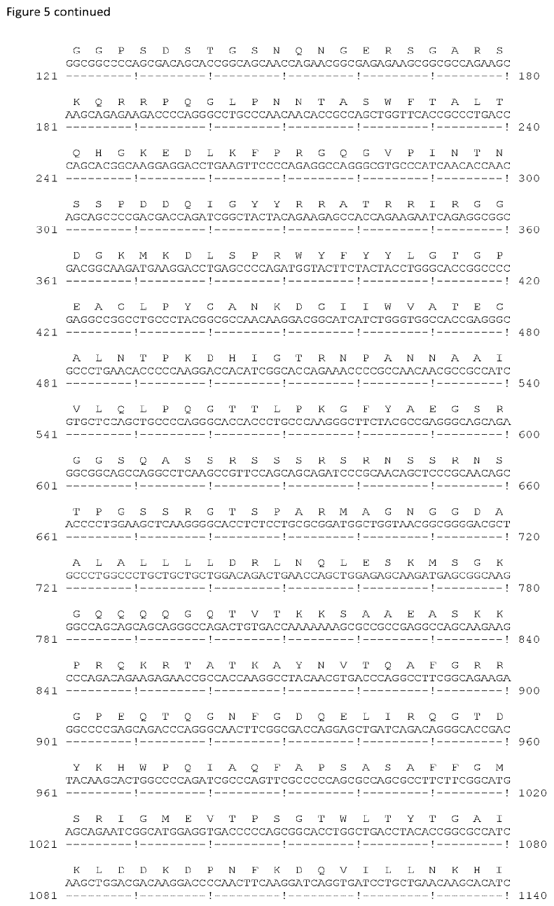

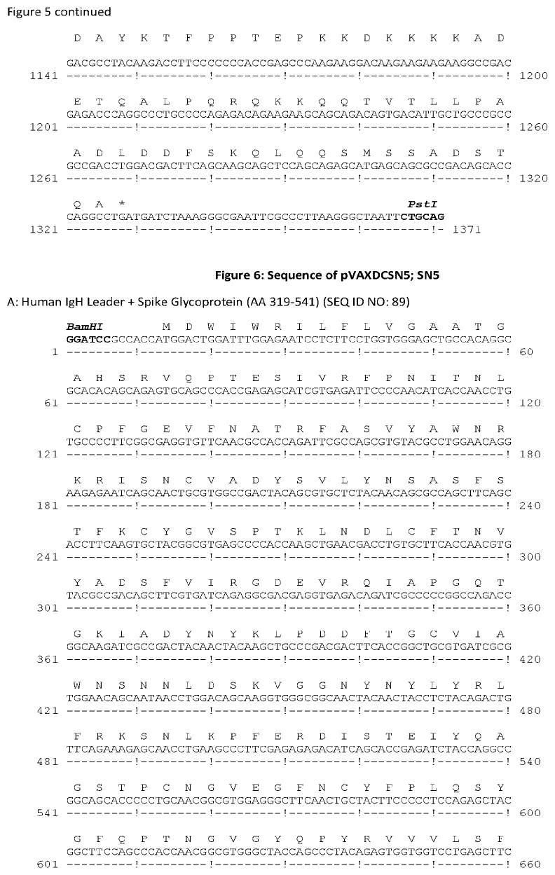

FIG. 5: Sequence of pVAXDCSN4; SN4

Nucleotide and amino acid sequence of the S and N full length chains within the expression vector pVAXDC. Both chains contain a murine IgK leader. The S glycoprotein chain encodes RBD amino acids 319-541 while the N chain encodes amino acids 2-419. The stop codon is depicted by an asterisk. The BamHI/XhoI and HindIII/PstI restriction sites utilised in transfer of both chains are highlighted.

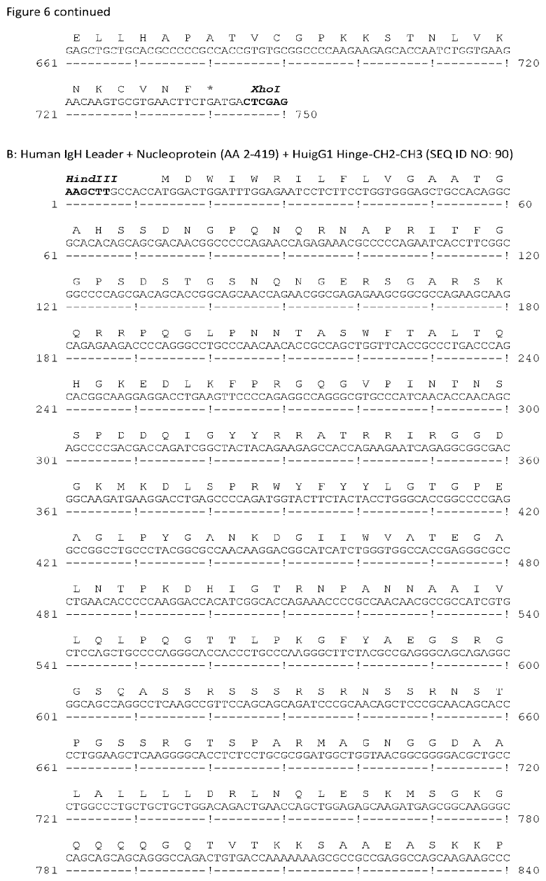

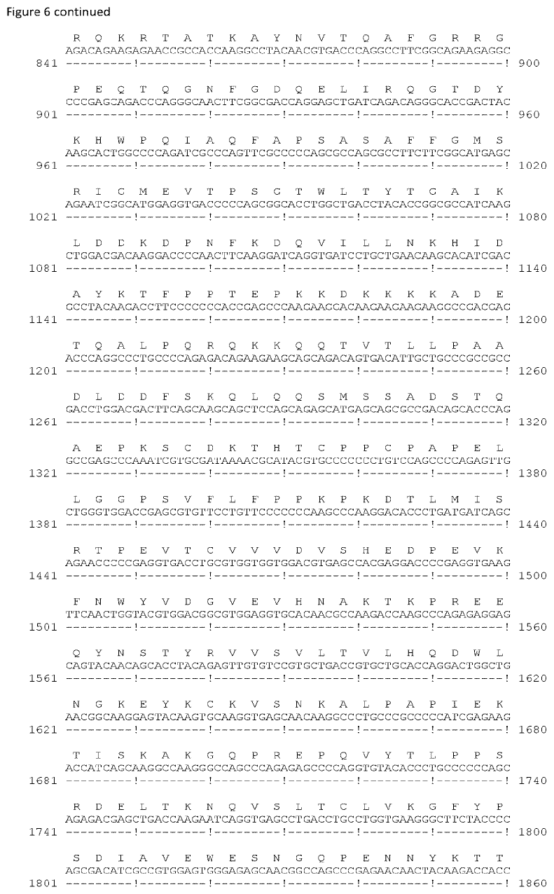

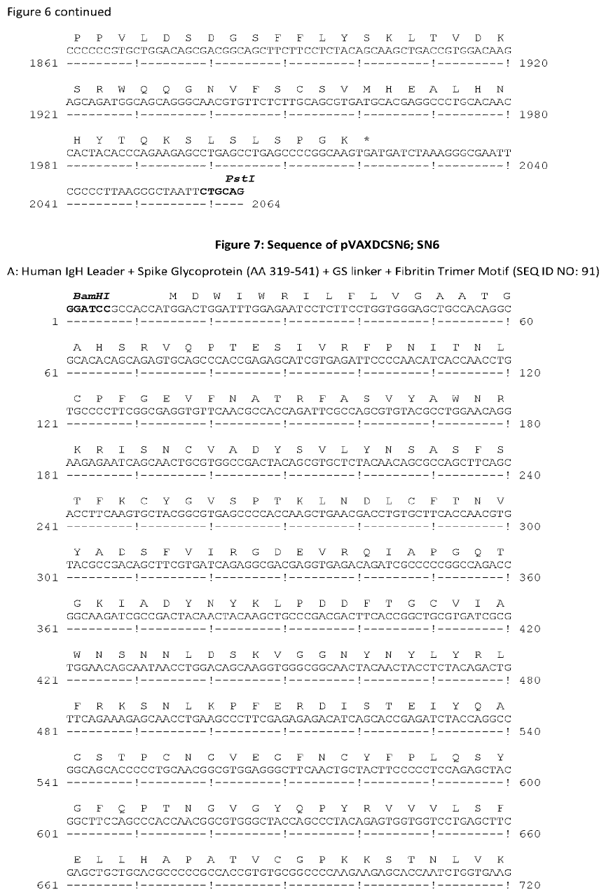

FIG. 6: Sequence of pVAXDCSN5; SN5

Nucleotide and amino acid sequence of the S and N full length chains within the expression vector pVAXDC. The S chain encodes RBD amino acids 319-541 and a human IgH leader. The N chain encodes amino acids 2-419 fused in frame with the human IgG1 hinge-CH2-CH3 along with the human IgH leader. The stop codon is depicted by an asterisk. The BamHI/XhoI and HindIII/PstI restriction sites utilised in transfer of both chains are highlighted.

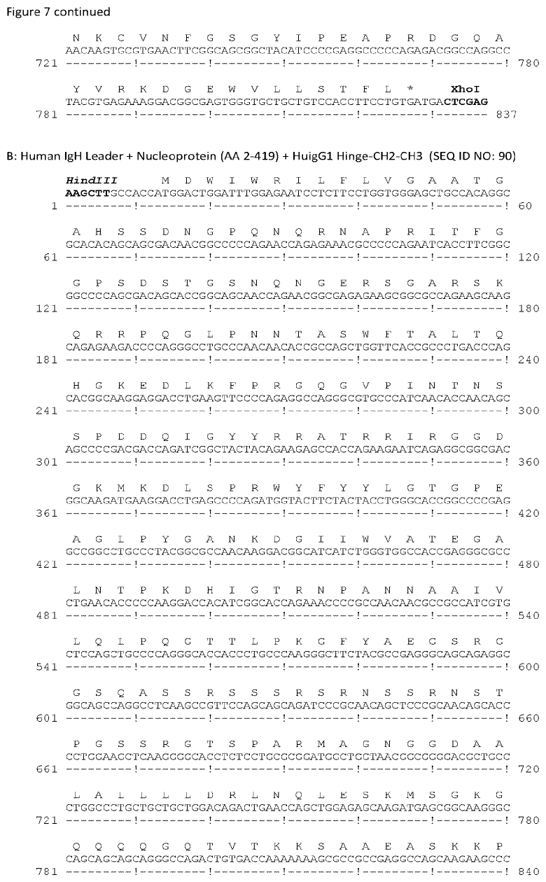

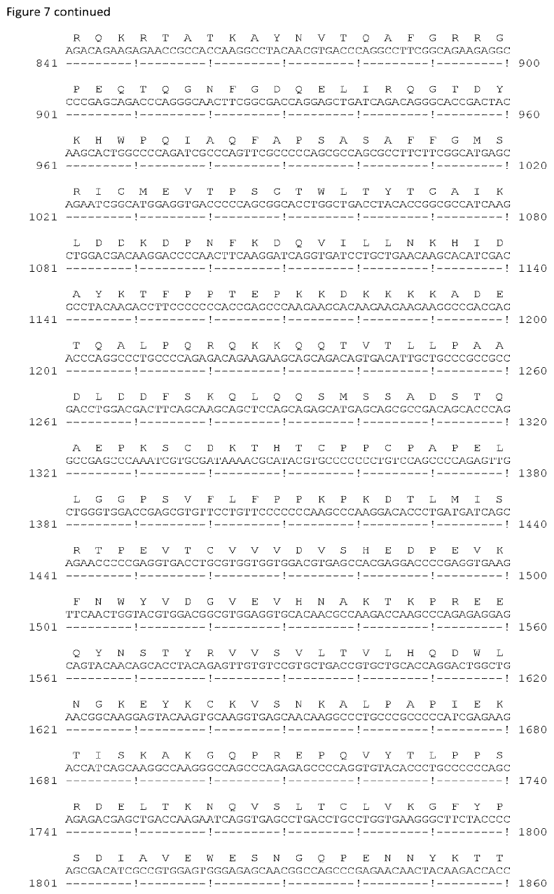

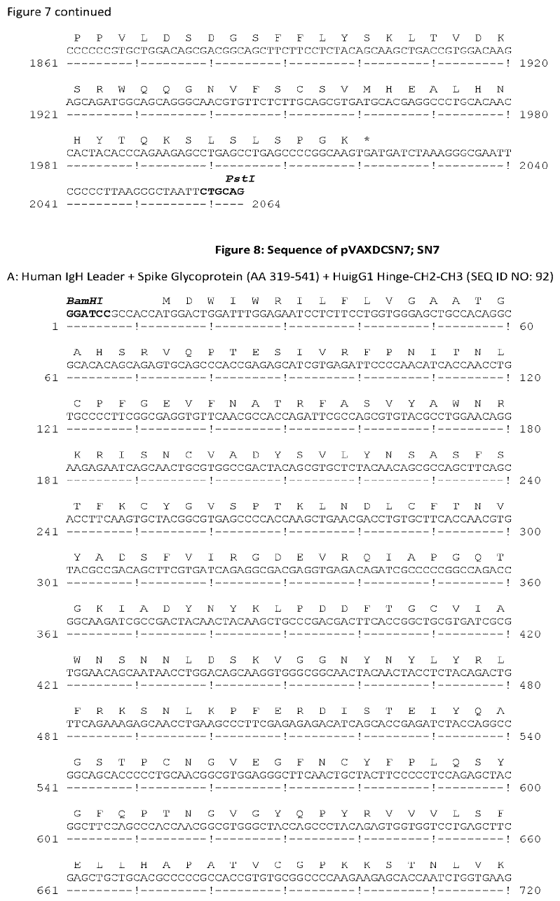

FIG. 7: Sequence of pVAXDCSN6; SN6

Nucleotide and amino acid sequence of the S and N full length chains within the expression vector pVAXDC. Both chains contain a human IgH leader. The S chain encodes RBD amino acids 319-541 linked via a glycine serine to a fibritin trimer motif. The N chain encodes amino acids 2-419 fused in frame with the human IgG1 hinge-CH2-CH3. The stop codon is depicted by an asterisk. The BamHI/XhoI and HindIII/PstI restriction sites utilised in transfer of both chains are highlighted.

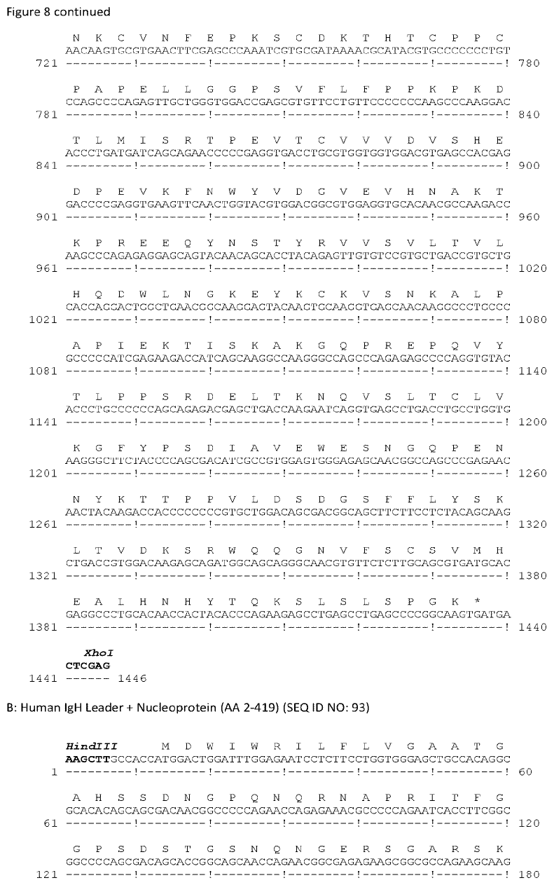

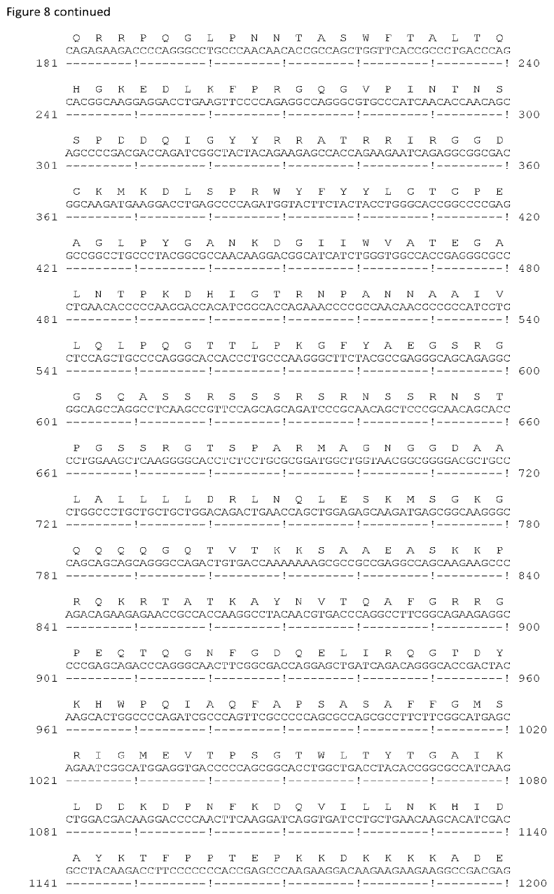

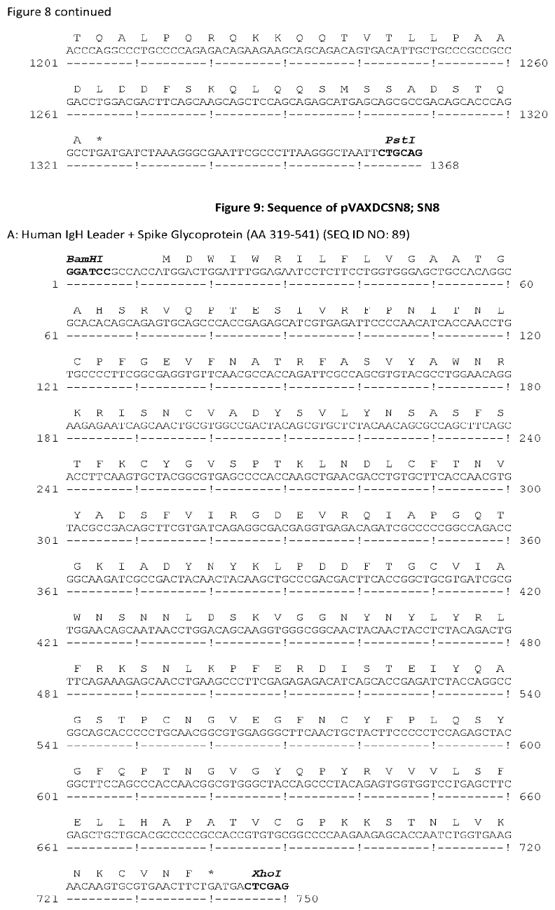

FIG. 8: Sequence of pVAXDCSN7; SN7

Nucleotide and amino acid sequence of the S and N full length chains within the expression vector pVAXDC. Both chains contain a human IgH leader. The S chain encodes RBD amino acids 319-541 fused in frame with the human IgG1 hinge-CH2-CH3. The N chain encodes amino acids 2-419. The stop codon is depicted by an asterisk. The BamHI/XhoI and HindIII/PstI restriction sites utilised in transfer of both chains are highlighted.

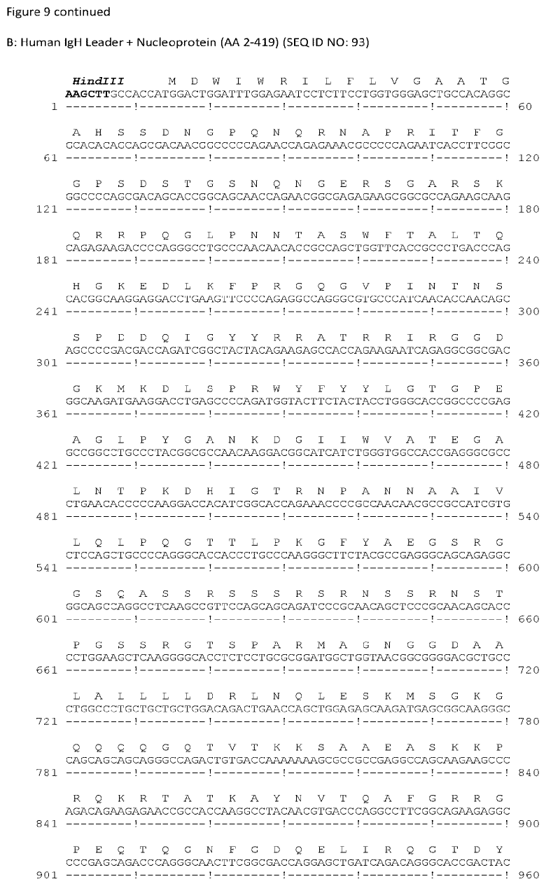

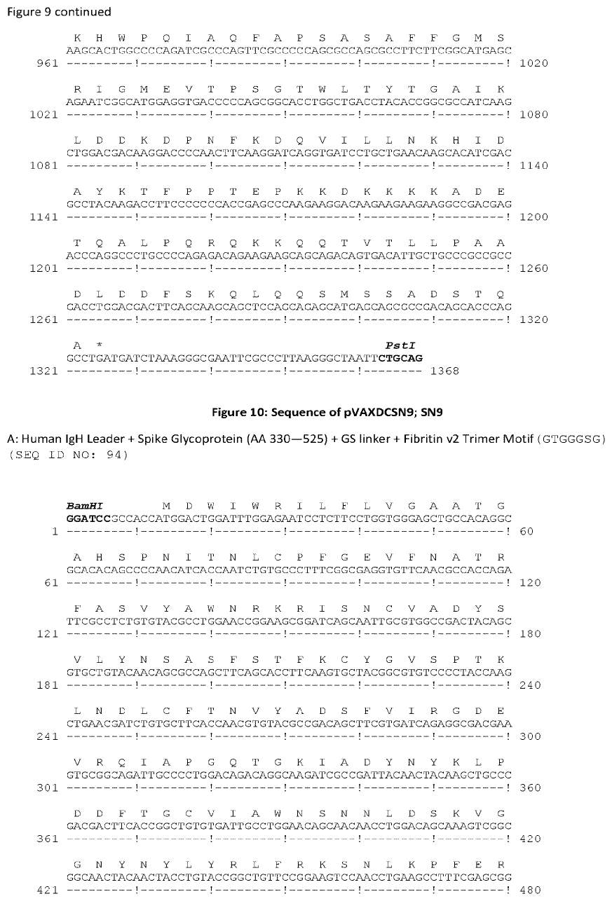

FIG. 9: Sequence of pVAXDCSN8; SN8

Nucleotide and amino acid sequence of the S and N full length chains within the expression vector pVAXDC. Both chains contain a human IgH leader. The S glycoprotein chain encodes RBD amino acids 319-541 while the N chain encodes amino acids 2-419. The stop codon is depicted by an asterisk. The BamHI/XhoI and HindIII/PstI restriction sites utilised in transfer of both chains are highlighted.

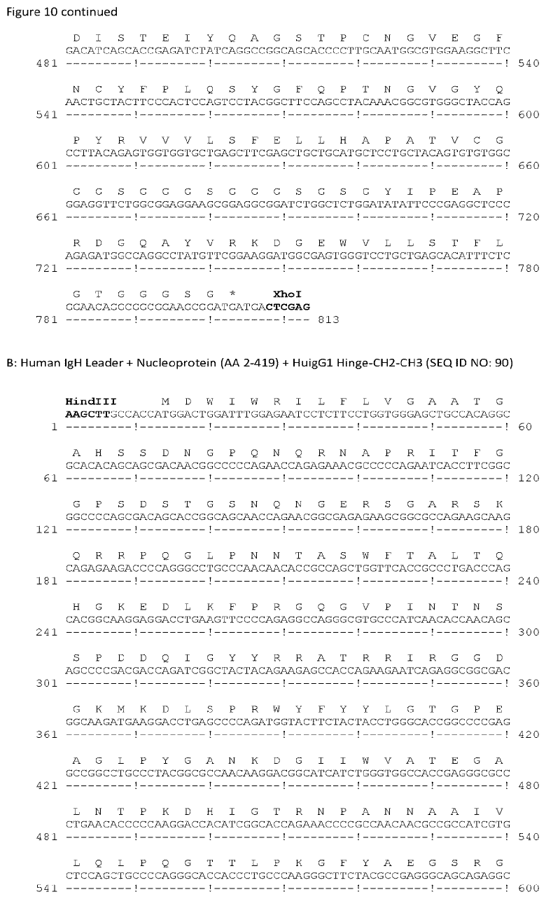

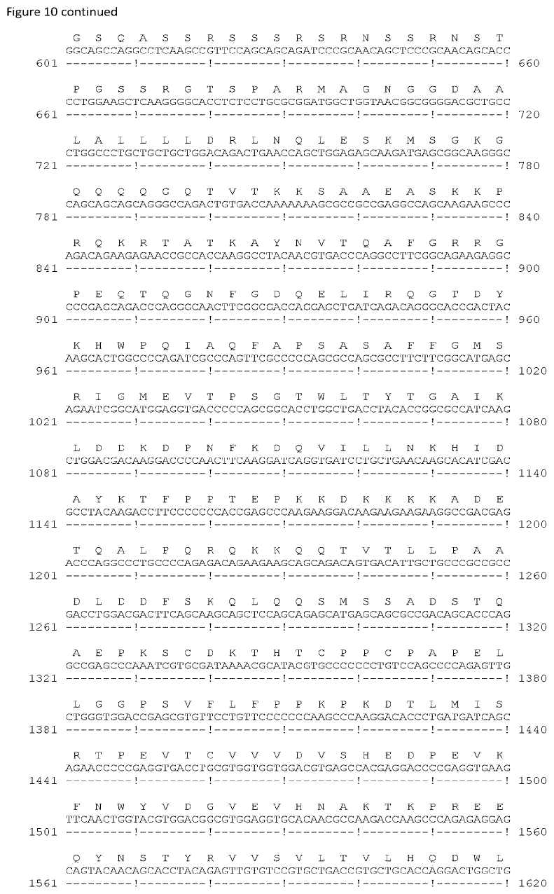

FIG. 10: Sequence of pVAXDCSN9; SN9

Nucleotide and amino acid sequence of the S and N full length chains within the expression vector pVAXDC. Both chains contain a human IgH leader. The S chain encodes RBD amino acids 330-525 attached via a longer (GGGS)₃GS glycine serine linker (SEQ ID NO: 74) to a fibritin trimer motif (GTGGGSG) (SEQ ID NO: 121). The N chain encodes amino acids 2-419 fused in frame with the human IgG1 hinge-CH2-CH3. The stop codon is depicted by an asterisk. The BamHI/XhoI and HindIII/PstI restriction sites utilised in transfer of both chains are highlighted.

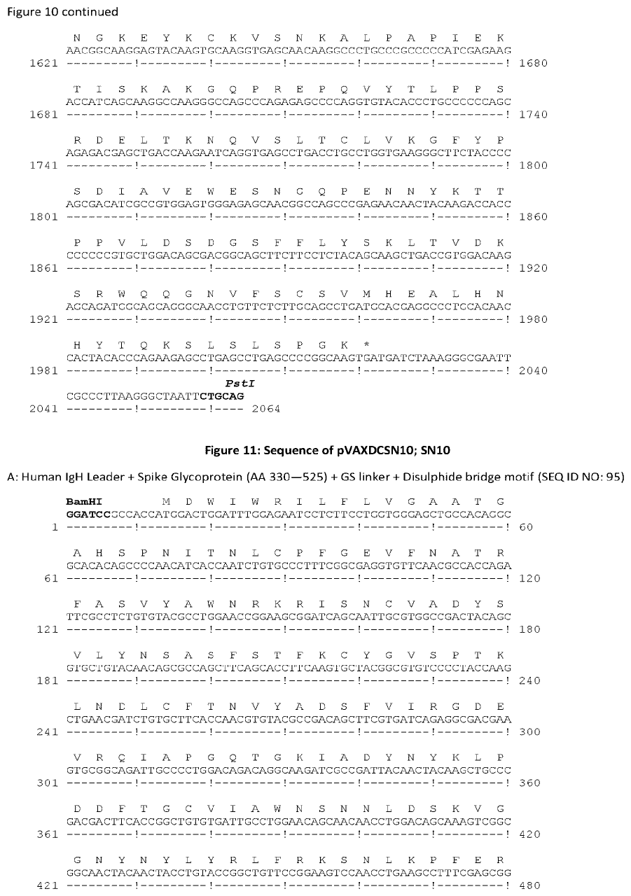

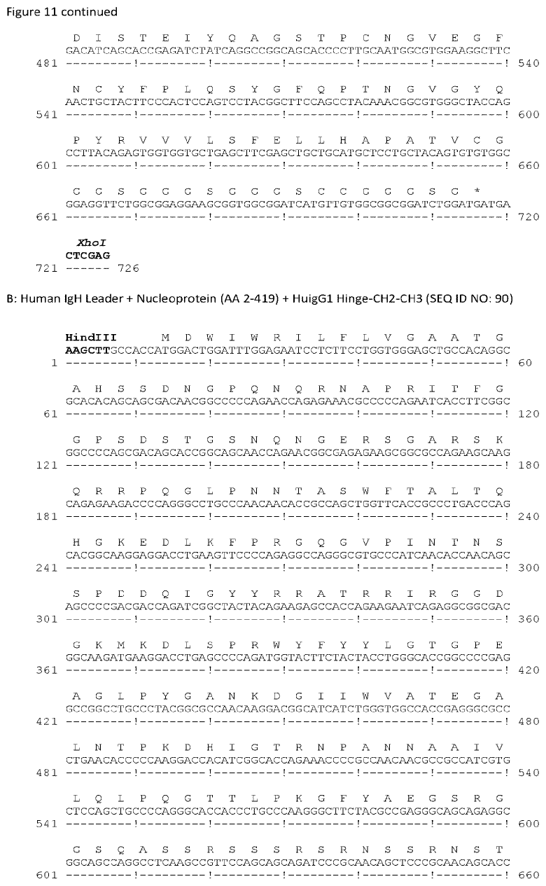

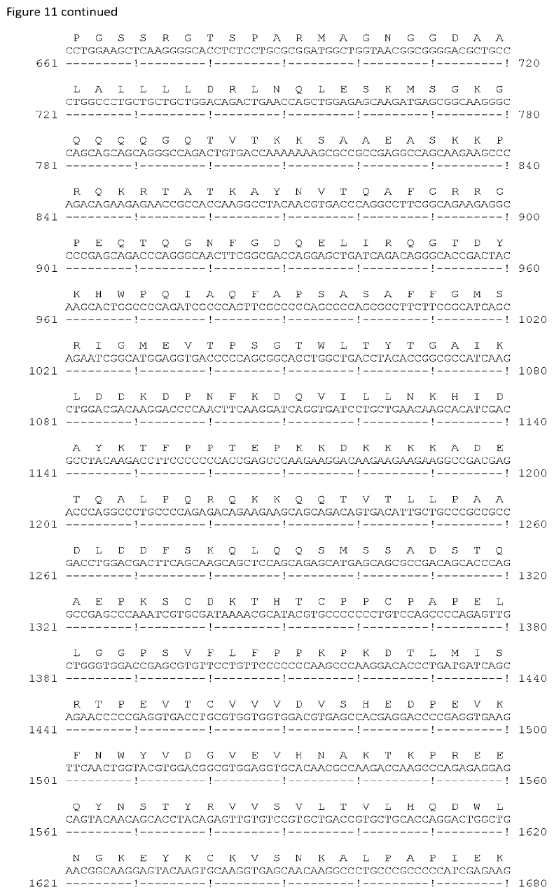

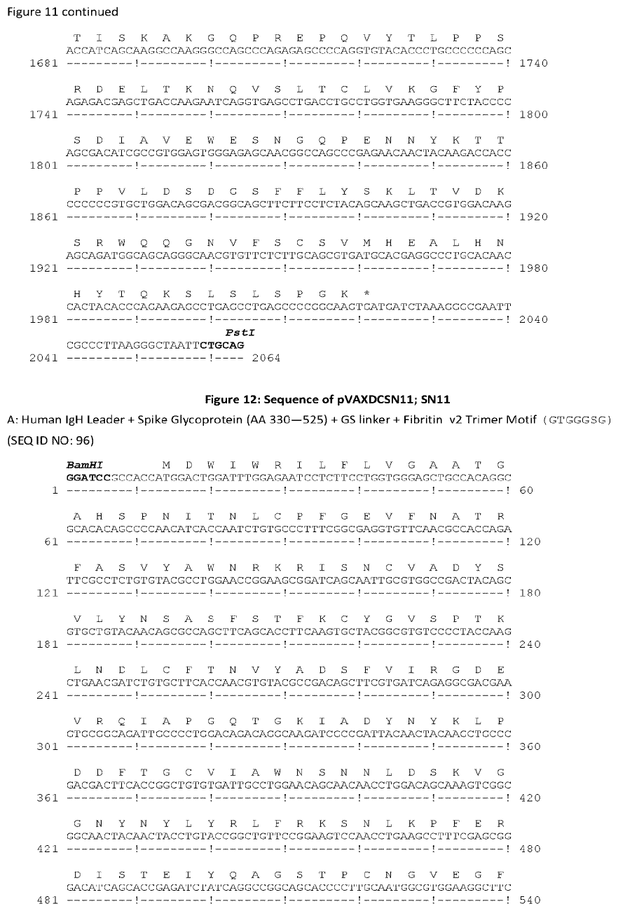

FIG. 11: Sequence of pVAXDCSN10; SN10

Nucleotide and amino acid sequence of the S and N full length chains within the expression vector pVAXDC. Both chains contain a human IgH leader. The S chain encodes RBD amino acids 330-525 attached via a (GGGS)₃ glycine serine linker (SEQ ID NO: 75) to a disulphide bridge motif. The N chain encodes amino acids 2-419 fused in frame with the human IgG1 hinge-CH2-CH3. The stop codon is depicted by an asterisk. The BamHI/XhoI and HindIII/PstI restriction sites utilised in transfer of both chains are highlighted.

FIG. 12: Sequence of pVAXDCSN11; SN11

Nucleotide and amino acid sequence of the S and N full length chains within the expression vector pVAXDC. Both chains contain a human IgH leader. The S chain encodes RBD amino acids 330-525 attached via a longer (GGGS)₃GS glycine serine linker (SEQ ID NO: 74) to a fibritin trimer motif (GTGGGSG) (SEQ ID NO: 121). The N chain encodes amino acids 2-419 fused in frame with the human IgG1 hinge-CH2-CH3 iV1 where murine IgG3 23 AA substitutions are highlighted in bold. The stop codon is depicted by an asterisk. The BamHI/XhoI and HindIII/PstI restriction sites utilised in transfer of both chains are highlighted.

FIG. 13: Sequence of pVAXDCSN12; SN12

Nucleotide and amino acid sequence of the S and N full length chains within the expression vector pVAXDC. Both chains contain a human IgH leader. The S chain encodes RBD amino acids 319-541 linked via a glycine serine to a fibritin trimer motif. The N chain encodes amino acids 2-419 fused in frame with the human IgG1 hinge-CH2-CH3 iV1 where murine IgG3 23 AA substitutions are highlighted in bold. The stop codon is depicted by an asterisk. The BamHI/XhoI and HindIII/PstI restriction sites utilised in transfer of both chains are highlighted.

FIG. 14: Sequence of pVAXDCSN13; SN13

Nucleotide and amino acid sequence of the S and N full length chains within the expression vector pVAXDC. Both chains contain a human IgH leader. The S chain encodes RBD amino acids 319-541 fused in frame with the human IgG1 hinge-CH2-CH3. The N chain encodes amino acids 2-419 fused in frame with the human IgG1 hinge-CH2-CH3 iV1 where murine IgG3 23 AA substitutions are highlighted in bold. The stop codon is depicted by an asterisk. The BamHI/XhoI and HindIII/PstI restriction sites utilised in transfer of both chains are highlighted.

FIG. 15: Sequence of pVAXDCSN14; SN14

Nucleotide and amino acid sequence of the S and N full length chains within the expression vector pVAXDC. Both chains contain a human IgH leader. The S chain encodes RBD amino acids 319-541 while the N chain encodes amino acids 2-419 both fused in frame with the human IgG1 hinge-CH2-CH3 iV1 constant region where murine IgG3 23 AA substitutions are highlighted in bold. To reduce homology in SN14 between the two codon-optimised human IgG1 hinge-CH2-CH3 iV1 constant regions the nucleotide sequences are not identical. The stop codon is depicted by an asterisk. The BamHI/XhoI and HindIII/PstI restriction sites utilised in transfer of both chains are highlighted.

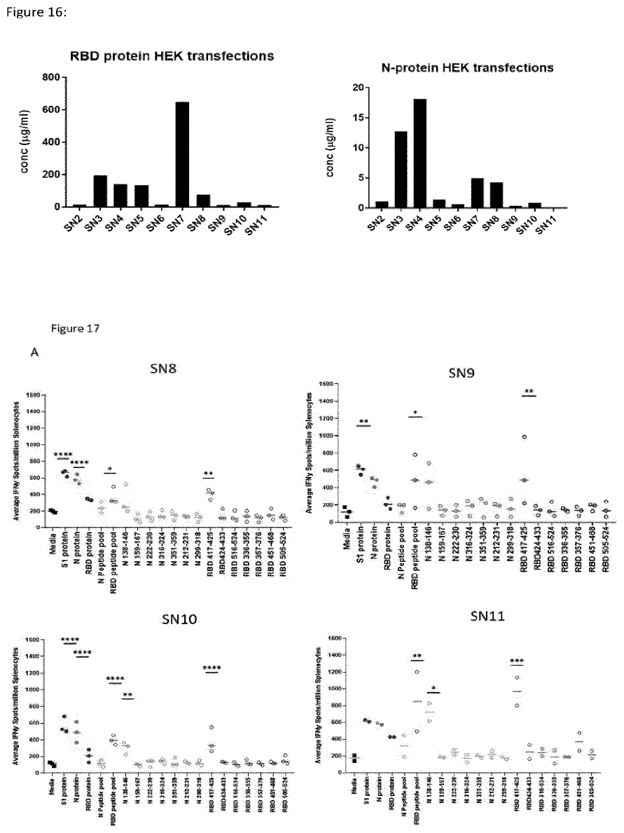

FIG. 16: RBD and N secretion levels (by sandwich ELISA) in conditioned medium six days after transient HEK293 transfections with respective SN constructs.

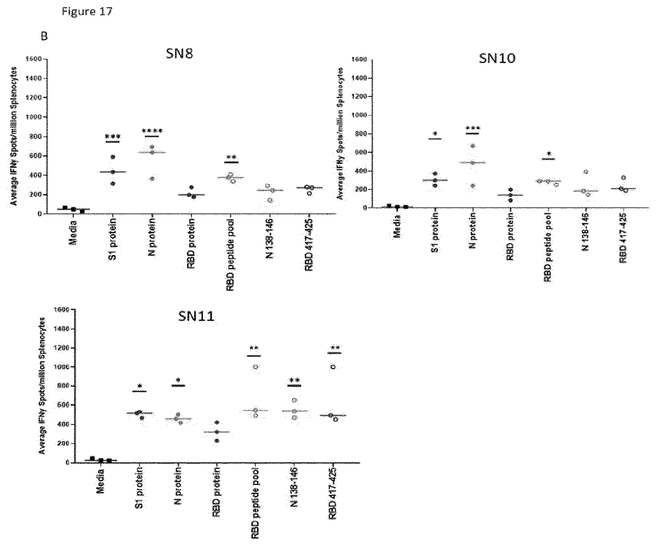

FIG. 17: HHDII mice immunised on days 1, 8 and 15 with SN8, SN9, SN10 and SN11 (A) or SN8, SN10 and SN11 (B) pDNA via gene gun. Splenocytes analysed at day 21 for IFNγ responses by ELISpot assay against media control, S1, RBD and N proteins, N and RBD peptide pools and individual N and RBD peptides. Responses displayed as average spots/million splenocytes. Open circles responses to peptide, closed circles responses to protein, red colour responses to N antigen, blue colour responses to S antigen, RBD, S1.

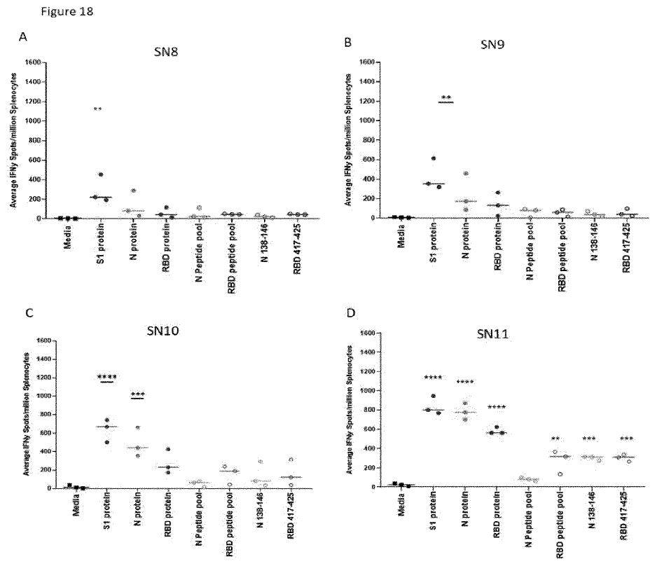

FIG. 18: HHDII/DR1 mice immunised on days 1, 8 and 15 with SN8 (A), SN9 (B), SN10 (C) and SN11 (D) pDNA via gene gun. Splenocytes analysed at day 21 for IFNγ responses by ELISpot assay against media control, S1, RBD and N proteins, N and RBD peptide pools and individual N and RBD peptides. Responses displayed as average spots/million splenocytes. Open circles responses to peptide, closed circles responses to protein, red colour responses to N antigen, blue colour responses to S antigen, RBD, S1.

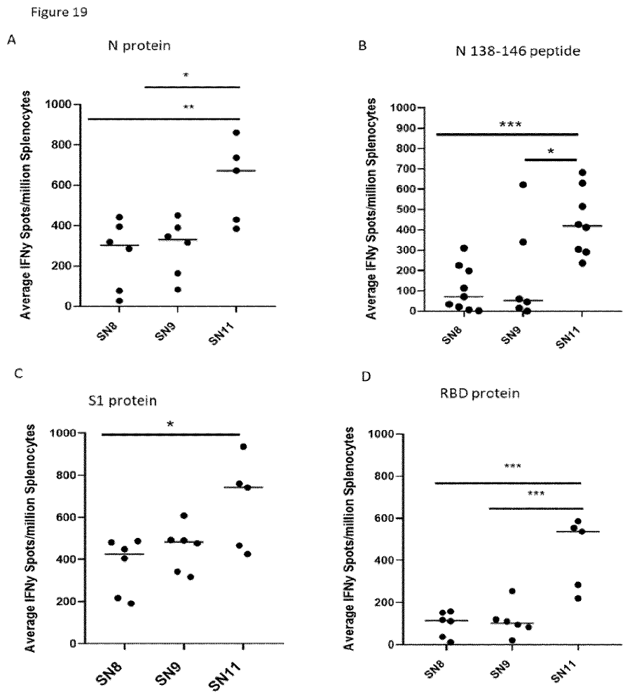

FIG. 19: Comparison of IFNγ responses in ELISpot to N protein (A), N 138-146 peptide (B), S1 protein (C) and RBD protein (D) from mice immunised with SN8, SN9 or SN11 pDNA via gene gun. Data collated from two independent studies. Responses normalised against background control. Responses displayed as average spots/million splenocytes.

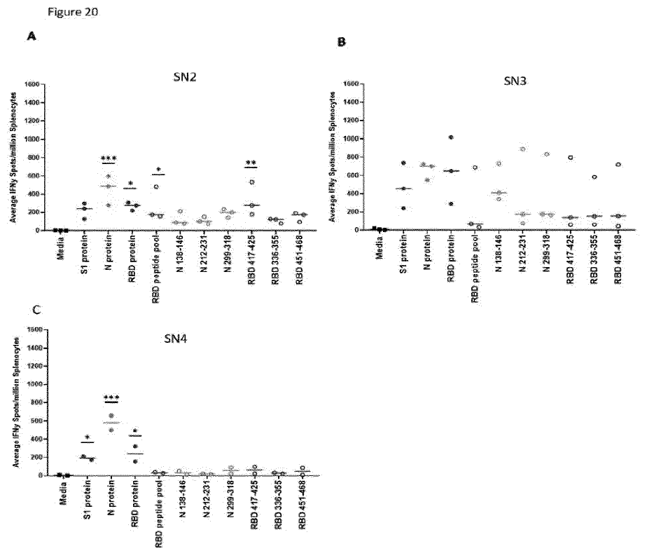

FIG. 20: HHDII/DP4 mice immunised on days 1, 15 and 29 with SN2 (A), SN3 (B) and SN4 (C) pDNA via gene gun. Splenocytes analysed at day 35 for IFNγ responses by ELISpot assay against media control, S1, RBD and N proteins, RBD peptide pool and individual N and RBD peptides. Responses displayed as average spots/million splenocytes. Open circles responses to peptide, closed circles responses to protein, red colour responses to N antigen, blue colour responses to S antigen, RBD, S1.

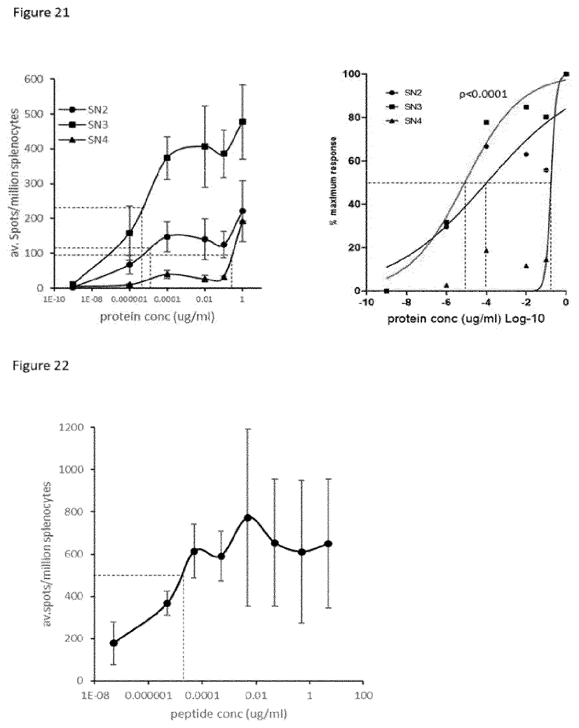

FIG. 21: HHDII/DP4 mice immunised on days 1, 15 and 29 with SN2, SN3 and SN4 pDNA via gene gun. Splenocytes analysed at day 35 for IFNγ responses to titrating amounts of S1 protein by ELISpot assay. Avidity calculated as protein concentration which elicits 50% maximal response. Titration curves shown as spots/million splenocytes and responses normalised to display as a % maximal response curve.

FIG. 22: HHDII mice immunised on days 1, 8 and 15 with SN11 pDNA via gene gun. Splenocytes analysed at day 21 for IFNγ responses to titrating amounts of RBD 417-425 peptide by ELISpot assay. Avidity calculated as protein concentration which elicits 50% maximal response. Titration curves shown as spots/million splenocytes.

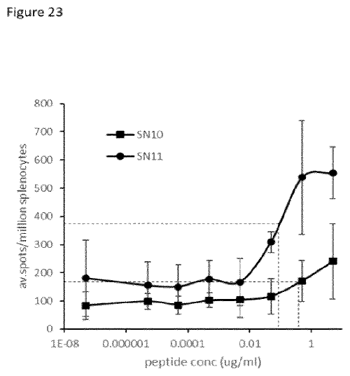

FIG. 23: HHDII mice immunised on days 1, 8 and 15 with SN10 or SN11 pDNA via gene gun. Splenocytes analysed at day 21 for IFNγ responses to titrating amounts of N 138-146 peptide by ELISpot assay. Avidity calculated as protein concentration which elicits 50% maximal response. Titration curves shown as spots/million splenocytes.

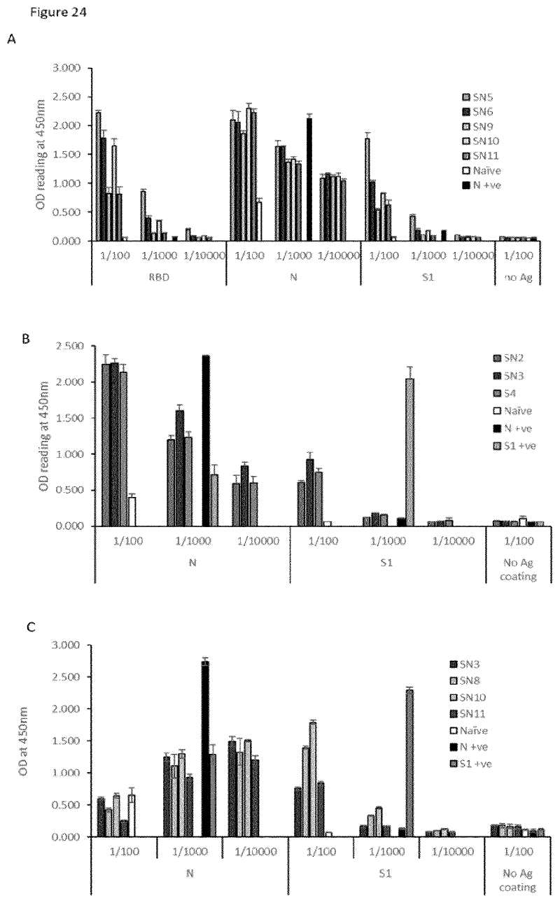

FIG. 24: HHDII/DP4 mice immunised on days 1, 15 and 29 with SN5, SN6, SN9, SN10 and SN11 (A) SN2, SN3 and SN4 (B) pDNA or on days 1, 8, 15 with SN3, SN8, SN10 and SN11 (C) pDNA via gene gun. Sera at 1/100, 1/1000 and 1/10000 dilutions analysed at day 35 (A and B) or day 21 (C) for antibody responses to S1, N and RBD proteins by ELISA.

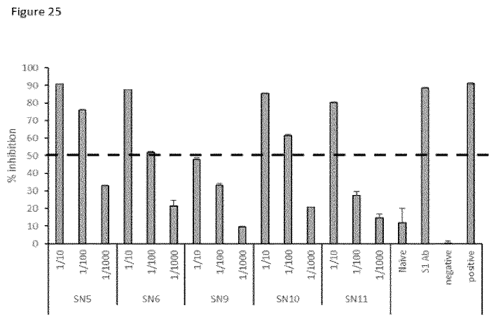

FIG. 25: Surrogate neutralisation assay (RBD binding inhibition assay) on sera taken at day 35 from HHDII/DP4 mice immunised with SN5, SN6, SN9, SN10 and SN11 via gene gun on days 1, 15 and 29. Sera from naive mice used as a negative control and murine S1 antibody (SINO BIOLOGICAL®) as an additional positive control.

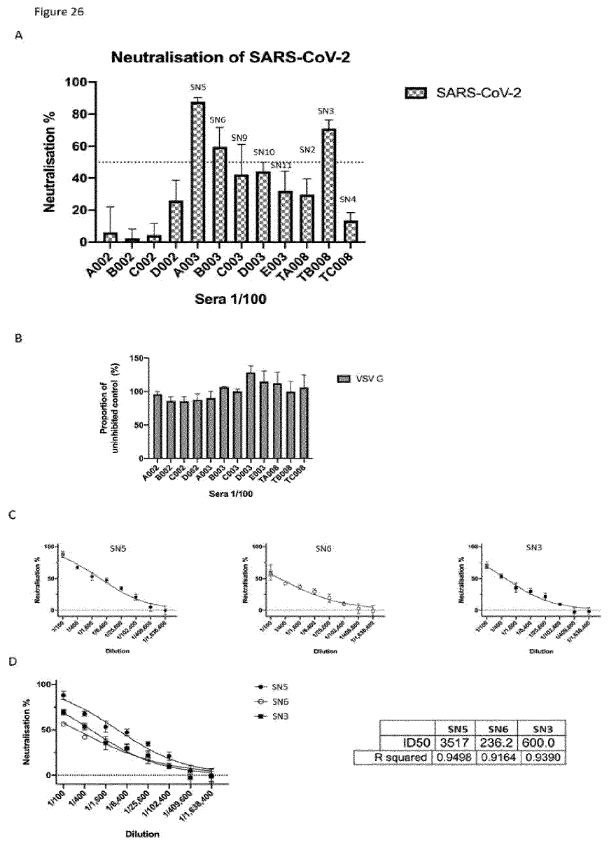

FIG. 26: Pseudovirus neutralisation assay. HHDII/DP4 mice immunised on days 1, 15 and 29 with SN5, SN6, SN9, SN10 and SN11, SN2, SN3 and SN4 pDNA via gene gun. Sera taken at day 35 was tested at 1/100 dilution for neutralisation of SARS-CoV-2 (A) or an irrelevant virus (VSV G) (B). Virus neutralisation was also analysed at different sera dilutions (C). 50% neutralisation titres (ID50) (D).

FIG. 27: Sequence of pVAXDCSN15; SN15

Nucleotide and amino acid sequence of the spike and nucleoprotein full length chains within the expression vector pVAXDC. Both chains contain a human IgH leader. The spike chain encodes amino acids 319-541. The nucleoprotein chain encodes amino acids 2-419 fused in frame with the human IgG1 hinge-CH2-CH3 iV1 where murine IgG3 23 AA substitutions are highlighted in bold. The stop codon is depicted by an asterisk. The BamHI/XhoI and HindIII/PstI restriction sites utilised in transfer of both chains are highlighted.

FIG. 28: Sequence of pVAXDCIB68 (SCIB1)

Nucleotide and amino acid sequence of the antibody heavy and light variable regions cloned in frame with the human IgG1 CH1-hinge-CH2-CH3 constant region and human kappa constant region respectively within the expression vector pVAXDC. Amino acids within boxes encodes the HLA-DR7, HLA-DR53 and HLA-DQ6 restricted gp100₁₇₃₋₁₉₀ CD4 epitope (GTGRAMLGTHTMEVTVYH) (SEQ ID NO: 29) in H1 and L3, the HLA-0201 TRP2₁₈₀₋₁₈₈ epitope (SVYDFFVWL) (SEQ ID NO: 30) in H2 and the HLA-DR4 restricted gp100₄₄₋₅₉ CD4 epitope in H3 and L1 (WNRQLYPEWTEAQRLD) (SEQ ID NO: 31). The HindIII/Afe I and BamHI/BsiWI restriction sites utilised in transfer of the variable heavy and light regions are highlighted. For direct replacement of human IgG1 constant domain (CH1-hinge-CH2-CH3) with the enhanced human IgG1 CH1-hinge-CH2-CH3 iV1 constant region Afe1 and EcoRI were utilised as shown. The stop codon is depicted by an asterisk.

FIG. 29: Sequence of pVAXDCIB68 iV1 (iSCIB1)

Nucleotide and amino acid sequence of the antibody heavy and light variable regions cloned in frame with the enhanced human IgG1 CH1-hinge-CH2-CH3 iV1 constant region (where murine IgG3 23 AA substitutions are highlighted in bold) and human kappa constant region respectively within the expression vector pVAXDC. Amino acids within boxes encode the HLA-DR7, HLA-DR53 and HLA-DQ6 restricted gp100₁₇₃₋₁₉₀ CD4 epitope (GTGRAMLGTHTMEVTVYH) (SEQ ID NO: 29) in H1 and L3, the HLA-0201 TRP2₁₈₀₋₁₈₈ epitope (SVYDFFVWL) (SEQ ID NO: 30) in H2 and the HLA-DR4 restricted gp100₄₄₋₅₉ CD4 epitope in H3 and L1 (WNRQLYPEWTEAQRLD) (SEQ ID NO: 31). The HindIII/Afe I and BamHI/BsiWI restriction sites utilised in transfer of the variable heavy and light regions are highlighted. For direct replacement of human IgG1 constant domain (CH1-hinge-CH2-CH3) with the enhanced human IgG1 CH1-hinge-CH2-CH3 iV1 constant region Afe1 and EcoRI where utilised as shown. The stop codon is depicted by an asterisk.

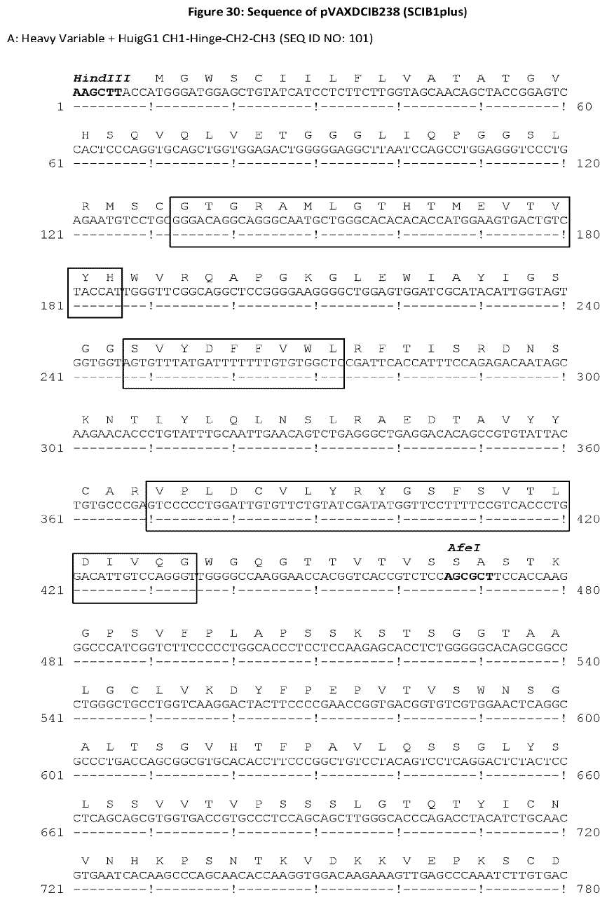

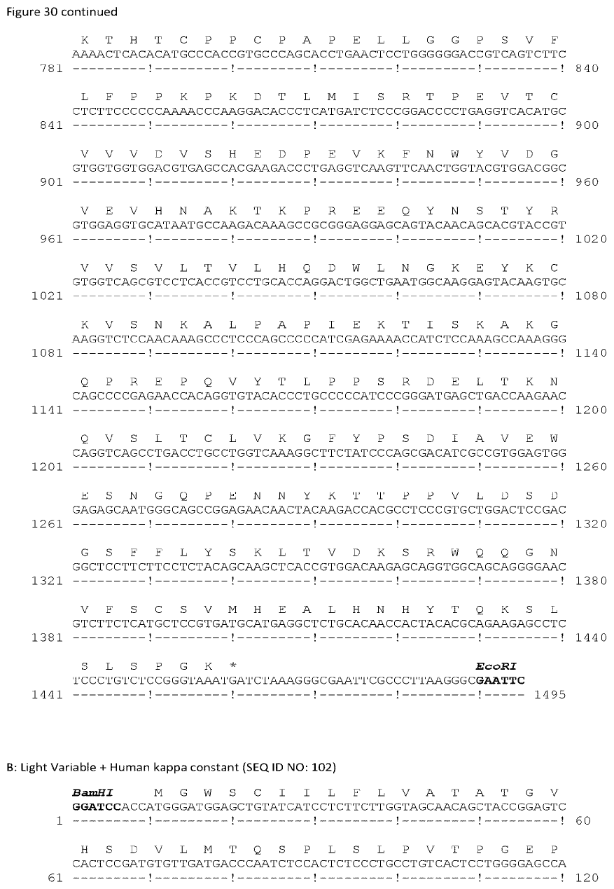

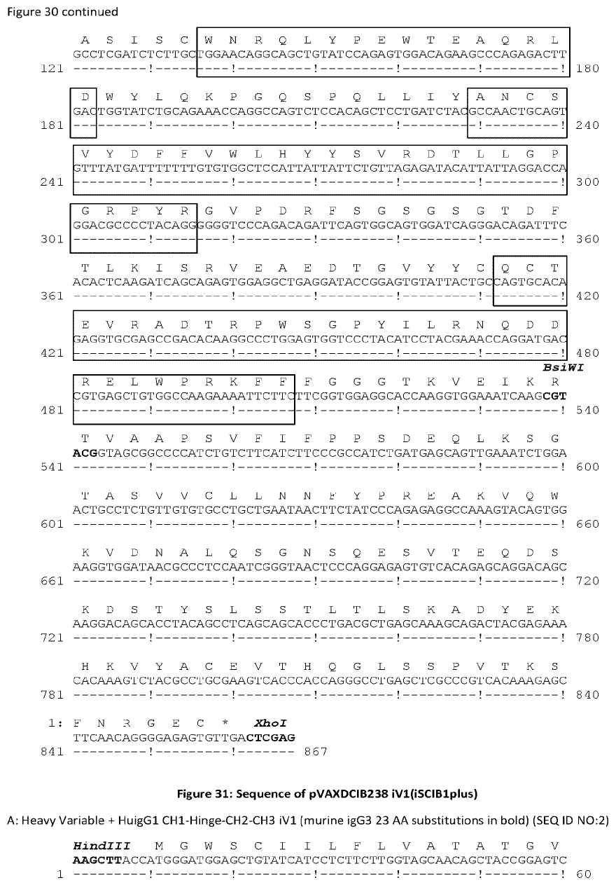

FIG. 30: Sequence of pVAXDCIB238 (SCIB1 plus)

Nucleotide and amino acid sequence of the antibody heavy and light variable regions cloned in frame with the human IgG1 CH1-hinge-CH2-CH3 constant region and human kappa constant region respectively within the expression vector pVAXDC. Amino acids within boxes represent the HLA-DR7, HLA-DR53 and HLA-DQ6 restricted gp100₁₇₃₋₁₉₀ CD4 epitope (GTGRAMLGTHTMEVTVYH) (SEQ ID NO: 29) in CDR H1, the HLA-0201 TRP2₁₈₀₋₁₈₈ epitope (SVYDFFVWL) (SEQ ID NO: 30) in H2 and the HLA-DR4 restricted gp100₄₄₋₅₉ CD4 epitope in L1 (WNRQLYPEWTEAQRLD) (SEQ ID NO: 31) retained from pVAXDCIB68. Additional epitopes include nested within the gp100₄₇₁₋₄₉₂ sequence inserted into the H3 site (VPLDCVLYRYGSFSVTLDIVQG) (SEQ ID NO: 32) a HLA-A1, B35 and predicted HLA-DP4 epitope. TRP2₁₇₇₋₂₀₅ and TRP2₆₀₋₉₁ sequences were grafted into the L2 and L3 sites of the variable light region respectively. These collectively contained an HLA-A2, A3, A31, A33, B35, B44, HLA-DR3 and another potential HLA-DP4 epitope as described elsewhere. The HindIII/Afe I and BamHI/BsiWI restriction sites utilised in transfer of the variable heavy and light regions are highlighted. For direct replacement of human IgG1 constant domain (CH1-hinge-CH2-CH3) with the enhanced human IgG1 CH1-hinge-CH2-CH3 iV1 constant region Afe1 and EcoRI were utilised as shown. The stop codon is depicted by an asterisk.

FIG. 31: Sequence of pVAXDCIB238 iV1 (iSCIB1 plus)

Nucleotide and amino acid sequence of the antibody heavy and light variable regions cloned in frame with the enhanced human IgG1 CH1-hinge-CH2-CH3 iV1 constant region (where murine IgG3 23 AA substitutions are highlighted in bold) and human kappa constant region respectively within the expression vector pVAXDC. Amino acids within boxes represent the HLA-DR7, HLA-DR53 and HLA-DQ6 restricted gp100₁₇₃₋₁₉₀ CD4 epitope (GTGRAMLGTHTMEVTVYH) (SEQ ID NO: 29) in CDR H1, the HLA-0201 TRP2₁₈₀₋₁₈₈ epitope (SVYDFFVWL) (SEQ ID NO: 30) in H2 and the HLA-DR4 restricted gp100₄₄₋₅₉ CD4 epitope in L1 (WNRQLYPEWTEAQRLD) (SEQ ID NO: 31) retained from pVAXDCIB68. Additional epitopes include, nested within the gp100₄₇₁₋₄₉₂ sequence inserted into the H3 site (VPLDCVLYRYGSFSVTLDIVQG) (SEQ ID NO: 32), a HLA-A1, B35 and predicted HLA-DP4 epitope. TRP2₁₇₇₋₂₀₅ and TRP2₆₀₋₉₁ sequences were grafted into the L2 and L3 sites of the variable light region respectively. These collectively contained an HLA-A2, A3, A31, A33, B35, B44, HLA-DR3 and another potential HLA-DP4 epitope. The HindIII/Afe I and BamHI/BsiWI restriction sites utilised in transfer of the variable heavy and light regions are highlighted. For direct replacement of human IgG1 constant domain (CH1-hinge-CH2-CH3) with the enhanced human IgG1 CH1-hinge-CH2-CH3 iV1 constant region Afe1 and EcoRI where utilised as shown. The stop codon is depicted by an asterisk.

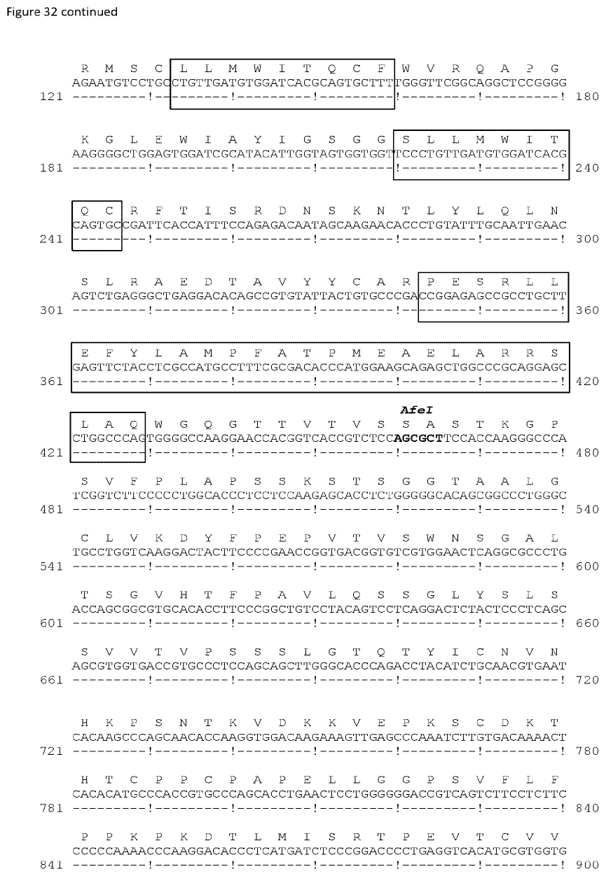

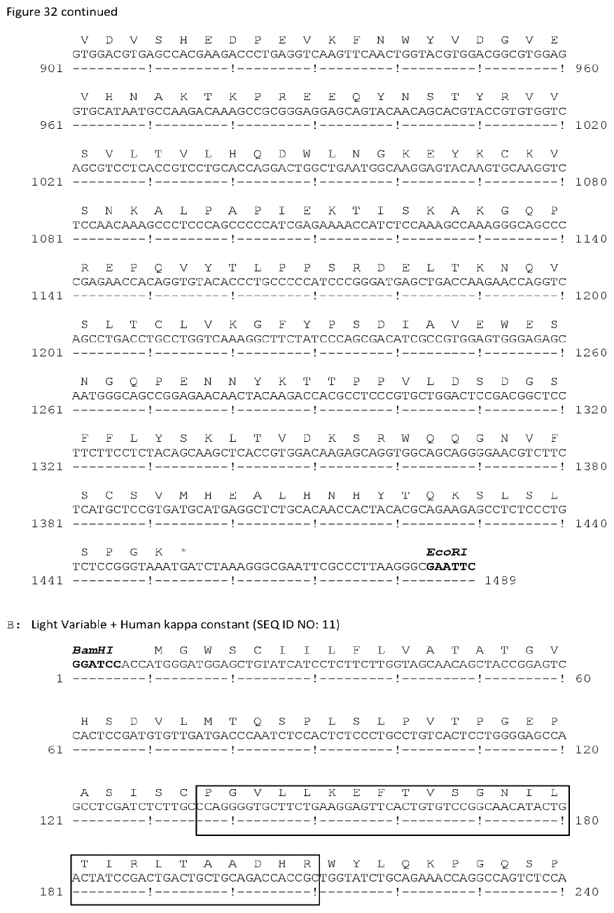

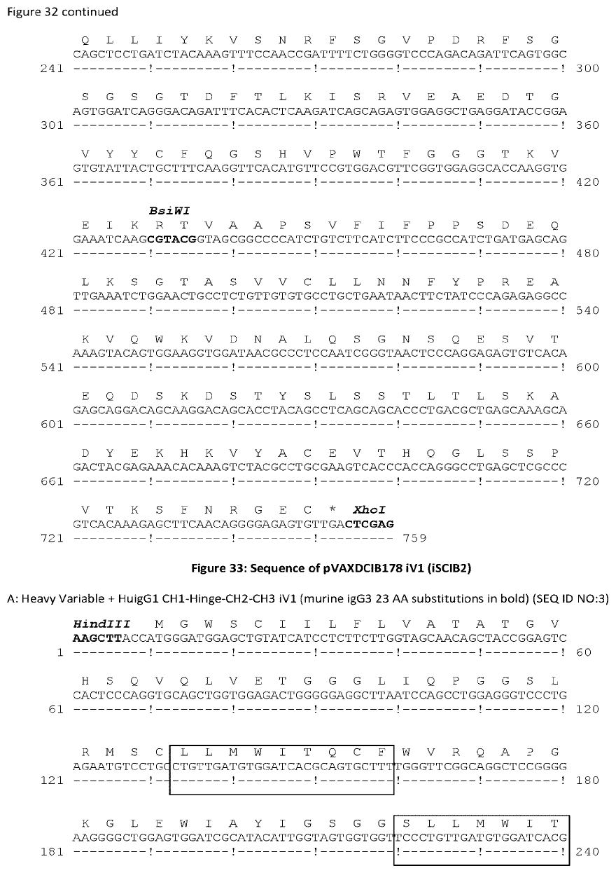

FIG. 32: Sequence of pVAXDCIB178 (SCIB2)

Nucleotide and amino acid sequence of the antibody heavy and light variable regions cloned in frame with the human IgG1 CH1-hinge-CH2-CH3 constant region and human kappa constant region respectively within the expression vector pVAXDC. Amino acids within boxes represent the NYESO-1₁₅₈₋₁₆₆ HLA-A24 epitope (LLMWITQCF) (SEQ ID NO: 35) and NYESO-1₁₅₇₋₁₆₅ HLA-A2-restricted epitope (SLLMWITQC) (SEQ ID NO: 36) in CDR H1 and H2. NY-ESO-1₈₃₋₁₁₁ amino acid sequence (PESRLLEFYLAMPFATPMEAELARRSLAQ) (SEQ ID NO: 37) and NY-ESO-1₁₁₉₋₁₄₃ (PGVLLKEFTVSGNILTIRLTAADHR) (SEQ ID NO: 38) where grafted into the CDR H3 and L1 site which collectively contains a number of nested additional epitopes as described elsewhere (Xue et al. ONCOIMMUNOLOGY 2016, VOL. 5, NO. 6, e1169353). The HindIII/Afe I and BamHI/BsiWI restriction sites utilised in transfer of the variable heavy and light regions are highlighted. For direct replacement of human IgG1 constant domain (CH1-hinge-CH2-CH3) with the enhanced human IgG1 CH1-hinge-CH2-CH3 iV1 constant region Afe1 and EcoRI were utilised as shown. The stop codon is depicted by an asterisk.

FIG. 33: Sequence of pVAXDCIB178 iV1 (iSCIB2)

Nucleotide and amino acid sequence of the antibody heavy and light variable regions cloned in frame with the enhanced human IgG1 CH1-hinge-CH2-CH3 iV1 constant region (where murine IgG3 23 AA substitutions are highlighted in bold) and human kappa constant region respectively within the expression vector pVAXDC. Amino acids within boxes represent the NYESO-1₁₅₈₋₁₆₆ HLA-A24 epitope (LLMWITQCF) (SEQ ID NO: 35) and NYESO-1₁₅₇₋₁₆₅ HLA-A2-restricted epitope (SLLMWITQC) (SEQ ID NO: 36) in CDR H1 and H2. NY-ESO-1₈₃₋₁₁₁ amino acid sequence (PESRLLEFYLAMPFATPMEAELARRSLAQ) (SEQ ID NO: 37) and NY-ESO-1₁₁₉₋₁₄₃ (PGVLLKEFTVSGNILTIRLTAADHR) (SEQ ID NO: 38) where grafted into the CDR H3 and L1 site which collectively contains a number of nested additional epitopes as described elsewhere [183]. The HindIII/Afe I and BamH1/BsiWI restriction sites utilised in transfer of the variable heavy and light regions are highlighted. For direct replacement of human IgG1 constant domain (CH1-hinge-CH2-CH3) with the enhanced human IgG1 CH1-hinge-CH2-CH3 iV1 constant region Afe1 and EcoRI where utilised as shown. The stop codon is depicted by an asterisk.

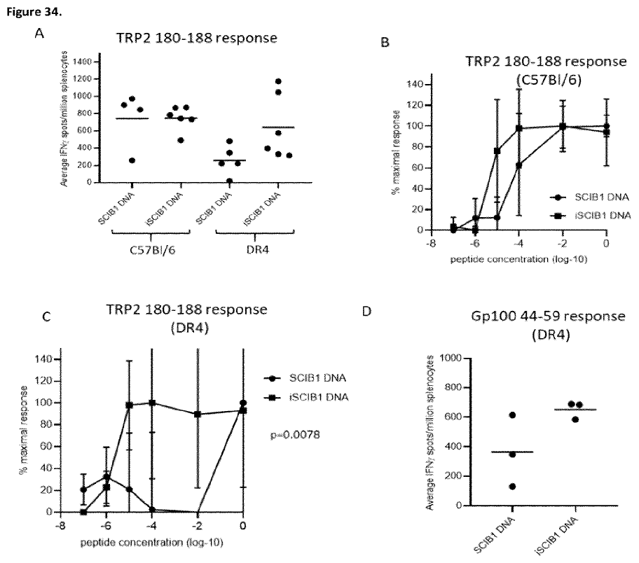

FIG. 34: C57Bl/6 or HLA-DR4 mice immunised on days 1, 8 and 15 with SCIB1 or iSCIB1 pDNA via gene gun. Splenocytes analysed at day 21 for IFNγ responses to TRP2 180-188 peptide (A-C) or gp100 44-59 peptide (D) by ELISpot assay. Frequency of TRP2 180-188 response compared in different mouse strains (A). B and C, TRP2 180-188 response avidity calculated as peptide concentration which elicits 50% maximal response. Titration curves shown as % maximal response. D, frequency of responses to 0.1 μg/ml gp100 44-59 peptide in HLA-DR4 mice.

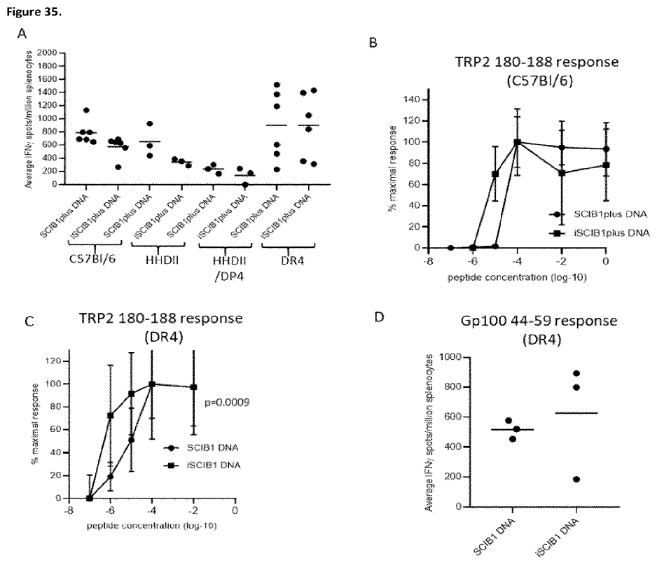

FIG. 35: C57Bl/6, HHDII, HHDII/DP4 or HLA-DR4 mice immunised on days 1, 8 and 15 with SCIB1plus or iSCIB1plus pDNA via gene gun. Splenocytes analysed at day 21 for IFNγ responses to TRP2 180-188 peptide (A-C) or gp100 44-59 peptide (D) by ELISpot assay. Frequency of responses compared in different mouse strains (A). B and C, TRP2 180-188 response avidity calculated as peptide concentration which elicits 50% maximal response. Titration curves shown as % maximal response. D, frequency of responses to 1 μg/ml gp100 44-59 peptide in HLA-DR4 mice.

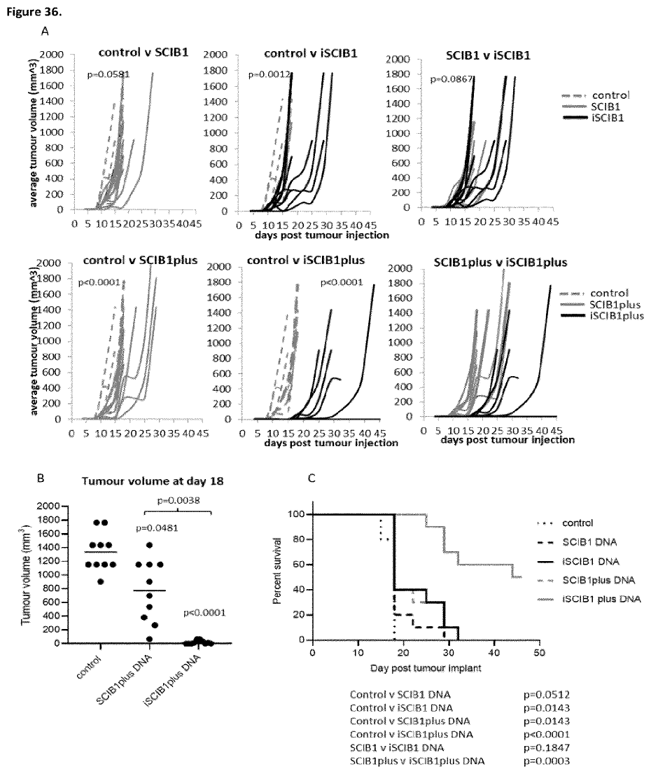

FIG. 36: C57B16 mice implanted with B16F1 tumour cells on day 1 followed by immunisation with pDNA SCIB1, iSCIB1, SCIB1plus or iSCIB1plus at days 4, 11 and 18. A, tumour growth curves. B, comparison of tumour volume at day 18. C, overall survival.

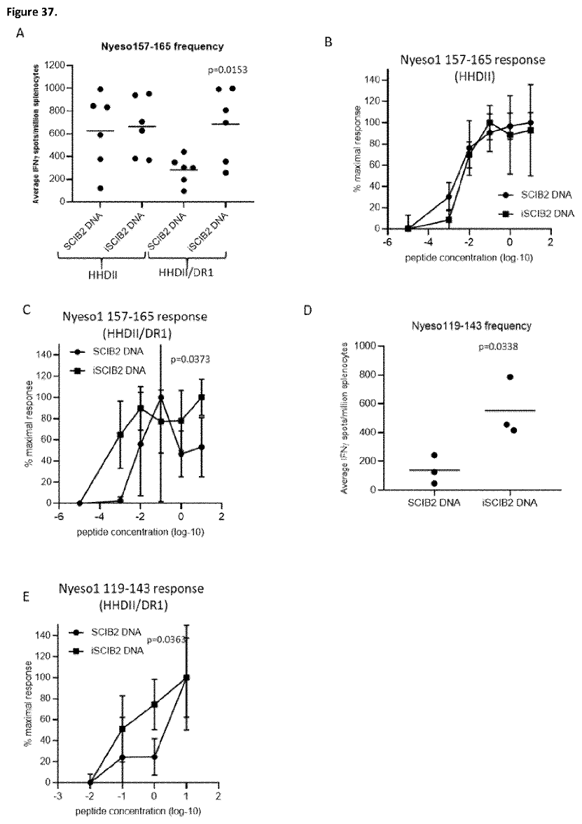

FIG. 37: HHDII or HHDII/DR1 mice immunised on days 1, 8 and 15 with SCIB2 or iSCIB2 pDNA via gene gun. Splenocytes analysed at day 21 for IFNγ responses to Nyeso1 157-165 peptide (A-C) or Nyeso1 119-143 peptide (D and E) by ELISpot assay. Frequency of Nyeso1 157-165 responses compared in different mouse strains (A). B and C, Nyeso1 157-165 response avidity calculated as peptide concentration which elicits 50% maximal response. Titration curves shown as % maximal response. D, frequency of responses to 10 μg/ml Nyeso1 119-143 peptide in HHDII/DR1 mice. E, Nyeso1 119-143 response avidity in HHDII/DR1 mice calculated as peptide concentration which elicits 50% maximal response. Titration curves shown as % maximal response.

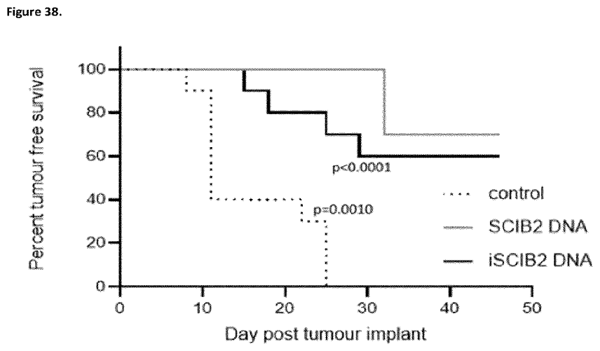

FIG. 38: HHDII mice implanted with B16 HHDII Nyeso tumour cells on day 1 followed by immunisation with pDNA SCIB2 or iSCIB2 at days 4, 8 and 11 and tumour free survival over time monitored.

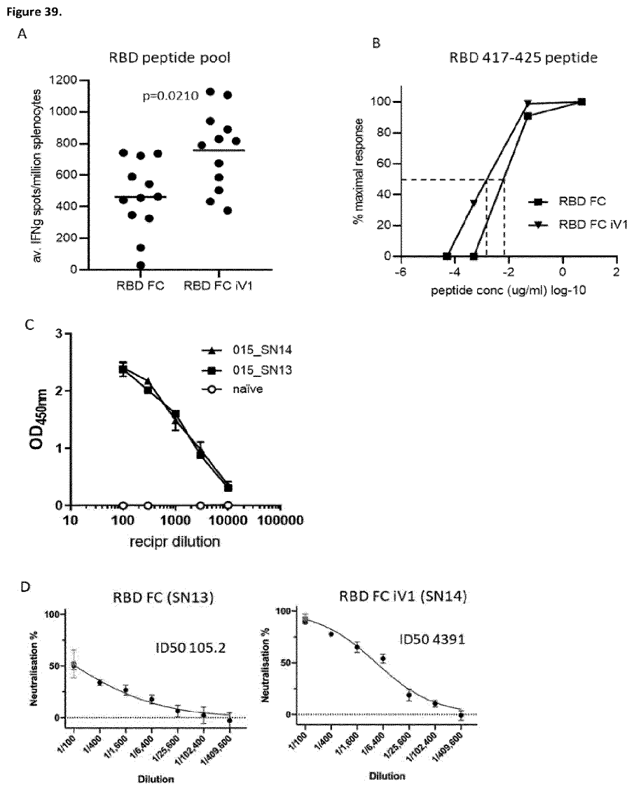

FIG. 39: HLA-A2 transgenic or Balb/c mice immunised on days 1, 8 and 15 with SN13 or SN14 pDNA via gene gun. Splenocytes analysed at day 21 for frequency of IFNγ responses to RBD peptide pool (A) or avidity by peptide titration to RBD 417-425 peptide by ELISpot assay. Avidity calculated as peptide concentration which elicits 50% maximal response. Titration curves shown as % maximal response. Sera dilutions analysed at day 21 for S1 protein specific antibody responses in ELISA assay (C) or for SARS-CoV-2 neutralising antibodies in pseudovirus neutralisation assay (D).

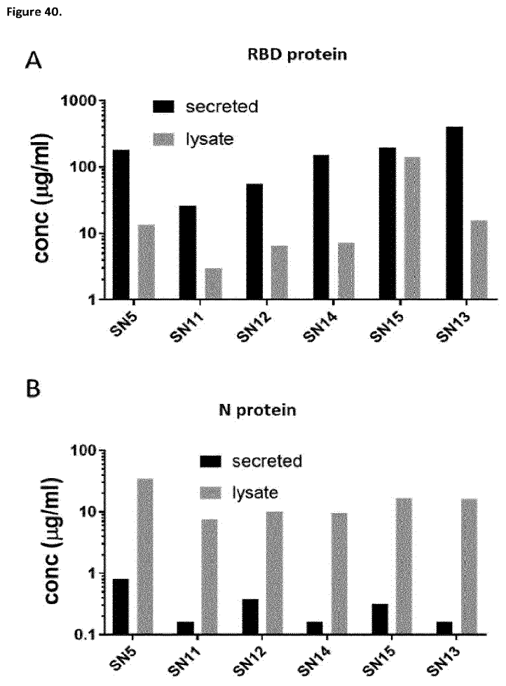

FIG. 40: RBD (A) and N (B) secretion levels (by sandwich ELISA) in conditioned medium and cell lysates six days after transient HEK293 transfections with respective SN constructs.

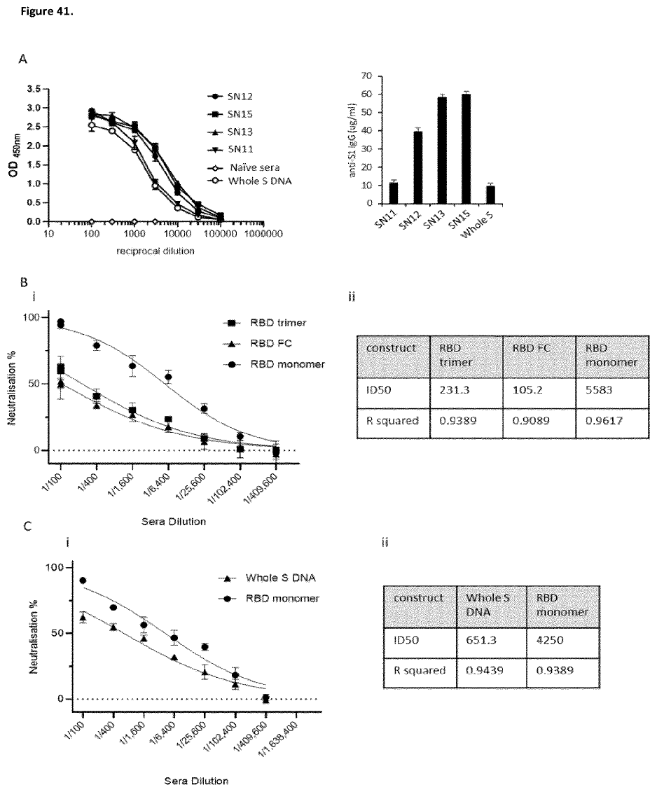

FIG. 41: C57Bl/6 (C) or Balb/c (A and B) mice immunised on days 1, 8 and 15 with SN11 (small RBD trimer), SN12 (RBD trimer), SN13 (RBD-Fc), SN15 (RBD monomer) all these constructs also contain the modified Fc-N or whole S pDNA via gene gun. Sera dilutions analysed at day 21 for S1 protein specific antibody responses in ELISA assay (A) or for SARS-CoV-2 neutralising antibodies in pseudovirus neutralisation assay (B and C).

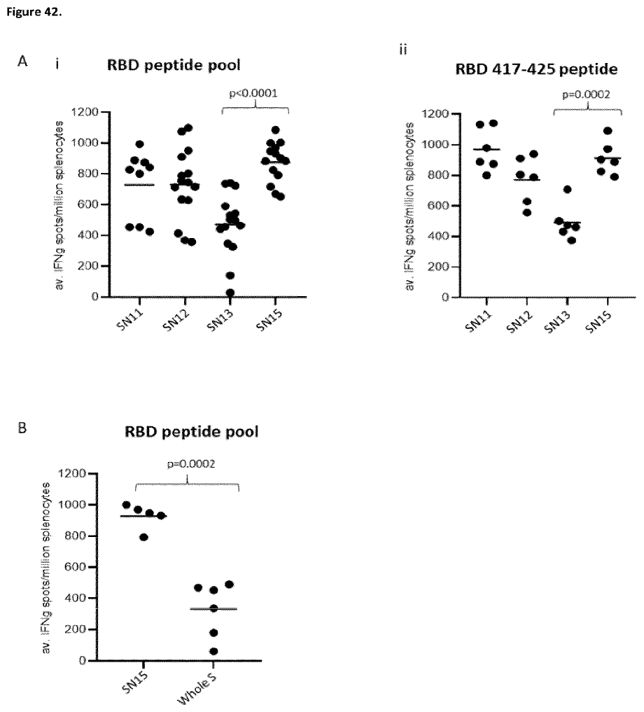

FIG. 42: HLA-A2 transgenic, C57Bl/6 or Balb/c mice immunised on days 1, 8 and 15 with SN11 (small RBD trimer), SN12 (RBD trimer), SN13 (RBD-Fc), SN15 (RBD monomer) all these constructs also contain the modified Fc-via gene gun. Splenocytes analysed at day 21 for frequency of IFNγ responses to RBD peptide pool (Ai and B) and to RBD 417-425 peptide (Aii) by ELISpot assay.

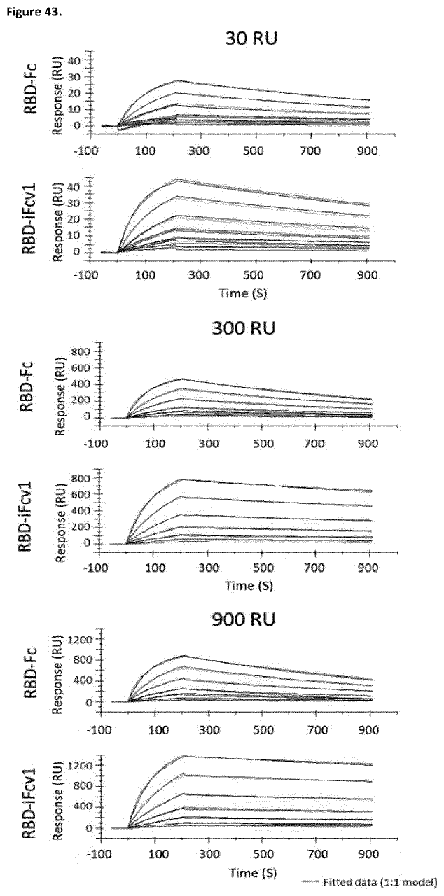

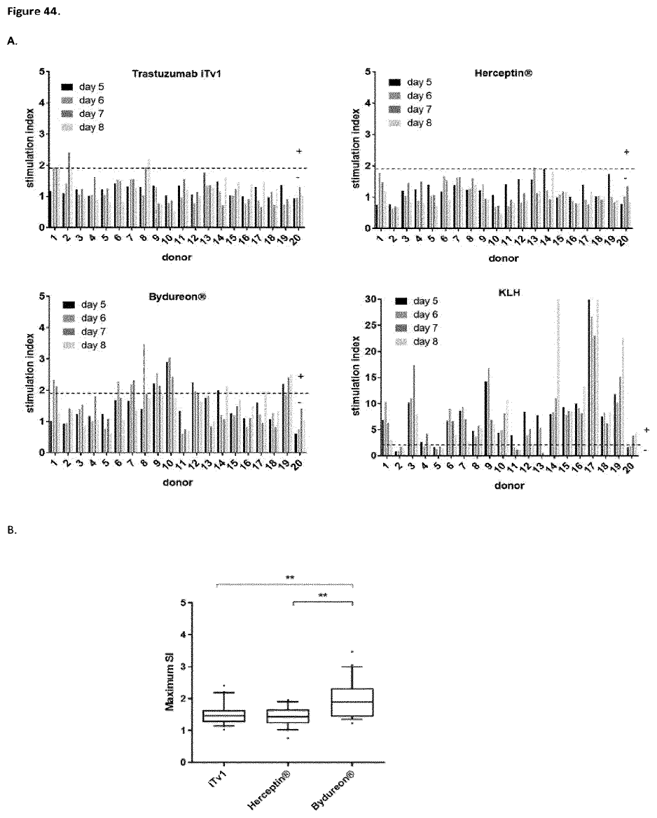

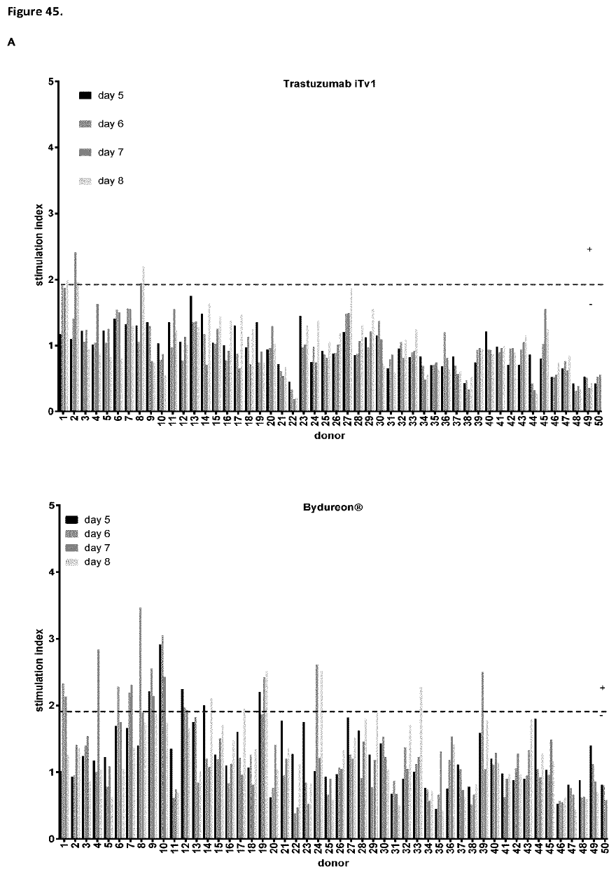

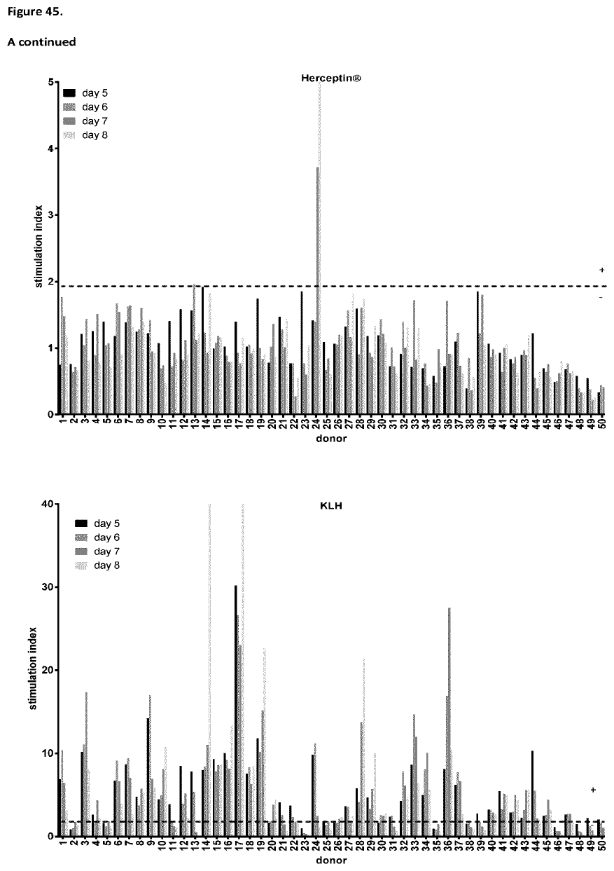

FIG. 43: Real-time binding curves (SPR, BiaT200) of the interaction of RBD-FC and RBD-iFCv1 at increasing levels of CD64 captured onto a CM5 chip.

FIG. 44: A. Healthy donor T cell proliferation responses (20-donor panel). PBMC from bulk cultures were sampled and assessed for proliferation on days 5, 6, 7 and 8 after incubation with: iTv1, Herceptin®, Bydureon® and KLH. Proliferation responses with an SI≥1.90 (indicated by red dotted line) that were significant (p<0.05) using an unpaired, two sample Student's t-test were considered positive. B. Box and whisker plots showing healthy donor T cell responses to the iTv1, Herceptin® and Bydureon®. Chart shows maximum proliferation of CD4+ T cells obtained over the time course. Bars represent the 10-90 percentile. Repeated measures one way ANOVA (Friedman test) using a Dunn's post-test pairs comparison are shown for statistical analysis. **p<0.01

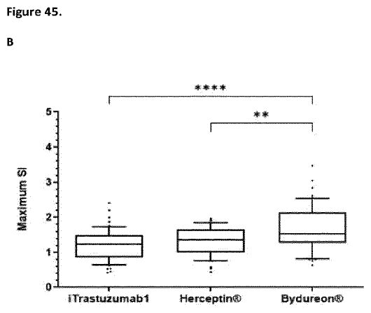

FIG. 45: A. 50 donor panel of healthy donor T cell proliferation responses. PBMC from bulk cultures were sampled and assessed for proliferation on days 5, 6, 7 and 8 after incubation with: iTv1, Herceptin®, Bydureon® and KLH. Proliferation responses with an SI≥1.90 (indicated by red dotted line) that were significant (p<0.05) using an unpaired, two sample Student's t-test were considered positive. B. Box and whisker plots showing healthy donor T cell responses to the iTv1, Herceptin® and Bydureon®. Chart shows maximum proliferation of CD4+ T cells obtained over the time course. Bars represent the 10-90 percentile. Repeated measures one way ANOVA (Friedman test) using a Dunn's post-test pairs comparison are shown for statistical analysis. **p<0.01, ****p<0.0001

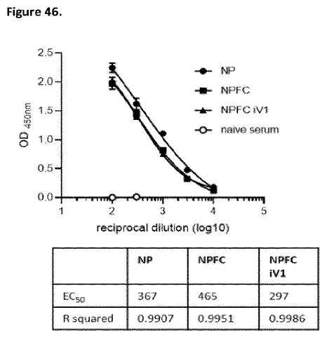

FIG. 46: Sera from mice immunised on days 1, 8 and 15 with NP, NPFc or NPFciV1 via gene gun analysed at day 21 for N protein specific antibody responses in ELISA assay.

FIG. 47: Sequence of pVAXDCSN16; SN16

Nucleotide and amino acid sequence of the spike and nucleoprotein full length chains within the expression vector pVAXDC. Both chains contain a human IgH leader. The spike chain encodes amino acids 319-541 carrying the N501Y mutation of the Kent variant/lineage B.1.1.7, UK-VOC 202012/01. The nucleoprotein chain encodes amino acids 2-419, which includes the D3L and S235F mutations from the variant, fused in frame with the human IgG1 hinge-CH2-CH3 iV1 where murine IgG3 23 AA substitutions are in bold. The stop codon is depicted by an asterisk. Mutations of the Kent variant/lineage B.1.1.7, UK-VOC 202012/01 are in bold and highlighted in grey. The BamHI/XhoI and HindIII/PstI restriction sites utilised in transfer of both chains are highlighted

FIG. 48: Sequence of pVAXDCSN17; SN17

Nucleotide and amino acid sequence of the spike and nucleoprotein full length chains within the expression vector pVAXDC. Both chains contain a human IgH leader. The spike chain encodes amino acids 319-541 carrying the K417N, E484K and N501Y mutations of the south African variant VOC 501Y.V2/B1.351. The nucleoprotein chain encodes amino acids 2-419, which includes the T2051 mutation from the variant, fused in frame with the human IgG1 hinge-CH2-CH3 iV1 where murine IgG3 23 AA substitutions are in bold. The stop codon is depicted by an asterisk. Mutations of the south African variant VOC 501Y.V2/B1.351 are in bold and highlighted in grey. The BamHI/XhoI and HindIII/PstI restriction sites utilised in transfer of both chains are highlighted.

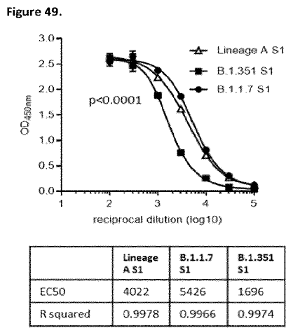

FIG. 49: Balb/c mice immunised on days 1, 8 and 15 with SN15 (RBD monomer and N linked to modified Fc (FciV1)) via gene gun. Sera analysed at day 21 for antibody reactivity to Lineage A (Wuhan), B.1.351 and B.1.1.7 S1 protein variants in ELISA at reciprocal sera dilutions. EC₅₀ values are shown.

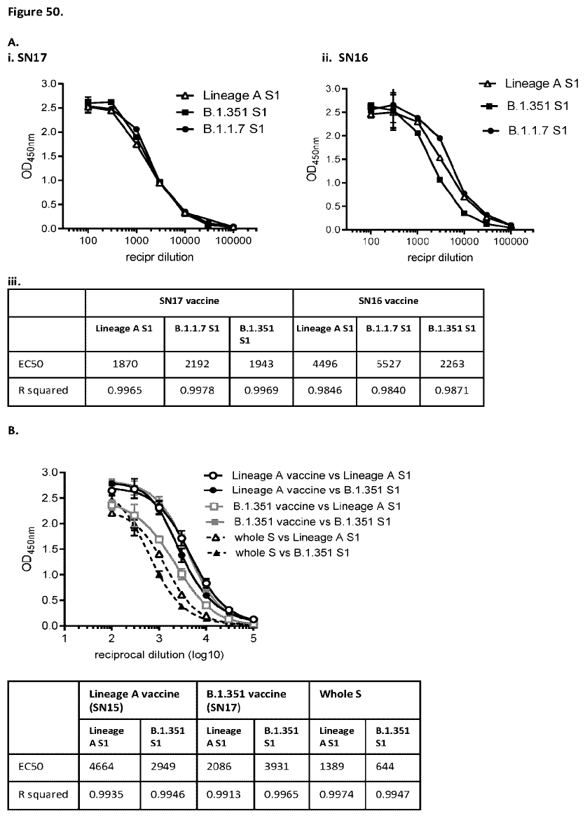

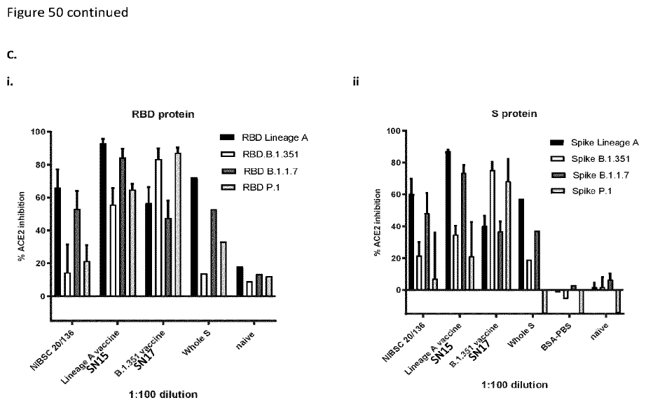

FIG. 50: Balb/c mice immunised on days 1, 8 and 15 with SN15 (RBD monomer and N linked to modified Fc (FciV1)), SN16 (RBD monomer and N linked to modified Fc (FciV1)—B.1.1.7 variant), SN17 (RBD monomer and N linked to modified Fc (FciV1)—B.1.351 variant), or whole S DNA via gene gun. Sera from SN16 and SN17 immunised mice (A) or SN15, SN17 and whole S DNA immunised mice (B) analysed at day 21 for antibody reactivity to Lineage A (Wuhan), B.1.351 and B.1.1.7 S1 protein variants in ELISA at reciprocal sera dilutions. EC₅₀ values are shown. C. Sera from SN15, SN17, whole S DNA, naive mice and control NIBSC 20/136 were assessed for ACE2 binding inhibition of variant RBD (i) and S1 (ii) proteins at 1 in 100 sera dilution using MSD technology. BSA-PBS was used as a negative control.

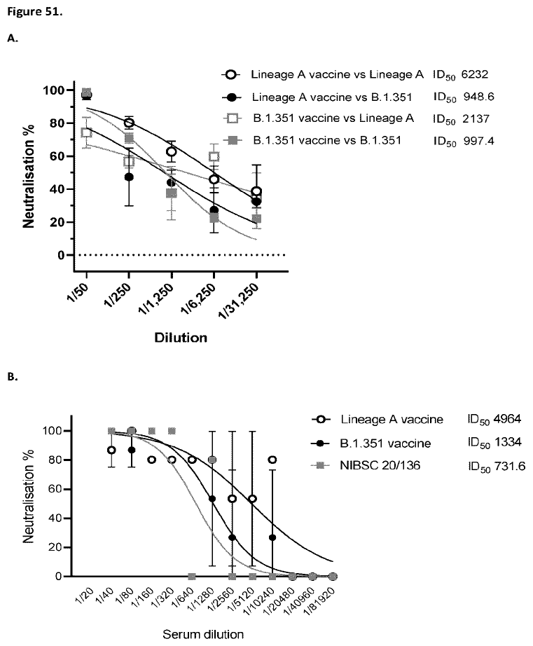

FIG. 51: BALB/c mice were immunised with SN15 or SN17 DNA constructs at days 1, 8 and 15 and sera taken at day 21 analysed in pseudotype neutralisation assay against Lineage A or B.1.351 pseudotype virus (A) or live virus neutralisation assay against Lineage A virus (B). Data are readings at different sera titrations and are representative of multiple experiments.

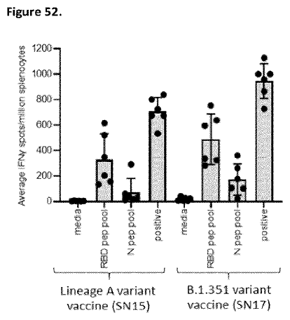

FIG. 52. BALB/c mice were immunised with SN15 or SN17 DNA constructs at days 1, 8 and 15 and splenocytes taken at day 21 and analysed for T cell responses by IFNγ ELISpot assay to RBD or N peptide pools. Symbols represent mean response for individual mice, line represents mean value between mice. Data are collated from multiple independent studies.

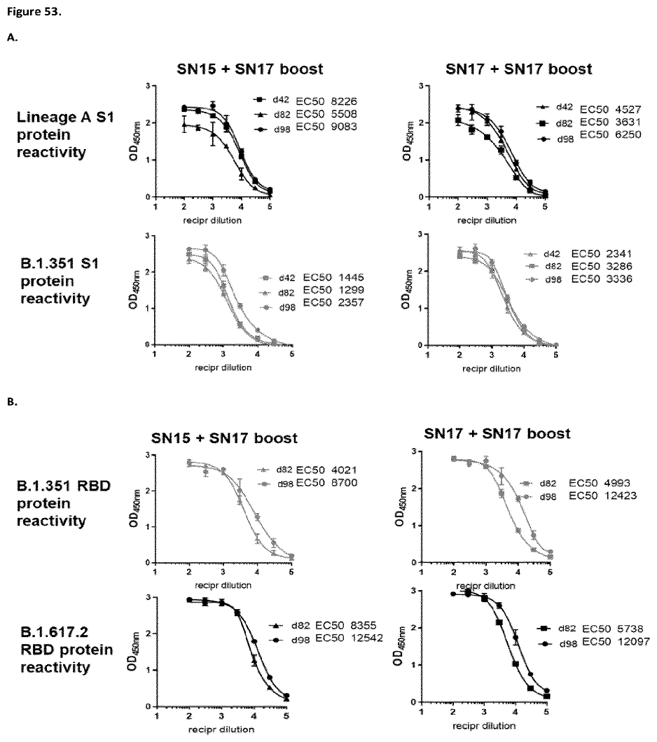

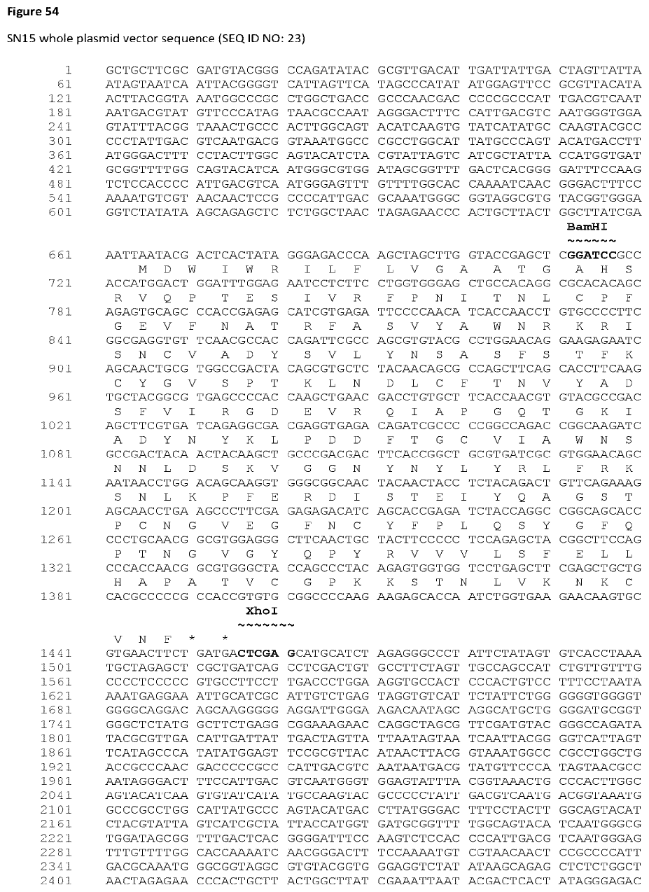

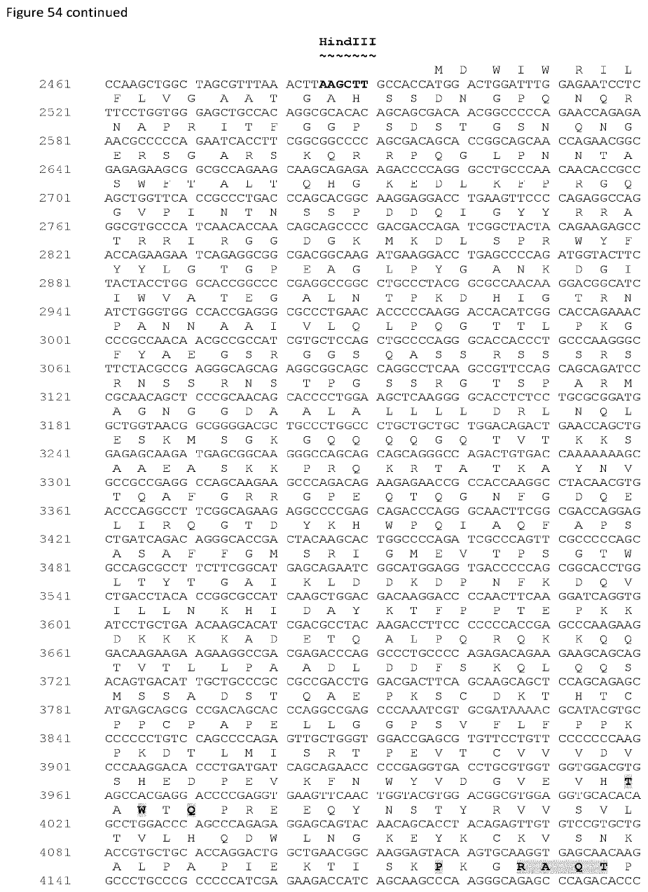

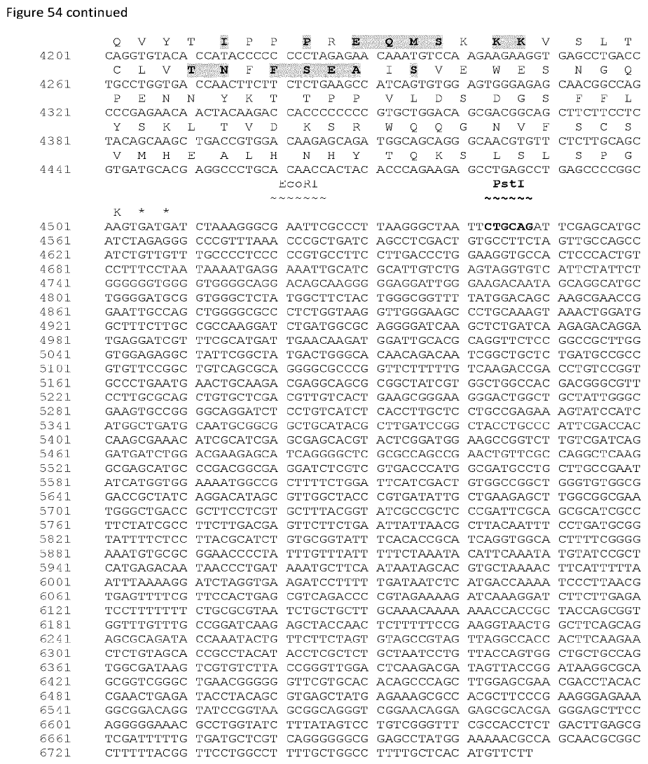

FIG. 53: Balb/c mice immunised on days 1 and 29 with either SN15 (RBD monomer and N linked to modified Fc (FciV1)) or SN17 (RBD monomer and N linked to modified Fc (FciV1)—B.1.351 variant), followed by a booster at day 85 with SN17 DNA. Sera from immunised mice analysed at day 42, 82 or 98 for antibody reactivity to Lineage A (Wuhan) and B.1.351 S1 protein variants (A) or B.1.351 and B.1.617.2 RBD protein variants (B) in ELISA at reciprocal sera dilutions. EC₅₀ values are shown.

FIG. 54: SN15 whole plasmid vector nucleotide sequence (SEQ ID NO: 23).

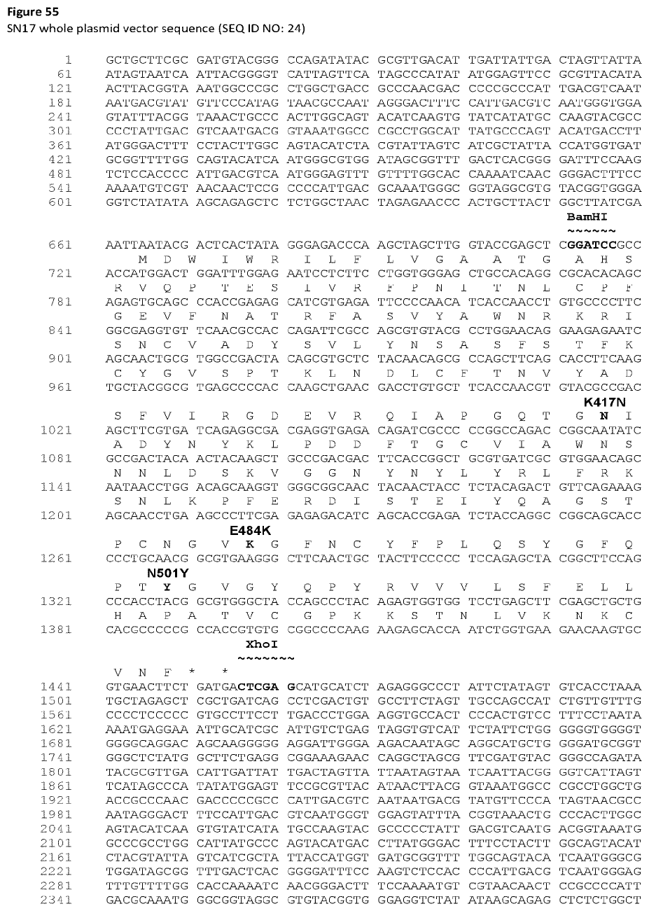

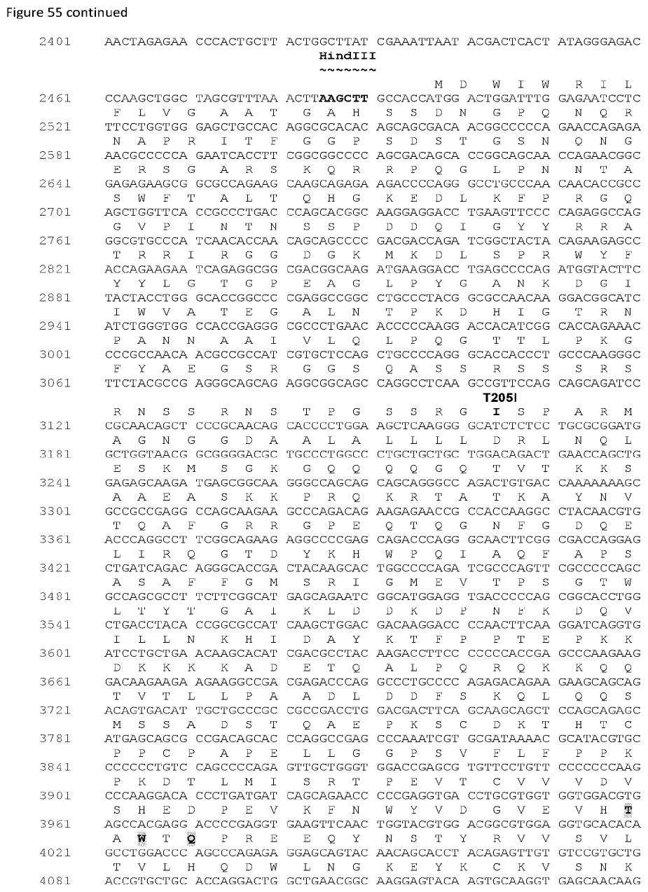

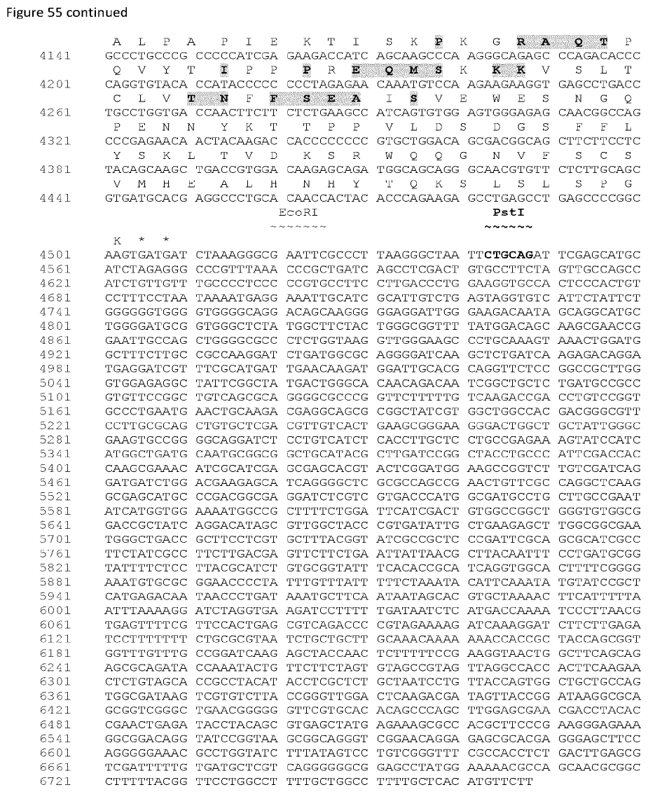

FIG. 55: SN17 whole plasmid vector sequence (SEQ ID NO: 24).

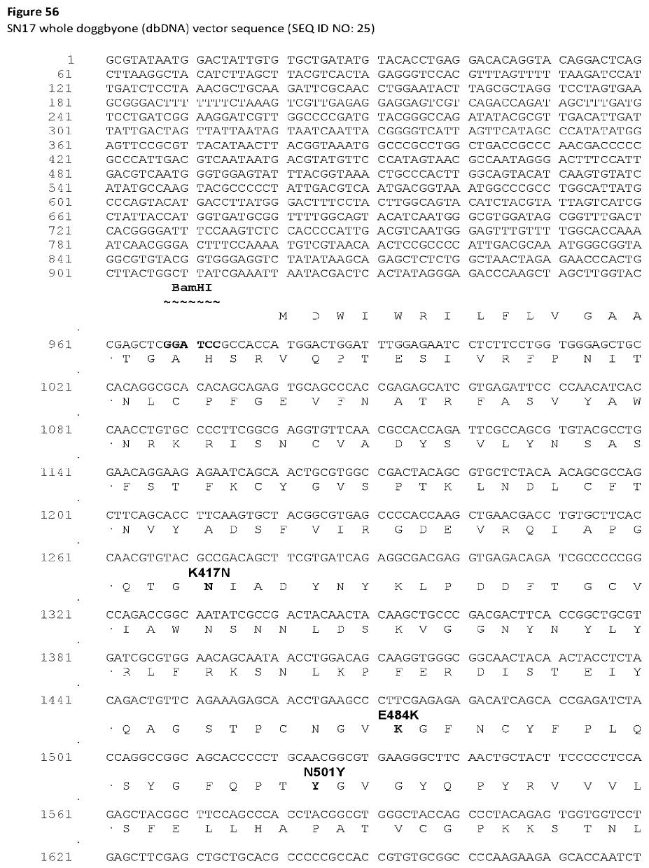

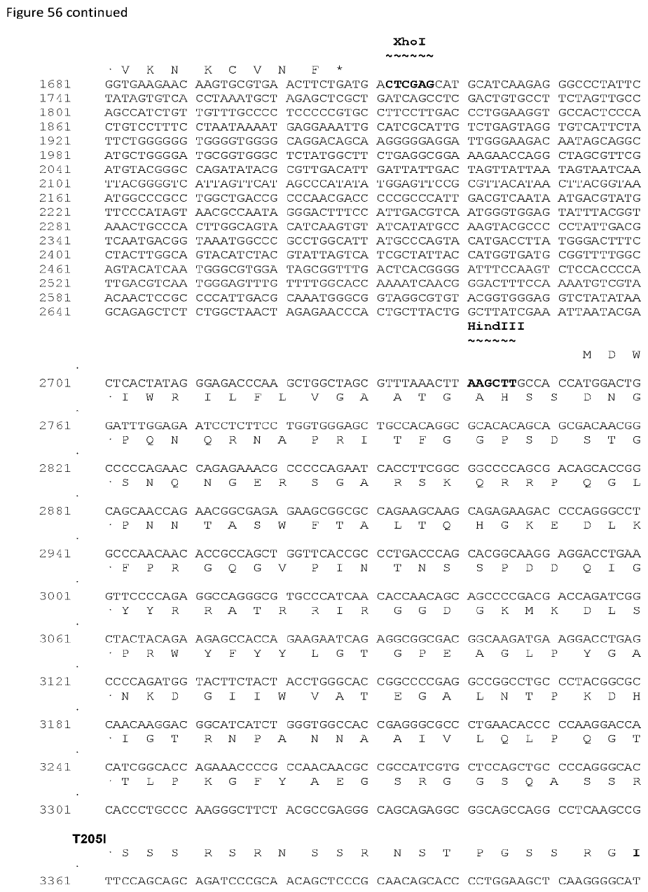

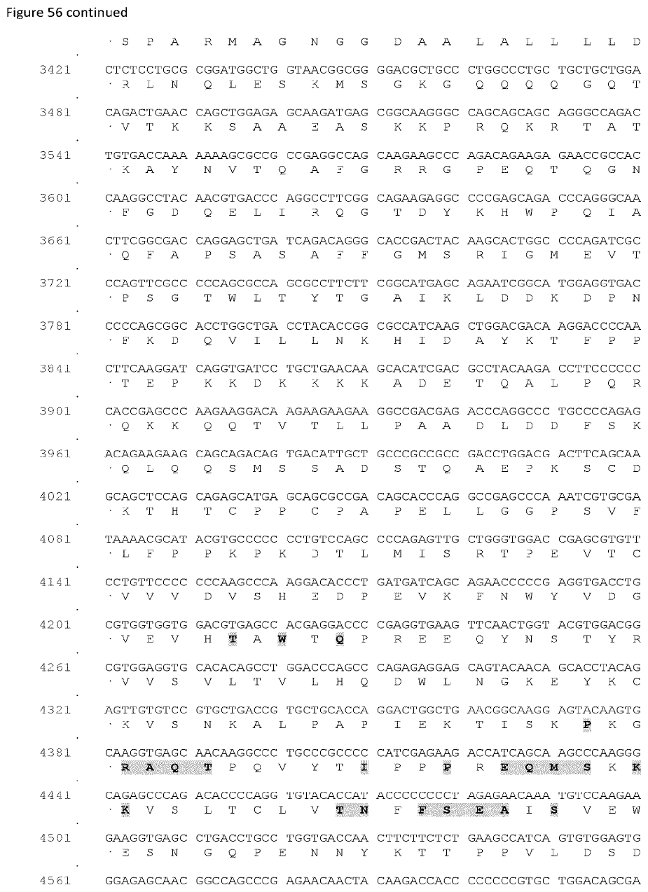

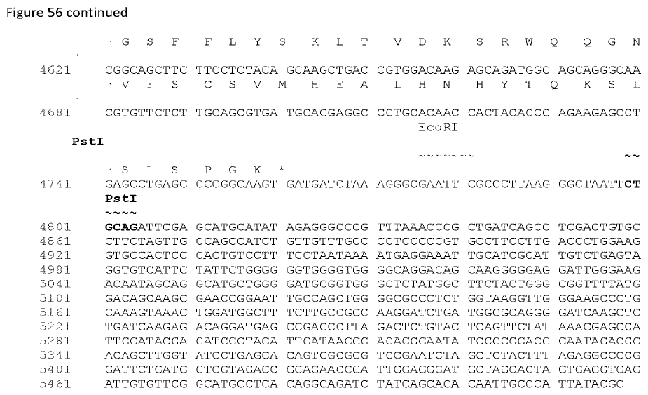

FIG. 56: SN17 whole doggbyone (dbDNA) vector sequence (SEQ ID NO: 25).

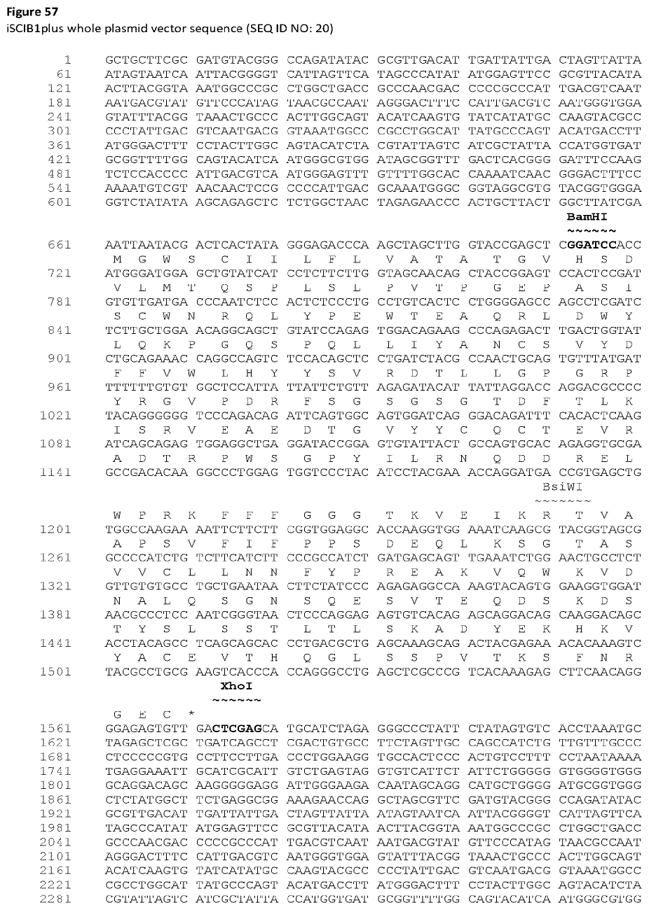

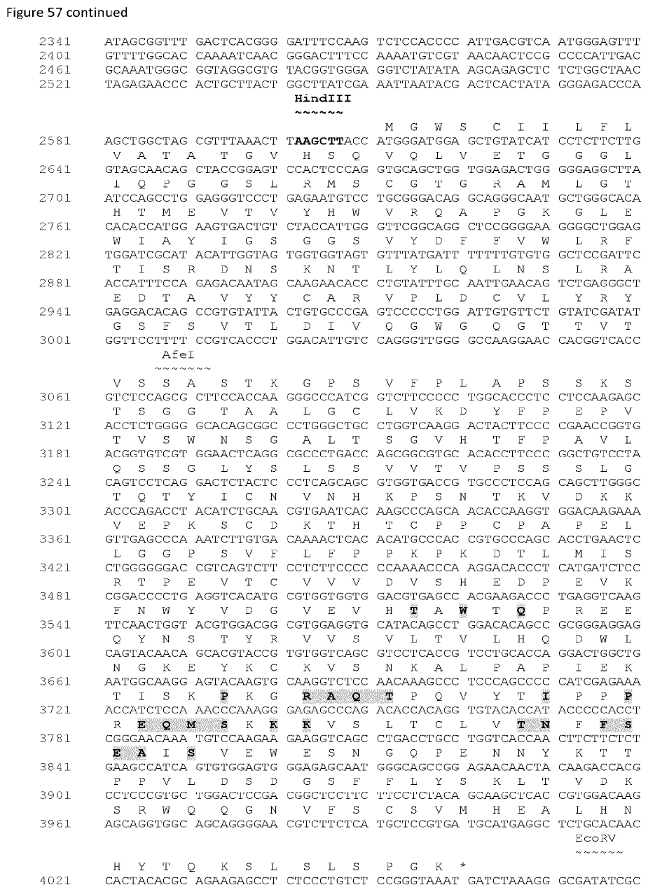

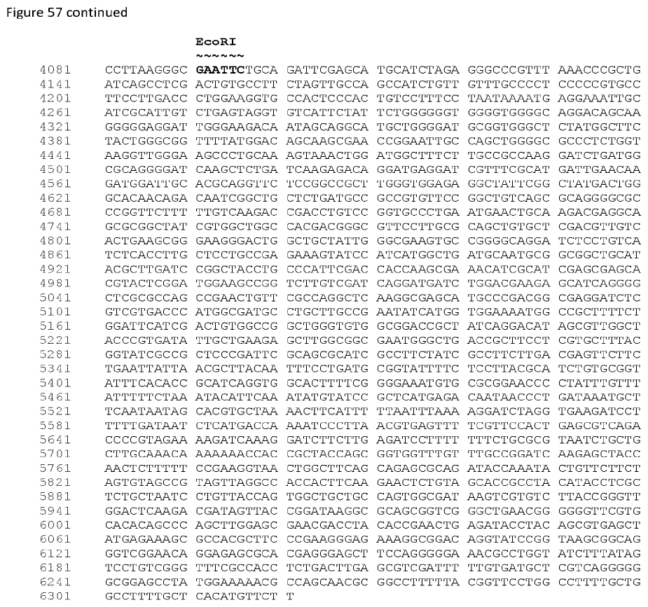

FIG. 57: iSCIB1plus whole plasmid vector sequence (SEQ ID NO: 20).

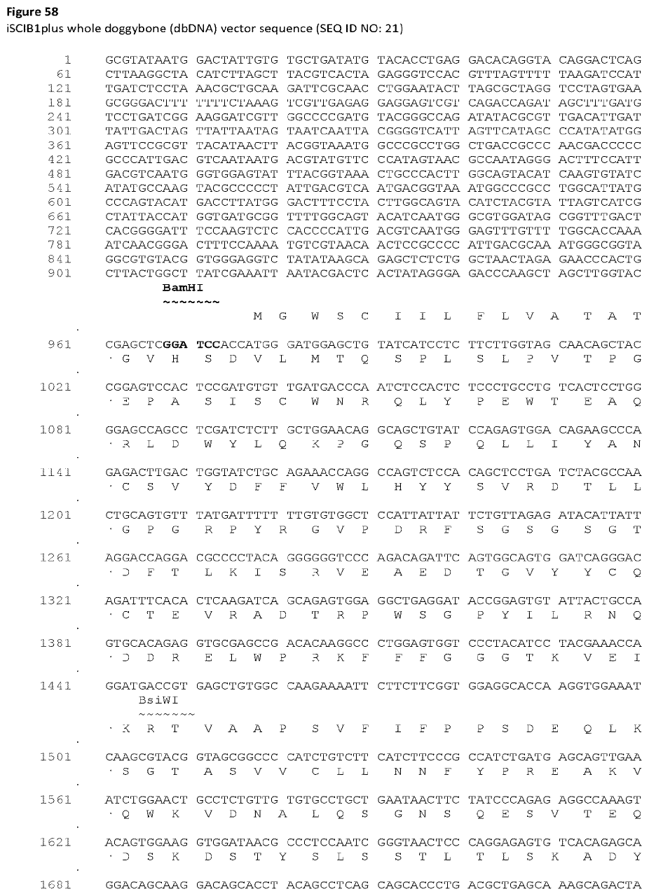

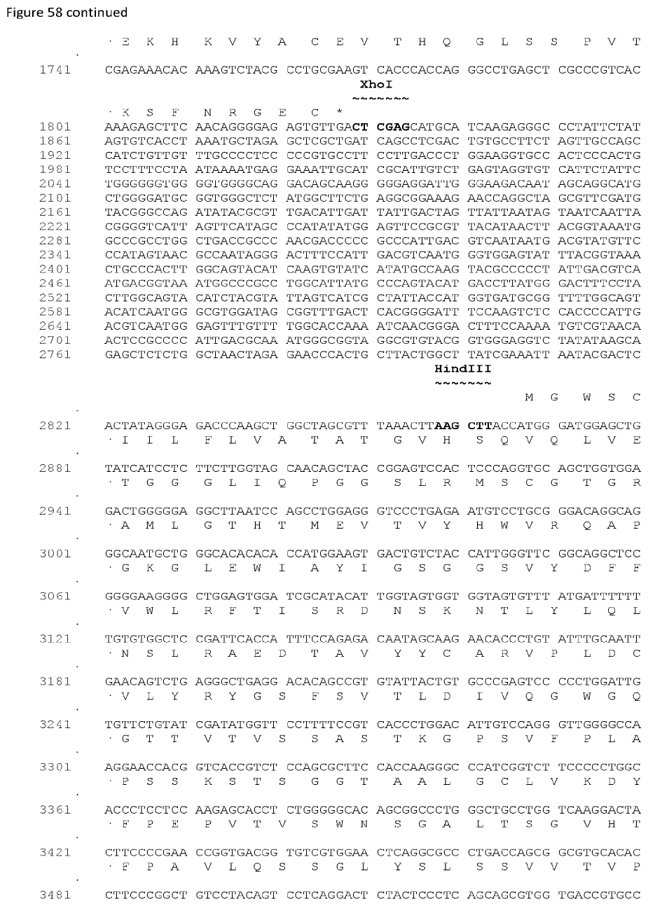

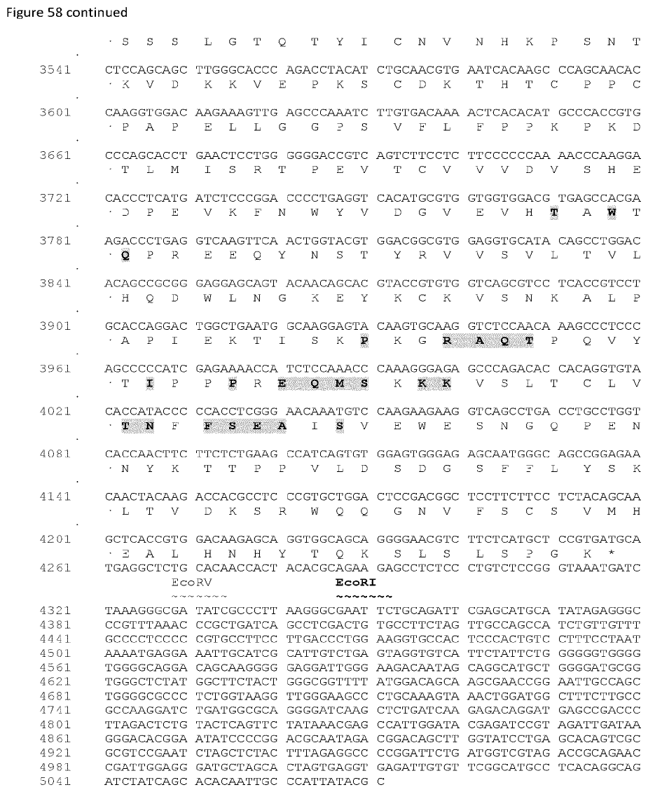

FIG. 58: iSCIB1plus whole doggybone (dbDNA) vector sequence (SEQ ID NO: 21).

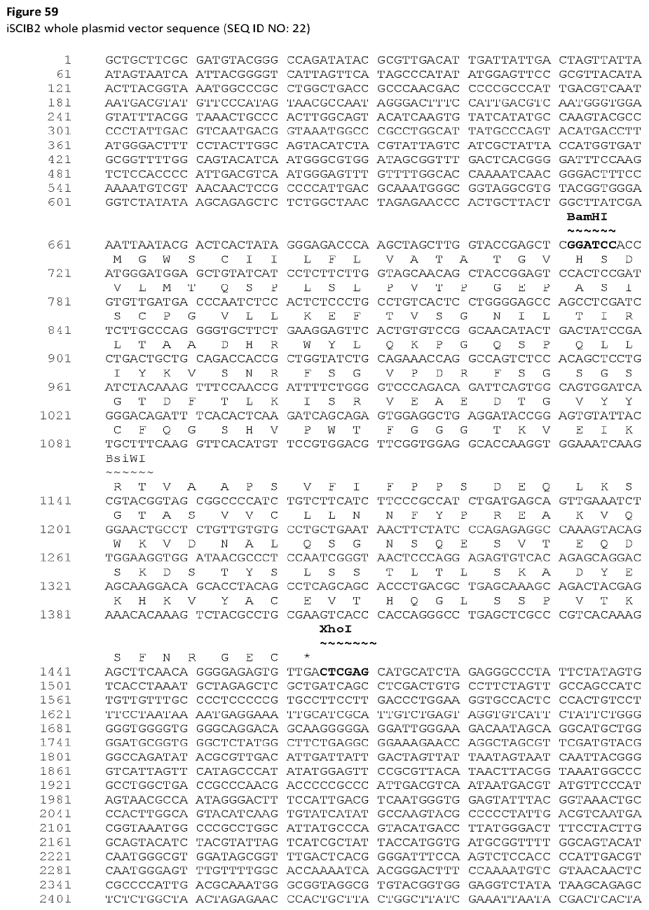

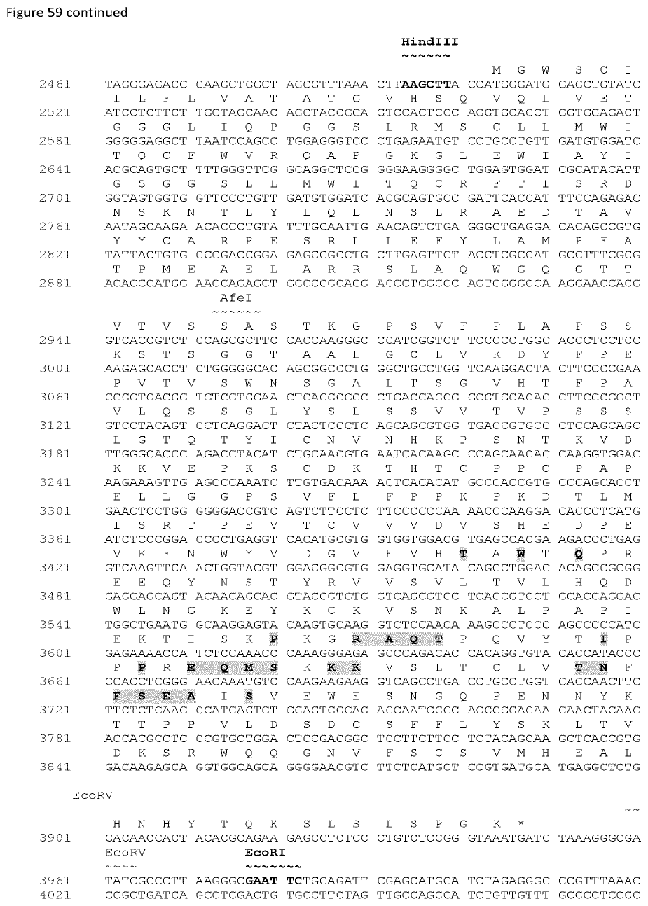

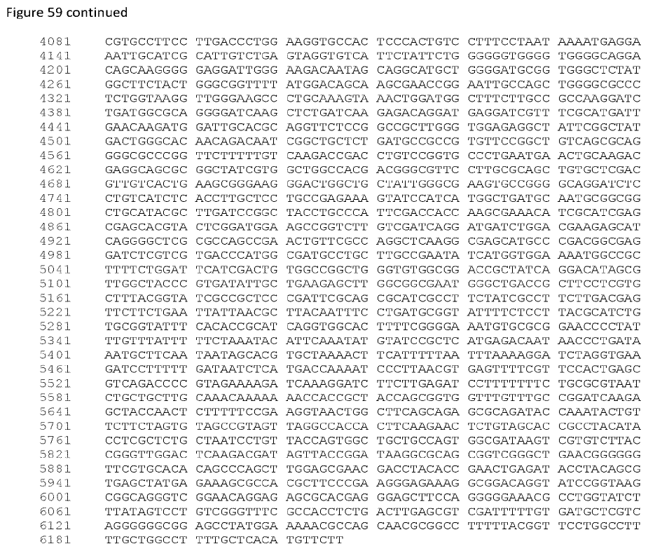

FIG. 59: iSCIB2 whole plasmid vector sequence (SEQ ID NO: 22).

### METHODS

### Materials, Animals, Cells and Antibodies

### Peptides and Proteins

Covid19 peptides were selected based on IEDB database (www.iedb.org/) binding predictions for HLA-A*0201, HLA-DR*0101 and HLA-DP*0401 and SYFPEITHI binding predictions (www.syfpeithi.de) for HLA-A*0201. Cancer antigen peptides were selected based on published sequences, IEDB database (www.iedb.org/) binding predictions and SYFPEITHI binding predictions (www.syfpeithi.de). Peptides (Table 2) were synthesized at >90% purity (GENSCRIPT®), aliquoted to single use vials and stored lyophilized at −80° C. then reconstituted in PBS on day of use. Recombinant N, S1 and His tagged RBD proteins were purchased from GENSCRIPT® (USA). N peptide pool was purchased from MILTENYI BIOTEC® (UK) and RBD peptide pool was purchased from JPT PEPTIDE TECHNOLOGIES™ (Germany).

TABLE 2
Covid19 T cell epitopes

| | | | Predicted | | | |
|---|---|---|---|---|---|---|
| | | | binding | | | |
| | | | score | Predicted binding | | |
| | | | (SYFPEITHI) | score (IEDB)* | | |
| Antigen | coordinates | sequence | HLA-A2 | HLA-A2 | HLA-DR1 | HLA-DP4 |
| Spike | 417-425 | KIADYNYKL (SEQ ID NO: 52) | 26 | 0.7 | — | — |
| glycoprotein | 424-433 | KLPDDFTGCV (SEQ ID NO: | 16 | 7.4 | — | — |
| RBD | | 53) | | | | |
| | 516-524 | ELLHAPATV (SEQ ID NO: 54) | 23 | 3.7 | — | — |
| | 336-355 | CPFGEVFNATRFASVTAWNR | 17 | 5.4 | 28.0 | 3.17 |
| | | (SEQ ID NO: 55) | | | | |
| | 357-376 | RISNCVADYSVLYNSASFST | 15 | 3.5 | 12.0 | 38.5 |
| | | (SEQ ID NO: 56) | | | | |
| | 451-468 | YLYRLFRKSNLKPFERDI (SEQ | 20 | 4.1 | 22.0 | 13.2 |
| | | ID NO: 57)^{¥} | | | | |
| | 505-524 | YQPYRVVVLSFELLHAPATV | 23 | 0.55 | 0.54 | 1.0 |
| | | (SEQ ID NO: 58)^{¥} | | | | |
| Nucleocapsid | 138-146 | ALNTPKDHI (SEQ ID NO: 59) | 22 | 16.0 | — | — |
| protein | 159-168 | LQLPQGTTL (SEQ ID NO: 60) | 18 | 9.0 | — | — |
| | 222-230 | LLLDRLNQL (SEQ ID NO: | 30 | 0.8 | — | — |
| | | 61)^{¥} | | | | |
| | 316-324 | GMSRIGMEV (SEQ ID NO: | 21 | 1.8 | — | — |
| | | 62) | | | | |
| | 351-357 | ILLNKHIDA (SEQ ID NO: 63)^{¥} | 19 | 4.5 | — | — |
| | 212-231 | GNGGDAALALLLLDRLNQLE | 30 | 0.8 | 47.0 | 20.0 |
| | | (SEQ ID NO: 64) | | | | |
| | 299-318 | KHWPQIAQFAPSASAFFGMS | 18 | 1.5 | 15.0 | 30.0 |
| | | (SEQ ID NO: 65)^{¥} | | | | |

*The MHCI binding predictions were made on 04/03/2020 using the IEDB analysis resource NetMHCpan (ver. 4.0) tool [184].
^{¥}Peptides published as known Covid epitopes

TABLE 3
Cancer antigens T cell epitopes

| | | | Predicted/ |
|---|---|---|---|
| Antigen | coordinates | sequence | known binding alleles |
| TRP-2 | 180-188 | SVYDFFVWL (SEQ ID NO: 30) | A2 |
| | 178-192 | NCSVYDFFVWLHYYS (SEQ ID NO: | A2/A31/A33/A3/DP4 |
| | | 119) | |
| Gp100 | 44-59 | WNRQLYPEWTEAQRLD (SEQ ID NO: | DR4 |
| | | 31) | |
| | 174-190 | TGRAMLGTHTMEVTVYH (SEQ ID | A2/DR7/DQ6/DR53 |
| | | NO: 120) | |
| | 178-186 | MLGTHTMEV (SEQ ID NO: 66) | A2 |
| | 476-490 | VLYRYGSFSVTLDIV (SEQ ID NO: 67) | A1/B35/DP4 |
| NY-ESO-1 | 157-165 | SLLMWITQC (SEQ ID NO: 36) | A2 |
| | 158-166 | LLMWITQCF (SEQ ID NO: 35) | A24 |
| | 87-111 | LLEFYLAMPFATPMEAELARRSLAQ | A24/B35/B52/Cw3/C12/B51/ |
| | | (SEQ ID NO: 46) | DR1/DR4/DP4/DR7/DR9 |
| | 119-143 | PGVLLKEFTVSGNILTIRLTAADHR | A68/B49/DR1/DR4/DR52b/DR7/ |
| | | (SEQ ID NO: 38) | DR8 |

### Plasmids

The backbone of all of the COVID-19 plasmids pVAXDCSN1-SN14 (SN1-15) are derived from the FDA regulatory compliant vector backbone of pVAX1 (INVITROGEN®) for use in humans. All nucleotide sections for insertion were codon optimised for expression in humans. SN1-SN4 contain a murine IgK leader while SN5-15 contain a human IgH leader. Codon optimised nucleotide sections encoding a leader, amino acids of the S glycoprotein RBD domain 319-541 or 330-525 (Accession Number YP_009724390) alone, fused in frame with either the Hinge-CH2-CH3 domain of the HuIgG1 constant domain (Accession Number P01857) or the variant Hinge-CH2-CH3iV1 (where 23 amino acids have been replaced with murine IgG3 residues) or attached to a fibritin trimer fold on or disulphide bridge motif via a glycine serine linker was synthesised with BamHI and XhoI sites inserted at the 5′ and 3′ends respectively. In first round of cloning these sections were inserted into the BamHI/XhoI sites of the pVAXDCIB68 construct depicted in FIG. 1 In direct replacement of the light kappa chain in the first expression cassette to generate intermediate plasmids.

In a second round of cloning codon optimised nucleotide sections encoding a leader, full length nucleoprotein amino acids 2-419 (Accession number YP_009724397) alone or fused in frame with the Hinge-CH2-CH3 domain of the HuIgG1 constant domain or the variant Hinge-CH2-CH3iV1 were synthesised and flanked with HindIII/PstI. The heavy chain was excised using HindIII/PstI from the intermediate vectors generated from the first round and replaced with the N sections in the second expression cassette alongside the appropriate S section depicted in FIG. 1.

To enhance the IMMUNOBODY® vectors pVAXDCIB68 (SCIB1), pVAXDCIB238 (SCIB1plus) and pVAXDCIB178 (SCIB2) the huIgG1 constant region of the antibody heavy chain encoding the CH1-Hinge-CH2-CH3 domains (Acc No: P01857 Amino acid 1-330) was replaced with the same section encoding the replaced 23 murine IgG3 residues at the specific sites. This was achieved by synthesis of the nucleotide section encoding CH1-Hinge-CH2-CH3 iV1 flanked by AfeI and EcoRI. The huIgG1 constant domain was excised from the vectors and the section inserted in frame with the heavy variable using these restriction sites.

The sequences of both chains within each expression cassette of the pVAXDC vector of SN1-15, pVAXDCIB68 iV1, pVAXDCIB238 iV1 and pVAXDCIB178 iV1 was confirmed by the dideoxy chain termination method [185]. DNA nucleotide and translated protein sequences for both chains encoded within plasmids SN1-SN15 and the enhanced IMMUNOBODY® vectors pVAXDCIB68 iV1, pVAXDCIB238 iV1 and pVAXDCIB178 iV1 are shown in FIGS. 2-15, 27 and 29, 31 and 33 respectively.

The plasmid pCMV3-2019-nCoV-Spike (S1+S2)-long utilised encoding full length Spike from SARS-COV2 amino acid 1-1273 (Accession number YP_009724390/QHD43416.1)

Was obtained From SINO BIOLOGICAL® (catalogue Number VG40589-UT). This contained codon optimised cDNA for expression of the protein in mammalian cells inserted into the KpnI/XbaI sites of the mammalian expression vector pCMV3-untagged under control of the high level expression mammalian human enhanced cytomegalovirus immediate early (CMV) promoter.

To construct pVAXDCSN16-17 (SN16-17), two consecutive rounds of cloning were required. Two codon optimised nucleotide sections encoding the spike chain comprising of a human IgH leader (MDWIWRILFLVGAATGAHS) (SEQ ID NO: 69) and the Spike glycoprotein RBD domain 319-541 amino acids (Accession Number YP_009724390) containing the N501Y mutation from the Kent variant/lineage B.1.1.7, UK-VOC 202012/01 for SN16 or the K417N, E484K and N501Y mutations from the South African variant/lineage VOC 501Y.V2/B1.351 for SN17 flanked by BamHI and XhoI sites at the 5′ and 3′ends respectively were synthesised. In the first round of cloning the sections were inserted into the BamHI/XhoI sites of the pVAXDCIB68 (SCIB1) plasmid in direct replacement of the SCIB1 light kappa chain in the first expression cassette to generate two intermediate plasmids. In a second round of cloning two codon optimised nucleotide sections encoding the Nucleoprotein chain comprising of the human IgH leader, full length Nucleoprotein amino acids 2-419 (Accession number YP_009724397) containing either the D3L and S235F mutations from the Kent variant/lineage B.1.1.7, UK-VOC 202012/01 for SN16 or the T2051 mutation from the South African variant/lineage VOC 501Y.V2/B1.351 for SN17, fused in frame with the improved variant Hinge-CH2-CH3 iV1 human IgG1 constant domain (where 23 Amino acids have been replaced with murine IgG3 residues) were synthesised and flanked with HindIII/PstI. The SCIB1 heavy huIgG1 chain was excised using HindIII/PstI from the intermediate plasmids generated from the first round and replaced with the Nucleoprotein section in the second expression cassette alongside the appropriate spike section resulting in SN16 and SN17.

The sequences of both chains within each expression cassette of the pVAXDC vector for SN16 and SN17 was confirmed by the dideoxy chain termination method. DNA nucleotide and translated protein sequences for both chains encoded within SN16 and SN17 are shown in FIGS. 47 and 48.

TABLE 4
COVID19 constructs

| FEATURE | AMINO ACID SEQUENCE |
|---|---|
| Murine IgK Leader | METDTLLLWVLLLWVPGSTG (SEQ ID NO: 68) |
| Human IgH Leader | MDWIWRILFLVGAATGAHS (SEQ ID NO: 69) |
| Spike Glycoprotein | RVQPTESIVRFPNITNLCPFGEVFNATRFASVYAWNRKRISNCVADYSVLYNSA |
| Acc No : YP_009724390 | SFSTFKCYGVSPTKLNDLCFTNVYADSFVIRGDEVRQIAPGQTGKIADYNYKLP |
| (AA 319-541) | DDFTGCVIAWNSNNLDSKVGGNYNYLYRLFRKSNLKPFERDISTEIYQAGSTPC |
| | NGVEGFNCYFPLQSYGFQPTNGVGYQPYRVVVLSFELLHAPATVCGPKKSTNLV |
| | KNKCVNF (SEQ ID NO: 8) |
| Spike Glycoprotein | PNITNLCPFGEVFNATRFASVYAWNRKRISNCVADYSVLYNSASFSTFKCYGVS |
| Acc No: YP_009724390 | PTKLNDLCFTNVYADSFVIRGDEVRQIAPGQTGKIADYNYKLPDDFTGCVIAWN |
| (AA 330-525) | SNNLDSKVGGNYNYLYRLFRKSNLKPFERDISTEIYQAGSTPCNGVEGFNCYFP |
| | LQSYGFQPTNGVGYQPYRVVVLSFELLHAPATVC (SEQ ID NO: 70) |
| Spike Glycoprotein | MFVFLVLLPLVSSQCVNLTTRTQLPPAYTNSFTRGVYYPDKVFRSSVLHSTQDL |
| Acc No: YP_009724390 | FLPFFSNVTWFHAIHVSGTNGTKRFDNPVLPFNDGVYFASTEKSNIIRGWIFGT |
| (AA 1-1273) | TLDSKTQSLLIVNNATNVVIKVCEFQFCNDPFLGVYYHKNNKSWMESEFRVYSS |
| | ANNCTFEYVSQPFLMDLEGKQGNFKNLREFVFKNIDGYFKIYSKHTPINLVRDL |
| | PQGFSALEPLVDLPIGINITRFQTLLALHRSYLTPGDSSSGWTAGAAAYYVGYL |
| | QPRTFLLKYNENGTITDAVDCALDPLSETKCTLKSFTVEKGIYQTSNFRVQPTE |
| | SIVRFPNITNLCPFGEVFNATRFASVYAWNRKRISNCVADYSVLYNSASFSTFK |
| | CYGVSPTKLNDLCFTNVYADSFVIRGDEVRQIAPGQTGKIADYNYKLPDDFTGC |
| | VIAWNSNNLDSKVGGNYNYLYRLFRKSNLKPFERDISTEIYQAGSTPCNGVEGF |
| | NCYFPLQSYGFQPTNGVGYQPYRVVVLSFELLHAPATVCGPKKSTNLVKNKCVN |
| | FNFNGLTGTGVLTESNKKFLPFQQFGRDIADTTDAVRDPQTLEILDITPCSFGG |
| | VSVITPGTNTSNQVAVLYQDVNCTEVPVAIHADQLTPTWRVYSTGSNVFQTRAG |
| | CLIGAEHVNNSYECDIPIGAGICASYQTQTNSPRRARSVASQSIIAYTMSLGAE |
| | NSVAYSNNSIAIPTNFTISVTTEILPVSMTKTSVDCTMYICGDSTECSNLLLQY |
| | GSFCTQLNRALTGIAVEQDKNTQEVFAQVKQIYKTPPIKDFGGFNFSQILPDPS |
| | KPSKRSFIEDLLFNKVTLADAGFIKQYGDCLGDIAARDLICAQKFNGLTVLPPL |
| | LTDEMIAQYTSALLAGTITSGWTFGAGAALQIPFAMQMAYRFNGIGVTQNVLYE |
| | NQKLIANQFNSAIGKIQDSLSSTASALGKLQDVVNQNAQALNTLVKQLSSNFGA |
| | ISSVLNDILSRLDKVEAEVQIDRLITGRLQSLQTYVTQQLIRAAEIRASANLAA |
| | TKMSECVLGQSKRVDFCGKGYHLMSFPQSAPHGVVFLHVTYVPAQEKNFTTAPA |
| | ICHDGKAHFPREGVFVSNGTHWFVTQRNFYEPQIITTDNTFVSGNCDVVIGIVN |
| | NTVYDPLQPELDSFKEELDKYFKNHTSPDVDLGDISGINASVVNIQKEIDRLNE |
| | VAKNLNESLIDLQELGKYEQYIKWPWYIWLGFIAGLIAIVMVTIMLCCMTSCCS |
| | CLKGCCSCGSCCKFDEDDSEPVLKGVKLHYT (SEQ ID NO: 71) |
| Fibritin Trimer Fold on | GYIPEAPRDGQAYVRKDGEWVLLSTFL (GTGGGSG) (SEQ ID NO: 72) |
| motif | |
| Disulphide bridge motif | CCGGGSG (SEQ ID NO: 73) |
| Glycine serine Linker | GS OR (GGGS)₃GS (SEQ ID NO: 74) OR (GGGS)₃ (SEQ ID NO: 75) |
| HuIgG1 Hinge-CH2-CH3 | EPKSCDKTHTCPPCPAPELLGGPSVFLFPPKPKDTLMISRTPEVTCVVVDVSHE |
| Acc No: P01857 | DPEVKFNWYVDGVEVHNAKTKPREEQYNSTYRVVSVLTVLHQDWLNGKEYKCKV |
| (AA 99-330) | SNKALPAPIEKTISKAKGQPREPQVYTLPPSRDELTKNQVSLTCLVKGFYPSDI |
| | AVEWESNGQPENNYKTTPPVLDSDGSFFLYSKLTVDKSRWQQGNVFSCSVMHEA |
| | LHNHYTQKSLSLSPGK (SEQ ID NO: 76) |
| HuIgG1 Hinge (AA 99-110) | EPKSCDKTHTCP (SEQ ID NO: 77) |
| HuIgG1 CH2 (AA 111-223) | PCPAPELLGGPSVFLFPPKPKDTLMISRTPEVTCVVVDVSHEDPEVKFNWYVDG |
| | VEVHNAKTKPREEQYNSTYRVVSVLTVLHQDWLNGKEYKCKVSNKALPAPIEKT |
| | ISKAK (SEQ ID NO: 78) |
| HuIgG1 CH3 (AA 224-330) | GQPREPQVYTLPPSRDELTKNQVSLTCLVKGFYPSDIAVEWESNGQPENNYKTT |
| | PPVLDSDGSFFLYSKLTVDKSRWQQGNVFSCSVMHEALHNHYTQKSLSLSPGK |
| | (SEQ ID NO: 79) |
| HuIgG1 Hinge-CH2-CH3 | EPKSCDKTHTCPPCPAPELLGGPSVFLFPPKPKDTLMISRTPEVTCVVVDVSHE |
| iV1 (murine IgG3 23 AA | DPEVKFNWYVDGVEVHTAWTQPREEQYNSTYRVVSVLTVLHQDWLNGKEYKCKV |
| substitutions in bold) | SNKALPAPIEKTISKPKGRAQTPQVYTIPPPREQMSKKKVSLTCLVTNFFSEAI |
| | SVEWESNGQPENNYKTTPPVLDSDGSFFLYSKLTVDKSRWQQGNVFSCSVMHEA |
| | LHNHYTQKSLSLSPGK (SEQ ID NO: 1) |
| HuIgG1 CH1-Hinge-CH2- | ASTKGPSVFPLAPSSKSTSGGTAALGCLVKDYFPEPVTVSWNSGALTSGVHTFP |
| CH3 | AVLQSSGLYSLSSVVTVPSSSLGTQTYICNVNHKPSNTKVDKKVEPKSCDKTHT |
| iV1 (murine IgG3 23 AA | CPPCPAPELLGGPSVFLFPPKPKDTLMISRTPEVTCVVVDVSHEDPEVKFNWYV |
| substitutions in bold) | DGVEVHTAWTQPREEQYNSTYRVVSVLTVLHQDWLNGKEYKCKVSNKALPAPIE |
| | KTISKPKGRAQTPQVYTIPPPREQMSKKKVSLTCLVTNFFSEAISVEWESNGQP |
| | ENNYKTTPPVLDSDGSFFLYSKLTVDKSRWQQGNVFSCSVMHEALHNHYTQKSL |
| | SLSPGK (SEQ ID NO: 80) |
| Nucleoprotein | SDNGPQNQRNAPRITFGGPSDSTGSNQNGERSGARSKQRRPQGLPNNTASWFTA |
| Acc No: YP_009724397 | LTQHGKEDLKFPRGQGVPINTNSSPDDQIGYYRRATRRIRGGDGKMKDLSPRWY |
| (AA 2-419) | FYYLGTGPEAGLPYGANKDGIIWVATEGALNTPKDHIGTRNPANNAAIVLQLPQ |
| | GTTLPKGFYAEGSRGGSQASSRSSSRSRNSSRNSTPGSSRGTSPARMAGNGGDA |
| | ALALLLLDRLNQLESKMSGKGQQQQGQTVTKKSAAEASKKPRQKRTATKAYNVT |
| | QAFGRRGPEQTQGNFGDQELIRQGTDYKHWPQIAQFAPSASAFFGMSRIGMEVT |
| | PSGTWLTYTGAIKLDDKDPNFKDQVILLNKHIDAYKTFPPTEPKKDKKKKADET |
| | QALPQRQKKQQTVTLLPAADLDDFSKQLQQSMSSADSTQA (SEQ ID NO: 4) |

### Animals and Cell Lines

C57Bl/6J, Balb/c (Charles River), HLA-DR4 mice (Model #4149, TACONIC®), HHDII/HLA-DP4 mice (EM:02221, European Mouse Mutant Archive), HHDII mice (Pasteur Institute) or HHDII/HLA-DR1 mice (Pasteur Institute) aged between 8 and 16 weeks old, were used. All work was carried out with ethical approval under a Home Office approved project license. For all the studies mice were randomised into different groups and not blinded to the investigators.

Cells, including B16 melanoma expressing relevant MHCI and II alleles (described previously [140, 183, 186, 187]), were cultured in RPMI medium 1640 with L-glutamine (2 mmol/l) with 10% FCS and appropriate antibiotics to maintain plasmids. HEK293T human embryonic kidney cells (ATCC® CRL1573) were propagated as described previously [188]. Murine splenocytes were cultured in RPMI-1640 with 10% FBS (a), 2 mM glutamine, 20 mM HEPES buffer, 100 units/ml penicillin, 100 mg/ml streptomycin and 10⁻⁵M 2-mercaptoethanol. Cell lines utilised were mycoplasma free, authenticated by suppliers (STR profiling), and used within ten passages.

### Transient HEK293 Transfection

Secretion levels from pDNA constructs were evaluated following transient transfections of Expi293F™ cells using the ExpiFectamine™ 293 Transfection kit (GIBCO®, LIFE TECHNOLOGIES®). Briefly, HEK293 cells in suspension (100 ml, 2×10⁶/ml) were transfected with 100 μg DNA and conditioned medium harvested at day six post-transfection. Conditioned supernatant was filtered through 0.22 μm bottle top filters (MERCK® MILLIPORE®) and sodium azide added to a final concentration of 0.2% (w/v). Cell pellets were lysed in an appropriate volume of RIPA buffer (SIGMA ALDRICH®, R0278) according to the manufacturer's instruction and clarified by centrifugation prior to analysis.

### Immunisation Protocol

Mice were immunised with 1 μg of DNA via gene gun intradermally on days 1, 8 and 15 or days 1, 15 and 29 and responses analysed on day 21 or 35 respectively unless stated otherwise.

For tumour therapy studies mice were implanted with 2.5×10⁴ B16F1 or B16 HHDII Nyeso1 tumour cells subcutaneously on day 1 followed by vaccinations on days 4, 11 and 18 or days 4, 8 and 11. Tumours were measured at 3-4 days intervals. Tumour growth in mice was analysed by measuring the tumour size with callipers (length and width). Volume was estimated by the formula below:

Volume=(π/6)×(width×length²)

### Pseudovirus Neutralisation Assays

SARS-CoV-2 spike protein plasmids were generated and cloned, and pseudoparticles generated following the methodology described for hepatitis C virus in [189]. Pseudoparticles generated in the absence of the plasmid were used as a negative control. For infectivity and neutralisation testing of SARS-CoV-2 pseudoparticles, HEK293T cells per well were plated in white 96-well tissue culture plates (CORNING®) and incubated overnight at 37° C. The following day, SARS-CoV-2 pseudoparticles were mixed with appropriate amounts of antibody and then incubated for 1 hr at 37° C. before adding to cells. After 72 hrs at 37° C., either 1001 μl BRIGHT-GLO™ (PROMEGA®) was added to each well and incubated for 2 mins or cells were lysed with cell lysis buffer (catalog no. E1500; PROMEGA®) and placed on a rocker for 15 mins. Luciferase activity was then measured in relative light units (RLUs) using either a SPECTRAMAX® M3 microplate reader (MOLECULAR DEVICES®) with the SOFTMAXPRO® 6 software (BRIGHT-GLO™ protocol), or wells were individually injected with 50 μl luciferase substrate and read using a FLUOSTAR® Omega plate reader (BMG LABTECH®) with the MARS software. Infection by SARS-CoV-2 pseudoparticles was measured in the presence of anti-SARS-CoV-2 mAbs, tested animal sera, preimmune animal sera, and nonspecific IgG at the same dilution. Each sample was tested in duplicate or triplicate. Neutralising activities were reported as 50% inhibitory dilution (ID₅₀) values and were calculated by nonlinear regression (GRAPHPAD PRISM® version 7), using lower and upper bounds (0% and 100% inhibition) as constraints to assist curve fitting.

### Live Virus Neutralisation Assay

SARS-CoV-2 infectious virus (CVR-GLA-1) was obtained from the National Centre For AIDS Reagents, NIBSC, UK.

Live virus neutralisation assays were performed using method previously described [190], except that 790 TCID₅₀/ml of the SARS-CoV-2 virus was added to each serum dilution. Additionally, for some experiments, the sera were diluted down to 1:81,920.

### ACE2 Binding Inhibition Assay

A V-PLEX COVID-19 ACE2 neutralisation kit from Meso Scale Diagnostics LLC was used to investigate the ability of vaccine-elicited antibodies to block the binding of ACE2 to RBD or whole S proteins. V-plex SARS-CoV-2 Panel 7 multispot plates containing S1 RBD and whole S proteins from Lineage A (originally identified in Wuhan) and variant (B1.1.7, B1.351, P.1) SARS-CoV-2 strains were blocked, followed by incubation with sera at 1:100 dilution and Sulfo-tagged human ACE2 protein, according to the manufacturer's instructions. Results are expressed as percentage inhibition of ACE2 binding via comparison of sera-incubated samples to diluent-containing wells (absence of inhibition).

### RBD Binding Inhibition Assay (Surrogate Neutralisation Assay)

RBD binding inhibition was assessed using kit purchased from GENSCRIPT® (USA). In brief, sera from immunised mice at various dilutions was mixed with recombinant HRP tagged RBD protein.

Mixtures were added to plates precoated with ACE2 receptor and RBD binding detected using TMB substrate. RBD binding inhibition was calculated as loss of colorimetric signal where the negative control was 0%.

### ELISpot Assays

ELISpot assays were performed using murine IFNγ capture and detection reagents according to the manufacturer's instructions (MABTECH®, Nacka Strand, Sweden). In brief, anti-IFNγ antibodies were coated onto wells of 96-well Immobilin-P plate and quadruplicate wells were seeded with 5×10⁵ splenocytes and final concentrations of 10 μg/ml synthetic peptides, 1 μg/ml recombinant protein or 1 μg/ml peptide pools were added unless stated otherwise. Plates were incubated at 37° C. for 40 hrs in an atmosphere of 5% CO₂. Following incubation, captured IFNγ was detected by a biotinylated anti-IFNγ antibody and development with a streptavidin alkaline phosphatase and chromogenic substrate. Spots were analysed and counted using an automated plate reader (Cellular Technologies Ltd Europe GmbH, Aalen, Germany). Functional avidity was calculated as the concentration mediating 50% maximal effector function using a graph of effector function versus peptide concentration.

### ELISA for Anti S and N Antibodies

Commercial N, S1 and RBD proteins (GENSCRIPT®, USA) were diluted in PBS and coated onto high protein binding 96-well plates at 0.5 μg/well overnight at 4° C. Plates were washed and blocked with casein blocker (THERMO SCIENTIFIC® Ref: 37528) at 200 μl/well for at least an hour at RT, followed by the addition of murine sera (at various dilutions) diluted in PBS 2% BSA for 1 hr at room temperature. Plates were washed and incubated with anti-mouse Ig HRP antibody in PBS 2% BSA (2-step ELISA) or anti-mouse Fc-biotin followed by strepativin-HRPO (3-step ELISA) for 1 hr at room temperature. Following washing TMB substrate added and reaction stopped with 1N H₂SO₄. Commercially available murine IgG N and S1 specific antibodies (SINO BIOLOGICAL®) were used as controls (N +ve and S1 +ve). Sera from naive mice was included as a negative control. Absorbance was read at 450 nm wavelength.

### Sandwich ELISA for Detection of Secreted RBD and N Proteins

Commercial kits/antibody pairs were used in both cases. NP was detected in conditioned medium and cell lysate (six days post-transfection) using the SARS-CoV-2 NP ELISA kit from Bioss, (cat #BSKV0001) according to supplier's instructions. Quantitation relied on the standard curve with NP standard supplied by the kit. RBD (secreted and in cell lysate) was detected using a sandwich ELISA consisting of a capture antibody: SARS-CoV-2 Spike neutralising mouse mAb (SINO BIOLOGICAL®, 40591-MM43) combined with a HRPO-labelled detection antibody from the SARS-CoV-2 S protein RBD Antibody Pair (Epigentek A73682). Capture antibody was coated at 200 ng/well; detection antibody was used at a dilution of 1:1000.

### SPR Analysis of CD64 Binding by Fc-Fusion Constructs

The analysis was performed on a BiaT200. His-tagged CD64 (ACROBIOSYSTEMS®, FCA-H52H1) was captured onto a CM5 chip coupled with an anti-His antibody. The chip contains 4 flow cells, three of which were used to capture increased densities of CD64, the fourth flow cell was a reference cell. Fc-constructs were titrated over 50.0 nM to 0.78 nM concentration range, and the association and dissociation monitored for 210 s and 700 s respectively, at a flow rate of 30 ul/'. Kinetic parameters were deduced by fitting the data to a 1:1 monovalent binding model.

Immunogenicity Analysis of the Modified Fc (iFcv1) Construct

The analysis was performed by ABZENA® (Cambridge) Ltd according to HTA standards. PBMCs were isolated from healthy community donor buffy coats (obtained under consent from commercial vendors). Cells were separated by density centrifugation using Lymphocyte separation medium (CORNING®, Amsterdam, The Netherlands) and CD8+ T cells were depleted using CD8+ RosetteSep™ (STEMCELL TECHNOLOGIES®, London, UK). Donors were characterised by identifying HLA-DR and HLA-DQ haplotypes to 4 digit resolution by HISTO Spot SSO HLA typing (MC Diagnostics, St. Asaph, UK). T cell responses to the neo-antigen KLH (INVITROGEN®, Paisley, UK) were also determined. PBMC were then frozen and stored in the vapour phase of nitrogen until required. A cohort of up to 50 donors was selected covering 77% of HLA alleles. PBMCs from each donor were thawed, counted and viability assessed using trypan blue (MERCK® Life Science UK Ltd, Gillingham, UK) dye exclusion. For each donor, bulk cultures were established in which 1 ml of the proliferation cell stock was added to the appropriate wells of a 24 well plate. 1 ml of the test sample (iTv1) was added to the PBMC to give a final sample concentration of 0.3 μM. For each donor, a reproducibility control well (cells incubated with 0.3 μM KLH), a clinical benchmark control well (cells incubated with 5 μM Bydureon®), a low immunogenicity control (cells incubated with 0.3 μM Herceptin®) and a culture medium only well were also included. Cultures were incubated for a total of 8 days at 37° C. with 5% CO₂. On days 5, 6, 7 and 8, the cells in each well were gently resuspended by mixing 5× using an electronic pipette and 3×100 μl aliquots transferred to each well of a round bottomed 96 well plate. The cultures were pulsed with 0.75 μCi [3H]-Thymidine (Perkin Elmer®, Beaconsfield, UK) in 100 μl AIM-V® culture medium and incubated for a further 18 hours before harvesting onto filter mats (Perkin Elmer®, Beaconsfield, UK) using a TomTec Mach III cell harvester. CPM for each well were determined by Meltilex™ (Perkin Elmer®, Beaconsfield, UK) scintillation counting on a 1450 Microbeta Wallac Trilux Liquid Scintillation Counter (Perkin Elmer®, Beaconsfield, UK) in paralux, low background counting. An empirical threshold of a SI equal to or greater than 1.9 (SI≥1.90) has been previously established whereby samples inducing responses above this threshold were deemed positive.

### Statistical Analysis

Statistical analysis of responses to the nucleic acid plasmids was performed using GRAPHPAD PRISM® software version 7. Comparative analysis of the ELISpot results was performed by applying paired or unpaired ANOVA or Student t test as appropriate with p values calculated accordingly. Comparison of avidity curves/survival was assessed by applying the F test using the GRAPHPAD PRISM® software, or log-rank test. P<0.05 values were considered statistically significant. Statistical analysis and comparison of neutralisation titres between groups (ID₅₀ values) was performed using Kruskal-Wallis analysis of variance (ANOVA) with Dunn's multiple-comparison test. Data analysis was not blinded. As above, differences were considered statistically significant at a p value of <0.05 and statistical analyses performed using the GRAPHPAD PRISM® 8 software.

### Preclinical Studies

### Example 1—RBD Protein and N Protein Secretion from Transient HEK293 Transfections with pDNA

In order to assess the transfection efficiency of the pDNA constructs, HEK293 cells were transiently transfected with the pDNA using THERMO FISHER SCIENTIFIC®'s Expi293 system and protein secretion in the medium evaluated using sandwich ELISAs for the RBD protein and Nucleoprotein (FIG. 16). This analysis indicated that for the RBD protein, the constructs containing the RBD-Fc fusion (SN3 and SN7), gave the highest secretion; closely followed by the unmodified RBD (SN4, SN5, SN8). Trimeric RBD (SN2, SN6, SN9, SN10, SN11) resulted in the lowest secretion levels. Nucleoprotein secretion was highest for constructs containing unmodified NP (SN3, SN4, SN7, SN8). As the latter target is more relevant for T cell responses compared to inducing neutralising antibodies; lower secretion levels (such as seen for the N Fc fusion proteins) resulting in more avid T cell responses are desirable.

### Example 2—T Cell Responses to the RBD and N Proteins (NP) with pDNA Delivered Via Gene Gun to HHDII Mice

T cell responses to pVAXDC SPIKE RBD+NP (SN8), pVAXDC SPIKE RBD v2 TRIMER+NPFC (SN9), pVAXDC SPIKE RBD v3 TRIMER+NPFC (SN10) and pVAXDC SPIKE RBD v2 TRIMER+NPFC iV1 (SN11) following three weekly immunisations of HHDII mice with pDNA administered via gene gun were measured. The frequency of the IFNγ ELISpot responses to all RBD constructs was measured using predicted or previously identified T cell epitopes and the whole S1 protein, the RBD recombinant protein and RBD peptide pool. All four constructs showed strong responses to the S1 protein as well as the RBD peptide pool and RBD aa417-425 peptide of which responses to RBD peptide pool and RBD aa417-425 peptide reached significance from all constructs (FIG. 17A). The frequency of the responses to N antigen were measured using an overlapping peptide pool, recombinant N protein and identified T cell epitopes. All constructs showed strong responses to the recombinant N protein but only constructs SN10 and SN11 showed significant responses to the N aa138-146 peptide (FIG. 17A).

Another study compared responses to pVAXDC SPIKE RBD+NP (SN8), pVAXDC SPIKE RBD v3 TRIMER+NPFC (SN10) and pVAXDC SPIKE RBD v2 TRIMER+NPFC iV1 (SN11) following three weekly immunisations of HHDII mice with pDNA administered via gene gun. All three constructs showed significant responses to the S1 protein as well as the RBD peptide pool but only SN11 immunised mice showed significant responses to RBD aa417-425 peptide (FIG. 17B). In regards to the N protein specific responses, all constructs show strong responses to the N protein but only mice immunised with SN11 show significant responses to the N 138-146 peptide (FIG. 17B).

### Example 3—T Cell Responses to the RBD and N Proteins (NP) with pDNA Delivered Via Gene Gun to HHDII/DR1 Mice

T cell responses to pVAXDC SPIKE RBD+NP (SN8), pVAXDC SPIKE RBD v2 TRIMER+NPFC (SN9), pVAXDC SPIKE RBD v3 TRIMER+NPFC (SN10) and pVAXDC SPIKE RBD v2 TRIMER+NPFC iV1 (SN11) following three weekly immunisations of HHDII/DR1 mice with pDNA administered via gene gun were measured. The frequency of the IFNγ ELISpot responses to all RBD constructs was measured using an identified T cell epitope (RBD aa417-425), the whole S1 protein, whole RBD protein and the RBD peptide pool. Constructs SN8, SN9 and SN10 showed significant responses only to the whole S1 protein whereas construct SN11 generated responses to the S1 and RBD proteins, RBD peptide pool and RBD aa417-425 peptide. (FIG. 18). The frequency of the responses to N antigen was measured using an overlapping peptide pool, N protein and an identified T cell epitope (N aa138-146). Constructs SN10 and SN11 generated significant responses to the whole N protein but only construct SN11 generated a significant response to the N aa138-146 peptide (FIG. 18).

### Example 4—Construct Containing NPFC iV1 Generates Higher Frequency Responses to N Protein

To compare the T cell responses from constructs containing N antigen (SN8), N linked to FC (SN9) or N linked to modified iV1 FC (SN11) data was combined and normalised against background control. Construct SN11 showed significantly enhanced responses to both N protein and the N 138-146 peptide when compared to SN8 or SN9 (** p<0.01 and * p<0.05 respectively) (FIGS. 19A and B). Interestingly responses to the S1 protein were also higher frequency from construct SN11 when compared to SN8 (*p<0.05) and to the RBD protein compared to SN8 and SN9 (***p<0.001) (FIGS. 19C and D).

### Example 5—T Cell Responses to the RBD and N Proteins with pDNA Delivered Via Gene Gun to HHDII/DP4 Mice

T cell responses to pVAXDC SPIKE RBD TRIMER+NPFC (SN2), pVAXDC SPIKE RBD FC+NP (SN3) and pVAXDC SPIKE RBD+NP (SN4) following three fortnightly immunisations of HHDII/DP4 mice with pDNA administered via gene gun were measured. The frequency of the IFNγ ELISpot responses to all RBD constructs was measured using predicted and identified T cell epitopes, the whole S1 protein and the RBD protein. Significant responses were seen from construct SN2 to the RBD aa417-425 peptide, RBD protein and RBD peptide pool and from construct SN4 to the S1 and RBD proteins (FIG. 20). The frequency of the ELISpot responses to N constructs was measured using N protein and predicted and identified T cell epitopes. Constructs SN2 and SN4 showed strong responses to the N protein (***p<0.001) but that for SN3 did not reach significance (FIG. 20).

### Example 6—Avidity of T Cell Responses to the 51 Protein with pDNA Delivered Via Gene Gun is Superior from RBD FC and RBD TRIMER Constructs

T cell responses to pVAXDC SPIKE RBD TRIMER+NPFC (SN2), pVAXDC SPIKE RBD FC+NP (SN3) and pVAXDC SPIKE RBD+NP (SN4) following three fortnightly immunisations of HHDII/DP4 mice with pDNA administered via gene gun were assessed for avidity to S1 protein titration. Responses in mice immunised with SN2 and SN3 show significantly higher avidity of responses (p<0.0001) compared to those immunised with SN4 (FIG. 21). Avidity measured using normalised data as the concentration that elicits 50% maximum response. This equates to 0.0001 μg/ml for SN2, 0.000008 μg/ml for SN3 and 0.17 μg/ml for SN4.

### Example 7—pDNA Immunisation Generates High Avidity Peptide Specific Responses to the RBD 417-425 Epitope

T cell responses to pVAXDC SPIKE RBD v2 TRIMER+NPFC iV1 (SN11) following three weekly immunisations of HHDII mice with pDNAs administered via gene gun were assessed for avidity to RBD 417-425 peptide by peptide titration. Responses in mice immunised with SN11 show high avidity responses of greater than 0.0001 ug/ml (FIG. 22).

### Example 8—Frequency and Avidity of T Cell Responses to the N 138-146 Peptide with pDNA Delivered Via Gene Gun is Superior from NPFC iV1 Construct

T cell responses to pVAXDC SPIKE RBD v3 TRIMER+NPFC (SN10), pVAXDC SPIKE RBD v2 TRIMER+NPFC iV1 (SN11) following three weekly immunisations of HHDII mice with pDNA administered via gene gun were assessed for avidity to N 138-146 peptide by peptide titration. Responses in mice immunised with SN11 show higher frequency as well as slightly higher avidity of responses compared to those immunised with SN10 (FIG. 23). Showing that the NP FC iV1 construct (SN11) has induced higher frequency and avidity T cells when compared to the NPFC construct (SN10).

### Example 9—Antibody Responses to the RBD and N Proteins with pDNA Delivered Via Gene Gun

Antibody responses to pVAXDC SPIKE RBD+NPFC (SN5), pVAXDC SPIKE RBD TRIMER+NPFC (SN6), pVAXDC SPIKE RBD v2 TRIMER+NPFC (SN9), pVAXDC SPIKE RBD v3 TRIMER+NPFC (SN10), pVAXDC SPIKE RBD v2 TRIMER+NPFC iV1 (SN11) or pVAXDC SPIKE RBD TRIMER+NPFC (SN2), pVAXDC SPIKE RBD FC+NP (SN3) and pVAXDC SPIKE RBD+NP (SN4) following three fortnightly immunisations of HHDII/DP4 mice with pDNA administered via gene gun were measured. The Ab titres to the S1, RBD and N proteins were compared in sera from mice immunised with the monomer RBD construct, the dimer RBD presented as an Fc fusion protein, the RBD construct as a trimer and the shorter RBD as a trimer both presented as a fibritin construct. Antibodies were assessed in sera at 1/100 to 1/10,000 dilution. Strong reactivity to the N protein was observed in sera from all immunised mice even at 1/10,000 dilution (FIG. 24). Reactivity to the S1 and RBD proteins was seen at 1/100 and 1/1000 dilutions of sera and was highest from constructs SN5, SN6 and SN10 containing the longer RBD construct either as a monomer (SN5) or trimer (SN6) and the shorter RBD v3 trimer (SN10) (FIG. 24A). Similar antibody responses to S1 and N protein were detected in sera from SN2, SN3 and SN4 immunised mice (FIG. 24B).

Antibody responses to pVAXDC SPIKE RBD FC+NP (SN3), pVAXDC SPIKE RBD+NP (SN8), pVAXDC SPIKE RBD v3 TRIMER+NPFC (SN10) and pVAXDC SPIKE RBD v2 TRIMER+NPFC iV1 (SN11) following three weekly immunisations of HHDII mice with pDNA administered via gene gun were measured in an ELISA assay. Antibodies were assessed in sera at 1/100 to 1/10,000 dilution. Strong reactivity to the N protein was observed in sera from all immunised mice even at 1/10,000 dilution. Reactivity to the S1 protein was seen at 1/100 and 1/1000 dilutions of sera and was highest from construct SN10 containing the shorter RBD v3 trimer (SN10) but also detectable at 1/1000 sera dilution from construct SN8 containing the full length RBD monomer (FIG. 24C).

### Example 10—pDNA Delivered Via Gene Gun Elicits Virus Neutralising Antibody Responses with Similar Titre to Total Antibody Measurement

Virus neutralising antibodies were assessed in a surrogate neutralisation assay for inhibition of RBD binding to plate bound ACE2 receptor. Sera from mice immunised with constructs SN5, SN6 and SN10 containing the longer RBD monomer, longer RBD trimer or shorter RBD v3 trimer, respectively, showed >50% inhibition at 1/100 dilution with lower titres seen from constructs SN9 and SN11 (FIG. 25). Sera samples were also tested for virus neutralisation in a pseudovirus neutralisation assay. Sera from SN2, SN3 and SN4 immunised mice were assessed after only two immunisations at day 21 whereas for SN5, SN6, SN9, SN10 and SN11 sera was assessed at termination (day 35). In this assay only sera from mice immunised with constructs SN3, SN5 and SN6 showed >50% virus neutralisation at 1/100 dilution with SN5 showing closer to 90% neutralisation and SN3 close to 80% neutralisation at 1/100 sera dilution (FIG. 26A). No virus neutralisation was seen against an irrelevant virus (FIG. 26B). Strong Ab titres against N protein are seen from NP, NPFC and NPFC iV1 containing constructs. Constructs containing RBD monomer or RBD dimer presented as an Fc fusion show the highest Ab titres targeting S protein as well as the highest virus neutralising antibodies which are of similar titre to total Ig antibodies.

Virus neutralisation was also analysed at different sera dilutions in the pseudovirus neutralisation assay (FIG. 26C). Titration data shows that sera from mice immunised with construct SN5 demonstrates a 50% neutralisation titre (ID50) at 1/3517 dilution of sera. Mice immunised with construct SN6 a titre at 1/236 dilution and those immunised with construct SN3 a titre at 1/600 dilution (FIG. 26D).

TABLE 5
Summary of COVID19 constructs

| | |
|---|---|
| Name | Description |

(1-4 murine Leader IgK)

| | |
|---|---|
| SN1 | pVAXDC Spike RBD + NPFC |
| SN2 | pVAXDC SPIKE RBD TRIMER + NPFC |
| SN3 | pVAXDC SPIKE RBD FC + NP |
| SN4 | pVAXDC SPIKE RBD + NP |

(5-15 Human Leader IgH)

| | |
|---|---|
| SN5 | pVAXDC HuL SPIKE RBD + HuL NPFC |
| SN6 | pVAXDC HuL SPIKE RBD TRIMER + HuL NPFC |
| SN7 | pVAXDC HuL SPIKE RBD FC + HuL NP |
| SN8 | pVAXDC HuL SPIKE RBD + HuL NP |
| SN9 | pVAXDC MOD HuL Spike RBD v2 TRIMER + HuL NPFC |
| SN10 | pVAXDC MOD HuL Spike RBD v3 TRIMER + HuL NPFC |
| SN11 | pVAXDC MOD HuL Spike RBD v2 TRIMER + HuL NPFC iV1 |
| SN12 | pVAXDC HuL SPIKE RBD TRIMER + HuL NPFC iV1 |
| SN13 | pVAXDC HuL SPIKE RBD FC + HuL NPFC iV1 |
| SN14 | pVAXDC HuL SPIKE RBD FC iV1 + HuL NPFC iV1 |
| SN15 | pVAXDC HuL SPIKE RBD + HuL NPFC iV1 |
| SN16 | pVAXDC HuL SPIKE RBD (B1.1.7 variant) + |
| | HuL NPFC iV1 (B1.1.7 variant) |
| SN17 | pVAXDC HuL SPIKE RBD (B1.351 variant) + |
| | HuL NPFC iV1 (B1.351 variant) |

TABLE 6
Summary of immune responses to COVID-19 constructs

| | N protein | | | | RBD | | | | | | | | | |
|---|---|---|---|---|---|---|---|---|---|---|---|---|---|---|
| | | | T | T | | | T | T | T | | | | | |
| | | | cell | cell | | | cells | cells | cells | | | | | p.Virus |
| | | | to N | to 138 | | | to RBD | to S1 | to 417 | | | | | VNAbs |
| | | | (spots/ | (spots/ | | | (spots/ | (spots/ | (spots/ | Avidity | Abs | Abs | ACE | % inh |
| | con- | Secretion | 10\hat{ }6 | 10\hat{ }6 | con- | secretion | 10\hat{ }6 | 10\hat{ }6 | 10\hat{ }6 | to 417 | To RBD | to S1 | VNAbs | at |
| SN | struct | (μg/ml) | cells) | cells) | struct | (μg/ml) | cells) | cells) | cells) | (ng/ml) | (titre) | (titre) | (titre) | 1/100 |
| 1 | Fc | | | | M | | | | | | | | | |
| 2 | Fc | 1 | | | T | 10 | 300 | 300 | 200 | 0.1 | | | | 30 |
| 3 | M | 14 | | | Fc | 200 | 400 | 600 | 200 | 0.0008 | | 100 | | 80 |
| 4 | M | 18 | | | M | 150 | 200 | 300 | | 170 | | | | 15 |
| 5 | Fc | 2 | | | M | 150 | | | | | 10,000 | 1000 | 100 | 90 |
| 6 | Fc | 1 | | | T | 10 | | | | | 1,000 | 100 | 100 | 60 |
| 7 | M | 5 | | | Fc | 600 | | | | | | | | |
| 8 | M | 5 | 300 | 50 | m | 100 | 400 | 100 | 400 | | | 1000 | | |
| 9 | Fc | 0.5 | 310 | 50 | St | 10 | 500 | 100 | 400 | | 100 | 100 | 10 | 40 |
| 10 | Fc | 1 | 450 | 100 | Cc | 20 | | | 300 | | 1000 | 100/1000 | 100 | 45 |
| 11 | Fcv1 | 0.5 | 700 | 450 | st | 10 | 750 | 700 | 1,000 | | 100 | 100 | 10 | 30 |
| 12 | Fcv1 | 0.37 | 210 | 550 | T | 54 | 740 | 200 | 780 | | | 10000 | | 60 |
| 13 | Fcv1 | 0.16 | 170 | 385 | Fc | 408 | 465 | 85 | 500 | | | 10000 | | 50 |
| 14 | Fcv1 | 0.16 | 270 | 720 | Fcv1 | 152 | 780 | 220 | 895 | | | 10000 | | 90 |
| 15 | Fcv1 | 0.32 | 160 | 700 | M | 195 | 880 | 200 | 900 | | | 10000 | | 90 |
| 16 | Fcv1 | | 142 | | M | | 588 | 152 | | | | 30000 | | |
| 17 | Fcv1 | | 563 | | M | | 673 | 367 | | | 10-100000 | 30-100000 | | 25-98 |

M monomer,
Fc Fc-fusion,
Fcv1 Fc-fusion enhanced,
T trimer,
sm small monomer,
st small trimer cc-extra cysteines

All COVID-19 constructs contained a S protein RBD, either as a monomer a trimer or an Fc fusion protein and an N protein, either as a monomer, an Fc fusion protein or an Fc fusion protein modified to allow non covalent association of antigen-Fc fusion protein at the cell surface (Tables 5 and 6).

SN11, which expresses the N protein fused to modified Fc gave significantly better T-cell responses to N protein and to the HLA-A2 epitope N 138-146 than N protein fused to unmodified Fc or to the N protein alone. Of even more interest was that this construct also gave superior responses to RBD, S1 and peptide RBD 417-425 than a similar construct expressing the same RBD construct but N-Fc. This suggests that the modified N-Fc is acting like an adjuvant and activating the APCs to also enhance the T-cell response to other antigens.

In contrast, the best VNAbs were stimulated to the RBD monomer (SN1, SN4, SN5, SN8, SN15), the RBD trimer (SN2, SN6, SN9, SN11, SN12) and the RBD-Fc (SN3, SN7, SN13, SN14). Constructs were produced comparing the RBD trimer to the RBD-Fc and the RBD-enhanced Fc in combination with the Fc modified N protein. The constructs containing either the RBD monomer and N protein fused separately to enhanced Fc regions (SN15) or the RBD and N protein fused separately to enhanced Fc regions (SN14) produced the strongest antibody and T cell responses.

The examples above show a vaccine that incorporates the RBD of the spike protein to stimulate neutralising antibodies and T cell responses but also the N protein to induce memory T cell responses that will confer protection against not only COVID19 but also any new emerging coronaviruses as the N protein is highly conserved and rarely mutates.

### Example 11. pDNA Encoding T Cell Epitopes within CDRs of Fc Modified HuIgG1 Construct (iSCIB1) Generate Strong T Cell Responses

Conventional C57Bl/6 or HLA transgenic mice (HLA-DR4) were immunised with pDNA encoding SCIB1 (WO2008/116937—FIG. 28) compared to iSCIB1 (FIG. 29) via gene gun and immune responses assessed by IFNγ ELISpot assay. FIG. 34A shows high frequency TRP2 180-188 responses generated from both SCIB1 and iSCIB1 DNA in immunised C57Bl/6 and HLA-DR4 mice. Analysis of the avidity of responses by peptide titration reveals iSCIB1 DNA immunisation to generate higher avidity TRP2 180-188 specific CD8 responses than SCIB1 DNA immunisation in C57Bl/6 and HLA-DR4 mice (FIGS. 35B and C). HLA-DR4 mice were also analysed for the frequency of responses to the gp100 44-59 epitope and show a trend to higher frequency responses in mice immunised with iSCIB1 DNA (FIG. 34D).

### Example 12. PDNA Encoding T Cell Epitopes within CDRs of Fc Modified HuIgG1 Construct (iSCIB1Plus) Generate Strong T Cell Responses

HLA transgenic mice (HLA-DR4, C57Bl/6 or HHDII/DP4) were immunised with pDNA encoding SCIB1plus (FIG. 30) compared to iSCIB1plus (FIG. 31) via gene gun and immune responses assessed by IFNγ ELISpot assay. FIG. 35A shows high frequency TRP2 180-188 responses generated from both SCIB1plus and iSCIB1plus DNA in immunised C57Bl/6, HHDII, HHDII/DP4 and HLA-DR4 mice. Analysis of the avidity of responses by peptide titration reveals iSCIB1plus DNA immunisation to generate higher avidity TRP2 180-188 specific CD8 responses than SCIB1plus DNA immunisation in C57Bl/6 and HLA-DR4 mice (FIGS. 35B and C). Analysis of the frequency of gp100 44-59 specific responses in HLA-DR4 mice show a trend to higher frequency responses in mice immunised with iSCIB1plus DNA (FIG. 35D).

### Example 13. pDNA Encoding T Cell Epitopes within CDRs of Fc Modified HuIgG1 Constructs (iSCIB1 and iSCIB1Plus) Mediate Efficient Tumour Therapy

Conventional C57Bl/6 or HLA transgenic mice (HHDII/DP4) were implanted with B16 melanoma cells expressing the appropriate MHC alleles followed by immunisation with pDNA encoding SCIB1 (FIG. 28) compared to iSCIB1 (FIG. 29) or SCIB1plus (FIG. 30) compared to iSCIB1plus (FIG. 31) respectively. Tumour growth and survival was monitored. Tumour therapy study in C57Bl/6 mice is shown in FIG. 36. Mice immunised with iSCIB1 DNA show evidence of slower tumour growth than mice immunised with SCIB1 DNA and significantly slower tumour growth than control mice (p=0.0012) (FIG. 36A). Mice immunised with SCIB1plus DNA or iSCIB1plus DNA show significantly slower tumour growth than controls (p<0.0001) (FIG. 36B). Analysis of tumour volume at day 18 demonstrates that immunisation with iSCIB1plus DNA results in slower tumour growth compared to SCIB1plus DNA (FIG. 36B). iSCIB1, SCIB1plus and iSCIB1plus DNA immunised mice show significantly enhanced overall survival compared to control mice (p=0.0143, p=0.0143 and p<0.0001 respectively) (FIG. 36C). Although immunisation with both SCIB1plus and iSCIB1plus provide tumour therapy, iSCIB1plus DNA immunisation demonstrates a significantly enhanced overall survival compared to SCIB1plus DNA (p=0.0003) (FIG. 36C).

### Example 14. pDNA Encoding T Cell Epitopes within CDRs of Fc Modified HuIgG1 Construct (iSCIB2) Generate Strong T Cell Responses

HLA transgenic mice (HHDII or HHDII/DR1) were immunised with pDNA encoding SCIB2 (FIG. 32) compared to iSCIB2 (FIG. 33) via gene gun and immune responses assessed by IFNγ ELISpot assay. FIG. 37A shows high frequency Nyeso-1 157-165 responses generated from both SCIB2 and iSCIB2 DNA in immunised HHDII and HHDII/DR1 mice. Analysis of the avidity of responses by peptide titration reveals SCIB2 and iSCIB2 DNA immunisation both generate high avidity Nyeso1 157-165 specific CD8 responses in HHDII mice but iSCIB2 generates higher avidity than SCIB2 in HHDII/DR1 mice (FIGS. 37B and C). The Nyeso119-143 sequence contains a known HLA-DR1 epitope therefore responses were analysed to the Nyeso 119-143 epitope in HHDII/DR1 mice. These showed significantly enhanced response frequency from iSCIB2 compared to SCIB2 (p=0.0338) (FIG. 37D). Avidity analysis shows that the Nyeso 119-143 response generated by iSCIB2 is also of higher avidity in HHDII/DR1 mice than that generated by SCIB2 (p=0.0363) (FIG. 37E).

### Example 15. pDNA Encoding T Cell Epitopes within CDRs of Fc Modified HuIgG1 Constructs (iSCIB2) Mediate Efficient Tumour Therapy

HLA transgenic mice (HHDII) were implanted with B16 melanoma cells expressing the appropriate MHCI allele followed by immunisation with pDNA encoding SCIB2 (FIG. 32) compared to iSCIB2 (FIG. 33) via gene gun. Tumour growth and survival was monitored. Mice immunised with both SCIB2 and iSCIB2 show significantly enhanced tumour free survival over control mice (p<0.0001 and p=0.0010 respectively) with no significant difference between SCIB2 and iSCIB2 immunised mice (FIG. 38).

### Example 16. COVID-19 Specific T Cell and Neutralising Antibody Responses from pDNA Delivered Via Gene Gun is Superior from RBD Fc iV1 Construct Compared to RBD Fc Construct

Balb/c and HLA-A2 transgenic mice were immunised with pDNA containing N protein fused to modified Fc (NPFC iV1) alongside either the RBD domain fused to Fc (RBD FC, SN13, FIG. 14) or RBD domain fused to modified Fc (RBD FC iV1, SN14, FIG. 15). T cell responses were assessed by IFNγ ELISpot assay and showed significantly higher frequency responses to a pool of overlapping peptides from the RBD protein in mice immunised with RBD Fc iV1, SN14 compared to RBD Fc, SN13 (FIG. 39A). Analysis of the avidity of the response to an HLA-A2 epitope from RBD (RBD 417-425) in HLA-A2 transgenic mice demonstrated a higher avidity response was generated in SN14 (RBD Fc iV1) immunised mice than in mice immunised with SN13 (RBD Fc) (FIG. 39B).

Antibody responses in immunised Balb/c mice were assessed by ELISA and showed similar titres of antibodies specific for the S1 protein in SN13 and SN14 immunised mice (FIG. 39C). Upon analysis of neutralising antibody responses in a pseudovirus neutralisation assay, sera from mice immunised with SN14 (RBD FC iV1) showed higher neutralisation ID50 titre compared to sera from mice immunised with SN13 (RBD FC) (FIG. 39D). This data provides evidence for both superior T cell and neutralising antibody responses from pDNA constructs containing antigen fused to modified Fc.

### Example 17. RBD Protein and N Protein Secretion from Transient HEK293 Transfections with SN11, 12, 13, 14 and 15 pDNA

In order to assess the transfection efficiency of the SN11, 12, 13, 14 and 15 pDNA constructs compared to SN5 pDNA, HEK293 cells were transiently transfected with the pDNA using THERMO FISHER SCIENTIFIC®'s Expi293 system and protein secretion in the medium and cell lysates evaluated using sandwich ELISAs for the RBD protein and Nucleoprotein FIG. 40. This analysis indicated that for the RBD protein, the constructs containing the RBD-Fc fusion (SN13) gave the highest secretion; closely followed by the unmodified RBD monomer (SN5 and SN15) and the RBD monomer linked to modified Fc (SN14) (FIG. 40A). The unmodified RBD monomer alongside the Nucleoprotein linked to modified Fc (SN15) showed the highest level of RBD protein in the cell lysates (FIG. 40A). Nucleoprotein in the lysates was similar for constructs (SN11, 12, 13, 14 and 15) but levels of secreted protein were slightly higher in the construct containing the RBD trimer (SN12) and the RBD monomer (SN15) (FIG. 40B).

### Example 18. COVID-19 Specific Neutralising Antibody Responses from SN15 pDNA Delivered Via Gene Gun is Superior to that from Whole S pDNA

Balb/c and C57Bl/6 mice were immunised with pDNA containing N protein fused to modified Fc (NPFC iV1) alongside either the RBD monomer (SN15), RBD trimer (SN12) or RBD monomer linked to Fc (SN13) or a with a whole S pDNA. Antibody responses in immunised Balb/c mice were assessed by ELISA and showed higher titres of antibodies and total IgG specific for the S1 protein in SN15 and SN13 compared to whole S DNA or SN11 immunised mice (FIG. 41A). Upon analysis of neutralising antibody responses in a pseudovirus neutralisation assay, sera from mice immunised with SN15 (RBD monomer and NPFCiV1) showed higher neutralisation ID50 titre compared to sera from mice immunised with SN12 (RBD trimer and NPFCiV1) or SN13 (RBD FC and NPFCiV1) (FIG. 41B). Comparison of antibody responses C57Bl/6 mice immunised with either SN15 or whole S pDNA in pseudovirus neutralisation assay revealed higher neutralisation titres in mice immunised with SN15 pDNA compared to whole S pDNA (FIG. 41C). Thus suggesting in two mouse models that superior neutralising antibodies are achieved following SN15 pDNA immunisation.

### Example 19. COVID-19 Specific T Cell Responses from SN15 pDNA Delivered Via Gene Gun are Superior to that from Whole S pDNA

Balb/c, HLA-A2 transgenic and C57Bl/6 mice were immunised with pDNA containing N protein fused to modified Fc (NPFC iv1) alongside either the RBD monomer (SN15), RBD trimer (SN12), RBD short trimer (SN11) or RBD monomer linked to Fc (SN13) or a with a whole S pDNA. T cell responses in immunised mice were assessed by IFNγ ELISpot assay and showed high frequency responses to a pool of overlapping peptides from the RBD protein in mice immunised with SN15, SN13, SN12 and SN11 pDNA (FIG. 42Ai). Responses in SN15, SN11 and SN12 immunised mice were significantly higher (p<0.0001) than those from SN13 immunised mice. A similar pattern of responses was observed upon analysis of a peptide specific response in the HLA-A2 transgenic mice (p=0.0002) (FIG. 42Aii). Comparison of Balb/c and C57Bl/6 mice immunised with SN15 pDNA to those immunised with whole S pDNA showed significantly enhanced responses (p=0.0002) to a pool of overlapping peptides from the RBD protein in mice immunised with SN15 (FIG. 42B). This data provides evidence for superior T cell responses from SN15 pDNA construct to that encoding the whole S protein.

### Example 20. Fc Modified HuIgG1 Constructs Show More Avid CD64 Binding Compared to Unmodified Fc

Targeting antigens to the high affinity FcgammaR1 (CD64) induces better humoral and T cell responses through a combination of enhanced antigen internalisation as well as improved APC activation. A purified RBD construct with a modified Fc shows prolonged interaction with CD64 compared to the unmodified RBD-Fc construct, a phenomenon more pronounced at higher CD64 receptor densities, suggesting increased avidity (FIG. 43, real-time SPR binding curves). This is evident from the kinetic parameters in Table 7 where at higher CD64 densities (300 and 900 RU) a slower dissociation (kd for RBD-iFcv1 is of the order of 10⁴ 1/s compared to 10⁻³ 1/s for the RBD-Fc) and a higher maximal binding (Rmax) are observed for the modified Fc compared to the unmodified Fc construct.

TABLE 7
kinetic constants of the interaction of Fc-containing constructs (Bia T200)

| | Receptor | | | | | |
|---|---|---|---|---|---|---|
| | Density | | | | | |
| construct | (RU) | ka (1/Ms) | kd (1/s) | KD (M) | RMAX | Chi² (RU²) |
| RBD-Fc | ~30 | 1.98E+05 | 7.97E−04 | 4.02E−09 | 32.9 | 2.76 |
| | ~300 | 2.26E+05 | 1.06E−03 | 4.68E−09 | 546.3 | 92.6 |
| | ~900 | 2.31E+05 | 1.05E−03 | 4.57E−09 | 1033.1 | 347 |
| RBD-iFcv1 | ~30 | 2.60E+05 | 6.08E−04 | 2.34E−09 | 47.6 | 1.7 |
| | ~300 | 1.89E+05 | 3.05E−04 | 1.61E−09 | 917.3 | 37.7 |
| | ~900 | 2.00E+05 | 1.93E−04 | 9.63E−10 | 1592.3 | 180 |

### Example 21. Lack of Immunogenicity of the Modified iFcv1 Construct in a 20-Donor Panel

The presence of residue changes in the modified Fc (iFcv1) theoretically has the capacity to induce immunogenicity when administered in the clinic to human volunteers. This was therefore assessed in studies conducted by ABZENA® (UK) using trastuzumab (Herceptin®) as the comparator. To assess whether the iFcv1 fusion construct has the potential to induce CD4 T cell responses in humans, an important driver of immunogenicity, a trastuzumab construct containing the modified iFcv1 was created: ‘iTv1’. The immunogenicity iTv1 was assessed in a proliferation assay (3H-thymidine uptake) using CD8 depleted peripheral blood mononuclear cells (PBMCs) from a panel of 20 donors representing the European and North American population, covering approximately 77% of HLA alleles. The T cell responses were assessed on days 5-8 following incubation with iTv1 or wild-type Trastuzumab (Herceptin®) and Bydureon® controls (FIG. 44A). The modified Fc construct, iTv1, generated a small increase in proliferation in 3/20 donors; this is comparable to the results seen with ABZENA®'s low immunogenicity control Herceptin®, where a small increase in proliferation was observed in 2/20 donors. Stimulation indices of 2 to 2.5 were seen for both, much lower than those observed with either the positive control KLH or Bydureon® which is known to induce anti-drug antibodies in 45% of patients. Comparison of the maximum proliferative responses of CD8-depleted PBMCs demonstrates that there is no significant difference between iTv1 and Herceptin® (FIG. 44B) indicating that the Fc modification in iTv1- and similarly in SN15—is unlikely to stimulate potent CD4 T cell responses in humans.

### Example 22. Lack of Immunogenicity of the Modified iFcv1 Construct in a 50-Donor Panel

In addition to the study performed in a 20-donor panel a repeat was performed to extend to a 50 donor panel spanning a broader range of HLA types. The modified Fc construct, iTv1, generated a small increase in proliferation in 3/50 donors; this is again comparable to the results seen with ABZENA®'s low immunogenicity control Herceptin®, where a small increase in proliferation was observed in 2/20 donors and a larger increase in 1/50 donors (FIG. 45A). Stimulation indices of 2 to 2.4 were seen for iTv1, much lower than those observed with either the positive control KLH or Bydureon® which is known to induce anti-drug antibodies in 45% of patients. Comparison of the maximum proliferative responses of CD8-depleted PBMCs demonstrates that there is no significant difference between iTv1 and Herceptin® (FIG. 45B) indicating that the Fc modification in iTv1—and similarly in SN15, SN17, iSCIB1, iSCIB1plus and iSCIB2—is unlikely to stimulate potent CD4 T cell responses in humans.

### Example 23. COVID-19 N Protein Specific Antibody Responses are Elicited from pDNA Encoding NP, NP Fc and NP FciV1 Delivered Via Gene Gun

Sera from mice immunised with NP, NP Fc or NP FciV1 encoding pDNA were analysed for antibody reactivity to SARS-CoV-2 N protein by ELISA. Similar antibody responses and EC₅₀ values were seen from all constructs irrespective of whether it was fused to Fc or not (FIG. 45.)

### Example 24. Antibody Responses Cross Reactive with Variant Strains are Induced by SN15 pDNA Vaccination

Balb/c mice were immunised with pDNA containing N protein fused to modified Fc (NPFC iV1) alongside the RBD monomer (SN15) and antibody responses in sera were assessed by ELISA to variant S1 proteins from Wuhan (Lineage A), B.1.351 and B.1.1.7 virus strains. Higher titres of antibodies were observed specific for the S1 protein from lineage A, B.1.1.7 and B.1.351 variants with detectable responses over background at down to 1 in 100,000 sera dilution. No significant difference in the reactivity to Lineage A and B.1.1.7 variant S1 proteins was seen (FIG. 49). A drop in EC₅₀ to B.1.351 variant is observed but this data demonstrates the induction of cross-reactive antibody responses to both the B.1.1.7 and B.1.351 variants.

### Example 25. Antibody Responses Cross Reactive with Variant Strains are Induced by SN15, SN16 and SN17 pDNA Vaccination that Also Inhibit ACE2 Binding

Balb/c mice were immunised with pDNA containing the RBD monomer (SN15), RBD monomer from B.1.1.7 variant (SN16, FIG. 47) or RBD monomer from B.1.351 variant (SN17, FIG. 48) alongside the relevant variant N proteins fused to modified Fc (NPFC iV1). Antibody responses in sera were assessed by ELISA to variant S1 proteins from Wuhan (Lineage A), B.1.351 and B.1.1.7 virus strains. High titres of antibodies were observed from both SN17 and SN16 specific for the S1 protein from lineage A, B.1.1.7 and B.1.351 variants with detectable responses over background at down to 1 in 100,000 sera dilution. No significant difference in the reactivity to Lineage A, B.1.351 and B.1.1.7 variant S1 proteins was seen from SN17 (FIG. 50Ai). Similar responses are observed from SN16 (FIG. 50Aii) to those from SN15 (FIG. 49). A drop in EC₅₀ to B.1.351 variant is observed (FIG. 50Aiii) but despite this data demonstrates the induction of cross-reactive antibody responses to both the B.1.1.7 and B.1.351 variants. Comparison of sera from mice immunised with Lineage A and B.1.351 variant vaccine constructs with that from mice immunised with whole S DNA shows higher titre responses to both the Lineage A S1 protein and the B.1.351 S1 protein (FIG. 50B).

The ability of sera from immunised mice to inhibit the binding of the ACE2 receptor to the variant RBD or whole S proteins was assessed using the MesoScale Discovery platform. Inhibition of RBD binding to ACE2 was higher for sera from mice vaccinated with the original Lineage A vaccine construct (SN15) and the B.1.351 variant vaccine construct (SN17) compared to that seen with the NIBSC 20/136 control (FIG. 50Ci). In contrast, sera from mice immunised with whole S DNA showed a lower capacity to inhibit ACE2 binding which was similar to that of the NIBSC 20/136 control. Sera from mice immunised with the original Lineage A vaccine construct (SN15) inhibited 80-100% of ACE2 binding to the original Lineage A RBD and B.1.1.7 variants with a drop to 50-60% against the B.1.351 and P.1 RBD variants. The reverse is seen in sera from mice immunised with the B.1.351 variant vaccine (SN17). Despite a reduction in the inhibition of ACE2 binding to the B.1.351 and P.1 RBD variants, the inhibition levels remain above those seen using sera from whole S DNA immunised mice. A similar trend is seen in the ACE2 receptor binding inhibition assay with the variant whole S proteins (FIG. 50Cii).

### Example 26. Sera from SN15 and SN17 Immunised Mice Show Virus Neutralisation in Pseudotype and Live Virus Neutralisation Assays

Sera from Balb/c mice immunised with the original Lineage A (SN15) or B.1.351 (SN17) variant vaccines were also assessed in pseudotype and live virus neutralisation tests against the original Lineage A and B.1.351 variants. Sera from mice immunised with the original variant vaccine showed potent neutralisation of the original Lineage A pseudotype, with reduced efficacy against the B.1.351 variant vaccine (ID₅₀ values of 6232 and 2137 respectively) (FIG. 51A). Sera from mice immunised with either vaccine showed neutralisation of the B.1.351 pseudotype variant, but little difference was noted (ID₅₀ values of 948 and 997, respectively). In the live virus neutralisation assay sera from mice immunised with either the original Lineage A (SN15) or the B.1.351 (SN17) variant vaccines neutralised of the original Lineage A virus with ID₅₀ values of 4964 and 1334 respectively and both are better than NIBSC controls (FIG. 51B).

### Example 27. T Cell Responses are not Impacted by Variations in the Virus Strains

To examine if the T cell responses were influenced by the mutations in the different virus variants, splenocytes from mice immunised with either the original Lineage A vaccine (SN15) or the B.1.351 vaccine (SN17) were stimulated ex vivo with RBD and N peptide pools derived from the original sequence. T cell responses specific for RBD and N were detected with little difference between the response induced by the different vaccine constructs (FIG. 52). These results suggest that mutations in the B.1.351 variant have less impact on the T cell responses.

### Example 28. Antibody Responses can be Efficiently Boosted by Vaccines Specific for Variant Strains and Show Cross Reactivity to B.1.351 and B.1.617.2 Variants

To examine if antibody responses elicited against the Lineage A virus with the SN15 vaccine could be boosted by a vaccine targeting the B.1.351 variant (SN17) Balb/c mice were immunised with the SN15 vaccine on days 1 and 29 followed by a boost at day 85 with SN17 vaccine. Antibody responses in sera samples taken at days 42, 82 and 98 were examined by ELISA for reactivity to the Lineage A and B.1.351 S1 proteins. As a comparison, mice were immunised with only SN17 vaccine. Antibody responses are detectable in both sets of sera down to 1 in 100,000 dilution (FIG. 53A). A drop in response titre is seen at day 82 compared to day 42 in both sets of mice although EC₅₀ values remain at >1 in 3500 against the Lineage A S1 protein. A booster at day 85 with the SN17 vaccine efficiently boosts responses to the Lineage A S1 protein by day 98 in both sets of mice suggesting that the SN17 vaccine is efficient at boosting responses primed by both SN15 and SN17 vaccines. Reactivity in both sets of mice is reduced to the B.1.351 S1 protein with lower EC₅₀ values compared to the Lineage A S1 protein and this is more apparent in mice immunised with the SN15+SN17 boost. Despite this the booster with the SN17 vaccine is able to elevate responses in the SN15 vaccine primed group to a similar level seen in mice receiving the SN17 prime.

Sera from mice immunised with these prime boost regimes were analysed for reactivity to the RBD proteins from the B.1.351 and B.1.617.2 variants by ELISA (FIG. 53B). Sera from both sets of mice show higher antibody titres and EC₅₀ values to the B.1.351 RBD protein compared to the B.1.351 S1 protein. Again, responses to the B.1.351 RBD protein in mice primed with either SN15 or SN17 are efficiently boosted by the SN17 booster shown at day 98 compared to day 82. A higher titre after the booster vaccine is noted in mice primed with the SN17 vaccine. Analysis of cross-reactivity to the RBD protein from the B.1.617.2 variant shows high titre responses at day 82 with EC₅₀ values of 8355 in mice primed with SN15 and 5738 in mice primed with SN17. This is higher reactivity than seen in the same sera to the B.1.351 RBD protein suggesting good cross reactivity to this variant from both vaccines. The booster with SN17 vaccine results in an increase in titre and EC₅₀ values to similar levels in both sets of mice suggesting the SN17 booster can boost the B.1.617.2 variant cross reactive RBD specific responses induced by both a SN15 and SN17 vaccine prime.

### REFERENCES

1. Ichim, C. V., Revisiting immunosurveillance and immunostimulation: Implications for cancer immunotherapy. J Transl Med, 2005. 3(1): p. 8.
2. Burnet, F. M., The concept of immunological surveillance. Prog Exp Tumor Res, 1970. 13: p. 1-27.
3. Swann, J. B. and M. J. Smyth, Immune surveillance of tumors. J Clin Invest, 2007. 117(5): p. 1137-46.
4. Shankaran, V., et al., IFNgamma and lymphocytes prevent primary tumour development and shape tumour immunogenicity. Nature, 2001. 410(6832): p. 1107-11.
5. Ferradini, L., et al., Analysis of T cell receptor variability in tumor-infiltrating lymphocytes from a human regressive melanoma. Evidence for in situ T cell clonal expansion. J Clin Invest, 1993. 91(3): p. 1183-90.
6. Zorn, E. and T. Hercend, A natural cytotoxic T cell response in a spontaneously regressing human melanoma targets a neoantigen resulting from a somatic point mutation. Eur J Immunol, 1999. 29(2): p. 592-601.
7. Schreiber, R. D., L. J. Old, and M. J. Smyth, Cancer immunoediting: integrating immunity's roles in cancer suppression and promotion. Science, 2011. 331(6024): p. 1565-70.
8. Driessens, G., J. Kline, and T. F. Gajewski, Costimulatory and coinhibitory receptors in anti-tumor immunity. Immunol Rev, 2009. 229(1): p. 126-44.
9. Capece, D., et al., Targeting costimulatory molecules to improve antitumor immunity. J Biomed Biotechnol, 2012. 2012: p. 926321.
10. Abdelmoula, M., et al., IgG3 is the major source of cryoglobulins in mice. J Immunol, 1989. 143(2): p. 526-32.
11. Cooper, L. J., et al., H chain C domains influence the strength of binding of IgG for streptococcal group A carbohydrate. J Immunol, 1991. 146(8): p. 2659-63.
12. Greenspan, N. S., D. A. Dacek, and L. J. Cooper, Cooperative binding of two antibodies to independent antigens by an Fc-dependent mechanism. FASEB J, 1989. 3(10): p. 2203-7.
13. Grey, H. M., J. W. Hirst, and M. Cohn, A new mouse immunoglobulin: IgG3. J Exp Med, 1971. 133(2): p. 289-304.
14. Greenspan, N. S., W. J. Monafo, and J. M. Davie, Interaction of IgG3 anti-streptococcal group A carbohydrate (GAC) antibody with streptococcal group A vaccine: enhancing and inhibiting effects of anti-GAC, anti-isotypic, and anti-idiotypic antibodies. J Immunol, 1987. 138(1): p. 285-92.
15. Greenspan, N. S. and L. J. Cooper, Cooperative binding by mouse IgG3 antibodies: implications for functional affinity, effector function, and isotype restriction. Springer Semin Immunopathol, 1993. 15(2-3): p. 275-91.
16. Greenspan, N. S. and L. J. Cooper, Intermolecular cooperativity: a clue to why mice have IgG3?Immunol Today, 1992. 13(5): p. 164-8.
17. Lefranc, M. P., et al., IMGT, the international ImMunoGeneTics information system. Nucleic Acids Res, 2009. 37 (Database issue): p. D1006-12.
18. Cui, J., F. Li, and Z. L. Shi, Origin and evolution of pathogenic coronaviruses. Nat Rev Microbiol, 2019. 17(3): p. 181-192.
19. Lefkowitz, E. J., et al., Virus taxonomy: the database of the International Committee on Taxonomy of Viruses (ICTV). Nucleic Acids Res, 2018. 46(D1): p. D708-D717.
20. Fehr, A. R. and S. Perlman, Coronaviruses: an overview of their replication and pathogenesis. Methods Mol Biol, 2015. 1282: p. 1-23.
21. Forni, D., et al., Molecular Evolution of Human Coronavirus Genomes. Trends Microbiol, 2017. 25(1): p. 35-48.
22. Su, S., et al., Epidemiology, Genetic Recombination, and Pathogenesis of Coronaviruses. Trends Microbiol, 2016. 24(6): p. 490-502.
23. Coronaviridae Study Group of the International Committee on Taxonomy of, V., The species Severe acute respiratory syndrome-related coronavirus: classifying 2019-nCoV and naming it SARS-CoV-2. Nat Microbiol, 2020. 5(4): p. 536-544.
24. Zhou, P., et al., A pneumonia outbreak associated with a new coronavirus of probable bat origin. Nature, 2020. 579(7798): p. 270-273.
25. Snijder, E. J., et al., Ultrastructure and origin of membrane vesicles associated with the severe acute respiratory syndrome coronavirus replication complex. J Virol, 2006. 80(12): p. 5927-40.
26. Hussain, S., et al., Identification of novel subgenomic RNAs and noncanonical transcription initiation signals of severe acute respiratory syndrome coronavirus. J Virol, 2005. 79(9): p. 5288-95.
27. Perlman, S. and J. Netland, Coronaviruses post-SARS: update on replication and pathogenesis. Nat Rev Microbiol, 2009. 7(6): p. 439-50.
28. Sawicki, S. G., D. L. Sawicki, and S. G. Siddell, A contemporary view of coronavirus transcription. J Virol, 2007. 81(1): p. 20-9.
29. Rothe, C., et al., Transmission of 2019-nCoV Infection from an Asymptomatic Contact in Germany. N Engl J Med, 2020. 382(10): p. 970-971.
30. Huang, C., et al., Clinical features of patients infected with 2019 novel coronavirus in Wuhan, China. Lancet, 2020. 395(10223): p. 497-506.
31. Guan, W. J., et al., Clinical Characteristics of Coronavirus Disease 2019 in China. N Engl J Med, 2020.
32. Lauer, S. A., et al., The Incubation Period of Coronavirus Disease 2019 (COVID-19) From Publicly Reported Confirmed Cases: Estimation and Application. Ann Intern Med, 2020.
33. Li, Q., et al., Early Transmission Dynamics in Wuhan, China, of Novel Coronavirus-Infected Pneumonia. N Engl J Med, 2020. 382(13): p. 1199-1207.
34. Pung, R., et al., Investigation of three clusters of COVID-19 in Singapore: implications for surveillance and response measures. Lancet, 2020. 395(10229): p. 1039-1046.
35. Pan, Y., et al., Viral load of SARS-CoV-2 in clinical samples. Lancet Infect Dis, 2020. 20(4): p. 411-412.
36. Kim, J. Y., et al., Viral Load Kinetics of SARS-CoV-2 Infection in First Two Patients in Korea. J Korean Med Sci, 2020. 35(7): p. e86.
37. Zou, L., et al., SARS-CoV-2 Viral Load in Upper Respiratory Specimens of Infected Patients. N Engl J Med, 2020. 382(12): p. 1177-1179.
38. Peiris, J. S., et al., Clinical progression and viral load in a community outbreak of coronavirus-associated SARS pneumonia: a prospective study. Lancet, 2003. 361(9371): p. 1767-72.
39. Wang, D., et al., Clinical Characteristics of 138 Hospitalized Patients With 2019 Novel Coronavirus-Infected Pneumonia in Wuhan, China. JAMA, 2020.
40. Wong, C. K., et al., Plasma inflammatory cytokines and chemokines in severe acute respiratory syndrome. Clin Exp Immunol, 2004. 136(1): p. 95-103.
41. Chen, N., et al., Epidemiological and clinical characteristics of 99 cases of 2019 novel coronavirus pneumonia in Wuhan, China: a descriptive study. Lancet, 2020. 395(10223): p. 507-513.
42. Li, F., Structure, Function, and Evolution of Coronavirus Spike Proteins. Annu Rev Virol, 2016. 3(1): p. 237-261.
43. Bosch, B. J., et al., The coronavirus spike protein is a class I virus fusion protein: structural and functional characterization of the fusion core complex. J Virol, 2003. 77(16): p. 8801-11.
44. Hoffmann, M., et al., SARS-CoV-2 Cell Entry Depends on ACE2 and TMPRSS2 and Is Blocked by a Clinically Proven Protease Inhibitor. Cell, 2020.
45. Walls, A. C., et al., Structure, Function, and Antigenicity of the SARS-CoV-2 Spike Glycoprotein. Cell, 2020. 181(2): p. 281-292.e6.
46. Simmons, G., et al., Inhibitors of cathepsin L prevent severe acute respiratory syndrome coronavirus entry. Proc Natl Acad Sci USA, 2005. 102(33): p. 11876-81.
47. Bosch, B. J., et al., Severe acute respiratory syndrome coronavirus (SARS-CoV) infection inhibition using spike protein heptad repeat-derived peptides. Proc Natl Acad Sci USA, 2004. 101(22): p. 8455-60.
48. Liu, S., et al., Interaction between heptad repeat 1 and 2 regions in spike protein of SARS-associated coronavirus: implications for virus fusogenic mechanism and identification of fusion inhibitors. Lancet, 2004. 363(9413): p. 938-47.
49. Beniac, D. R., et al., Architecture of the SARS coronavirus prefusion spike. Nat Struct Mol Biol, 2006. 13(8): p. 751-2.
50. Delmas, B. and H. Laude, Assembly of coronavirus spike protein into trimers and its role in epitope expression. J Virol, 1990. 64(11): p. 5367-75.
51. Nal, B., et al., Differential maturation and subcellular localization of severe acute respiratory syndrome coronavirus surface proteins S, M and E. J Gen Virol, 2005. 86(Pt 5): p. 1423-1434.
52. Neuman, B. W., et al., A structural analysis of M protein in coronavirus assembly and morphology. J Struct Biol, 2011. 174(1): p. 11-22.
53. DeDiego, M. L., et al., A severe acute respiratory syndrome coronavirus that lacks the E gene is attenuated in vitro and in vivo. J Virol, 2007. 81(4): p. 1701-13.
54. Nieto-Torres, J. L., et al., Severe acute respiratory syndrome coronavirus envelope protein ion channel activity promotes virus fitness and pathogenesis. PLoS Pathog, 2014. 10(5): p. e1004077.
55. Chang, C. K., et al., Modular organization of SARS coronavirus nucleocapsid protein. J Biomed Sci, 2006. 13(1): p. 59-72.
56. Hurst, K. R., C. A. Koetzner, and P. S. Masters, Identification of in vivo-interacting domains of the murine coronavirus nucleocapsid protein. J Virol, 2009. 83(14): p. 7221-34.
57. Chen, Y., et al., Structure analysis of the receptor binding of 2019-nCoV. Biochem Biophys Res Commun, 2020.
58. Wrapp, D., et al., Cryo-EM structure of the 2019-nCoV spike in the prefusion conformation. Science, 2020. 367(6483): p. 1260-1263.
59. Coutard, B., et al., The spike glycoprotein of the new coronavirus 2019-nCoV contains a furin-like cleavage site absent in CoV of the same clade. Antiviral Res, 2020. 176: p. 104742.
60. Xu, Z., et al., Pathological findings of COVID-19 associated with acute respiratory distress syndrome. Lancet Respir Med, 2020. 8(4): p. 420-422.
61. Li, T., et al., Significant changes of peripheral T lymphocyte subsets in patients with severe acute respiratory syndrome. J Infect Dis, 2004. 189(4): p. 648-51.
62. Mahallawi, W. H., et al., MERS-CoV infection in humans is associated with a pro-inflammatory Th1 and Th17 cytokine profile. Cytokine, 2018. 104: p. 8-13.
63. Channappanavar, R. and S. Perlman, Pathogenic human coronavirus infections: causes and consequences of cytokine storm and immunopathology. Semin Immunopathol, 2017. 39(5): p. 529-539.
64. Kindler, E., V. Thiel, and F. Weber, Interaction of SARS and MERS Coronaviruses with the Antiviral Interferon Response. Adv Virus Res, 2016. 96: p. 219-243.
65. Wu, F., et al., A new coronavirus associated with human respiratory disease in China. Nature, 2020. 579(7798): p. 265-269.
66. Wang, F., et al., Characteristics of peripheral lymphocyte subset alteration in COVID-19 pneumonia. J Infect Dis, 2020.
67. Shokri, S., et al., Modulation of the immune response by Middle East respiratory syndrome coronavirus. J Cell Physiol, 2019. 234(3): p. 2143-2151.
68. Ahmed, R., et al., Selection of genetic variants of lymphocytic choriomeningitis virus in spleens of persistently infected mice. Role in suppression of cytotoxic T lymphocyte response and viral persistence. J Exp Med, 1984. 160(2): p. 521-40.
69. Chen, W. H., et al., The SARS-CoV-2 Vaccine Pipeline: an Overview. Curr Trop Med Rep, 2020: p. 1-4.
70. Shin, H. and E. J. Wherry, CD8 T cell dysfunction during chronic viral infection. Curr Opin Immunol, 2007. 19(4): p. 408-15.
71. Zheng, H. Y., et al., Elevated exhaustion levels and reduced functional diversity of T cells in peripheral blood may predict severe progression in COVID-19 patients. Cell Mol Immunol, 2020. 17(5): p. 541-543.
72. Ouyang, Y., et al., Down-regulated gene expression spectrum and immune responses changed during the disease progression in COVID-19 patients. Clin Infect Dis, 2020.
73. Li, C. K., et al., T cell responses to whole SARS coronavirus in humans. J Immunol, 2008. 181(8): p. 5490-500.
74. Tseng, C. T., et al., Immunization with SARS coronavirus vaccines leads to pulmonary immunopathology on challenge with the SARS virus. PLoS One, 2012. 7(4): p. e35421.
75. Wang, F., et al., The laboratory tests and host immunity of COVID-19 patients with different severity of illness. JCI Insight, 2020.
76. Channappanavar, R., et al., Virus-specific memory CD8 T cells provide substantial protection from lethal severe acute respiratory syndrome coronavirus infection. J Virol, 2014. 88(19): p. 11034-44.
77. Zhao, J., et al., Airway Memory CD4(+) T Cells Mediate Protective Immunity against Emerging Respiratory Coronaviruses. Immunity, 2016. 44(6): p. 1379-91.
78. Chen, J., et al., Cellular immune responses to severe acute respiratory syndrome coronavirus (SARS-CoV) infection in senescent BALB/c mice: CD4+ T cells are important in control of SARS-CoV infection. J Virol, 2010. 84(3): p. 1289-301.
79. Zhao, J., J. Zhao, and S. Perlman, T cell responses are required for protection from clinical disease and for virus clearance in severe acute respiratory syndrome coronavirus-infected mice. J Virol, 2010. 84(18): p. 9318-25.
80. Peng, H., et al., Long-lived memory T lymphocyte responses against SARS coronavirus nucleocapsid protein in SARS-recovered patients. Virology, 2006. 351(2): p. 466-75.
81. Yang, L. T., et al., Long-lived effector/central memory T-cell responses to severe acute respiratory syndrome coronavirus (SARS-CoV) S antigen in recovered SARS patients. Clin Immunol, 2006. 120(2): p. 171-8.
82. Ng, O. W., et al., Memory T cell responses targeting the SARS coronavirus persist up to 11 years post-infection. Vaccine, 2016. 34(17): p. 2008-14.
83. Ahmed, S. F., A. A. Quadeer, and M. R. McKay, Preliminary Identification of Potential Vaccine Targets for the COVID-19 Coronavirus (SARS-CoV-2) Based on SARS-CoV Immunological Studies. Viruses, 2020. 12(3).
84. Grifoni, A., et al., A Sequence Homology and Bioinformatic Approach Can Predict Candidate Targets for Immune Responses to SARS-CoV-2. Cell Host Microbe, 2020. 27(4): p. 671-680 e2.
85. Bhattacharya, M., et al., Development of epitope-based peptide vaccine against novel coronavirus 2019 (SARS-COV-2): Immunoinformatics approach. J Med Virol, 2020.
86. Nguyen, A., et al., Human leukocyte antigen susceptibility map for SARS-CoV-2. J Virol, 2020.
87. Grifoni, A., et al., Targets of T cell responses to SARS-CoV-2 coronavirus in humans with COVID-19 disease and unexposed individuals. Cell, 2020.
88. Tang, F., et al., Lack of peripheral memory B cell responses in recovered patients with severe acute respiratory syndrome: a six-year follow-up study. J Immunol, 2011. 186(12): p. 7264-8.
89. Xu, X. and X. Gao, Immunological responses against SARS-coronavirus infection in humans. Cell Mol Immunol, 2004. 1(2): p. 119-22.
90. Thevarajan, I., et al., Breadth of concomitant immune responses prior to patient recovery: a case report of non-severe COVID-19. Nat Med, 2020. 26(4): p. 453-455.
91. Koutsakos, M., et al., Circulating TFH cells, serological memory, and tissue compartmentalization shape human influenza-specific B cell immunity. Sci Transl Med, 2018. 10(428).
92. Long, Q. X., et al., Antibody responses to SARS-CoV-2 in patients with COVID-19. Nat Med, 2020.
93. Ferretti, A. P., et al., Unbiased Screens Show CD8(+) T Cells of COVID-19 Patients Recognize Shared Epitopes in SARS-CoV-2 that Largely Reside outside the Spike Protein. Immunity, 2020. 53(5): p. 1095-1107.e3.
94. Qu, J., et al., Profile of IgG and IgM antibodies against severe acute respiratory syndrome coronavirus 2 (SARS-CoV-2). Clin Infect Dis, 2020.
95. Liu, L., et al., Anti-spike IgG causes severe acute lung injury by skewing macrophage responses during acute SARS-CoV infection. JCI Insight, 2019. 4(4).
96. Zhang, L., et al., Antibody responses against SARS coronavirus are correlated with disease outcome of infected individuals. J Med Virol, 2006. 78(1): p. 1-8.
97. Caddy, S. L., et al., Viral nucleoprotein antibodies activate TRIM21 and induce T cell immunity. EMBO J, 2020: p. e106228.
98. Cohen, J., Vaccine designers take first shots at COVID-19. Science, 2020. 368(6486): p. 14-16.
99. Saphire, E. O., et al., Antibody-mediated protection against Ebola virus. Nat Immunol, 2018. 19(11): p. 1169-1178.
100. Gunn, B. M., et al., Survivors of Ebola Virus Disease Develop Polyfunctional Antibody Responses. J Infect Dis, 2020. 221(1): p. 156-161.
101. Sheridan, C., Convalescent serum lines up as first-choice treatment for coronavirus. Nat Biotechnol, 2020.
102. Yasui, F., et al., Prior immunization with severe acute respiratory syndrome (SARS)-associated coronavirus (SARS-CoV) nucleocapsid protein causes severe pneumonia in mice infected with SARS-CoV. J Immunol, 2008. 181(9): p. 6337-48.
103. Xu, J., et al., Antibodies and vaccines against Middle East respiratory syndrome coronavirus. Emerg Microbes Infect, 2019. 8(1): p. 841-856.
104. He, Y., et al., Receptor-binding domain of severe acute respiratory syndrome coronavirus spike protein contains multiple conformation-dependent epitopes that induce highly potent neutralizing antibodies. J Immunol, 2005. 174(8): p. 4908-15.
105. Yuan, M., et al., A highly conserved cryptic epitope in the receptor binding domains of SARS-CoV-2 and SARS-CoV. Science, 2020. 368(6491): p. 630-633.
106. Shim, B. S., et al., Zika Virus-Immune Plasmas from Symptomatic and Asymptomatic Individuals Enhance Zika Pathogenesis in Adult and Pregnant Mice. mBio, 2019. 10(4).
107. Iwasaki, A. and Y. Yang, The potential danger of suboptimal antibody responses in COVID-19. Nat Rev Immunol, 2020.
108. Cavanagh, D., Severe acute respiratory syndrome vaccine development: experiences of vaccination against avian infectious bronchitis coronavirus. Avian Pathol, 2003. 32(6): p. 567-82.
109. Gao, W., et al., Effects of a SARS-associated coronavirus vaccine in monkeys. Lancet, 2003. 362(9399): p. 1895-6.
110. Kim, E., et al., Immunogenicity of an adenoviral-based Middle East Respiratory Syndrome coronavirus vaccine in BALB/c mice. Vaccine, 2014. 32(45): p. 5975-82.
111. Jiang, S., L. Lu, and L. Du, Development of SARS vaccines and therapeutics is still needed. Future Virol, 2013. 8(1): p. 1-2.
112. Oh, S., et al., Selective induction of high avidity CTL by altering the balance of signals from APC. J Immunol, 2003. 170(5): p. 2523-30.
113. Xu, S., et al., Rapid high efficiency sensitization of CD8+ T cells to tumor antigens by dendritic cells leads to enhanced functional avidity and direct tumor recognition through an IL-12-dependent mechanism. J Immunol, 2003. 171(5): p. 2251-61.
114. Renard, V., et al., CD8 beta increases CD8 coreceptor function and participation in TCR-ligand binding. J Exp Med, 1996. 184(6): p. 2439-44.
115. Kroger, C. J. and M. A. Alexander-Miller, Dose-dependent modulation of CD8 and functional avidity as a result of peptide encounter. Immunology, 2007. 122(2): p. 167-78.
116. Cawthon, A. G., H. Lu, and M. A. Alexander-Miller, Peptide requirement for CTL activation reflects the sensitivity to CD3 engagement: correlation with CD8α/β versus CD8αα expression. J Immunol, 2001. 167: p. 2577-2584.
117. Yee, C., et al., Isolation of high avidity melanoma-reactive CTL from heterogenous populations using peptide-MHC tetramers. J Immunol, 1999. 162: p. 2227-2234.
118. Hodge, J. W., et al., Multiple costimulatory modalities enhance CTL avidity. J Immunol, 2005. 174(10): p. 5994-6004.
119. Dhodapkar, K. M., et al., Selective blockade of inhibitory Fcgamma receptor enables human dendritic cell maturation with IL-12p70 production and immunity to antibody-coated tumor cells. Proc Natl Acad Sci USA, 2005. 102(8): p. 2910-5.
120. Bolles, M., et al., A double-inactivated severe acute respiratory syndrome coronavirus vaccine provides incomplete protection in mice and induces increased eosinophilic proinflammatory pulmonary response upon challenge. J Virol, 2011. 85(23): p. 12201-15.
121. Jarmalavicius, S., Y. Welte, and P. Walden, High immunogenicity of the human leukocyte antigen peptidomes of melanoma tumor cells. J Biol Chem, 2012. 287(40): p. 33401-11.
122. Lennerz, V., et al., The response of autologous T cells to a human melanoma is dominated by mutated neoantigens. Proc Natl Acad Sci USA, 2005. 102(44): p. 16013-8.
123. Rizvi, N. A., et al., Cancer immunology. Mutational landscape determines sensitivity to PD-1 blockade in non-small cell lung cancer. Science, 2015. 348(6230): p. 124-8.
124. tuksza M, R. N., Makarov V, Balachandran V P, Hellman H, Solovyov A., Rizvi N A, Merghoub T, Levine A J, and W. J. Chan T A, Greenbaum B D, A neoantigen fitness model predicts tumor response to checkpoint blockade immunotherapy. Nature, In press.
125. Bassani-Sternberg, M., et al., Direct identification of clinically relevant neoepitopes presented on native human melanoma tissue by mass spectrometry. Nat Commun, 2016. 7: p. 13404.
126. Carreno, B. M., et al., Cancer immunotherapy. A dendritic cell vaccine increases the breadth and diversity of melanoma neoantigen-specific T cells. Science, 2015. 348(6236): p. 803-8.
127. Sahin, U., et al., Personalized RNA mutanome vaccines mobilize poly-specific therapeutic immunity against cancer. Nature, 2017. 547(7662): p. 222-226.
128. Ott, P. A., et al., An immunogenic personal neoantigen vaccine for patients with melanoma. Nature, 2017. 547(7662): p. 217-221.
129. Kreiter, S., et al., Mutant MHC class II epitopes drive therapeutic immune responses to cancer. Nature, 2015. 520(7549): p. 692-6.
130. Verdegaal, E. M., et al., Neoantigen landscape dynamics during human melanoma-T cell interactions. Nature, 2016. 536(7614): p. 91-5.
131. Boon, T. and P. van der Bruggen, Human tumor antigens recognized by T lymphocytes. J Exp Med, 1996. 183(3): p. 725-9.
132. Khong, H. T. and S. A. Rosenberg, Pre-existing immunity to tyrosinase-related protein (TRP)-2, a new TRP-2 isoform, and the NY-ESO-1 melanoma antigen in a patient with a dramatic response to immunotherapy. J Immunol, 2002. 168(2): p. 951-6.
133. Gnjatic, S., et al., NY-ESO-1 DNA vaccine induces T-cell responses that are suppressed by regulatory T cells. Clin Cancer Res, 2009. 15(6): p. 2130-9.
134. Jiang, J., et al., Fusion of Hsp70 to Mage-α1 enhances the potency of vaccine-specific immune responses. J Transl Med, 2013. 11: p. 300.
135. Dowling, J. K. and A. Mansell, Toll-like receptors: the swiss army knife of immunity and vaccine development. Clin Transl Immunology, 2016. 5(5): p. e85.
136. Zom, G. G., et al., Two in one: improving synthetic long peptide vaccines by combining antigen and adjuvant in one molecule. Oncoimmunology, 2014. 3(7): p. e947892.
137. Zom, G. G., et al., TLR2 ligand-synthetic long peptide conjugates effectively stimulate tumor-draining lymph node T cells of cervical cancer patients. Oncotarget, 2016. 7(41): p. 67087-67100.
138. Cho, H. I., et al., BiVax: a peptide/poly-IC subunit vaccine that mimics an acute infection elicits vast and effective anti-tumor CD8 T-cell responses. Cancer Immunol Immunother, 2013. 62(4): p. 787-99.
139. Kumai, T., et al., Optimization of Peptide Vaccines to Induce Robust Antitumor CD4 T-cell Responses. Cancer Immunol Res, 2017. 5(1): p. 72-83.
140. Pudney, V. A., et al., DNA vaccination with T-cell epitopes encoded within Ab molecules induces high-avidity anti-tumor CD8+ T cells. Eur J Immunol, 2010. 40(3): p. 899-910.
141. Alexander-Miller, M. A., G. R. Leggatt, and J. A. Berzofsky, Selective expansion of high- or low-avidity cytotoxic T lymphocytes and efficacy for adoptive immunotherapy. Proc Natl Acad Sci USA, 1996. 93(9): p. 4102-7.
142. Dutoit, V., et al., Heterogeneous T-cell response to MAG-A10₂₅₄₋₂₆₂: High avidity-specific cytolytic T lymphocytes show superior antitumour activity. Cancer Res, 2001. 61: p. 5850-5853.
143. Zeh, H. J., et al., High avidity CTLs for two self-antigens demonstrate superior in vitro and in vivo antitumor efficacy. J Immuno, 1999. 162((2)): p. 989-994.
144. Ayyoub, M., et al., Tumor-reactive, SSX-2-specific CD8+ T cells are selectively expanded during immune responses to antigen-expressing tumors in melanoma patients. Cancer Res, 2003. 63(17): p. 5601-6.
145. Durrant, L. G., et al., Vaccines as early therapeutic interventions for cancer therapy: neutralising the immunosuppressive tumour environment and increasing T cell avidity may lead to improved responses. Expert Opin Biol Ther, 2010. 10(5): p. 735-48.
146. Sandberg, J. K., et al., T cell tolerance based on avidity thresholds rather than complete deletion allows maintenance of maximal repertoire diversity. J Immunol, 2000. 165: p. 25-33.
147. Gallimore, A., et al., Induction and Exhaustion of Lymphocytic Choriomeningitis Virus-specific Cytotoxic T Lymphocytes Visualized Using Soluble Tetrameric Major Histocompatibility Complex Class I-Peptide Complexes. J. Exp. Med., 1998. 187(9): p. 1383-1393.
148. Sedlik, C., et al., In vivo induction of a high-avidity, high-frequency cytotoxic T-lymphocyte response is associated with antiviral protective immunity. J Virol, 2000. 74(13): p. 5769-75.
149. Bijker, M. S., et al., CD8+ CTL priming by exact peptide epitopes in incomplete Freund's adjuvant induces a vanishing CTL response, whereas long peptides induce sustained CTL reactivity. J Immunol, 2007. 179(8): p. 5033-40.
150. Sabbatini, P., et al., Phase I trial of overlapping long peptides from a tumor self-antigen and poly-ICLC shows rapid induction of integrated immune response in ovarian cancer patients. Clin Cancer Res, 2012. 18(23): p. 6497-508.
151. Tsuji, T., et al., Effect of Montanide and poly-ICLC adjuvant on human self/tumor antigen-specific CD4+ T cells in phase I overlapping long peptide vaccine trial. Cancer Immunol Res, 2013. 1(5): p. 340-50.
152. Rahma, O. E., et al., The immunological and clinical effects of mutated ras peptide vaccine in combination with IL-2, GM-CSF, or both in patients with solid tumors. J Transl Med, 2014. 12: p. 55.
153. Schuster, J., et al., A phase II, multicenter trial of rindopepimut (CDX-110) in newly diagnosed glioblastoma: the ACT III study. Neuro Oncol, 2015. 17(6): p. 854-61.
154. Shang, N., et al., Dendritic cells based immunotherapy. Am J Cancer Res, 2017. 7(10): p. 2091-2102.
155. Handy, C. E. and E. S. Antonarakis, Sipuleucel-T for the treatment of prostate cancer: novel insights and future directions. Future Oncol, 2017.
156. Wimmers, F., et al., Paradigm Shift in Dendritic Cell-Based Immunotherapy: From in vitro Generated Monocyte-Derived DCs to Naturally Circulating DC Subsets. Front Immunol, 2014. 5: p. 165.
157. Sambrook, J., E. F. Fritsch, and T. Maniatis, Molecular Cloning: a laboratory manual. 1989, Cold Spring Harbor, N. Y.: Cold Spring Harbor Laboratory, 1989.
158. Ausubel, F., Short protocols in molecular biology. 2nd ed. 1992: John Wiley & Sons.
159. Iwasaki, A. and R. Medzhitov, Toll-like receptor control of the adaptive immune responses. Nat Immunol, 2004. 5(10): p. 987-95.
160. Yanai, H., T. Ban, and T. Taniguchi, High-mobility group box family of proteins: ligand and sensor for innate immunity. Trends Immunol, 2012. 33(12): p. 633-40.
161. Ishii, K. J., et al., A Toll-like receptor-independent antiviral response induced by double-stranded B-form DNA. Nat Immunol, 2006. 7(1): p. 40-8.
162. Zhang, Z., et al., The helicase DDX41 senses intracellular DNA mediated by the adaptor STING in dendritic cells. Nat Immunol, 2011. 12(10): p. 959-65.
163. Tang, C. K. and G. A. Pietersz, Intracellular detection and immune signaling pathways of DNA vaccines. Expert Rev Vaccines, 2009. 8(9): p. 1161-70.
164. Takaoka, A., et al., DAI (DLM-1/ZBP1) is a cytosolic DNA sensor and an activator of innate immune response. Nature, 2007. 448(7152): p. 501-5.
165. Unterholzner, L., et al., IFI16 is an innate immune sensor for intracellular DNA. Nat Immunol, 2010. 11(11): p. 997-1004.
166. Yanai, H., et al., HMGB proteins function as universal sentinels for nucleic-acid-mediated innate immune responses. Nature, 2009. 462(7269): p. 99-103.
167. Venereau, E., et al., Mutually exclusive redox forms of HMGB1 promote cell recruitment or proinflammatory cytokine release. J Exp Med, 2012. 209(9): p. 1519-28.
168. Apetoh, L., et al., Toll-like receptor 4-dependent contribution of the immune system to anticancer chemotherapy and radiotherapy. Nat Med, 2007. 13(9): p. 1050-9.
169. Kang, R., et al., Apoptosis to autophagy switch triggered by the MHC class III-encoded receptor for advanced glycation endproducts (RAGE). Autophagy, 2011. 7(1): p. 91-3.
170. Campana, L., et al., Requirement of HMGB1 for stromal cell-derived factor-1/CXCL12-dependent migration of macrophages and dendritic cells. J Leukoc Biol, 2009. 86(3): p. 609-15.
171. Weide, B., et al., Results of the first phase I/II clinical vaccination trial with direct injection of mRNA. J Immunother, 2008. 31(2): p. 180-8.
172. Petsch, B., et al., Protective efficacy of in vitro synthesized, specific mRNA vaccines against influenza A virus infection. Nat Biotechnol, 2012. 30(12): p. 1210-6.
173. Kallen, K. J., et al., A novel, disruptive vaccination technology: self-adjuvanted RNActive((R)) vaccines. Hum Vaccin Immunother, 2013. 9(10): p. 2263-76.
174. Pardi, N., et al., Zika virus protection by a single low-dose nucleoside-modified mRNA vaccination. Nature, 2017. 543(7644): p. 248-251.
175. Eppstein, D. A., et al., Biological activity of liposome-encapsulated murine interferon gamma is mediated by a cell membrane receptor. Proc Natl Acad Sci USA, 1985. 82(11): p. 3688-92.
176. Hwang, K. J., K. F. Luk, and P. L. Beaumier, Hepatic uptake and degradation of unilamellar sphingomyelin/cholesterol liposomes: a kinetic study. Proc Natl Acad Sci USA, 1980. 77(7): p. 4030-4.
177. Stewart, J. M. and J. D. Young, Solid phase peptide synthesis. 1984, Pierce Chemical Company: Rockford, Illinois.
178. Bodanzsky, M. and A. Bodanzsky, The practice of peptide synthesis. 1984, New York: Springer Verlag.
179. Pluckthun, A., Antibody engineering: advances from the use of Escherichia coli expression systems. Biotechnology (N Y), 1991. 9(6): p. 545-51.
180. Reff, M. E., High-level production of recombinant immunoglobulins in mammalian cells. Curr Opin Biotechnol, 1993. 4(5): p. 573-6.
181. Trill, J. J., A. R. Shatzman, and S. Ganguly, Production of monoclonal antibodies in COS and CHO cells. Curr Opin Biotechnol, 1995. 6(5): p. 553-60.
182. Ausubel, F. M., et al., Short protocols in molecular biology: a compendium of methods from Current protocols in molecular biology. 2nd ed. 1992: Brooklyn, NY: Green Pub. Associates: New York, NY: Wiley.
183. Xue, W., et al., SCIB2, an antibody DNA vaccine encoding NY-ESO-1 epitopes, induces potent antitumor immunity which is further enhanced by checkpoint blockade. Oncoimmunology, 2016. 5(6): p. e1169353.
184. Jurtz, V., et al., NetMHCpan-4.0: Improved Peptide-MHC Class I Interaction Predictions Integrating Eluted Ligand and Peptide Binding Affinity Data. J Immunol, 2017. 199(9): p. 3360-3368.
185. Sanger, F., S. Nicklen, and A. R. Coulson, DNA sequencing with chain-terminating inhibitors. Proc Natl Acad Sci USA, 1977. 74(12): p. 5463-7.
186. Brentville, V. A., et al., Citrullinated Vimentin Presented on MHC-II in Tumor Cells Is a Target for CD4+ T-Cell-Mediated Antitumor Immunity. Cancer Res, 2016. 76(3): p. 548-60.
187. Brentville, V. A., et al., T cell repertoire to citrullinated self-peptides in healthy humans is not confined to the HLA-DR SE alleles; Targeting of citrullinated self-peptides presented by HLA-DP4for tumour therapy. Oncoimmunology, 2019. 8(5): p. e1576490.
188. Clayton, R. F., et al., Analysis of antigenicity and topology of E2 glycoprotein present on recombinant hepatitis C virus-like particles. J Virol, 2002. 76(15): p. 7672-82.
189. Pestka, J. M., et al., Rapid induction of virus-neutralizing antibodies and viral clearance in a single-source outbreak of hepatitis C. Proc Natl Acad Sci USA, 2007. 104(14): p. 6025-30.
190. Coleman, C. M., et al., Purified coronavirus spike protein nanoparticles induce coronavirus neutralizing antibodies in mice. Vaccine, 2014. 32(26): p. 3169-3174.

1.-57. (canceled)

58. A combination of a first nucleic acid and a second nucleic acid, wherein:
(a) the first nucleic acid encodes a polypeptide comprising:
(i) a modified Fc region comprising:
(I) the amino acid sequence provided in SEQ ID NO: 1; or
(II) an amino acid sequence having at least 90% identity to SEQ ID NO: 1 and comprising all of the following modifications: N286T, K288W, K290Q, A339P, Q342R, P343A, R344Q, E345T, L351I, S354P, D356E, E357Q, L358M, T359S, N361K, Q362K, K370T, G371N, Y373F, P374S, S375E, D376A, and A378S according to the IMGT system for numbering antibody sequences; and
(ii) a heavy chain variable region comprising the following heterologous antigens substituted into the CDR1, CDR2 and CDR3 respectively: GTGRAMLGTHTMEVTVYH (SEQ ID NO: 29), SVYDFFVWL (SEQ ID NO: 30) and VPLDCVLYRYGSFSVTLDIVQG (SEQ ID NO: 32); and
(b) the second nucleic acid encodes an antibody light chain comprising the following heterologous antigens substituted into the CDR1, CDR2 and CDR3 respectively:
(SEQ ID NO: 31)
WNRQLYPEWTEAQRLD,
(SEQ ID NO: 33)
ANCSVYDFFVWLHYYSVRDTLLGPGRPYR
and
(SEQ ID NO: 34)
QCTEVRADTRPWSGPYILRNQDDRELWPRKFF.

59. The combination of claim 58, wherein the polypeptide encoded by the first nucleic acid comprises the amino acid sequence provided in SEQ ID NO: 2.

60. The combination of claim 58, wherein the antibody light chain encoded by the second nucleic acid comprises the amino acid sequence provided in SEQ ID NO: 10.

61. The combination of claim 58, wherein the polypeptide encoded by the first nucleic acid comprises the amino acid sequence provided in SEQ ID NO: 2 and wherein the antibody light chain encoded by the second nucleic acid comprises the amino acid sequence provided in SEQ ID NO: 10.

62. A vector comprising the combination of first and second nucleic acids of claim 58.

63. The vector of claim 62, which comprises the nucleotide sequences provided in SEQ ID NO: 12 and SEQ ID NO: 13.

64. The vector of claim 62, which comprises the nucleotide sequence provided in SEQ ID NO: 20 or SEQ ID NO: 21.

65. The vector of claim 62, which is a DNA plasmid or doggybone (dbDNA) vector.

66. A polypeptide encoded by the combination of first and second nucleic acids of claim 58, wherein polypeptide comprises the amino acid sequence provided in SEQ ID NO: 2 and wherein the antibody light chain encoded by the second nucleic acid comprises the amino acid sequence provided in SEQ ID NO: 10.

67. A vaccine composition comprising the combination of first and second nucleic acids of claim 58.

68. The vaccine composition of claim 67, wherein the vaccine composition further comprises an adjuvant.

69. A method of treating a subject, the method comprising administering the combination of a first nucleic acid and a second nucleic acid of claim 58.

70. The method of claim 69, wherein the method is for the prevention or treatment of cancer in the subject.

71. The method of claim 70, wherein the cancer is melanoma.

72. The method of claim 69, wherein the administration is via needle-free injection.
